(19) United States
(12) Patent Application Publication
(10) Pub. No.: US 2026/0117244 A1
(43) Pub. Date: Apr. 30, 2026

(54) INHIBITORY RNA FOR THE CONTROL OF PHYTOPATHOGENS

(71) Applicant: The Regents of the University of California, Oakland, CA (US)

(72) Inventors: Mary C. Wildermuth, Berkeley, CA (US); Jyoti Taneja, Berkeley, CA (US); Amanda G. Mcrae, Berkeley, CA (US)

(73) Assignee: The Regents of the University of California, Oakland, CA (US)

(21) Appl. No.: 18/555,503

(22) PCT Filed: Apr. 19, 2022
(86) PCT No.: PCT/US2022/025330

§ 371 Date: Oct. 13, 2023

Related U.S. Application Data
(60) Provisional application No. 63/176,795, filed on Apr. 19, 2021.

(51) Int. Cl. C12N 15/82; C12N 15/113

(52) U.S. Cl. CPC C12N 15/8282; C12N 15/113; C12N 15/8218; C12N 15/8243; C12N 2310/141; C12N 2310/531

(57) ABSTRACT

In various embodiments, target phytopathogen genes are identified whose downregulation can facilitate control or prevention of an infection of a plant by a phytopathogen (e.g., a phytopathogenic fungus). In certain embodiments, double-stranded RNAs are provided to effectively downregulate the target genes.

### CROSS-REFERENCE TO RELATED APPLICATIONS

This application claims benefit of and priority to U.S. Ser. No. 63/176,795, filed on Apr. 19, 2021, which is incorporated herein by reference in its entirety for all purposes.

### STATEMENT OF GOVERNMENTAL SUPPORT

This invention was made with government support under Grant Numbers 1617020, and 1919244 awarded by the National Science Foundation. The Government has certain rights in the invention.

### INCORPORATION BY REFERENCE OF SEQUENCE LISTING PROVIDED AS A TEXT FILE

[Not Applicable]

### BACKGROUND

Powdery mildews are widespread obligate biotrophic pathogens of plants. There are hundreds of species that together infect over 10,000 plants including crops like wheat, barley, grape, and cucurbits, and ornamentals like rose (Agrios (2004) Plant Pathology, 5th ed. Elsevier Inc.; Kuhn et al. (2016) Biotrophy at Its Best: Novel Findings and Unsolved Mysteries of the Arabidopsis-Powdery Mildew Pathosystem. The Arabidopsis Book 14, e0184. doi.org/10.1199/tab.0184). Although they do not kill their host, they can significantly reduce crop yield and quality. Furthermore, infection by powdery mildew often increases likelihood or severity of secondary infections. Resistant varieties or costly integrated pest management strategies are frequently used to prevent infection in susceptible plants. Managing powdery mildew of California grapevine, for example, is quite costly with 74% of pesticide use directed to its control (Fuller et al. (2014) Economics and Policy, 3: 90-107). There are mixed opinions among wine grape growers on the use of powdery mildew-resistant varietals. Although their use would save growers time and resources in managing powdery mildew, there are concerns about taste and having to label wine varietals differently (Id.). With powdery mildew fungicide resistance emerging in several regions and the need to apply fungicides every 1-2 weeks, new options are desired (Frenkel et al. (2015) Phytopathology, 105: 370-377).

Powdery mildews are filamentous ascomycetes belonging to the order Erysiphales, a monophyletic group within Leotiomycetes (Belanger et al. (2003) The Powdery mildews: a comprehensive treatise, Choice Reviews Online. American Phytopathological Society, St. Paul. doi.org/10.5860/choice.40-5806; Glawe (2008) Ann. Rev. Phytopath. 46: 27-51). The Erysiphales are divided into 5 tribes based on host range and history (Takamatsu (2004) Mycoscience, 45: 147-157) and individual species have been renamed and classified as more molecular-based relationships have been uncovered. As obligate biotrophic pathogens, they must infect and feed from a living host in order to obtain nutrients and reproduce. The powdery mildew fungus, Golovinomyces orontii, historically known as Erysiphe orontii, infects the model plant Arabidopsis thaliana, tomato, tomatillo, sunflower and eggplant (Kallamadi & Mulpuri (2020) Biotech, 10: 1-6. doi.org/10.1007/s13205-020-02224-2; Utkhede et al. (2001) Canadian J. Plant Sci. 81: 179-182: Plotnikova et al. (1998) Mycologia 90: 1009-1016). The life cycle of G. orontii begins when a primary germ tube grows from the spore and develops into an appressorium which pierces through the tough cuticle surface of the leaf using turgor pressure. Unlike other pathogenic fungi, powdery mildews do not encode many cell wall degrading enzymes which are often secreted to facilitate entry into the plant (Spanu et al. (2010) Science, 330: 1543-1546). This limits the amount of cell wall breakdown products produced upon entry which can elicit a defense response from the host. Before penetration into the plant, glycogen and lipids stored in the conidium are consumed to fuel its development (Both et al. (2005) Plant Cell, 17(7); 2107-2122). After the powdery mildew enters the plant in the epidermal cell layer, a feeding structure (haustorium) develops and is enveloped by the plant cell membrane (Koh et al. (2005) Plant J. 44: 516-529). This is the primary site of nutrient uptake where glucose and other metabolites are acquired by the fungus. Hyphae grow epiphytically, branching out from the initial germinating conidium, with additional feeding sites supporting growth as the colony expands. Most powdery mildews develop in this fashion; however, there are two known species of powdery mildew that enter leaves through stomata to infect mesophyll cells instead of epidermal cells. By 5 days post infection (dpi), the G. orontii lifecycle is complete with the emergence of fully formed asexual reproductive structures (conidiophores). These conidiophores are stalk-like structures that grow from hyphae. They contain a basal cell and a chain of conidia (spores) that protrudes up and away from the leaf surface. The number of conidia per conidiophore varies with powdery mildew species; for G. orontii MGH1, approximately 5 conidia per conidiophore are typically observed at 5 dpi (Chandran et al. (2010) Proc. Natl. Acad. Sci. USA, 107: 460-465).

For G. orontii infection of Arabidopsis used in laboratory research, only asexual reproduction is observed. At 5 dpi, the diameter of G. orontii colonies can be over 1 mm. Days later, the mass of spores produced becomes visible to the naked eye. In nature, these infective spores can be transferred by wind or animals, but in the lab, plants are often infected by dusting through mesh, brushing, or spraying spore suspensions (Id.; Micali et al. (2008) The Powdery Mildew Disease of Arabidopsis: A Paradigm for the Interaction between Plants and Biotrophic Fungi. The Arabidopsis Book 6, e0115. doi.org/10.1199/tab.0115). Asexual reproduction propagates powdery mildew disease over the growing season in the field. However, sexual reproduction can occur to remain viable during overwinter periods (Kuhn et al. (2016) Biotrophy at Its Best: Novel Findings and Unsolved Mysteries of the Arabidopsis-Powdery Mildew Pathosystem. The Arabidopsis Book 14, e0184. doi.org/10.1199/tab.0184).

Sequence analysis of powdery mildews has revealed significant gene loss and genome expansion compared to other related plant pathogenic fungi (Spanu et al. (2010) Science, 330: 1543-1546). As obligate biotrophs, powdery mildews have evolved an intimate relationship with their hosts to acquire nutrients. This parasitism has led to the reliance on the host to provide specific metabolites, reducing the evolutionary pressure to maintain genes and complete metabolic pathways often conserved in ascomycetes. Other plant obligate biotrophs from diverse lineages also exhibit significant gene loss and shared loss of the same metabolic pathways or pathway components (Spanu (2012) Ann. Rev. Phytopath. 50: 91-109). Within published powdery mildew genomes, there is a high degree of gene conservation with ˜71% of powdery mildew genes conserved in all powdery mildews and ˜81% conserved among dicot powdery mildews (Wu et al. (2018) BMC Genomics, 19: 705. Though the number of genes is reduced, the genome size of the powdery mildews is significantly larger than other fungal genomes due to the proliferation of retrotransposons and the loss of mechanisms to control transposon propagation. Powdery mildew genomes can be over 120 Mb while other related fungal pathogens have an average genome size of 37 Mb. This proliferation has been left unchecked by the loss of genes responsible for repeat-induced point mutation mechanisms. Because their large genomes are full of repetitive elements, it has been difficult to sequence powdery mildew genomes.

As powdery mildews cannot be cultured (grown independent of the plant host), it has been difficult to study the function of specific genes in powdery mildew colonization, growth, and reproduction. Consequently, development of genomic tools to regulate powdery mildew colonization and/or growth has proven challenging.

### SUMMARY

As described herein gene targets are identified that when down-regulated or inhibited result in reproducible and statistically significant reduction phytopathogen growth and/or infectivity and, in particular, powdery mildew. Accordingly, in certain embodiments, effective dsRNA (long dsRNA and/or short interfering RNA (siRNA) are provided that can be used for topical application (e.g., spray-induced gene silencing (SIGS), soil or root application, or host-induced gene silencing (HIGS) to regulate plant pathogens (e.g., to control or prevent an infection of a plant by a phytopathogen (e.g., a phytopathogenic fungus)).

In particular, the gene targets described herein are highly conserved and believed to function in metabolism, regulation, manipulation of the plant host and essential cellular processes, and are important, inter alia, to powdery mildew (and other phytopathogen) growth and reproduction.

In various embodiments, use of the identified novel powdery mildew gene targets for fungicide development or resistant plant development by any means are provided. Additionally, the methodology (screening system) for the rapid identification of suitable target genes is provided. Additionally, methods for prioritizing target genes for screening are provided.

Accordingly, various embodiments provided herein may include, but need not be limited to, one or more of the following:

Embodiment 1: An isolated double stranded RNA (dsRNA) for controlling or preventing an infection of a plant by a phytopathogen, wherein said dsRNA is capable of inhibiting or downregulating (e.g., downregulates) expression of one or more target genes selected from the group consisting of a gene involved in lipid metabolism and/or acquisition, a gene involved in triacylglycerol (TAG)/glycerolipid metabolism, a gene involved in the pentose phosphate pathway (PPP), a gene involved in the tricarboxylic acid (TCA) and/or glyoxylate cycle, a gene involved in fatty acid synthesis and/or transport, a gene involved in lipid catabolism, a gene involved in fatty acid degradation including fatty acid B-oxidation, a gene involved in carbohydrate metabolism, a gene involved in glycogen metabolism, a gene involved in glycolysis, a gene involved in carbohydrate utilization, a gene involved in amino acid and/or nucleotide metabolism and salvage and/or cofactor metabolism, a gene involved in amino acid metabolism and/or salvage, a gene involved in cofactor metabolism, a gene involved in nucleotide metabolism and/or salvage, a gene involved in an essential fungal-specific process, a gene involved in sterol biosynthesis, a gene involved in mitochondrial electron transport and/or ATP synthesis, a gene involved in mitochondrial electron transport chain, a gene involved in transcriptional regulation, a gene involved in protein regulation, a gene involved in nucleic acid translation, a gene involved in signaling, a gene involved in producing a secreted protein, a gene associated with the plant TCP interaction network, a gene associated with a plant hormone pathway, a gene involved in jasmonic acid (oxylipin) metabolism, acquisition, or signaling, a gene involved in abscisic acid metabolism, acquisition, or signaling, a gene involved in cell cycle and DNA replication, a gene involved in vesicles, autophagy, and phagocytosis, a gene involved in autophagy and phagocytosis, a gene involved in ion/metal transport and osmotic homeostasis, and single copy genes in G. orontii MGH1 following recent whole genome duplication including those with unknown function.

Embodiment 2: The isolated dsRNA of embodiment 1, wherein said dsRNA is capable of downregulating (e.g., downregulates) expression of one or more target genes selected from the group consisting of a gene involved in lipid metabolism, a gene involved in fatty acid (FA) synthesis and/or transport, a gene involved in carbohydrate metabolism, a gene involved in amino acid and/or nucleotide metabolism and salvage and/or cofactor metabolism, a gene involved in nucleotide metabolism/salvage, a gene involved in an essential fungal-specific process, a gene involved in mitochondrial electron transport, a gene involved in transcriptional regulation, a gene involved in nucleic acid translation, a gene involved in signaling, a gene involved in producing a secreted protein, a gene involved in plant hormone metabolism, a gene involved in cell cycle and replication, a gene involved in autophagy and phagocytosis, and a gene involved in cell volume control.

Embodiment 3: The isolated dsRNA according to any one of embodiments 1-2, wherein said dsRNA inhibits or is capable of downregulating expression of one or more target genes shown in Table 1 or Table 2 (SEQ ID NOS:1-211), and SEQ ID NOs:255-411, and/or orthologues thereof.

Embodiment 4: The isolated dsRNA of embodiment 5, wherein said target genes and/or orthologs comprise one or more target genes of SEQ ID Nos:1-84, 86-211, and 255-411 and/or orthologs thereof.

Embodiment 5: The isolated dsRNA according to any one of embodiments 1-2, wherein said dsRNA inhibits or is capable of downregulating expression of one or more target genes shown in Table 1 or Table 2 (SEQ ID NOS:1-211).

Embodiment 6: The isolated dsRNA of embodiment 5, wherein said target genes and/or orthologs comprise one or more target genes of SEQ ID Nos:1-84, and 86-211 and/or orthologs thereof.

Embodiment 7: The isolated dsRNA of embodiment 5, wherein said target genes and/or orthologs comprise one or more target genes of SEQ ID Nos:1-178 and/or orthologs thereof.

Embodiment 8: The isolated dsRNA of embodiment 7, wherein said target genes and/or orthologs comprise one or more target genes of SEQ ID Nos:1-84, and 86-178 and/or orthologs thereof.

Embodiment 9: The isolated dsRNA according to any one of embodiments 1-5, wherein said target genes and/or orthologs comprise one or more target genes independently selected from the group consisting of SEQ ID NO:1, SEQ ID NO:2, SEQ ID NO:15, SEQ ID NO:20, SEQ ID NO:21, SEQ ID NO:22, SEQ ID NO:32, SEQ ID NO:63, SEQ ID NO:64, SEQ ID NO:70, SEQ ID NO:71, SEQ ID NO:79, SEQ ID NO:87, SEQ ID NO:92, SEQ ID NO:104, SEQ ID NO:122, SEQ ID NO:123, SEQ ID NO:124, SEQ ID NO:140, SEQ ID NO:141, SEQ ID NO:145, SEQ ID NO:146, SEQ ID NO:148, SEQ ID NO:149, SEQ ID NO:163, SEQ ID NO:164, SEQ ID NO:167, SEQ ID NO:169, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, and SEQ ID NO:85 and/or orthologs thereof.

Embodiment 10: The isolated dsRNA of embodiment 9, wherein said target genes and/or orthologs comprise one or more target genes independently selected from (Table 2) the group consisting of SEQ ID NO:1, SEQ ID NO:2, SEQ ID NO:15, SEQ ID NO:20, SEQ ID NO:21, SEQ ID NO:22, SEQ ID NO:32, SEQ ID NO:63, SEQ ID NO:64, SEQ ID NO:70, SEQ ID NO:71, SEQ ID NO:79, SEQ ID NO:87, SEQ ID NO:92, SEQ ID NO:104, SEQ ID NO:122, SEQ ID NO:123, SEQ ID NO:124, SEQ ID NO:140, SEQ ID NO:141, SEQ ID NO:145, SEQ ID NO:146, SEQ ID NO:148, SEQ ID NO:149, SEQ ID NO:163, SEQ ID NO:164, SEQ ID NO:167, SEQ ID NO:169, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, and SEQ ID NO:211, and/or orthologs thereof.

Embodiment 11: The isolated dsRNA of embodiment 10, wherein said target genes and/or orthologs comprise one or more target genes independently selected from (Original Table 2) the group consisting of SEQ ID NO:1, SEQ ID NO:2, SEQ ID NO:15, SEQ ID NO:20, SEQ ID NO:21, SEQ ID NO:22, SEQ ID NO:32, SEQ ID NO:63, SEQ ID NO:64, SEQ ID NO:70, SEQ ID NO:71, SEQ ID NO:79, SEQ ID NO:87, SEQ ID NO:92, SEQ ID NO:104, SEQ ID NO:122, SEQ ID NO:123, SEQ ID NO:124, SEQ ID NO:140, SEQ ID NO:141, SEQ ID NO:145, SEQ ID NO:146, SEQ ID NO:149, SEQ ID NO:164, and SEQ ID NO:85, and/or orthologs thereof.

Embodiment 12: The isolated dsRNA of embodiment 11, wherein said target genes and/or orthologs comprise one or more target genes independently selected from (Original Table 2) the group consisting of SEQ ID NO:1, SEQ ID NO:2, SEQ ID NO:15, SEQ ID NO:20, SEQ ID NO:21, SEQ ID NO:22, SEQ ID NO:32, SEQ ID NO:63, SEQ ID NO:64, SEQ ID NO:70, SEQ ID NO:71, SEQ ID NO:79, SEQ ID NO:87, SEQ ID NO:92, SEQ ID NO:104, SEQ ID NO:122, SEQ ID NO:123, SEQ ID NO:124, SEQ ID NO:140, SEQ ID NO:141, SEQ ID NO:145, SEQ ID NO:146, SEQ ID NO:149, and SEQ ID NO:164, and/or orthologs thereof.

Embodiment 13: The isolated dsRNA according to any one of embodiments 1-5, wherein said target genes and/or orthologs comprise one or more G. orontii genes independently selected from the group consisting of Cytochrome P450 monoxygenase (CYP51) (SEQ ID NO:85), TAG lipase 1, caroboxyesterase domain (SEQ ID NO:1), TAG lipase A, extracellular lipase (SEQ ID NO:2), Transaldolase (SEQ ID NO:15), Isocitrate lyase (ICL) (SEQ ID NO:20), Malate synthase (MS) (SEQ ID NO:21), Citrate (Si) synthase (SEQ ID NO:22), Trans-2-enoyl CoA reductase (SEQ ID NO:32), Glutamate dehydrogenase (SEQ ID NO:63), Threonine aldolase (SEQ ID NO:64), Pf02548-Ketopantoate hydroxymethyltransferase (SEQ ID NO:70), Tetrahydrofolate synthase (SEQ ID NO:71), Thymidine synthase (SEQ ID NO:79), Pf8238-Sel1 repeat (SEQ ID NO:87), Apoptosis antagonizing transcription factor (AATF) (SEQ ID NO:92), Pf10453—Nuclear fragile X mental retardation-interacting protein 1 (NUFIP1) (SEQ ID NO:104), G. orontii effector candidate 60/61 (SEQ ID NO:122), G. orontii effector candidate 70 (SEQ ID NO:123), G. orontii effector candidate 14 (SEQ ID NO:124), Effector protein EC2 (SEQ ID NO:140), Heat shock protein 70 family (HSP70) (SEQ ID NO:141), β-carotene 15,15′-dioxygenase (BCDO) (SEQ ID NO:145), Xanthin oxidase (SEQ ID NO:146), ABA G-protein coupled receptor (SEQ ID NO: 148), Acyl-CoA oxidase (ACX_(SEQ ID NO:149), Anaphase promoting complex/20S cyclosome subunit (SEQ ID NO: 163), Pf04109—Autophagy protein Apg9 (SEQ ID NO:164), Pf03517 Regulator of cell volume after swelling (SEQ ID NO: 167), Ctr family Cu2+ transporter (SEQ ID NO: 169), Single copy gene, unknown function (SEQ ID NO: 179), Cyclin-like protein (SEQ ID NO: 180), and Alanine-glyoxylate aminotransferase (SEQ ID NO: 181), and/or orthologs thereof.

Embodiment 14: The isolated dsRNA of embodiment 13, wherein said target genes and/or orthologs comprise one or more G. orontii genes independently selected from the group consisting of TAG lipase 1, caroboxyesterase domain (SEQ ID NO:1), TAG lipase A, extracellular lipase (SEQ ID NO:2), Transaldolase (SEQ ID NO:15), Isocitrate lyase (ICL) (SEQ ID NO:20), Malate synthase (MS) (SEQ ID NO:21), Citrate (Si) synthase (SEQ ID NO:22), Trans-2-enoyl CoA reductase (SEQ ID NO:32), Glutamate dehydrogenase (SEQ ID NO:63), Threonine aldolase (SEQ ID NO:64), Pf02548-Ketopantoate hydroxymethyltransferase (SEQ ID NO:70), Tetrahydrofolate synthase (SEQ ID NO:71), Thymidine synthase (SEQ ID NO:79), Pf8238-Sel1 repeat (SEQ ID NO:87), Apoptosis antagonizing transcription factor (AATF) (SEQ ID NO:92), Pf10453—Nuclear fragile X mental retardation-interacting protein 1 (NUFIP1) (SEQ ID NO:104), G. orontii effector candidate 60/61 (SEQ ID NO:122), G. orontii effector candidate 70 (SEQ ID NO:123), G. orontii effector candidate 14 (SEQ ID NO:124), Effector protein EC2 (SEQ ID NO:140), Heat shock protein 70 family (HSP70) (SEQ ID NO:141), β-carotene 15,15′-dioxygenase (BCDO) (SEQ ID NO:145), Xanthin oxidase (SEQ ID NO:146), ABA G-protein coupled receptor (SEQ ID NO: 148), Acyl-CoA oxidase (ACX_(SEQ ID NO:149), Anaphase promoting complex/20S cyclosome subunit (SEQ ID NO: 163), Pf04109—Autophagy protein Apg9 (SEQ ID NO:164), Pf03517 Regulator of cell volume after swelling (SEQ ID NO: 167), Ctr family Cu2+ transporter (SEQ ID NO: 169), Single copy gene, unknown function (SEQ ID NO: 179), Cyclin-like protein (SEQ ID NO: 180), and Alanine-glyoxylate aminotransferase (SEQ ID NO: 181), and/or orthologs thereof.

Embodiment 15: The isolated dsRNA according to any one of embodiments 11, wherein said target genes and/or orthologs comprise one or more target E. necator genes selected from the group consisting of Cytochrome P450 monoxygenase (CYP51) (SEQ ID NO:182), TAG lipase 1, caroboxyesterase domain (SEQ ID NO:183), TAG lipase A, extracellular lipase (SEQ ID NO:184), Transaldolase (SEQ ID NO:185), Isocitrate lyase (ICL) (SEQ ID NO:186-187), Malate synthase (MS) (SEQ ID NO:188), Citrate (Si) synthase (SEQ ID NO:189), Trans-2-enoyl CoA reductase (SEQ ID NO:190), Glutamate dehydrogenase (SEQ ID NO:191), Threonine aldolase (SEQ ID NO:192), Pf02548-Ketopantoate hydroxymethyltransferase (SEQ ID NO:193), Tetrahydrofolate synthase (SEQ ID NO:194), Thymidine synthase (SEQ ID NO:195), Pf8238-Sel1 repeat (SEQ ID NO:196), Apoptosis antagonizing transcription factor (AATF) (SEQ ID NO:197), Pf10453-Nuclear fragile X mental retardation-interacting protein 1 (NUFIP1) (SEQ ID NO:198), G. orontii effector candidate 60/61 (SEQ ID NO:199), G. orontii effector candidate 70 (SEQ ID NO:200), effector protein EC2 (SEQ ID NO:201), Heat shock protein 70 family (HSP70) (SEQ ID NO:202), β-carotene 15,15′-dioxygenase (BCDO) (SEQ ID NO:203), Xanthin oxidase (SEQ ID NO:204), ABA G-protein coupled receptor (SEQ ID NO: 205), Acyl-CoA oxidase (ACX_(SEQ ID NO:206), Anaphase promoting complex/20S cyclosome subunit (SEQ ID NO: 207), Pf04109—Autophagy protein Apg9 (SEQ ID NO:208), Pf03517 Regulator of cell volume after swelling (SEQ ID NO: 209), Ctr family Cu2+ transporter (SEQ ID NO: 210), and Cyclin-like protein (SEQ ID NO: 211), and/or orthologs thereof.

Embodiment 16: The isolated dsRNA according to any one of embodiments 1-5, wherein said target genes and/or orthologs comprise one or more target genes selected from the group consisting of CYP51 (Seq ID NO: 85, 182), lipase 1 (SEQ ID NO: 1, 183), lipase A (SEQ ID NO: 2, 184), isocitrate lyase (SEQ ID NO: 20, 186-187), malate synthase (SEQ ID: 21, 188), citrate (Si) synthase (SEQ ID NO: 22, 189), trans-2-enoyl-coA reductase (SEQ ID NO: 32, 190), glutamate dehydrogenase (SEQ ID NO: 63, 191), threonine aldolase (SEQ ID NO: 64,192), tetrahydrofolate synthase (SEQ ID NO: 71, 194), thymidylate synthase (SEQ ID NO: 79, 195), apoptosis-antagonizing transcription factor (AATF) (SEQ ID NO: 92, 197), Effector candidate 2 (EC2) (SEQ ID NO: 140,201), β-carotene 15,15′-dioxygenase (BCDO) (SEQ ID NO: 145, 203), xanthoxin oxidase (SEQ ID NO: 146, 204), ABA G-coupled receptor (SEQ ID NO: 148, 205), acyl-coA oxidase (ACX) (SEQ ID NO: 149, 206), Autophagy protein Apg9 (SEQ ID NO: 164, 208), Ctr family CU2+ transporter (SEQ ID NO: 169,210), cyclin-like protein (SEQ ID NO: 180,211), and alanine-glyoxylate amino transferase (SEQ ID NO: 181), and/or orthologs thereof.

Embodiment 17: The isolated dsRNA of embodiment 16, wherein said target genes and/or orthologs comprise one or more target genes selected from the group consisting of lipase 1 (SEQ ID NO: 1, 183), lipase A (SEQ ID NO: 2, 184), isocitrate lyase (SEQ ID NO: 20, 186-187), malate synthase (SEQ ID: 21, 188), citrate (Si) synthase (SEQ ID NO: 22, 189), trans-2-enoyl-coA reductase (SEQ ID NO: 32, 190), glutamate dehydrogenase (SEQ ID NO: 63, 191), threonine aldolase (SEQ ID NO: 64,192), tetrahydrofolate synthase (SEQ ID NO: 71, 194), thymidylate synthase (SEQ ID NO: 79, 195), apoptosis-antagonizing transcription factor (AATF) (SEQ ID NO: 92, 197), Effector candidate 2 (EC2) (SEQ ID NO: 140, 201), β-carotene 15,15′-dioxygenase (BCDO) (SEQ ID NO: 145, 203), xanthoxin oxidase (SEQ ID NO: 146, 204), ABA G-coupled receptor (SEQ ID NO: 148, 205), acyl-coA oxidase (ACX) (SEQ ID NO: 149, 206), Autophagy protein Apg9 (SEQ ID NO: 164, 208), Ctr family CU2+ transporter (SEQ ID NO: 169,210), cyclin-like protein (SEQ ID NO: 180,211), and alanine-glyoxylate amino transferase (SEQ ID NO: 181), and/or orthologs thereof.

Embodiment 18: The isolated dsRNA according to any one of embodiments 1-5, wherein said target genes and/or orthologs comprise one or more target genes selected from the group consisting of CYP51 (Seq ID NO: 85, 182), lipase 1 (SEQ ID NO: 1, 183), lipase A (SEQ ID NO: 2, 184), isocitrate lyase (SEQ ID NO: 20, 186-187), malate synthase (SEQ ID: 21, 188), apoptosis-antagonizing transcription factor (AATF) (SEQ ID NO: 92, 197), Effector candidate 2 (EC2) (SEQ ID NO: 140, 201), β-carotene 15,15′-dioxygenase (BCDO) (SEQ ID NO: 145, 203), and Autophagy protein Apg9 (SEQ ID NO: 164, 208), and/or orthologs thereof.

Embodiment 19: The isolated dsRNA of embodiment 18, wherein said target genes and/or orthologs comprise one or more target genes selected from the group consisting of lipase 1 (SEQ ID NO: 1, 183), lipase A (SEQ ID NO: 2, 184), isocitrate lyase (SEQ ID NO: 20, 186-187), malate synthase (SEQ ID: 21, 188), apoptosis-antagonizing transcription factor (AATF) (SEQ ID NO: 92, 197), Effector candidate 2 (EC2) (SEQ ID NO: 140,201), β-carotene 15,15′-dioxygenase (BCDO) (SEQ ID NO: 145, 203), and Autophagy protein Apg9 (SEQ ID NO: 164, 208), and/or orthologs thereof.

Embodiment 20: The isolated dsRNA according to any one of embodiments 1-5, wherein said target genes and/or orthologs comprise one or more target genes selected from the group consisting of CYP51 (Seq ID NO: 85, 182), lipase 1 (SEQ ID NO: 1, 183), isocitrate lyase (SEQ ID NO: 20, 186-187), malate synthase (SEQ ID: 21, 188), and Autophagy protein Apg9 (SEQ ID NO: 164, 208), and/or orthologs thereof.

Embodiment 21: The isolated dsRNA of embodiment 20, wherein said target genes and/or orthologs comprise one or more target genes selected from the group consisting of lipase 1 (SEQ ID NO: 1, 183), isocitrate lyase (SEQ ID NO: 20, 186-187), malate synthase (SEQ ID: 21, 188), and Autophagy protein Apg9 (SEQ ID NO: 164, 208), and/or orthologs thereof.

Embodiment 22: The isolated dsRNA according to any one of embodiments 1-5, wherein said dsRNA inhibits or is capable of downregulating expression of a gene involved in lipid metabolism and/or acquisition.

Embodiment 23: The isolated dsRNA of embodiment 22, wherein said dsRNA is capable of downregulating expression of one or more target genes selected from the group consisting of SEQ ID Nos:1-51, 149-151, 181, 183-190, 206, or orthologues thereof.

Embodiment 24: The isolated dsRNA of embodiment 22, wherein said dsRNA inhibits or is capable of downregulating expression of a gene involved in lipid metabolism.

Embodiment 25: The isolated dsRNA of embodiment 24, wherein said dsRNA is capable of downregulating expression of one or more target genes selected from the group consisting of SEQ ID Nos:1-51, or orthologues thereof where said orthologs are found in species of phytopathogen other than the species shown in Table 1.

Embodiment 26: The isolated dsRNA according to any one of embodiments 1-5, wherein said dsRNA inhibits or is capable of downregulating expression of a gene involved in triacylglycerol (TAG)/glycerolipid metabolism.

Embodiment 27: The isolated dsRNA of embodiment 26, wherein said dsRNA is capable of downregulating expression of one or more target genes selected from the group consisting of SEQ ID Nos: 1-14 and 183-184, and/or orthologs thereof.

Embodiment 28: The isolated dsRNA according to any one of embodiments 1-5, wherein said dsRNA inhibits or is capable of downregulating expression of a gene involved in pentose phosphate pathway (PPP).

Embodiment 29: The isolated dsRNA of embodiment 28, wherein said dsRNA is capable of downregulating expression of one or more target genes selected from the group consisting of SEQ ID Nos: 15-19 and 185, and/or orthologs thereof.

Embodiment 30: The isolated dsRNA according to any one of embodiments 1-5, wherein said dsRNA inhibits or is capable of downregulating expression of a gene involved in the tricarboxylic acid (TCA) and/or glyoxylate cycle.

Embodiment 31: The isolated dsRNA of embodiment 30, wherein said dsRNA is capable of downregulating expression of one or more target genes selected from the group consisting of SEQ ID Nos: 20-31, 181, 186-189 shown in Tables 1 and 2 or orthologs thereof.

Embodiment 32: The isolated dsRNA according to any one of embodiments 1-5, wherein said dsRNA inhibits or is capable of downregulating expression of a gene involved in fatty acid synthesis and/or transport.

Embodiment 33: The isolated dsRNA of embodiment 32, wherein said dsRNA is capable of downregulating expression of one or more target genes selected from the group consisting of SEQ ID Nos: 32-41 and 190, and/or orthologs thereof.

Embodiment 34: The isolated dsRNA according to any one of embodiments 1-5, wherein said dsRNA inhibits or is capable of downregulating expression of a gene involved in lipid catabolism.

Embodiment 35: The isolated dsRNA of embodiment 34, wherein said dsRNA is capable of downregulating expression of one or more target genes selected from the group consisting of SEQ ID Nos: 42-50, 149-151, 206, _{and/or} or orthologs thereof.

Embodiment 36: The isolated dsRNA according to any one of embodiments 1-5, wherein said dsRNA inhibits or is capable of downregulating expression of a gene involved in fatty acid breakdown including fatty acid B-oxidation.

Embodiment 37: The isolated dsRNA of embodiment 36, wherein said dsRNA is capable of downregulating expression of one or more target genes selected from the group consisting of SEQ ID Nos: 42-50, and/or orthologs thereof.

Embodiment 38: The isolated dsRNA according to any one of embodiments 1-5, wherein said dsRNA inhibits or is capable of downregulating expression of a gene involved in phospholipid metabolism.

Embodiment 39: The isolated dsRNA of embodiment 38, wherein said dsRNA is capable of downregulating expression of one or more target genes selected from the group consisting of SEQ ID Nos:51, and 95, and/or orthologs thereof.

Embodiment 40: The isolated dsRNA according to any one of embodiments 1-5, wherein said dsRNA inhibits or is capable of downregulating expression of a gene involved in carbohydrate metabolism.

Embodiment 41: The isolated dsRNA of embodiment 40, wherein said dsRNA is capable of downregulating expression of one or more target genes selected from the group consisting of SEQ ID Nos:52-61, and/or orthologs thereof.

Embodiment 42: The isolated dsRNA according to any one of embodiments 1-5, wherein said dsRNA inhibits or is capable of downregulating expression of a gene involved in glycogen metabolism.

Embodiment 43: The isolated dsRNA of embodiment 42, wherein said dsRNA is capable of downregulating expression of one or more target genes selected from the group consisting of SEQ ID Nos:52, 53, and 94, and/or orthologs thereof.

Embodiment 44: The isolated dsRNA according to any one of embodiments 1-5, wherein said dsRNA inhibits or is capable of downregulating expression of a gene involved in glycolysis.

Embodiment 45: The isolated dsRNA of embodiment 44, wherein said dsRNA is capable of downregulating expression of one or more target genes selected from the group consisting of SEQ ID Nos:54-57, and/or orthologs thereof.

Embodiment 46: The isolated dsRNA according to any one of embodiments 1-5, wherein said dsRNA inhibits or is capable of downregulating expression of a gene involved in carbohydrate utilization.

Embodiment 47: The isolated dsRNA of embodiment 46, wherein said dsRNA is capable of downregulating expression of one or more target genes selected from the group consisting of SEQ ID Nos: 58-61, and/or orthologs thereof.

Embodiment 48: The isolated dsRNA according to any one of embodiments 1-5, wherein said dsRNA inhibits or is capable of downregulating expression of a gene involved in amino acid and/or nucleotide metabolism and salvage and/or cofactor metabolism.

Embodiment 49: The isolated dsRNA of embodiment 48, wherein said dsRNA is capable of downregulating expression of one or more target genes selected from the group consisting of SEQ ID Nos:62-84 and 191-195, and/or orthologs thereof.

Embodiment 50: The isolated dsRNA according to any one of embodiments 1-5, wherein wherein said dsRNA inhibits or is capable of downregulating expression of a gene involved in amino acid metabolism and/or salvage.

Embodiment 51: The isolated dsRNA of embodiment 50, wherein said dsRNA is capable of downregulating expression of one or more target genes selected from the group consisting of SEQ ID Nos:62-69 and 191-192, and/or orthologs thereof.

Embodiment 52: The isolated dsRNA according to any one of embodiments 1-5, wherein said dsRNA inhibits or is capable of downregulating expression of a gene involved in cofactor metabolism.

Embodiment 53: The isolated dsRNA of embodiment 52, wherein said dsRNA is capable of downregulating expression of one or more target genes selected from the group consisting of SEQ ID Nos:70-78 and 193-194, and/or orthologs thereof.

Embodiment 54: The isolated dsRNA according to any one of embodiments 1-5, wherein said dsRNA inhibits or is capable of downregulating expression of a gene involved in nucleotide metabolism and/or salvage.

Embodiment 55: The isolated dsRNA of embodiment 54, wherein said dsRNA is capable of downregulating expression of one or more target genes selected from the group consisting of SEQ ID Nos:79-84 and 195, and/or orthologs thereof.

Embodiment 56: The isolated dsRNA according to any one of embodiments 1-5, wherein said dsRNA inhibits or is capable of downregulating expression of a gene involved in essential fungal-specific processes. such as sterol biosynthesis or fungal reproduction.

Embodiment 57: The isolated dsRNA of embodiment 56, wherein said dsRNA is capable of downregulating expression of one or more target genes selected from the group consisting of SEQ ID Nos: 85-86 and 97-98, and/or orthologs thereof.

Embodiment 58: The isolated dsRNA according to any one of embodiments 1-5, wherein said dsRNA inhibits or is capable of downregulating expression of a gene involved in sterol biosynthesis.

Embodiment 59: The isolated dsRNA of embodiment 58, wherein said dsRNA is capable of downregulating expression of one or more target genes selected from the group consisting of SEQ ID Nos: 85 and 178, and/or orthologs thereof.

Embodiment 60: The isolated dsRNA according to any one of embodiments 1-5, wherein said dsRNA inhibits or is capable of downregulating expression of a gene involved in fungal reproduction/compatibility.

Embodiment 61: The isolated dsRNA of embodiment 60, wherein said dsRNA is capable of downregulating expression of one or more target genes selected from the group consisting of SEQ ID Nos: 86 and 97, and/or orthologs thereof.

Embodiment 62: The isolated dsRNA according to any one of embodiments 1-5, wherein said dsRNA inhibits or is capable of downregulating expression of a gene involved in mitochondrial electron transport and ATP synthesis.

Embodiment 63: The isolated dsRNA of embodiment 62, said dsRNA is capable of downregulating expression of one or more target genes selected from the group consisting of SEQ ID Nos: 87-91 and 196, and/or orthologs thereof.

Embodiment 64: The isolated dsRNA according to any one of embodiments 1-5, wherein said dsRNA inhibits or is capable of downregulating expression of a gene involved in the alternative respiratory pathway.

Embodiment 65: The isolated dsRNA of embodiment 64, wherein said dsRNA is capable of downregulating expression of one or more target genes selected from the group consisting of SEQ ID Nos: 90-91, and/or orthologs thereof.

Embodiment 66: The isolated dsRNA according to any one of embodiments 1-5, wherein said dsRNA inhibits or is capable of downregulating expression of a gene involved in transcriptional regulation.

Embodiment 67: The isolated dsRNA of embodiment 66, wherein said dsRNA is capable of downregulating expression of one or more target genes selected from the group consisting of SEQ ID Nos:92-103 and 197, and/or orthologs thereof.

Embodiment 68: The isolated dsRNA according to any one of embodiments 1-5, wherein said dsRNA inhibits or is capable of downregulating expression of a gene involved in transcriptional regulation of stress.

Embodiment 69: The isolated dsRNA of embodiment 68, wherein said dsRNA is capable of downregulating expression of one or more target genes selected from the group consisting of SEQ ID Nos:92-93 and 197, and/or orthologs thereof.

Embodiment 70: The isolated dsRNA according to any one of embodiments 1-5, wherein said dsRNA inhibits or is capable of downregulating expression of a gene involved in transcriptional regulation of metabolism.

Embodiment 71: The isolated dsRNA of embodiment 70, wherein said dsRNA is capable of downregulating expression of one or more target genes selected from the group consisting of SEQ ID Nos:94-96, and/or orthologs thereof.

Embodiment 72: The isolated dsRNA according to any one of embodiments 1-5, wherein said dsRNA inhibits or is capable of downregulating expression of a gene involved in transcriptional regulation of development.

Embodiment 73: The isolated dsRNA of embodiment 72, wherein said dsRNA is capable of downregulating expression of one or more target genes selected from the group consisting of SEQ ID Nos:97-99, and/or orthologs thereof.

Embodiment 74: The isolated dsRNA according to any one of embodiments 1-5, wherein said dsRNA inhibits or is capable of downregulating expression of a gene involved in transcription and/or RNA-binding.

Embodiment 75: The isolated dsRNA of embodiment 74, wherein said dsRNA is capable of downregulating expression of one or more target genes selected from the group consisting of SEQ ID Nos: 100-103, and/or orthologs thereof.

Embodiment 76: The isolated dsRNA according to any one of embodiments 1-5, wherein said dsRNA inhibits or is capable of downregulating expression of a gene involved in translation.

Embodiment 77: The isolated dsRNA of embodiment 76, wherein said dsRNA is capable of downregulating expression of one or more target genes selected from the group consisting of SEQ ID Nos:104-109 and 198, and/or orthologs thereof.

Embodiment 78: The isolated dsRNA according to any one of embodiments 1-5, wherein said dsRNA inhibits or is capable of downregulating expression of a gene involved in protein modification and/or turnover.

Embodiment 79: The isolated dsRNA of embodiment 78, wherein said dsRNA is capable of downregulating expression of one or more target genes selected from the group consisting of SEQ ID No:110, and/or orthologs thereof.

Embodiment 80: The isolated dsRNA according to any one of embodiments 1-5, wherein said dsRNA inhibits or is capable of downregulating expression of a gene involved in protein localization and/or folding.

Embodiment 81: The isolated dsRNA of embodiment 80, wherein said dsRNA is capable of downregulating expression of one or more target genes selected from the group consisting of SEQ ID No:111-113, 141 and 202, and/or orthologs thereof.

Embodiment 82: The isolated dsRNA according to any one of embodiments 1-5, wherein said dsRNA inhibits or is capable of downregulating expression of a gene involved in signaling.

Embodiment 83: The isolated dsRNA of embodiment 82, wherein said dsRNA is capable of downregulating expression of one or more target genes selected from the group consisting of SEQ ID Nos:114-121, 148, and 205, and/or orthologs thereof.

Embodiment 84: The isolated dsRNA according to any one of embodiments 1-5, wherein said dsRNA inhibits or is capable of downregulating expression of a gene encoding a known or predicted secreted protein.

Embodiment 85: The isolated dsRNA of embodiment 84, wherein said dsRNA is capable of downregulating expression of one or more target genes selected from the group consisting of SEQ ID Nos:122-144, and 199-202, and/or orthologs thereof.

Embodiment 86: The isolated dsRNA according to any one of embodiments 1-5, wherein said dsRNA inhibits or is capable of downregulating expression of a gene encoding a known or predicted secreted protein associated with the TCP network.

Embodiment 87: The isolated dsRNA of embodiment 86, wherein said dsRNA is capable of downregulating expression of one or more target genes selected from the group consisting of SEQ ID Nos:122-139 and 199-200, and/or orthologs thereof.

Embodiment 88: The isolated dsRNA according to any one of embodiments 1-5, wherein, wherein said dsRNA inhibits or is capable of downregulating expression of a gene encoding a known or predicted secreted protein highly expressed during powdery mildew infection.

Embodiment 89: The isolated dsRNA of embodiment 88, wherein said dsRNA is capable of downregulating expression of one or more target genes selected from the group consisting of SEQ ID Nos:140-144 and 201-202, and/or orthologs thereof.

Embodiment 90: The isolated dsRNA according to any one of embodiments 1-5, wherein, wherein said dsRNA inhibits or is capable of downregulating expression of a gene predicted to be involved in plant hormone metabolism.

Embodiment 91: The isolated dsRNA of embodiment 90, wherein said dsRNA is capable of downregulating expression of one or more target genes selected from the group consisting of SEQ ID Nos:145-154 and 203-206, and/or orthologs thereof.

Embodiment 92: The isolated dsRNA according to any one of embodiments 1-5, wherein said dsRNA inhibits or is capable of downregulating expression of a gene predicted to be involved in carotenoid metabolism, including abscicic acid.

Embodiment 93: The isolated dsRNA of embodiment 92, wherein said dsRNA is capable of downregulating expression of one or more target genes selected from the group consisting of SEQ ID Nos:145-148 and 203-205, and/or orthologs thereof.

Embodiment 94: The isolated dsRNA according to any one of embodiments 1-5, wherein said dsRNA inhibits or is capable of downregulating expression of a gene predicted to be involved in oxylipin (e.g. jasmonic acid) metabolism, acquisition, or signaling.

Embodiment 95: The isolated dsRNA of embodiment 94, wherein said dsRNA is capable of downregulating expression of one or more target genes selected from the group consisting of SEQ ID Nos:149-151 and 206, and/or orthologs thereof.

Embodiment 96: The isolated dsRNA according to any one of embodiments 1-5, wherein said dsRNA inhibits or is capable of downregulating expression of a gene predicted to be involved chorismate and chorismate-derived plant hormone metabolism, including salicylic acid and auxin metabolism.

Embodiment 97: The isolated dsRNA of embodiment 96, wherein said dsRNA is capable of downregulating expression of one or more target genes selected from the group consisting of SEQ ID Nos:152-154, or orthologs thereof.

Embodiment 98: The isolated dsRNA according to any one of embodiments 1-5, wherein said dsRNA inhibits or is capable of downregulating expression of a gene involved in the cell cycle and/or DNA replication and repair.

Embodiment 99: The isolated dsRNA of embodiment 98, wherein said dsRNA is capable of downregulating expression of one or more target genes selected from the group consisting of SEQ ID Nos:155-163, 180, 207, and 211, or orthologs thereof.

Embodiment 100: The isolated dsRNA according to any one of embodiments 1-5, wherein said dsRNA inhibits or is capable of downregulating expression of a gene involved vesicles, and/or autophagy, and/or phagocytosis.

Embodiment 101: The isolated dsRNA of embodiment 100, wherein said dsRNA is capable of downregulating expression of one or more target genes selected from the group consisting of SEQ ID Nos:164-166 and 208, and/or orthologs thereof.

Embodiment 102: The isolated dsRNA according to any one of embodiments 1-5, wherein said dsRNA inhibits or is capable of downregulating expression of a gene involved in osmotic homoeostasis and/or metal/ion transport.

Embodiment 103: The isolated dsRNA of embodiment 102, wherein said dsRNA is capable of downregulating expression of one or more target genes selected from the group consisting of SEQ ID Nos:167-169 and 209-201, or orthologues thereof.

Embodiment 104: The isolated dsRNA according to any one of embodiments 1-5, wherein said dsRNA inhibits or is capable of downregulating expression of a gene involved in cell volume control.

Embodiment 105: The isolated dsRNA of embodiment 104, wherein said dsRNA is capable of downregulating expression of one or more target genes selected from the group consisting of SEQ ID Nos:167 and 209, and/or orthologues thereof.

Embodiment 106: The isolated dsRNA according to any one of embodiments 1-5, wherein, wherein said dsRNA inhibits or is capable of downregulating expression of a gene involved in metal/ion transport.

Embodiment 107: The isolated dsRNA of embodiment 106, wherein said dsRNA is capable of downregulating expression of one or more target genes selected from the group consisting of SEQ ID Nos:168-169 and 210, or orthologues thereof.

Embodiment 108: The isolated dsRNA according to any one of embodiments 1-5, wherein, wherein said dsRNA inhibits or is capable of downregulating expression of a gene identified as a single copy gene following whole genome duplication in G. orontii MGH1.

Embodiment 109: The isolated dsRNA of embodiment 108, wherein said dsRNA is capable of downregulating expression of one or more target genes selected from the group consisting of SEQ ID Nos:255-411.

Embodiment 110: The isolated dsRNA of embodiment 108, wherein said dsRNA is capable of downregulating expression of one or more target genes selected from the group consisting of SEQ ID Nos: 15, 40, 51, 54, 58, 59-61, 63-66, 68, 70, 72, 78, 83, 87, 88, 93, 98-99, 104-113, 145, 154-180 and 185, 191-193, 196, 198, 207-211, and/or orthologs thereof.

Embodiment 111: The isolated dsRNA of embodiment 108, wherein said dsRNA is capable of downregulating expression of one or more target genes selected from the group consisting of SEQ ID Nos:170-179, and/or orthologs thereof.

Embodiment 112: The isolated dsRNA of embodiment 108, wherein said dsRNA is capable of downregulating expression of one or more target genes selected from the group consisting of SEQ ID Nos:15, 63-64, 70, 87, 104, 145, 164, 167, 169, 179, and 180, and/or orthologs thereof.

Embodiment 113: The isolated dsRNA according to any one of embodiments 1-5, wherein said dsRNA inhibits or is capable of downregulating expression of a gene identified as interacting with the TCP network.

Embodiment 114: The isolated dsRNA of embodiment 113, wherein said dsRNA is capable of downregulating expression of one or more target genes selected from the group consisting of SEQ ID Nos:103, 122-139, 163 and 199-200, and/or orthologs thereof.

Embodiment 115: The isolated dsRNA according to any one of embodiments 1-5, wherein said dsRNA inhibits or is capable of downregulating expression of a gene involved in carbohydrate metabolism.

Embodiment 116: The composition according to any one of embodiment 115, wherein said dsRNA is capable of downregulating expression of one or more target genes selected from the group consisting of SEQ ID Nos:52-61, and/or orthologs thereof.

Embodiment 117: The isolated dsRNA according to any one of embodiments 1-5, wherein said dsRNA inhibits or is capable of downregulating expression of a gene involved in amino acid and/or nucleotide metabolism and/or salvage.

Embodiment 118: The composition according to any one of embodiment 117, wherein said dsRNA is capable of downregulating expression of one or more target genes selected from the group consisting of SEQ ID Nos:62-84, and/or orthologs thereof.

Embodiment 119: The isolated dsRNA according to any one of embodiments 1-5, wherein said dsRNA inhibits or is capable of downregulating expression of a gene involved in an essential fungal process such as sterol biosynthesis or fungal sexual reproduction.

Embodiment 120: The composition according to any one of embodiment 119, wherein said dsRNA is capable of downregulating expression of one or more target genes selected from the group consisting of SEQ ID Nos:85-86, and/or orthologs thereof.

Embodiment 121: The isolated dsRNA according to any one of embodiments 1-5, wherein said dsRNA inhibits or is capable of downregulating expression of a gene involved in mitochondrial electron transport.

Embodiment 122: The composition according to any one of embodiment 121, wherein said dsRNA is capable of downregulating expression of one or more target genes selected from the group consisting of SEQ ID Nos:87-91, and/or orthologs thereof.

Embodiment 123: The isolated dsRNA according to any one of embodiments 1-5, wherein said dsRNA inhibits or is capable of downregulating expression of a gene involved in transcriptional regulation.

Embodiment 124: The composition according to any one of embodiment 123, wherein said dsRNA is capable of downregulating expression of one or more target genes selected from the group consisting of SEQ ID Nos:92-103, and/or orthologs thereof.

Embodiment 125: The isolated dsRNA according to any one of embodiments 1-5, wherein said dsRNA inhibits or is capable of downregulating expression of a gene involved in translation.

Embodiment 126: The composition according to any one of embodiment 125, wherein said dsRNA is capable of downregulating expression of one or more target genes selected from the group consisting of SEQ ID Nos:104-113, and/or orthologs thereof.

Embodiment 127: The isolated dsRNA according to any one of embodiments 1-5, wherein said dsRNA inhibits or is capable of downregulating expression of a gene involved in signaling.

Embodiment 128: The composition according to any one of embodiment 127, wherein said dsRNA is capable of downregulating expression of one or more target genes selected from the group consisting of SEQ ID Nos:114-121, and/or orthologs thereof.

Embodiment 129: The isolated dsRNA according to any one of embodiments 1-5, wherein said dsRNA inhibits or is capable of downregulating expression of a gene involved protein secretion.

Embodiment 130: The composition according to any one of embodiment 129, wherein said dsRNA is capable of downregulating expression of one or more target genes selected from the group consisting of SEQ ID Nos:122-144, and/or orthologs thereof.

Embodiment 131: The isolated dsRNA according to any one of embodiments 1-130, wherein said dsRNA inhibits or is capable of downregulating expression of a gene involved plant hormone metabolism.

Embodiment 132: The composition according to any one of embodiment 131, wherein said dsRNA is capable of downregulating expression of one or more target genes selected from the group consisting of SEQ ID Nos:145-154, and/or orthologs thereof.

Embodiment 133: The isolated dsRNA according to any one of embodiments 1-5, wherein said dsRNA inhibits or is capable of downregulating expression of a gene involved cell cycle and/or DNA replication.

Embodiment 134: The composition according to any one of embodiment 133, wherein said dsRNA is capable of downregulating expression of one or more target genes selected from the group consisting of SEQ ID Nos:155-163, and/or orthologs thereof.

Embodiment 135: The isolated dsRNA according to any one of embodiments 1-5, wherein said dsRNA inhibits or is capable of downregulating expression of a gene involved vesicles, and/or autophagy, and/or phagocytosis.

Embodiment 136: The composition according to any one of embodiment 135, wherein said dsRNA is capable of downregulating expression of one or more target genes selected from the group consisting of SEQ ID Nos:164-166, and/or orthologs thereof.

Embodiment 137: The isolated dsRNA according to any one of embodiments 1-5, wherein said dsRNA inhibits or is capable of downregulating expression of a gene involved in cell volume control and/or metal ion transport.

Embodiment 138: The composition according to any one of embodiment 137, wherein said dsRNA is capable of downregulating expression of one or more target genes selected from the group consisting of SEQ ID Nos:167-169, and/or orthologs thereof.

Embodiment 139: The isolated dsRNA according to any one of embodiments 1-5, wherein said dsRNA is capable of downregulating expression of one or more target genes selected from the group consisting of SEQ ID Nos:170-178, and/or orthologs thereof.

Embodiment 140: The isolated dsRNA according to any one of embodiment 1, wherein said dsRNA inhibits or is capable of downregulating (e.g., downregulates) expression of one or more target genes selected from the group consisting of G. orontii lipase A, G. orontii lipase 1, G. orontii transaldolase, G. orontii isocitrate lyase, G. orontii malate synthase, G. orontii citrate (Si) synthase, alanine-glyoxylate aminotransferase, and G. orontii trans-2-enoyl-CoA reductase, and/or an ortholog thereof.

Embodiment 141: The isolated dsRNA of embodiment 1, wherein said target gene is selected from the group consisting of G. orontii glutamate dehydrogenase, G. orontii threonine aldolase, G. orontii Ketopantoate hydroxymethyltransferase (KPHMT), G. orontii tetrahydrofolate synthase, and G. orontii thymidylate synthase, and/or an ortholog thereof.

Embodiment 142: The isolated dsRNA of embodiment 1, wherein said target gene is selected from the group consisting of pf08238-Sel1 repeat (SEQ NO. 87,196), and/or an ortholog thereof.

Embodiment 143: The isolated dsRNA of embodiment 1 wherein said target gene is selected from the group consisting of G. orontii apotosis antagonizing transcription factor (AATF) and G. orontii nuclear fragile X mental retardation interacting protein 1 (NUFIP1), and/or an ortholog thereof.

Embodiment 144: The isolated dsRNA of embodiment 1, wherein said target gene is selected from the group consisting of G. orontii OEC60/61, G. orontii OEC70, and G. orontii OEC14, and/or an ortholog thereof.

Embodiment 145: The isolated dsRNA of embodiment 1, wherein said target gene is selected from the group consisting of G. orontii effector candidate 2 (EC2) and G. orontii HSP70 family, and/or an ortholog thereof.

Embodiment 146: The isolated dsRNA of embodiment 1, wherein said target gene is selected from the group consisting of G. orontii β-carotene 15,15′-dioxygenase (BCDO), G. orontii xanthoxin oxidase, G. orontii ABA G-coupled receptor and G. orontii acyl-CoA oxidase, and/or an ortholog thereof.

Embodiment 147: The isolated dsRNA according to any one of embodiments 1-5, wherein said target gene is selected from the group consisting of G. orontii β-carotene 15,15′-dioxygenase (BCDO), G. orontii xanthoxin oxidase, and G. orontii ABAR, and/or an ortholog thereof.

Embodiment 148: The isolated dsRNA according to any one of embodiments 1-5, wherein said dsRNA is capable of downregulating (e.g., downregulates) expression of one or more target G. orontii genes selected from the group consisting of Cytochrome P450 monoxygenase (CYP51) (SEQ ID NO:85), TAG lipase 1, caroboxyesterase domain (SEQ ID NO:1), TAG lipase A, extracellular lipase (SEQ ID NO:2), Transaldolase (SEQ ID NO:15), Isocitrate lyase (ICL) (SEQ ID NO:20), Malate synthase (MS) (SEQ ID NO:21), Citrate (Si) synthase (SEQ ID NO:22), Trans-2-enoyl CoA reductase (SEQ ID NO:32), Glutamate dehydrogenase (SEQ ID NO:63), Threonine aldolase (SEQ ID NO:64), Pf02548—Ketopantoate hydroxymethyltransferase (SEQ ID NO:70), Tetrahydrofolate synthase (SEQ ID NO:71), Thymidine synthase (SEQ ID NO:79), Pf8238-Sel1 repeat (SEQ ID NO:87), Apoptosis antagonizing transcription factor (AATF) (SEQ ID NO:92), Pf10453—Nuclear fragile X mental retardation-interacting protein 1 (NUFIP1) (SEQ ID NO:104), G. orontii effector candidate 60/61 (SEQ ID NO:122), G. orontii effector candidate 70 (SEQ ID NO:123), G. orontii effector candidate 14 (SEQ ID NO:124), Effector protein EC2 (SEQ ID NO:140), Heat shock protein 70 family (HSP70) (SEQ ID NO:141), β-carotene 15,15′-dioxygenase (BCDO) (SEQ ID NO:145), Xanthin oxidase (SEQ ID NO:146), Acyl-CoA oxidase (ACX_(SEQ ID NO:149), and Pf04109—Autophagy protein Apg9 (SEQ ID NO:164), or an ortholog thereof where said ortholog is found in other species of phytopathogen.

Embodiment 149: The composition according to any one of embodiment 1, wherein said target gene is selected from the group consisting of an ortholog of a gene selected from the group consisting of G. orontii lipase a, G. orontii lipase 1, G. orontii (3-carotene 15,15′-dioxygenase, G. orontii apoptosis-antagonizing transcription factor (AATF), effector candidate 2, OEC14, OEC60/OEC61, and OEC70.

Embodiment 150: The composition according to any one of embodiment 1, wherein said target gene is selected from the group consisting of an ortholog of a gene selected from the group consisting of TAG lipase 1, caroboxyesterase domain (SEQ ID NO:1), TAG lipase A, extracellular lipase (SEQ ID NO:2), Transaldolase (SEQ ID NO:15), Isocitrate lyase (ICL) (SEQ ID NO:20), Malate synthase (MS) (SEQ ID NO:21), Citrate (Si) synthase (SEQ ID NO:22), Trans-2-enoyl CoA reductase (SEQ ID NO:32), Glutamate dehydrogenase (SEQ ID NO:63), Threonine aldolase (SEQ ID NO:64), Pf02548-Ketopantoate hydroxymethyltransferase (SEQ ID NO:70), Tetrahydrofolate synthase (SEQ ID NO:71), Thymidine synthase (SEQ ID NO:79), Pf8238-Sel1 repeat (SEQ ID NO:87), Apoptosis antagonizing transcription factor (AATF) (SEQ ID NO:92), Pf10453—Nuclear fragile X mental retardation-interacting protein 1 (NUFIP1) (SEQ ID NO:104), G. orontii effector candidate 60/61 (SEQ ID NO:122), G. orontii effector candidate 70 (SEQ ID NO:123), G. orontii effector candidate 14 (SEQ ID NO:124), Effector protein EC2 (SEQ ID NO:140), Heat shock protein 70 family (HSP70) (SEQ ID NO:141), β-carotene 15,15′-dioxygenase (BCDO) (SEQ ID NO:145), Xanthin oxidase (SEQ ID NO:146), Acyl-CoA oxidase (ACX_(SEQ ID NO:149), and Pf04109—Autophagy protein Apg9 (SEQ ID NO:164).

Embodiment 151: The composition according to any one of embodiments 1-5, wherein said target gene is selected from the group consisting of an ortholog of a gene selected from the group consisting of MS (SEQ ID NO:21), ICL1 (SEQ ID NO:20), ICL2 (SEQ ID NO:20), LIP1 (SEQ ID NO:1), LIPA (SEQ ID NO:2), BCDO (SEQ ID NO:145), ACX (SEQ ID NO:149), AATF (SEQ ID NO:92), EC2 (SEQ ID NO:140), OEC60/OEC61 (SEQ ID NO:122), OEC70 (SEQ ID NO:123), and OEC14 (SEQ ID NO:124).

Embodiment 152: The isolated dsRNA of embodiment 151, wherein said target gene is an orgolog of G. orontii target gene MS (SEQ ID NO:21).

Embodiment 153: The composition according to any one of embodiment 151, wherein said target gene is an orgolog of G. orontii target gene ICL1 (SEQ ID NO:20).

Embodiment 154: The composition according to any one of embodiment 151, wherein said target gene is an orgolog of G. orontii target gene ICL2 (SEQ ID NO:20).

Embodiment 155: The composition according to any one of embodiment 151, wherein said target gene is an orgolog of G. orontii target gene LIP1 (SEQ ID NO:1).

Embodiment 156: The composition according to any one of embodiment 151, wherein said target gene is an orgolog of G. orontii target gene LIPA (SEQ ID NO:2).

Embodiment 157: The composition according to any one of embodiment 151, wherein said target gene is an orgolog of G. orontii target gene BCDO (SEQ ID NO:145).

Embodiment 158: The composition according to any one of embodiment 151, wherein said target gene is an orgolog of G. orontii target gene ACX (SEQ ID NO:149).

Embodiment 159: The composition according to any one of embodiment 151, wherein said target gene is an orgolog of G. orontii target gene AATF (SEQ ID NO:92).

Embodiment 160: The composition according to any one of embodiment 151, wherein said target gene is an orgolog of G. orontii target gene EC2 (SEQ ID NO:140).

Embodiment 161: The composition according to any one of embodiment 151, wherein said target gene is an orgolog of G. orontii target gene OEC60/OEC61 (SEQ ID NO:122).

Embodiment 162: The composition according to any one of embodiment 151, wherein said target gene is an orgolog of G. orontii target gene OEC70 (SEQ ID NO:123).

Embodiment 163: The composition according to any one of embodiment 151, wherein said target gene is an orgolog of G. orontii target gene OEC14 (SEQ ID NO:124).

Embodiment 164: The isolated dsRNA according to any one of embodiments 1-163, wherein said target gene is an ortholog of one of said G. orontii genes.

Embodiment 165: The isolated dsRNA according to any one of embodiments 1-3, wherein said target genes and/or orthologs comprise the target gene of SEQ ID NO:1, and/or an ortholog thereof.

Embodiment 166: The isolated dsRNA of embodiment 165, wherein said target genes and/or orthologs comprise the target gene of SEQ ID NO:2, and/or an ortholog thereof.

Embodiment 167: The isolated dsRNA according to any one of embodiments 165-166, wherein said target genes and/or orthologs comprise the target gene of SEQ ID NO:15, and/or an ortholog thereof.

Embodiment 168: The isolated dsRNA according to any one of embodiments 165-167, wherein said target genes and/or orthologs comprise the target gene of SEQ ID NO:20, and/or an ortholog thereof.

Embodiment 169: The isolated dsRNA according to any one of embodiments 165-168, wherein said target genes and/or orthologs comprise the target gene of SEQ ID NO:21, and/or an ortholog thereof.

Embodiment 170: The isolated dsRNA according to any one of embodiments 165-169, wherein said target genes and/or orthologs comprise the target gene of SEQ ID NO:22, and/or an ortholog thereof.

Embodiment 171: The isolated dsRNA according to any one of embodiments 165-170, wherein said target genes and/or orthologs comprise the target gene of SEQ ID NO:32, and/or an ortholog thereof.

Embodiment 172: The isolated dsRNA according to any one of embodiments 165-171, wherein said target genes and/or orthologs comprise the target gene of SEQ ID NO:63, and/or an ortholog thereof.

Embodiment 173: The isolated dsRNA according to any one of embodiments 165-172, wherein said target genes and/or orthologs comprise the target gene of SEQ ID NO:64, and/or an ortholog thereof.

Embodiment 174: The isolated dsRNA according to any one of embodiments 165-173, wherein said target genes and/or orthologs comprise the target gene of SEQ ID NO:70, and/or an ortholog thereof.

Embodiment 175: The isolated dsRNA according to any one of embodiments 165-174, wherein said target genes and/or orthologs comprise the target gene of SEQ ID NO:71, and/or an ortholog thereof.

Embodiment 176: The isolated dsRNA according to any one of embodiments 165-175, wherein said target genes and/or orthologs comprise the target gene of SEQ ID NO:79, and/or an ortholog thereof.

Embodiment 177: The isolated dsRNA according to any one of embodiments 165-176, wherein said target genes and/or orthologs comprise the target gene of SEQ ID NO:87, and/or an ortholog thereof.

Embodiment 178: The isolated dsRNA according to any one of embodiments 165-177, wherein said target genes and/or orthologs comprise the target gene of SEQ ID NO:92, and/or an ortholog thereof.

Embodiment 179: The isolated dsRNA according to any one of embodiments 165-178, wherein said target genes and/or orthologs comprise the target gene of SEQ ID NO:104, and/or an ortholog thereof.

Embodiment 180: The isolated dsRNA according to any one of embodiments 165-179, wherein said target genes and/or orthologs comprise the target gene of SEQ ID NO:122, and/or an ortholog thereof.

Embodiment 181: The isolated dsRNA according to any one of embodiments 165-180, wherein said target genes and/or orthologs comprise the target gene of SEQ ID NO:123, and/or an ortholog thereof.

Embodiment 182: The isolated dsRNA according to any one of embodiments 165-181, wherein said target genes and/or orthologs comprise the target gene of SEQ ID NO:124, and/or an ortholog thereof.

Embodiment 183: The isolated dsRNA according to any one of embodiments 165-182, wherein said target genes and/or orthologs comprise the target gene of SEQ ID NO:140, and/or an ortholog thereof.

Embodiment 184: The isolated dsRNA according to any one of embodiments 165-183, wherein said target genes and/or orthologs comprise the target gene of SEQ ID NO:141, and/or an ortholog thereof.

Embodiment 185: The isolated dsRNA according to any one of embodiments 165-184, wherein said target genes and/or orthologs comprise the target gene of SEQ ID NO:145, and/or an ortholog thereof.

Embodiment 186: The isolated dsRNA according to any one of embodiments 165-185, wherein said target genes and/or orthologs comprise the target gene of SEQ ID NO:145, and/or an ortholog thereof.

Embodiment 187: The isolated dsRNA according to any one of embodiments 165-186, wherein said target genes and/or orthologs comprise the target gene of SEQ ID NO:146, and/or an ortholog thereof.

Embodiment 188: The isolated dsRNA according to any one of embodiments 165-187, wherein said target genes and/or orthologs comprise the target gene of SEQ ID NO:148, and/or an ortholog thereof.

Embodiment 189: The isolated dsRNA according to any one of embodiments 165-188, wherein said target genes and/or orthologs comprise the target gene of SEQ ID NO:149, and/or an ortholog thereof.

Embodiment 190: The isolated dsRNA according to any one of embodiments 165-189, wherein said target genes and/or orthologs comprise the target gene of SEQ ID NO:163, and/or an ortholog thereof.

Embodiment 191: The isolated dsRNA according to any one of embodiments 165-190, wherein said target genes and/or orthologs comprise the target gene of SEQ ID NO:164, and/or an ortholog thereof.

Embodiment 192: The isolated dsRNA according to any one of embodiments 165-191, wherein said target genes and/or orthologs comprise the target gene of SEQ ID NO:167, and/or an ortholog thereof.

Embodiment 193: The isolated dsRNA according to any one of embodiments 165-192, wherein said target genes and/or orthologs comprise the target gene of SEQ ID NO:169, and/or an ortholog thereof.

Embodiment 194: The isolated dsRNA according to any one of embodiments 165-193, wherein said target genes and/or orthologs comprise the target gene of SEQ ID NO:179, and/or an ortholog thereof.

Embodiment 195: The isolated dsRNA according to any one of embodiments 165-194, wherein said target genes and/or orthologs comprise the target gene of SEQ ID NO:180, and/or an ortholog thereof.

Embodiment 196: The isolated dsRNA according to any one of embodiments 165-195, wherein said target genes and/or orthologs comprise the target gene of SEQ ID NO:181, and/or an ortholog thereof.

Embodiment 197: The isolated dsRNA according to any one of embodiments 165-196, wherein said target genes and/or orthologs comprise the target gene of SEQ ID NO:182, and/or an ortholog thereof.

Embodiment 198: The isolated dsRNA according to any one of embodiments 165-197, wherein said target genes and/or orthologs comprise the target gene of SEQ ID NO:183, and/or an ortholog thereof.

Embodiment 199: The isolated dsRNA according to any one of embodiments 165-198, wherein said target genes and/or orthologs comprise the target gene of SEQ ID NO:184, and/or an ortholog thereof.

Embodiment 200: The isolated dsRNA according to any one of embodiments 165-199, wherein said target genes and/or orthologs comprise the target gene of SEQ ID NO:185, and/or an ortholog thereof.

Embodiment 201: The isolated dsRNA according to any one of embodiments 165-200, wherein said target genes and/or orthologs comprise the target gene of SEQ ID NO:186, and/or an ortholog thereof.

Embodiment 202: The isolated dsRNA according to any one of embodiments 165-201, wherein said target genes and/or orthologs comprise the target gene of SEQ ID NO:187, and/or an ortholog thereof.

Embodiment 203: The isolated dsRNA according to any one of embodiments 165-202, wherein said target genes and/or orthologs comprise the target gene of SEQ ID NO:188, and/or an ortholog thereof.

Embodiment 204: The isolated dsRNA according to any one of embodiments 165-203, wherein said target genes and/or orthologs comprise the target gene of SEQ ID NO:189, and/or an ortholog thereof.

Embodiment 205: The isolated dsRNA according to any one of embodiments 165-204, wherein said target genes and/or orthologs comprise the target gene of SEQ ID NO:190, and/or an ortholog thereof.

Embodiment 206: The isolated dsRNA according to any one of embodiments 165-205, wherein said target genes and/or orthologs comprise the target gene of SEQ ID NO:191, and/or an ortholog thereof.

Embodiment 207: The isolated dsRNA according to any one of embodiments 165-206, wherein said target genes and/or orthologs comprise the target gene of SEQ ID NO:192, and/or an ortholog thereof.

Embodiment 208: The isolated dsRNA according to any one of embodiments 165-207, wherein said target genes and/or orthologs comprise the target gene of SEQ ID NO:193, and/or an ortholog thereof.

Embodiment 209: The isolated dsRNA according to any one of embodiments 165-208, wherein said target genes and/or orthologs comprise the target gene of SEQ ID NO:194, and/or an ortholog thereof.

Embodiment 210: The isolated dsRNA according to any one of embodiments 165-209, wherein said target genes and/or orthologs comprise the target gene of SEQ ID NO:195, and/or an ortholog thereof.

Embodiment 211: The isolated dsRNA according to any one of embodiments 165-210, wherein said target genes and/or orthologs comprise the target gene of SEQ ID NO:196, and/or an ortholog thereof.

Embodiment 212: The isolated dsRNA according to any one of embodiments 165-211, wherein said target genes and/or orthologs comprise the target gene of SEQ ID NO:197, and/or an ortholog thereof.

Embodiment 213: The isolated dsRNA according to any one of embodiments 165-212, wherein said target genes and/or orthologs comprise the target gene of SEQ ID NO:198, and/or an ortholog thereof.

Embodiment 214: The isolated dsRNA according to any one of embodiments 165-213, wherein said target genes and/or orthologs comprise the target gene of SEQ ID NO:199, and/or an ortholog thereof.

Embodiment 215: The isolated dsRNA according to any one of embodiments 165-214, wherein said target genes and/or orthologs comprise the target gene of SEQ ID NO:200, and/or an ortholog thereof.

Embodiment 216: The isolated dsRNA according to any one of embodiments 165-215, wherein said target genes and/or orthologs comprise the target gene of SEQ ID NO:201, and/or an ortholog thereof.

Embodiment 217: The isolated dsRNA according to any one of embodiments 165-216, wherein said target genes and/or orthologs comprise the target gene of SEQ ID NO:202, and/or an ortholog thereof.

Embodiment 218: The isolated dsRNA according to any one of embodiments 165-217, wherein said target genes and/or orthologs comprise the target gene of SEQ ID NO:203, and/or an ortholog thereof.

Embodiment 219: The isolated dsRNA according to any one of embodiments 165-218, wherein said target genes and/or orthologs comprise the target gene of SEQ ID NO:204, and/or an ortholog thereof.

Embodiment 220: The isolated dsRNA according to any one of embodiments 165-219, wherein said target genes and/or orthologs comprise the target gene of SEQ ID NO:205, and/or an ortholog thereof.

Embodiment 221: The isolated dsRNA according to any one of embodiments 165-220, wherein said target genes and/or orthologs comprise the target gene of SEQ ID NO:206, and/or an ortholog thereof.

Embodiment 222: The isolated dsRNA according to any one of embodiments 165-221, wherein said target genes and/or orthologs comprise the target gene of SEQ ID NO:207, and/or an ortholog thereof.

Embodiment 223: The isolated dsRNA according to any one of embodiments 165-222, wherein said target genes and/or orthologs comprise the target gene of SEQ ID NO:208, and/or an ortholog thereof.

Embodiment 224: The isolated dsRNA according to any one of embodiments 165-223, wherein said target genes and/or orthologs comprise the target gene of SEQ ID NO:209, and/or an ortholog thereof.

Embodiment 225: The isolated dsRNA according to any one of embodiments 165-224, wherein said target genes and/or orthologs comprise the target gene of SEQ ID NO:210, and/or an ortholog thereof.

Embodiment 226: The isolated dsRNA according to any one of embodiments 165-225, wherein said target genes and/or orthologs comprise the target gene of SEQ ID NO:211, and/or an ortholog thereof.

Embodiment 227: The isolated dsRNA according to any one of embodiments 165-226, wherein said target genes and/or orthologs comprise the target gene of SEQ ID NO:85, and/or an ortholog thereof.

Embodiment 228: The isolated dsRNA according to any one of embodiments 1-227, wherein said dsRNA inhibits or is capable of downregulating expression of orthologs of said one or more target where said orthologs are found in species of phytopathogen other than the species shown in Table 1 (G. orontii).

Embodiment 229: The isolated dsRNA according to any one of embodiments 1-228, wherein said dsRNA inhibits or is capable of downregulating expression of orthologs said one or more target genes where said orthologs are found in species of phytopathogen other than the species shown in Table 1 and Table 2 (G. orontii and E. necator).

Embodiment 230: The isolated dsRNA according to any one of embodiments 1-227, wherein said dsRNA inhibits or is capable of downregulating expression of said one or more target genes.

Embodiment 231: The isolated dsRNA according to any one of embodiments 1-230, wherein said ortholog is an ortholog found in an obligate biotroph.

Embodiment 232: The isolated dsRNA according to any one of embodiments 1-230, wherein said ortholog is an ortholog found in a powdery mildew.

Embodiment 233: The isolated dsRNA of embodiment 232, wherein said ortholog is an ortholog found in a powdery mildew selected from the powdery mildew genus Golovinomyces including G. orontii and G. cichorachearum (of curcubits), Erysiphe including E. necator (or Uncinula necator) (powdery mildew of grapes), Blumeria including B. graminis f. sp. tritici (causes powdery mildew of wheat), f. sp. hordei (powdery mildew of barley), Microsphaera including M. diffusa (powdery mildew of legumes, e.g. soybean), Leveillula including L. taurica (also known as Oidiopsis taurica) (powdery mildew of onion or artichoke), Podosphaera including P. leucotricha (powdery mildew of apples and pears), P. macularis (powdery mildew of hemp and cannabis), P. xanthii (powdery mildew of cucurbits: cucumbers, squashes (including pumpkins), luffas, melons, and watermelons), and P. pannosa (powdery mildew of roses).

Embodiment 234: The isolated dsRNA of embodiment 232, wherein said ortholog is an ortholog found in a powdery mildew selected from the powdery mildew genus Golovinomyces including G. orontii and G. cichorachearum (of curcubits), Erysiphe including E. necator (or Uncinula necator) (powdery mildew of grapes), Microsphaera including M. diffusa (powdery mildew of legumes, e.g. soybean), Leveillula including L. taurica (also known as Oidiopsis taurica) (powdery mildew of onion or artichoke), Oidium including O. neolycopersici (powdery mildew of tomato), Podosphaera including P. leucotricha (powdery mildew of apples and pears), P. macularis (powdery mildew of hemp and cannabis), P. xanthii (powdery mildew of cucurbits: cucumbers, squashes (including pumpkins), luffas, melons, and watermelons), and P. pannosa (also known as Sphaerotheca pannosa, powdery mildew of roses).

Embodiment 235: The isolated dsRNA of embodiment 232, wherein said ortholog is an ortholog found in a powdery mildew selected from the group consisting of Erysiphe necator (or Uncinula necator) (powdery mildew of grapes), Erysiphe pisi (powdery mildew of pea), Blumeria graminis f. sp. tritici (causes powdery mildew of wheat), f. sp. hordei (powdery mildew of barley), f. sp. avenae (powdery mildew of oats), f. sp. secalis (powdery mildew of rye), Microsphaera diffusa (powdery mildew of legumes, e.g. soybean), Leveillula taurica (also known as Oidiopsis taurica) (powdery mildew of onion or artichoke), Oidium neolycopersici (powdery mildew of tomato), Podosphaera leucotricha (powdery mildew of apples and pears), Podosphaera macularis (powdery mildew of hops, hemp and cannabis), Podosphaera aphanis (powdery mildew of strawberry), Podosphaera fusca and Podosphaera xanthii (powdery mildew of cucurbits: cucumbers, squashes (including pumpkins), luffas, melons, and watermelons), Podosphaera pannosa (powdery mildew of roses), and G. cichoracearum, G. orontii, G. ambrosiae, G. spadiceus (powdery mildews of cucurbits, cannabis, hemp and sunflower).

Embodiment 236: The isolated dsRNA of embodiment 232, wherein said ortholog is an ortholog found in a powdery mildew selected from the group consisting of Erysiphe necator (or Uncinula necator) (powdery mildew of grapes), Blumeria graminis f sp. tritici (causes powdery mildew of wheat), f. sp. hordei (powdery mildew of barley), Microsphaera diffusa (powdery mildew of legumes, e.g. soybean), Leveillula taurica (also known as Oidiopsis taurica) (powdery mildew of onion or artichoke), Podosphaera leucotricha (powdery mildew of apples and pears), and Podosphaera xanthii (powdery mildew of cucurbits: cucumbers, squashes (including pumpkins), luffas, melons, and watermelons).

Embodiment 237: The isolated dsRNA of embodiment 232, wherein said ortholog is an ortholog found in Erysiphe necator (or Uncinula necator).

Embodiment 238: The isolated dsRNA according to any one of embodiments 1-230, wherein said ortholog is an ortholog found in a plant rust.

Embodiment 239: The composition according to any one of embodiment of embodiment 238, wherein said ortholog is an ortholog found in a plant rust selected from the group consisting of Cronartium ribicola (White pine blister rust), Gymnosporangium juniperi-virginianae (Cedar-apple rust), Hemileia vastatrix (Coffee rust), Phakopsora meibomiae and P. pachyrhizi (Soybean rust), Puccinia coronata (Crown Rust of Oats and Ryegrass), Puccinia graminis (Stem rust of wheat and Kentucky bluegrass, or black rust of cereals), Puccinia hemerocallidis (Daylily rust), Puccinia triticina (Brown Wheat Rust), Puccinia sorghi (Common Rust of Corn), Puccinia striiformis (Yellow Rust) of cereals, Uromyces appendiculatus (Bean Rust), Puccinia melanocephala (Brown Rust of Sugarcane), and Puccinia kuehnii (Orange rust of Sugar cane).

Embodiment 240: The isolated dsRNA according to any one of embodiments 1-230, wherein said ortholog is an ortholog found in an ascomycete selected from the group consisting of Botrytis sp. (grey mold), Fusarium spp. (Fusarium wilt disease), Thielaviopsis spp. (canker rot, black root rot, Thielaviopsis root rot), Verticillium spp., Magnaporthe grisea (rice blast), and Sclerotinia sclerotiorum (cottony rot).

Embodiment 241: The isolated dsRNA according to any one of embodiments 1-230, wherein said ortholog is an ortholog found in a Basidiomycete selected from the group consisting of Ustilago spp. (smuts) smut of cereals, Tilletia sp. Rhizoctonia spp., and Armillaria spp. (honey fungus species, virulent pathogens of trees).

Embodiment 242: The isolated dsRNA according to any one of embodiments 1-241, wherein said target gene is not Cytochrome P450 monoxygenase (CYP51) or an ortholog thereof.

Embodiment 243: The isolated dsRNA according to any one of embodiments 1-242, wherein said ortholog shares at least 35% sequence identity, or at least 40% sequence identity, or at least 50% sequence identity, or at least 80% sequence identity, or at least 85% sequence identity, or at least 90% sequence identity, or at least 95% sequence identity with the corresponding G. orontii gene.

Embodiment 244: The isolated dsRNA according to any one of embodiments 1-243, wherein said ortholog shares at least 50% sequence identity, or at least 60% sequence identity, or at least 70% sequence identity, or at least 80% sequence identity, or at least 85% sequence identity, or at least 90% sequence identity, or at least 95% sequence identity with the corresponding G. orontii gene.

Embodiment 245: The isolated dsRNA according to any one of embodiments 1-244, wherein said ortholog encodes a protein that shares at least 35% sequence identity, or at least 40% sequence identity, or at least 50% sequence identity, or at least 80% sequence identity, or at least 85% sequence identity, or at least 90% sequence identity, or at least 95% sequence identity or at least 98% sequence identity with a protein expressed by the corresponding G. orontii gene.

Embodiment 246: The isolated dsRNA according to any one of embodiments 1-245, wherein said ortholog shares at least 50% sequence identity, or at least 60% sequence identity, or at least 70% sequence identity, or at least 80% sequence identity, or at least 85% sequence identity, or at least 90% sequence identity, or at least 95% sequence identity with the corresponding G. orontii gene.

Embodiment 247: The isolated dsRNA according to any one of embodiments 1-246, wherein said ortholog encodes a protein that shares at least 50% sequence identity, or at least 75% sequence identity, or at least 80% sequence identity, or at least 85% sequence identity, or at least 90% sequence identity, or at least 95% sequence identity, or at least 98% sequence identity with a protein expressed by the corresponding G. orontii gene.

Embodiment 248: The isolated dsRNA molecule according to any one of embodiments 1-247, wherein the dsRNA molecule comprises two annealed complementary RNA strands.

Embodiment 249: The isolated dsRNA molecule according to any one of embodiments 1-248, wherein said dsRNA molecule comprises a nucleotide sequence that is complementary to at least 17 contiguous nucleotides of one or more of said target G. orontii genes or target G. orontii gene orthologs, or an RNA transcribed therefrom.

Embodiment 250: The isolated dsRNA molecule according to any one of embodiments 1-248, wherein said dsRNA molecule comprises a long dsRNA, a small inhibitory RNA (siRNA), a small hairpin RNA (shRNA), tasiRNA, or a miRNA.

Embodiment 251: The isolated dsRNA molecule of embodiment 250, wherein said dsRNA comprises a long dsRNA.

Embodiment 252: The isolated dsRNA molecule of embodiment 250, wherein said dsRNA comprises a small inhibitory RNA (siRNA).

Embodiment 253: The isolated dsRNA molecule of embodiment 250, wherein said dsRNA comprises a small hairpin RNA (shRNA).

Embodiment 254: The isolated dsRNA molecule according to any one of embodiments 1-253, wherein said dsRNA molecule comprises a nucleotide sequence that is complementary to about 17 to 21 contiguous nucleotides, or complementary to about 17 to 50 contiguous nucleotides, or complementary to about 50 to 100 contiguous nucleotides, or complementary to about 50 to 250 contiguous nucleotides, or complementary to about 250-600 contiguous nucleotides, or complementary to about 500-1,000 contiguous nucleotides of one or more of said target G. orontii genes or target G. orontii gene orthologs, or an RNA transcribed therefrom.

Embodiment 255: The isolated dsRNA molecule according to any one of embodiments 1-254, wherein said dsRNA molecule comprises a nucleic acid sequence complementary to about 17 to 21 contiguous nucleotides, or complementary to about 17 to 50 contiguous nucleotides, or complementary to about 50 to 100 contiguous nucleotides, or complementary to about 50 to 250 contiguous nucleotides, or complementary to about 250-600 contiguous nucleotides, or complementary to about 500-1,000 contiguous nucleotides of the protein coding region of one or more of said target G. orontii genes or target G. orontii gene orthologs or an RNA transcribed therefrom.

Embodiment 256: The isolated dsRNA molecule of any one of embodiments 1-255, wherein said dsRNA molecule comprises a nucleic acid sequence complementary to about about 17 to 21 contiguous nucleotides, or complementary to about 17 to 50 contiguous nucleotides, or complementary to about 50 to 100 contiguous nucleotides, or complementary to about 50 to 250 contiguous nucleotides, or complementary to about 250-600 contiguous nucleotides, or complementary to about 500-1,000 contiguous nucleotides of the 5′ UTR region or the 3′ UTR region said target G. orontii genes or target G. orontii gene orthologs.

Embodiment 257: The isolated dsRNA molecule of any one of embodiments 1-256, wherein said dsRNA molecule has length ranging from about 17 nt, or from about 21 nt, or from about 50 nt, or from about 100 nu up to about 500 nt, or up to about 400 nt, or up to about 200 nt, or up to about 200 nt, or up to about 150 nt.

Embodiment 258: The isolated dsRNA molecule of embodiment 257, wherein said dsRNA molecule has a length ranging from about 150 nt up to about 500 nt.

Embodiment 259: The isolated dsRNA molecule of any one of embodiments 1-258, wherein said dsRNA molecule comprises a nucleic acid sequence complementary to a contiguous region comprising at least about 0.1%, 0.5%, 1%, 5%, 10%, 25%, 50%, 60%, 70%, 80%, 90%, 95%, 98%, 99%, or 100% of the length of the target gene sequence protein coding region, 5′ UTR region, or 3′ UTR region of said target G. orontii genes or target G. orontii gene orthologs.

Embodiment 260: The isolated dsRNA molecule of any one of embodiments 1-259, wherein the dsRNA molecule comprises a single RNA strand comprising an inversely repeated sequence with a spacer in between and wherein the single RNA strand can anneal to itself to form a hairpin loop structure.

Embodiment 261: The isolated dsRNA molecule of embodiment 260, wherein the dsRNA is about 30 nucleotides or shorter in length.

Embodiment 262: The isolated dsRNA molecule of embodiment 261, wherein said dsRNA ranges in length from about 25-29 nucleotides in length.

Embodiment 263: The isolated dsRNA molecule according to any one of embodiments 260-262, wherein said dsRNA comprises a duplex length ranging from about 17-21 nucleotides or from about 18-23 nucleotides, or from about 19-21 nucleotides.

Embodiment 264: The isolated dsRNA molecule according to any one of embodiments 260-263 wherein said dsRNA comprises a loop sequence ranging from 3 to 9 nucleotides in length or has a loop length of 5, 7, or 9 nucleotides.

Embodiment 265: The isolated dsRNA molecule of any one of embodiments 1-264, wherein said dsRNA is capable of reducing spore count of said phytopathogenic fungus when infecting a plant comprising said dsRNA as compared to a plant without said dsRNA.

Embodiment 266: The isolated dsRNA molecule of any one of embodiments 1-265, wherein said dsRNA is capable of reducing hyphal length of said phytopathogenic fungus when infecting a plant comprising said dsRNA as compared to a plant without said dsRNA.

Embodiment 267: The isolated dsRNA molecule of any one of embodiments 1-266, wherein said dsRNA is capable of reducing germination of said phytopathogenic fungus when infecting a plant comprising said dsRNA as compared to a plant without said dsRNA.

Embodiment 268: The isolated dsRNA molecule of any one of embodiments 1-266, wherein said dsRNA is capable of reducing penetration of the host plant by the pathogen when infecting a plant comprising said dsRNA as compared to a plant without said dsRNA.

Embodiment 269: The isolated dsRNA molecule of any one of embodiments 1-268, wherein said dsRNA comprises a nucleotide sequence selected from the group consisting of SEQ ID NO: 212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, and SEQ ID NO:255.

Embodiment 270: The isolated dsRNA of embodiment 269, wherein said dsRNA comprises the sequence of SEQ ID NO:212.

Embodiment 271: The isolated dsRNA according to any one of embodiments 269, wherein said dsRNA comprises the sequence of SEQ ID NO:213.

Embodiment 272: The isolated dsRNA according to any one of embodiments 269, wherein said dsRNA comprises the sequence of SEQ ID NO:214.

Embodiment 273: The isolated dsRNA according to any one of embodiments 269, wherein said dsRNA comprises the sequence of SEQ ID NO:215.

Embodiment 274: The isolated dsRNA according to any one of embodiments 269, wherein said dsRNA comprises the sequence of SEQ ID NO:216.

Embodiment 275: The isolated dsRNA according to any one of embodiments 269, wherein said dsRNA comprises the sequence of SEQ ID NO:217.

Embodiment 276: The isolated dsRNA according to any one of embodiments 269, wherein said dsRNA comprises the sequence of SEQ ID NO:218.

Embodiment 277: The isolated dsRNA according to any one of embodiments 269, wherein said dsRNA comprises the sequence of SEQ ID NO:219.

Embodiment 278: The isolated dsRNA according to any one of embodiments 269, wherein said dsRNA comprises the sequence of SEQ ID NO:220.

Embodiment 279: The isolated dsRNA according to any one of embodiments 269, wherein said dsRNA comprises the sequence of SEQ ID NO:221.

Embodiment 280: The isolated dsRNA according to any one of embodiments 269, wherein said dsRNA comprises the sequence of SEQ ID NO:222.

Embodiment 281: The isolated dsRNA according to any one of embodiments 269, wherein said dsRNA comprises the sequence of SEQ ID NO:223.

Embodiment 282: The isolated dsRNA according to any one of embodiments 269, wherein said dsRNA comprises the sequence of SEQ ID NO:224.

Embodiment 283: The isolated dsRNA according to any one of embodiments 269, wherein said dsRNA comprises the sequence of SEQ ID NO:225.

Embodiment 284: The isolated dsRNA according to any one of embodiments 269, wherein said dsRNA comprises the sequence of SEQ ID NO:226.

Embodiment 285: The isolated dsRNA according to any one of embodiments 269, wherein said dsRNA comprises the sequence of SEQ ID NO:227.

Embodiment 286: The isolated dsRNA according to any one of embodiments 269, wherein said dsRNA comprises the sequence of SEQ ID NO:228.

Embodiment 287: The isolated dsRNA according to any one of embodiments 269, wherein said dsRNA comprises the sequence of SEQ ID NO:229.

Embodiment 288: The isolated dsRNA according to any one of embodiments 269, wherein said dsRNA comprises the sequence of SEQ ID NO:230.

Embodiment 289: The isolated dsRNA according to any one of embodiments 269, wherein said dsRNA comprises the sequence of SEQ ID NO:231.

Embodiment 290: The isolated dsRNA according to any one of embodiments 269, wherein said dsRNA comprises the sequence of SEQ ID NO:232.

Embodiment 291: The isolated dsRNA according to any one of embodiments 269, wherein said dsRNA comprises the sequence of SEQ ID NO:233.

Embodiment 292: The isolated dsRNA according to any one of embodiments 269, wherein said dsRNA comprises the sequence of SEQ ID NO:234.

Embodiment 293: The isolated dsRNA according to any one of embodiments 269, wherein said dsRNA comprises the sequence of SEQ ID NO:235.

Embodiment 294: The isolated dsRNA according to any one of embodiments 269, wherein said dsRNA comprises the sequence of SEQ ID NO:236.

Embodiment 295: The isolated dsRNA according to any one of embodiments 269, wherein said dsRNA comprises the sequence of SEQ ID NO:237.

Embodiment 296: The isolated dsRNA according to any one of embodiments 269, wherein said dsRNA comprises the sequence of SEQ ID NO:238.

Embodiment 297: The isolated dsRNA according to any one of embodiments 269, wherein said dsRNA comprises the sequence of SEQ ID NO:239.

Embodiment 298: The isolated dsRNA according to any one of embodiments 269, wherein said dsRNA comprises the sequence of SEQ ID NO:240.

Embodiment 299: The isolated dsRNA according to any one of embodiments 269, wherein said dsRNA comprises the sequence of SEQ ID NO:241.

Embodiment 300: The isolated dsRNA according to any one of embodiments 269, wherein said dsRNA comprises the sequence of SEQ ID NO:242.

Embodiment 301: The isolated dsRNA according to any one of embodiments 269, wherein said dsRNA comprises the sequence of SEQ ID NO:243.

Embodiment 302: The isolated dsRNA according to any one of embodiments 269, wherein said dsRNA comprises the sequence of SEQ ID NO:244.

Embodiment 303: The isolated dsRNA according to any one of embodiments 269, wherein said dsRNA comprises the sequence of SEQ ID NO:245.

Embodiment 304: The isolated dsRNA according to any one of embodiments 269, wherein said dsRNA comprises the sequence of SEQ ID NO:246.

Embodiment 305: The isolated dsRNA according to any one of embodiments 269, wherein said dsRNA comprises the sequence of SEQ ID NO:247.

Embodiment 306: The isolated dsRNA according to any one of embodiments 269, wherein said dsRNA comprises the sequence of SEQ ID NO:248.

Embodiment 307: The isolated dsRNA according to any one of embodiments 269, wherein said dsRNA comprises the sequence of SEQ ID NO:249.

Embodiment 308: The isolated dsRNA according to any one of embodiments 269, wherein said dsRNA comprises the sequence of SEQ ID NO:250.

Embodiment 309: The isolated dsRNA according to any one of embodiments 269, wherein said dsRNA comprises the sequence of SEQ ID NO:251.

Embodiment 310: The isolated dsRNA according to any one of embodiments 269, wherein said dsRNA comprises the sequence of SEQ ID NO:252.

Embodiment 311: The isolated dsRNA according to any one of embodiments 269, wherein said dsRNA comprises the sequence of SEQ ID NO:253.

Embodiment 312: The isolated dsRNA according to any one of embodiments 269, wherein said dsRNA comprises the sequence of SEQ ID NO:254.

Embodiment 313: The isolated dsRNA according to any one of embodiments 269, wherein said dsRNA comprises the sequence of SEQ ID NO:255.

Embodiment 314: A composition for controlling or preventing an infection of a plant by a phytopathogenic fungus, said composition comprising one or more of said isolated dsRNA molecules according to any one of embodiments 1-313.

Embodiment 315: The composition of embodiment 314, wherein said composition comprises a dsRNA that down regulates one of said genes or orthologs.

Embodiment 316: The composition of embodiment 314, wherein said composition comprises dsRNA(s) that downregulate 2, or 3, or 4, or 5, or 6, or 7, or 8, or 9, or 10, or more of said genes or orthologs.

Embodiment 317: The composition of embodiment 316, wherein said composition comprises two or more dsRNA(s) that inhibit or are capable of downregulating expression of the two or more target genes independently selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO: 108, SEQ ID NO: 109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO: 133, SEQ ID NO: 134, SEQ ID NO:135, SEQ ID NO:136, SEQ ID NO:137, SEQ ID NO: 138, SEQ ID NO: 139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO: 148, SEQ ID NO: 149, SEQ ID NO:150, SEQ ID NO:151, SEQ ID NO:152, SEQ ID NO: 153, SEQ ID NO: 154, SEQ ID NO:155, SEQ ID NO:156, SEQ ID NO:157, SEQ ID NO: 158, SEQ ID NO: 159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO: 168, SEQ ID NO: 169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO: 178, SEQ ID NO: 179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO: 198, SEQ ID NO: 199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, and SEQ ID NO:255.

Embodiment 318: The composition of embodiment 317, wherein said composition comprises a dsRNA that inhibits or is capable of downregulating expression of the target gene of SEQ ID NO:1 or an orthologue thereof.

Embodiment 319: The composition according to any one of embodiments 317-318, wherein said composition comprises a dsRNA that inhibits or is capable of downregulating expression of the target gene of SEQ ID NO:2 or an orthologue thereof.

Embodiment 320: The composition according to any one of embodiments 317-319, wherein said composition comprises a dsRNA that inhibits or is capable of downregulating expression of the target gene of SEQ ID NO:3 or an orthologue thereof.

Embodiment 321: The composition according to any one of embodiments 317-320, wherein said composition comprises a dsRNA that inhibits or is capable of downregulating expression of the target gene of SEQ ID NO:4 or an orthologue thereof.

Embodiment 322: The composition according to any one of embodiments 317-321, wherein said composition comprises a dsRNA that inhibits or is capable of downregulating expression of the target gene of SEQ ID NO:5 or an orthologue thereof.

Embodiment 323: The composition according to any one of embodiments 317-322, wherein said composition comprises a dsRNA that inhibits or is capable of downregulating expression of the target gene of SEQ ID NO:6 or an orthologue thereof.

Embodiment 324: The composition according to any one of embodiments 317-323, wherein said composition comprises a dsRNA that inhibits or is capable of downregulating expression of the target gene of SEQ ID NO:7 or an orthologue thereof.

Embodiment 325: The composition according to any one of embodiments 317-324, wherein said composition comprises a dsRNA that inhibits or is capable of downregulating expression of the target gene of SEQ ID NO:8 or an orthologue thereof.

Embodiment 326: The composition according to any one of embodiments 317-325, wherein said composition comprises a dsRNA that inhibits or is capable of downregulating expression of the target gene of SEQ ID NO:9 or an orthologue thereof.

Embodiment 327: The composition according to any one of embodiments 317-326, wherein said composition comprises a dsRNA that inhibits or is capable of downregulating expression of the target gene of SEQ ID NO:10 or an orthologue thereof.

Embodiment 328: The composition according to any one of embodiments 317-327, wherein said composition comprises a dsRNA that inhibits or is capable of downregulating expression of the target gene of SEQ ID NO:11 or an orthologue thereof.

Embodiment 329: The composition according to any one of embodiments 317-328, wherein said composition comprises a dsRNA that inhibits or is capable of downregulating expression of the target gene of SEQ ID NO:12 or an orthologue thereof.

Embodiment 330: The composition according to any one of embodiments 317-329, wherein said composition comprises a dsRNA that inhibits or is capable of downregulating expression of the target gene of SEQ ID NO:13 or an orthologue thereof.

Embodiment 331: The composition according to any one of embodiments 317-330, wherein said composition comprises a dsRNA that inhibits or is capable of downregulating expression of the target gene of SEQ ID NO:14 or an orthologue thereof.

Embodiment 332: The composition according to any one of embodiments 317-331, wherein said composition comprises a dsRNA that inhibits or is capable of downregulating expression of the target gene of SEQ ID NO:15 or an orthologue thereof.

Embodiment 333: The composition according to any one of embodiments 317-332, wherein said composition comprises a dsRNA that inhibits or is capable of downregulating expression of the target gene of SEQ ID NO:16 or an orthologue thereof.

Embodiment 334: The composition according to any one of embodiments 317-333, wherein said composition comprises a dsRNA that inhibits or is capable of downregulating expression of the target gene of SEQ ID NO:17 or an orthologue thereof.

Embodiment 335: The composition according to any one of embodiments 317-334, wherein said composition comprises a dsRNA that inhibits or is capable of downregulating expression of the target gene of SEQ ID NO:18 or an orthologue thereof.

Embodiment 336: The composition according to any one of embodiments 317-335, wherein said composition comprises a dsRNA that inhibits or is capable of downregulating expression of the target gene of SEQ ID NO:19 or an orthologue thereof.

Embodiment 337: The composition according to any one of embodiments 317-336, wherein said composition comprises a dsRNA that inhibits or is capable of downregulating expression of the target gene of SEQ ID NO:20 or an orthologue thereof.

Embodiment 338: The composition according to any one of embodiments 317-337, wherein said composition comprises a dsRNA that inhibits or is capable of downregulating expression of the target gene of SEQ ID NO:21 or an orthologue thereof.

Embodiment 339: The composition according to any one of embodiments 317-338, wherein said composition comprises a dsRNA that inhibits or is capable of downregulating expression of the target gene of SEQ ID NO:22 or an orthologue thereof.

Embodiment 340: The composition according to any one of embodiments 317-339, wherein said composition comprises a dsRNA that inhibits or is capable of downregulating expression of the target gene of SEQ ID NO:23 or an orthologue thereof.

Embodiment 341: The composition according to any one of embodiments 317-340, wherein said composition comprises a dsRNA that inhibits or is capable of downregulating expression of the target gene of SEQ ID NO:24 or an orthologue thereof.

Embodiment 342: The composition according to any one of embodiments 317-341, wherein said composition comprises a dsRNA that inhibits or is capable of downregulating expression of the target gene of SEQ ID NO:25 or an orthologue thereof.

Embodiment 343: The composition according to any one of embodiments 317-342, wherein said composition comprises a dsRNA that inhibits or is capable of downregulating expression of the target gene of SEQ ID NO:26 or an orthologue thereof.

Embodiment 344: The composition according to any one of embodiments 317-343, wherein said composition comprises a dsRNA that inhibits or is capable of downregulating expression of the target gene of SEQ ID NO:27 or an orthologue thereof.

Embodiment 345: The composition according to any one of embodiments 317-344, wherein said composition comprises a dsRNA that inhibits or is capable of downregulating expression of the target gene of SEQ ID NO:28 or an orthologue thereof.

Embodiment 346: The composition according to any one of embodiments 317-345, wherein said composition comprises a dsRNA that inhibits or is capable of downregulating expression of the target gene of SEQ ID NO:29 or an orthologue thereof.

Embodiment 347: The composition according to any one of embodiments 317-346, wherein said composition comprises a dsRNA that inhibits or is capable of downregulating expression of the target gene of SEQ ID NO:30 or an orthologue thereof.

Embodiment 348: The composition according to any one of embodiments 317-347, wherein said composition comprises a dsRNA that inhibits or is capable of downregulating expression of the target gene of SEQ ID NO:31 or an orthologue thereof.

Embodiment 349: The composition according to any one of embodiments 317-348, wherein said composition comprises a dsRNA that inhibits or is capable of downregulating expression of the target gene of SEQ ID NO:32 or an orthologue thereof.

Embodiment 350: The composition according to any one of embodiments 317-349, wherein said composition comprises a dsRNA that inhibits or is capable of downregulating expression of the target gene of SEQ ID NO:33 or an orthologue thereof.

Embodiment 351: The composition according to any one of embodiments 317-350, wherein said composition comprises a dsRNA that inhibits or is capable of downregulating expression of the target gene of SEQ ID NO:34 or an orthologue thereof.

Embodiment 352: The composition according to any one of embodiments 317-351, wherein said composition comprises a dsRNA that inhibits or is capable of downregulating expression of the target gene of SEQ ID NO:35 or an orthologue thereof.

Embodiment 353: The composition according to any one of embodiments 317-352, wherein said composition comprises a dsRNA that inhibits or is capable of downregulating expression of the target gene of SEQ ID NO:36 or an orthologue thereof.

Embodiment 354: The composition according to any one of embodiments 317-353, wherein said composition comprises a dsRNA that inhibits or is capable of downregulating expression of the target gene of SEQ ID NO:37 or an orthologue thereof.

Embodiment 355: The composition according to any one of embodiments 317-354, wherein said composition comprises a dsRNA that inhibits or is capable of downregulating expression of the target gene of SEQ ID NO:38 or an orthologue thereof.

Embodiment 356: The composition according to any one of embodiments 317-355, wherein said composition comprises a dsRNA that inhibits or is capable of downregulating expression of the target gene of SEQ ID NO:39 or an orthologue thereof.

Embodiment 357: The composition according to any one of embodiments 317-356, wherein said composition comprises a dsRNA that inhibits or is capable of downregulating expression of the target gene of SEQ ID NO:40 or an orthologue thereof.

Embodiment 358: The composition according to any one of embodiments 317-357, wherein said composition comprises a dsRNA that inhibits or is capable of downregulating expression of the target gene of SEQ ID NO:41 or an orthologue thereof.

Embodiment 359: The composition according to any one of embodiments 317-358, wherein said composition comprises a dsRNA that inhibits or is capable of downregulating expression of the target gene of SEQ ID NO:42 or an orthologue thereof.

Embodiment 360: The composition according to any one of embodiments 317-359, wherein said composition comprises a dsRNA that inhibits or is capable of downregulating expression of the target gene of SEQ ID NO:43 or an orthologue thereof.

Embodiment 361: The composition according to any one of embodiments 317-360, wherein said composition comprises a dsRNA that inhibits or is capable of downregulating expression of the target gene of SEQ ID NO:44 or an orthologue thereof.

Embodiment 362: The composition according to any one of embodiments 317-361, wherein said composition comprises a dsRNA that inhibits or is capable of downregulating expression of the target gene of SEQ ID NO:45 or an orthologue thereof.

Embodiment 363: The composition according to any one of embodiments 317-362, wherein said composition comprises a dsRNA that inhibits or is capable of downregulating expression of the target gene of SEQ ID NO:46 or an orthologue thereof.

Embodiment 364: The composition according to any one of embodiments 317-363, wherein said composition comprises a dsRNA that inhibits or is capable of downregulating expression of the target gene of SEQ ID NO:47 or an orthologue thereof.

Embodiment 365: The composition according to any one of embodiments 317-364, wherein said composition comprises a dsRNA that inhibits or is capable of downregulating expression of the target gene of SEQ ID NO:48 or an orthologue thereof.

Embodiment 366: The composition according to any one of embodiments 317-365, wherein said composition comprises a dsRNA that inhibits or is capable of downregulating expression of the target gene of SEQ ID NO:49 or an orthologue thereof.

Embodiment 367: The composition according to any one of embodiments 317-366, wherein said composition comprises a dsRNA that inhibits or is capable of downregulating expression of the target gene of SEQ ID NO:50 or an orthologue thereof.

Embodiment 368: The composition according to any one of embodiments 317-367, wherein said composition comprises a dsRNA that inhibits or is capable of downregulating expression of the target gene of SEQ ID NO:51 or an orthologue thereof.

Embodiment 369: The composition according to any one of embodiments 317-368, wherein said composition comprises a dsRNA that inhibits or is capable of downregulating expression of the target gene of SEQ ID NO:52 or an orthologue thereof.

Embodiment 370: The composition according to any one of embodiments 317-369, wherein said composition comprises a dsRNA that inhibits or is capable of downregulating expression of the target gene of SEQ ID NO:53 or an orthologue thereof.

Embodiment 371: The composition according to any one of embodiments 317-370, wherein said composition comprises a dsRNA that inhibits or is capable of downregulating expression of the target gene of SEQ ID NO:54 or an orthologue thereof.

Embodiment 372: The composition according to any one of embodiments 317-371, wherein said composition comprises a dsRNA that inhibits or is capable of downregulating expression of the target gene of SEQ ID NO:55 or an orthologue thereof.

Embodiment 373: The composition according to any one of embodiments 317-372, wherein said composition comprises a dsRNA that inhibits or is capable of downregulating expression of the target gene of SEQ ID NO:56 or an orthologue thereof.

Embodiment 374: The composition according to any one of embodiments 317-373, wherein said composition comprises a dsRNA that inhibits or is capable of downregulating expression of the target gene of SEQ ID NO:57 or an orthologue thereof.

Embodiment 375: The composition according to any one of embodiments 317-374, wherein said composition comprises a dsRNA that inhibits or is capable of downregulating expression of the target gene of SEQ ID NO:58 or an orthologue thereof.

Embodiment 376: The composition according to any one of embodiments 317-375, wherein said composition comprises a dsRNA that inhibits or is capable of downregulating expression of the target gene of SEQ ID NO:59 or an orthologue thereof.

Embodiment 377: The composition according to any one of embodiments 317-376, wherein said composition comprises a dsRNA that inhibits or is capable of downregulating expression of the target gene of SEQ ID NO:60 or an orthologue thereof.

Embodiment 378: The composition according to any one of embodiments 317-377, wherein said composition comprises a dsRNA that inhibits or is capable of downregulating expression of the target gene of SEQ ID NO:61 or an orthologue thereof.

Embodiment 379: The composition according to any one of embodiments 317-378, wherein said composition comprises a dsRNA that inhibits or is capable of downregulating expression of the target gene of SEQ ID NO:62 or an orthologue thereof.

Embodiment 380: The composition according to any one of embodiments 317-379, wherein said composition comprises a dsRNA that inhibits or is capable of downregulating expression of the target gene of SEQ ID NO:63 or an orthologue thereof.

Embodiment 381: The composition according to any one of embodiments 317-380, wherein said composition comprises a dsRNA that inhibits or is capable of downregulating expression of the target gene of SEQ ID NO:64 or an orthologue thereof.

Embodiment 382: The composition according to any one of embodiments 317-381, wherein said composition comprises a dsRNA that inhibits or is capable of downregulating expression of the target gene of SEQ ID NO:65 or an orthologue thereof.

Embodiment 383: The composition according to any one of embodiments 317-382, wherein said composition comprises a dsRNA that inhibits or is capable of downregulating expression of the target gene of SEQ ID NO:66 or an orthologue thereof.

Embodiment 384: The composition according to any one of embodiments 317-383, wherein said composition comprises a dsRNA that inhibits or is capable of downregulating expression of the target gene of SEQ ID NO:67 or an orthologue thereof.

Embodiment 385: The composition according to any one of embodiments 317-384, wherein said composition comprises a dsRNA that inhibits or is capable of downregulating expression of the target gene of SEQ ID NO:68 or an orthologue thereof.

Embodiment 386: The composition according to any one of embodiments 317-385, wherein said composition comprises a dsRNA that inhibits or is capable of downregulating expression of the target gene of SEQ ID NO:69 or an orthologue thereof.

Embodiment 387: The composition according to any one of embodiments 317-386, wherein said composition comprises a dsRNA that inhibits or is capable of downregulating expression of the target gene of SEQ ID NO:70 or an orthologue thereof.

Embodiment 388: The composition according to any one of embodiments 317-387, wherein said composition comprises a dsRNA that inhibits or is capable of downregulating expression of the target gene of SEQ ID NO:71 or an orthologue thereof.

Embodiment 389: The composition according to any one of embodiments 317-388, wherein said composition comprises a dsRNA that inhibits or is capable of downregulating expression of the target gene of SEQ ID NO:72 or an orthologue thereof.

Embodiment 390: The composition according to any one of embodiments 317-389, wherein said composition comprises a dsRNA that inhibits or is capable of downregulating expression of the target gene of SEQ ID NO:73 or an orthologue thereof.

Embodiment 391: The composition according to any one of embodiments 317-390, wherein said composition comprises a dsRNA that inhibits or is capable of downregulating expression of the target gene of SEQ ID NO:74 or an orthologue thereof.

Embodiment 392: The composition according to any one of embodiments 317-391, wherein said composition comprises a dsRNA that inhibits or is capable of downregulating expression of the target gene of SEQ ID NO:75 or an orthologue thereof.

Embodiment 393: The composition according to any one of embodiments 317-392, wherein said composition comprises a dsRNA that inhibits or is capable of downregulating expression of the target gene of SEQ ID NO:76 or an orthologue thereof.

Embodiment 394: The composition according to any one of embodiments 317-393, wherein said composition comprises a dsRNA that inhibits or is capable of downregulating expression of the target gene of SEQ ID NO:77 or an orthologue thereof.

Embodiment 395: The composition according to any one of embodiments 317-394, wherein said composition comprises a dsRNA that inhibits or is capable of downregulating expression of the target gene of SEQ ID NO:78 or an orthologue thereof.

Embodiment 396: The composition according to any one of embodiments 317-395, wherein said composition comprises a dsRNA that inhibits or is capable of downregulating expression of the target gene of SEQ ID NO:79 or an orthologue thereof.

Embodiment 397: The composition according to any one of embodiments 317-396, wherein said composition comprises a dsRNA that inhibits or is capable of downregulating expression of the target gene of SEQ ID NO:80 or an orthologue thereof.

Embodiment 398: The composition according to any one of embodiments 317-397, wherein said composition comprises a dsRNA that inhibits or is capable of downregulating expression of the target gene of SEQ ID NO:81 or an orthologue thereof.

Embodiment 399: The composition according to any one of embodiments 317-398, wherein said composition comprises a dsRNA that inhibits or is capable of downregulating expression of the target gene of SEQ ID NO:82 or an orthologue thereof.

Embodiment 400: The composition according to any one of embodiments 317-399, wherein said composition comprises a dsRNA that inhibits or is capable of downregulating expression of the target gene of SEQ ID NO:83 or an orthologue thereof.

Embodiment 401: The composition according to any one of embodiments 317-400, wherein said composition comprises a dsRNA that inhibits or is capable of downregulating expression of the target gene of SEQ ID NO:84 or an orthologue thereof.

Embodiment 402: The composition according to any one of embodiments 317-401, wherein said composition comprises a dsRNA that inhibits or is capable of downregulating expression of the target gene of SEQ ID NO:85 or an orthologue thereof.

Embodiment 403: The composition according to any one of embodiments 317-402, wherein said composition comprises a dsRNA that inhibits or is capable of downregulating expression of the target gene of SEQ ID NO:86 or an orthologue thereof.

Embodiment 404: The composition according to any one of embodiments 317-403, wherein said composition comprises a dsRNA that inhibits or is capable of downregulating expression of the target gene of SEQ ID NO:87 or an orthologue thereof.

Embodiment 405: The composition according to any one of embodiments 317-404, wherein said composition comprises a dsRNA that inhibits or is capable of downregulating expression of the target gene of SEQ ID NO:88 or an orthologue thereof.

Embodiment 406: The composition according to any one of embodiments 317-405, wherein said composition comprises a dsRNA that inhibits or is capable of downregulating expression of the target gene of SEQ ID NO:89 or an orthologue thereof.

Embodiment 407: The composition according to any one of embodiments 317-406, wherein said composition comprises a dsRNA that inhibits or is capable of downregulating expression of the target gene of SEQ ID NO:90 or an orthologue thereof.

Embodiment 408: The composition according to any one of embodiments 317-407, wherein said composition comprises a dsRNA that inhibits or is capable of downregulating expression of the target gene of SEQ ID NO:91 or an orthologue thereof.

Embodiment 409: The composition according to any one of embodiments 317-408, wherein said composition comprises a dsRNA that inhibits or is capable of downregulating expression of the target gene of SEQ ID NO:92 or an orthologue thereof.

Embodiment 410: The composition according to any one of embodiments 317-409, wherein said composition comprises a dsRNA that inhibits or is capable of downregulating expression of the target gene of SEQ ID NO:93 or an orthologue thereof.

Embodiment 411: The composition according to any one of embodiments 317-410, wherein said composition comprises a dsRNA that inhibits or is capable of downregulating expression of the target gene of SEQ ID NO:94 or an orthologue thereof.

Embodiment 412: The composition according to any one of embodiments 317-411, wherein said composition comprises a dsRNA that inhibits or is capable of downregulating expression of the target gene of SEQ ID NO:95 or an orthologue thereof.

Embodiment 413: The composition according to any one of embodiments 317-412, wherein said composition comprises a dsRNA that inhibits or is capable of downregulating expression of the target gene of SEQ ID NO:96 or an orthologue thereof.

Embodiment 414: The composition according to any one of embodiments 317-413, wherein said composition comprises a dsRNA that inhibits or is capable of downregulating expression of the target gene of SEQ ID NO:97 or an orthologue thereof.

Embodiment 415: The composition according to any one of embodiments 317-414, wherein said composition comprises a dsRNA that inhibits or is capable of downregulating expression of the target gene of SEQ ID NO:98 or an orthologue thereof.

Embodiment 416: The composition according to any one of embodiments 317-415, wherein said composition comprises a dsRNA that inhibits or is capable of downregulating expression of the target gene of SEQ ID NO:99 or an orthologue thereof.

Embodiment 417: The composition according to any one of embodiments 317-416, wherein said composition comprises a dsRNA that inhibits or is capable of downregulating expression of the target gene of SEQ ID NO:100 or an orthologue thereof.

Embodiment 418: The composition according to any one of embodiments 317-417, wherein said composition comprises a dsRNA that inhibits or is capable of downregulating expression of the target gene of SEQ ID NO:101 or an orthologue thereof.

Embodiment 419: The composition according to any one of embodiments 317-418, wherein said composition comprises a dsRNA that inhibits or is capable of downregulating expression of the target gene of SEQ ID NO:102 or an orthologue thereof.

Embodiment 420: The composition according to any one of embodiments 317-419, wherein said composition comprises a dsRNA that inhibits or is capable of downregulating expression of the target gene of SEQ ID NO:103 or an orthologue thereof.

Embodiment 421: The composition according to any one of embodiments 317-420, wherein said composition comprises a dsRNA that inhibits or is capable of downregulating expression of the target gene of SEQ ID NO:104 or an orthologue thereof.

Embodiment 422: The composition according to any one of embodiments 317-421, wherein said composition comprises a dsRNA that inhibits or is capable of downregulating expression of the target gene of SEQ ID NO:105 or an orthologue thereof.

Embodiment 423: The composition according to any one of embodiments 317-422, wherein said composition comprises a dsRNA that inhibits or is capable of downregulating expression of the target gene of SEQ ID NO:106 or an orthologue thereof.

Embodiment 424: The composition according to any one of embodiments 317-423, wherein said composition comprises a dsRNA that inhibits or is capable of downregulating expression of the target gene of SEQ ID NO:107 or an orthologue thereof.

Embodiment 425: The composition according to any one of embodiments 317-424, wherein said composition comprises a dsRNA that inhibits or is capable of downregulating expression of the target gene of SEQ ID NO:108 or an orthologue thereof.

Embodiment 426: The composition according to any one of embodiments 317-425, wherein said composition comprises a dsRNA that inhibits or is capable of downregulating expression of the target gene of SEQ ID NO:109 or an orthologue thereof.

Embodiment 427: The composition according to any one of embodiments 317-426, wherein said composition comprises a dsRNA that inhibits or is capable of downregulating expression of the target gene of SEQ ID NO:110 or an orthologue thereof.

Embodiment 428: The composition according to any one of embodiments 317-427, wherein said composition comprises a dsRNA that inhibits or is capable of downregulating expression of the target gene of SEQ ID NO:111 or an orthologue thereof.

Embodiment 429: The composition according to any one of embodiments 317-428, wherein said composition comprises a dsRNA that inhibits or is capable of downregulating expression of the target gene of SEQ ID NO:112 or an orthologue thereof.

Embodiment 430: The composition according to any one of embodiments 317-429, wherein said composition comprises a dsRNA that inhibits or is capable of downregulating expression of the target gene of SEQ ID NO:113 or an orthologue thereof.

Embodiment 431: The composition according to any one of embodiments 317-430, wherein said composition comprises a dsRNA that inhibits or is capable of downregulating expression of the target gene of SEQ ID NO:114 or an orthologue thereof.

Embodiment 432: The composition according to any one of embodiments 317-431, wherein said composition comprises a dsRNA that inhibits or is capable of downregulating expression of the target gene of SEQ ID NO:115 or an orthologue thereof.

Embodiment 433: The composition according to any one of embodiments 317-432, wherein said composition comprises a dsRNA that inhibits or is capable of downregulating expression of the target gene of SEQ ID NO:116 or an orthologue thereof.

Embodiment 434: The composition according to any one of embodiments 317-433, wherein said composition comprises a dsRNA that inhibits or is capable of downregulating expression of the target gene of SEQ ID NO:117 or an orthologue thereof.

Embodiment 435: The composition according to any one of embodiments 317-434, wherein said composition comprises a dsRNA that inhibits or is capable of downregulating expression of the target gene of SEQ ID NO:118 or an orthologue thereof.

Embodiment 436: The composition according to any one of embodiments 317-435, wherein said composition comprises a dsRNA that inhibits or is capable of downregulating expression of the target gene of SEQ ID NO:119 or an orthologue thereof.

Embodiment 437: The composition according to any one of embodiments 317-436, wherein said composition comprises a dsRNA that inhibits or is capable of downregulating expression of the target gene of SEQ ID NO:120 or an orthologue thereof.

Embodiment 438: The composition according to any one of embodiments 317-437, wherein said composition comprises a dsRNA that inhibits or is capable of downregulating expression of the target gene of SEQ ID NO:121 or an orthologue thereof.

Embodiment 439: The composition according to any one of embodiments 317-438, wherein said composition comprises a dsRNA that inhibits or is capable of downregulating expression of the target gene of SEQ ID NO:122 or an orthologue thereof.

Embodiment 440: The composition according to any one of embodiments 317-439, wherein said composition comprises a dsRNA that inhibits or is capable of downregulating expression of the target gene of SEQ ID NO:123 or an orthologue thereof.

Embodiment 441: The composition according to any one of embodiments 317-440, wherein said composition comprises a dsRNA that inhibits or is capable of downregulating expression of the target gene of SEQ ID NO:124 or an orthologue thereof.

Embodiment 442: The composition according to any one of embodiments 317-441, wherein said composition comprises a dsRNA that inhibits or is capable of downregulating expression of the target gene of SEQ ID NO:125 or an orthologue thereof.

Embodiment 443: The composition according to any one of embodiments 317-442, wherein said composition comprises a dsRNA that inhibits or is capable of downregulating expression of the target gene of SEQ ID NO:126 or an orthologue thereof.

Embodiment 444: The composition according to any one of embodiments 317-443, wherein said composition comprises a dsRNA that inhibits or is capable of downregulating expression of the target gene of SEQ ID NO:127 or an orthologue thereof.

Embodiment 445: The composition according to any one of embodiments 317-444, wherein said composition comprises a dsRNA that inhibits or is capable of downregulating expression of the target gene of SEQ ID NO:128 or an orthologue thereof.

Embodiment 446: The composition according to any one of embodiments 317-445, wherein said composition comprises a dsRNA that inhibits or is capable of downregulating expression of the target gene of SEQ ID NO:129 or an orthologue thereof.

Embodiment 447: The composition according to any one of embodiments 317-446, wherein said composition comprises a dsRNA that inhibits or is capable of downregulating expression of the target gene of SEQ ID NO:130 or an orthologue thereof.

Embodiment 448: The composition according to any one of embodiments 317-447, wherein said composition comprises a dsRNA that inhibits or is capable of downregulating expression of the target gene of SEQ ID NO:131 or an orthologue thereof.

Embodiment 449: The composition according to any one of embodiments 317-448, wherein said composition comprises a dsRNA that inhibits or is capable of downregulating expression of the target gene of SEQ ID NO:132 or an orthologue thereof.

Embodiment 450: The composition according to any one of embodiments 317-449, wherein said composition comprises a dsRNA that inhibits or is capable of downregulating expression of the target gene of SEQ ID NO:133 or an orthologue thereof.

Embodiment 451: The composition according to any one of embodiments 317-450, wherein said composition comprises a dsRNA that inhibits or is capable of downregulating expression of the target gene of SEQ ID NO:134 or an orthologue thereof.

Embodiment 452: The composition according to any one of embodiments 317-451, wherein said composition comprises a dsRNA that inhibits or is capable of downregulating expression of the target gene of SEQ ID NO:135 or an orthologue thereof.

Embodiment 453: The composition according to any one of embodiments 317-452, wherein said composition comprises a dsRNA that inhibits or is capable of downregulating expression of the target gene of SEQ ID NO:136 or an orthologue thereof.

Embodiment 454: The composition according to any one of embodiments 317-453, wherein said composition comprises a dsRNA that inhibits or is capable of downregulating expression of the target gene of SEQ ID NO:137 or an orthologue thereof.

Embodiment 455: The composition according to any one of embodiments 317-454, wherein said composition comprises a dsRNA that inhibits or is capable of downregulating expression of the target gene of SEQ ID NO:138 or an orthologue thereof.

Embodiment 456: The composition according to any one of embodiments 317-455, wherein said composition comprises a dsRNA that inhibits or is capable of downregulating expression of the target gene of SEQ ID NO:139 or an orthologue thereof.

Embodiment 457: The composition according to any one of embodiments 317-456, wherein said composition comprises a dsRNA that inhibits or is capable of downregulating expression of the target gene of SEQ ID NO:140 or an orthologue thereof.

Embodiment 458: The composition according to any one of embodiments 317-457, wherein said composition comprises a dsRNA that inhibits or is capable of downregulating expression of the target gene of SEQ ID NO:141 or an orthologue thereof.

Embodiment 459: The composition according to any one of embodiments 317-458, wherein said composition comprises a dsRNA that inhibits or is capable of downregulating expression of the target gene of SEQ ID NO:142 or an orthologue thereof.

Embodiment 460: The composition according to any one of embodiments 317-459, wherein said composition comprises a dsRNA that inhibits or is capable of downregulating expression of the target gene of SEQ ID NO:143 or an orthologue thereof.

Embodiment 461: The composition according to any one of embodiments 317-460, wherein said composition comprises a dsRNA that inhibits or is capable of downregulating expression of the target gene of SEQ ID NO:144 or an orthologue thereof.

Embodiment 462: The composition according to any one of embodiments 317-461, wherein said composition comprises a dsRNA that inhibits or is capable of downregulating expression of the target gene of SEQ ID NO:145 or an orthologue thereof.

Embodiment 463: The composition according to any one of embodiments 317-462, wherein said composition comprises a dsRNA that inhibits or is capable of downregulating expression of the target gene of SEQ ID NO:146 or an orthologue thereof.

Embodiment 464: The composition according to any one of embodiments 317-463, wherein said composition comprises a dsRNA that inhibits or is capable of downregulating expression of the target gene of SEQ ID NO:147 or an orthologue thereof.

Embodiment 465: The composition according to any one of embodiments 317-464, wherein said composition comprises a dsRNA that inhibits or is capable of downregulating expression of the target gene of SEQ ID NO:148 or an orthologue thereof.

Embodiment 466: The composition according to any one of embodiments 317-465, wherein said composition comprises a dsRNA that inhibits or is capable of downregulating expression of the target gene of SEQ ID NO:149 or an orthologue thereof.

Embodiment 467: The composition according to any one of embodiments 317-466, wherein said composition comprises a dsRNA that inhibits or is capable of downregulating expression of the target gene of SEQ ID NO:150 or an orthologue thereof.

Embodiment 468: The composition according to any one of embodiments 317-467, wherein said composition comprises a dsRNA that inhibits or is capable of downregulating expression of the target gene of SEQ ID NO:151 or an orthologue thereof.

Embodiment 469: The composition according to any one of embodiments 317-468, wherein said composition comprises a dsRNA that inhibits or is capable of downregulating expression of the target gene of SEQ ID NO:152 or an orthologue thereof.

Embodiment 470: The composition according to any one of embodiments 317-469, wherein said composition comprises a dsRNA that inhibits or is capable of downregulating expression of the target gene of SEQ ID NO:153 or an orthologue thereof.

Embodiment 471: The composition according to any one of embodiments 317-470, wherein said composition comprises a dsRNA that inhibits or is capable of downregulating expression of the target gene of SEQ ID NO:154 or an orthologue thereof.

Embodiment 472: The composition according to any one of embodiments 317-471, wherein said composition comprises a dsRNA that inhibits or is capable of downregulating expression of the target gene of SEQ ID NO:155 or an orthologue thereof.

Embodiment 473: The composition according to any one of embodiments 317-472, wherein said composition comprises a dsRNA that inhibits or is capable of downregulating expression of the target gene of SEQ ID NO:156 or an orthologue thereof.

Embodiment 474: The composition according to any one of embodiments 317-473, wherein said composition comprises a dsRNA that inhibits or is capable of downregulating expression of the target gene of SEQ ID NO:157 or an orthologue thereof.

Embodiment 475: The composition according to any one of embodiments 317-474, wherein said composition comprises a dsRNA that inhibits or is capable of downregulating expression of the target gene of SEQ ID NO:158 or an orthologue thereof.

Embodiment 476: The composition according to any one of embodiments 317-475, wherein said composition comprises a dsRNA that inhibits or is capable of downregulating expression of the target gene of SEQ ID NO:159 or an orthologue thereof.

Embodiment 477: The composition according to any one of embodiments 317-476, wherein said composition comprises a dsRNA that inhibits or is capable of downregulating expression of the target gene of SEQ ID NO:160 or an orthologue thereof.

Embodiment 478: The composition according to any one of embodiments 317-477, wherein said composition comprises a dsRNA that inhibits or is capable of downregulating expression of the target gene of SEQ ID NO:161 or an orthologue thereof.

Embodiment 479: The composition according to any one of embodiments 317-478, wherein said composition comprises a dsRNA that inhibits or is capable of downregulating expression of the target gene of SEQ ID NO:162 or an orthologue thereof.

Embodiment 480: The composition according to any one of embodiments 317-479, wherein said composition comprises a dsRNA that inhibits or is capable of downregulating expression of the target gene of SEQ ID NO:163 or an orthologue thereof.

Embodiment 481: The composition according to any one of embodiments 317-480, wherein said composition comprises a dsRNA that inhibits or is capable of downregulating expression of the target gene of SEQ ID NO:164 or an orthologue thereof.

Embodiment 482: The composition according to any one of embodiments 317-481, wherein said composition comprises a dsRNA that inhibits or is capable of downregulating expression of the target gene of SEQ ID NO:165 or an orthologue thereof.

Embodiment 483: The composition according to any one of embodiments 317-482, wherein said composition comprises a dsRNA that inhibits or is capable of downregulating expression of the target gene of SEQ ID NO:166 or an orthologue thereof.

Embodiment 484: The composition according to any one of embodiments 317-483, wherein said composition comprises a dsRNA that inhibits or is capable of downregulating expression of the target gene of SEQ ID NO:167 or an orthologue thereof.

Embodiment 485: The composition according to any one of embodiments 317-484, wherein said composition comprises a dsRNA that inhibits or is capable of downregulating expression of the target gene of SEQ ID NO:168 or an orthologue thereof.

Embodiment 486: The composition according to any one of embodiments 317-485, wherein said composition comprises a dsRNA that inhibits or is capable of downregulating expression of the target gene of SEQ ID NO:169 or an orthologue thereof.

Embodiment 487: The composition according to any one of embodiments 317-486, wherein said composition comprises a dsRNA that inhibits or is capable of downregulating expression of the target gene of SEQ ID NO:170 or an orthologue thereof.

Embodiment 488: The composition according to any one of embodiments 317-487, wherein said composition comprises a dsRNA that inhibits or is capable of downregulating expression of the target gene of SEQ ID NO:171 or an orthologue thereof.

Embodiment 489: The composition according to any one of embodiments 317-488, wherein said composition comprises a dsRNA that inhibits or is capable of downregulating expression of the target gene of SEQ ID NO:172 or an orthologue thereof.

Embodiment 490: The composition according to any one of embodiments 317-489, wherein said composition comprises a dsRNA that inhibits or is capable of downregulating expression of the target gene of SEQ ID NO:173 or an orthologue thereof.

Embodiment 491: The composition according to any one of embodiments 317-490, wherein said composition comprises a dsRNA that inhibits or is capable of downregulating expression of the target gene of SEQ ID NO:174 or an orthologue thereof.

Embodiment 492: The composition according to any one of embodiments 317-491, wherein said composition comprises a dsRNA that inhibits or is capable of downregulating expression of the target gene of SEQ ID NO:175 or an orthologue thereof.

Embodiment 493: The composition according to any one of embodiments 317-492, wherein said composition comprises a dsRNA that inhibits or is capable of downregulating expression of the target gene of SEQ ID NO:176 or an orthologue thereof.

Embodiment 494: The composition according to any one of embodiments 317-493, wherein said composition comprises a dsRNA that inhibits or is capable of downregulating expression of the target gene of SEQ ID NO:177 or an orthologue thereof.

Embodiment 495: The composition according to any one of embodiments 317-494, wherein said composition comprises a dsRNA that inhibits or is capable of downregulating expression of the target gene of SEQ ID NO:178 or an orthologue thereof.

Embodiment 496: The composition according to any one of embodiments 317-495, wherein said composition comprises a dsRNA that inhibits or is capable of downregulating expression of the target gene of SEQ ID NO:179 or an orthologue thereof.

Embodiment 497: The composition according to any one of embodiments 317-496, wherein said composition comprises a dsRNA that inhibits or is capable of downregulating expression of the target gene of SEQ ID NO:180 or an orthologue thereof.

Embodiment 498: The composition according to any one of embodiments 317-497, wherein said composition comprises a dsRNA that inhibits or is capable of downregulating expression of the target gene of SEQ ID NO:181 or an orthologue thereof.

Embodiment 499: The composition according to any one of embodiments 317-498, wherein said composition comprises a dsRNA that inhibits or is capable of downregulating expression of the target gene of SEQ ID NO:182 or an orthologue thereof.

Embodiment 500: The composition according to any one of embodiments 317-499, wherein said composition comprises a dsRNA that inhibits or is capable of downregulating expression of the target gene of SEQ ID NO:183 or an orthologue thereof.

Embodiment 501: The composition according to any one of embodiments 317-500, wherein said composition comprises a dsRNA that inhibits or is capable of downregulating expression of the target gene of SEQ ID NO:184 or an orthologue thereof.

Embodiment 502: The composition according to any one of embodiments 317-501, wherein said composition comprises a dsRNA that inhibits or is capable of downregulating expression of the target gene of SEQ ID NO:185 or an orthologue thereof.

Embodiment 503: The composition according to any one of embodiments 317-502, wherein said composition comprises a dsRNA that inhibits or is capable of downregulating expression of the target gene of SEQ ID NO:186 or an orthologue thereof.

Embodiment 504: The composition according to any one of embodiments 317-503, wherein said composition comprises a dsRNA that inhibits or is capable of downregulating expression of the target gene of SEQ ID NO:187 or an orthologue thereof.

Embodiment 505: The composition according to any one of embodiments 317-504, wherein said composition comprises a dsRNA that inhibits or is capable of downregulating expression of the target gene of SEQ ID NO:188 or an orthologue thereof.

Embodiment 506: The composition according to any one of embodiments 317-505, wherein said composition comprises a dsRNA that inhibits or is capable of downregulating expression of the target gene of SEQ ID NO:189 or an orthologue thereof.

Embodiment 507: The composition according to any one of embodiments 317-506, wherein said composition comprises a dsRNA that inhibits or is capable of downregulating expression of the target gene of SEQ ID NO:190 or an orthologue thereof.

Embodiment 508: The composition according to any one of embodiments 317-507, wherein said composition comprises a dsRNA that inhibits or is capable of downregulating expression of the target gene of SEQ ID NO:191 or an orthologue thereof.

Embodiment 509: The composition according to any one of embodiments 317-508, wherein said composition comprises a dsRNA that inhibits or is capable of downregulating expression of the target gene of SEQ ID NO:192 or an orthologue thereof.

Embodiment 510: The composition according to any one of embodiments 317-509, wherein said composition comprises a dsRNA that inhibits or is capable of downregulating expression of the target gene of SEQ ID NO:193 or an orthologue thereof.

Embodiment 511: The composition according to any one of embodiments 317-510, wherein said composition comprises a dsRNA that inhibits or is capable of downregulating expression of the target gene of SEQ ID NO:194 or an orthologue thereof.

Embodiment 512: The composition according to any one of embodiments 317-511, wherein said composition comprises a dsRNA that inhibits or is capable of downregulating expression of the target gene of SEQ ID NO:195 or an orthologue thereof.

Embodiment 513: The composition according to any one of embodiments 317-512, wherein said composition comprises a dsRNA that inhibits or is capable of downregulating expression of the target gene of SEQ ID NO:196 or an orthologue thereof.

Embodiment 514: The composition according to any one of embodiments 317-513, wherein said composition comprises a dsRNA that inhibits or is capable of downregulating expression of the target gene of SEQ ID NO:197 or an orthologue thereof.

Embodiment 515: The composition according to any one of embodiments 317-514, wherein said composition comprises a dsRNA that inhibits or is capable of downregulating expression of the target gene of SEQ ID NO:198 or an orthologue thereof.

Embodiment 516: The composition according to any one of embodiments 317-515, wherein said composition comprises a dsRNA that inhibits or is capable of downregulating expression of the target gene of SEQ ID NO:199 or an orthologue thereof.

Embodiment 517: The composition according to any one of embodiments 317-516, wherein said composition comprises a dsRNA that inhibits or is capable of downregulating expression of the target gene of SEQ ID NO:200 or an orthologue thereof.

Embodiment 518: The composition according to any one of embodiments 317-517, wherein said composition comprises a dsRNA that inhibits or is capable of downregulating expression of the target gene of SEQ ID NO:201 or an orthologue thereof.

Embodiment 519: The composition according to any one of embodiments 317-518, wherein said composition comprises a dsRNA that inhibits or is capable of downregulating expression of the target gene of SEQ ID NO:202 or an orthologue thereof.

Embodiment 520: The composition according to any one of embodiments 317-519, wherein said composition comprises a dsRNA that inhibits or is capable of downregulating expression of the target gene of SEQ ID NO:203 or an orthologue thereof.

Embodiment 521: The composition according to any one of embodiments 317-520, wherein said composition comprises a dsRNA that inhibits or is capable of downregulating expression of the target gene of SEQ ID NO:204 or an orthologue thereof.

Embodiment 522: The composition according to any one of embodiments 317-521, wherein said composition comprises a dsRNA that inhibits or is capable of downregulating expression of the target gene of SEQ ID NO:205 or an orthologue thereof.

Embodiment 523: The composition according to any one of embodiments 317-522, wherein said composition comprises a dsRNA that inhibits or is capable of downregulating expression of the target gene of SEQ ID NO:206 or an orthologue thereof.

Embodiment 524: The composition according to any one of embodiments 317-523, wherein said composition comprises a dsRNA that inhibits or is capable of downregulating expression of the target gene of SEQ ID NO:207 or an orthologue thereof.

Embodiment 525: The composition according to any one of embodiments 317-524, wherein said composition comprises a dsRNA that inhibits or is capable of downregulating expression of the target gene of SEQ ID NO:208 or an orthologue thereof.

Embodiment 526: The composition according to any one of embodiments 317-525, wherein said composition comprises a dsRNA that inhibits or is capable of downregulating expression of the target gene of SEQ ID NO:209 or an orthologue thereof.

Embodiment 527: The composition according to any one of embodiments 317-526, wherein said composition comprises a dsRNA that inhibits or is capable of downregulating expression of the target gene of SEQ ID NO:210 or an orthologue thereof.

Embodiment 528: The composition according to any one of embodiments 317-527, wherein said composition comprises a dsRNA that inhibits or is capable of downregulating expression of the target gene of SEQ ID NO:211 or an orthologue thereof.

Embodiment 529: The composition of embodiment 314, wherein said composition comprises two or more dsRNAs comprising nucleotide sequences corresponding to (encoded by) a sequences independently elected from the group consisting of SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO: and SEQ ID NO:255.

Embodiment 530: The composition of embodiment 529, wherein said dsRNA comprises a nucleotide sequence corresponding to (encoded by) the sequence of SEQ ID NO:212.

Embodiment 531: The composition according to any one of embodiments 529-530, wherein said dsRNA comprises a nucleotide sequence corresponding to (encoded by) the sequence of SEQ ID NO:213.

Embodiment 532: The composition according to any one of embodiments 529-531, wherein said dsRNA comprises a nucleotide sequence corresponding to (encoded by) the sequence of SEQ ID NO:214.

Embodiment 533: The composition according to any one of embodiments 529-532, wherein said dsRNA comprises a nucleotide sequence corresponding to (encoded by) the sequence of SEQ ID NO:215.

Embodiment 534: The composition according to any one of embodiments 529-533, wherein said dsRNA comprises a nucleotide sequence corresponding to (encoded by) the sequence of SEQ ID NO:216.

Embodiment 535: The composition according to any one of embodiments 529-534, wherein said dsRNA comprises a nucleotide sequence corresponding to (encoded by) the sequence of SEQ ID NO:217.

Embodiment 536: The composition according to any one of embodiments 529-535, wherein said dsRNA comprises a nucleotide sequence corresponding to (encoded by) the sequence of SEQ ID NO:218.

Embodiment 537: The composition according to any one of embodiments 529-536, wherein said dsRNA comprises a nucleotide sequence corresponding to (encoded by) the sequence of SEQ ID NO:219.

Embodiment 538: The composition according to any one of embodiments 529-537, wherein said dsRNA comprises a nucleotide sequence corresponding to (encoded by) the sequence of SEQ ID NO:220.

Embodiment 539: The composition according to any one of embodiments 529-538, wherein said dsRNA comprises a nucleotide sequence corresponding to (encoded by) the sequence of SEQ ID NO:221.

Embodiment 540: The composition according to any one of embodiments 529-539, wherein said dsRNA comprises a nucleotide sequence corresponding to (encoded by) the sequence of SEQ ID NO:222.

Embodiment 541: The composition according to any one of embodiments 529-540, wherein said dsRNA comprises a nucleotide sequence corresponding to (encoded by) the sequence of SEQ ID NO:223.

Embodiment 542: The composition according to any one of embodiments 529-541, wherein said dsRNA comprises a nucleotide sequence corresponding to (encoded by) the sequence of SEQ ID NO:224.

Embodiment 543: The composition according to any one of embodiments 529-542, wherein said dsRNA comprises a nucleotide sequence corresponding to (encoded by) the sequence of SEQ ID NO:225.

Embodiment 544: The composition according to any one of embodiments 529-543, wherein said dsRNA comprises a nucleotide sequence corresponding to (encoded by) the sequence of SEQ ID NO:226.

Embodiment 545: The composition according to any one of embodiments 529-544, wherein said dsRNA comprises a nucleotide sequence corresponding to (encoded by) the sequence of SEQ ID NO:227.

Embodiment 546: The composition according to any one of embodiments 529-545, wherein said dsRNA comprises a nucleotide sequence corresponding to (encoded by) the sequence of SEQ ID NO:228.

Embodiment 547: The composition according to any one of embodiments 529-546, wherein said dsRNA comprises a nucleotide sequence corresponding to (encoded by) the sequence of SEQ ID NO:229.

Embodiment 548: The composition according to any one of embodiments 529-547, wherein said dsRNA comprises a nucleotide sequence corresponding to (encoded by) the sequence of SEQ ID NO:230.

Embodiment 549: The composition according to any one of embodiments 529-548, wherein said dsRNA comprises a nucleotide sequence corresponding to (encoded by) the sequence of SEQ ID NO:231.

Embodiment 550: The composition according to any one of embodiments 529-549, wherein said dsRNA comprises a nucleotide sequence corresponding to (encoded by) the sequence of SEQ ID NO:232.

Embodiment 551: The composition according to any one of embodiments 529-550, wherein said dsRNA comprises a nucleotide sequence corresponding to (encoded by) the sequence of SEQ ID NO:233.

Embodiment 552: The composition according to any one of embodiments 529-551, wherein said dsRNA comprises a nucleotide sequence corresponding to (encoded by) the sequence of SEQ ID NO:234.

Embodiment 553: The composition according to any one of embodiments 529-552, wherein said dsRNA comprises a nucleotide sequence corresponding to (encoded by) the sequence of SEQ ID NO:235.

Embodiment 554: The composition according to any one of embodiments 529-553, wherein said dsRNA comprises a nucleotide sequence corresponding to (encoded by) the sequence of SEQ ID NO:236.

Embodiment 555: The composition according to any one of embodiments 529-554, wherein said dsRNA comprises a nucleotide sequence corresponding to (encoded by) the sequence of SEQ ID NO:237.

Embodiment 556: The composition according to any one of embodiments 529-555, wherein said dsRNA comprises a nucleotide sequence corresponding to (encoded by) the sequence of SEQ ID NO:238.

Embodiment 557: The composition according to any one of embodiments 529-556, wherein said dsRNA comprises a nucleotide sequence corresponding to (encoded by) the sequence of SEQ ID NO:239.

Embodiment 558: The composition according to any one of embodiments 529-557, wherein said dsRNA comprises a nucleotide sequence corresponding to (encoded by) the sequence of SEQ ID NO:240.

Embodiment 559: The composition according to any one of embodiments 529-558, wherein said dsRNA comprises a nucleotide sequence corresponding to (encoded by) the sequence of SEQ ID NO:241.

Embodiment 560: The composition according to any one of embodiments 529-559, wherein said dsRNA comprises a nucleotide sequence corresponding to (encoded by) the sequence of SEQ ID NO:242.

Embodiment 561: The composition according to any one of embodiments 529-560, wherein said dsRNA comprises a nucleotide sequence corresponding to (encoded by) the sequence of SEQ ID NO:243.

Embodiment 562: The composition according to any one of embodiments 529-561, wherein said dsRNA comprises a nucleotide sequence corresponding to (encoded by) the sequence of SEQ ID NO:244.

Embodiment 563: The composition according to any one of embodiments 529-562, wherein said dsRNA comprises a nucleotide sequence corresponding to (encoded by) the sequence of SEQ ID NO:245.

Embodiment 564: The composition according to any one of embodiments 529-563, wherein said dsRNA comprises a nucleotide sequence corresponding to (encoded by) the sequence of SEQ ID NO:246.

Embodiment 565: The composition according to any one of embodiments 529-564, wherein said dsRNA comprises a nucleotide sequence corresponding to (encoded by) the sequence of SEQ ID NO:247.

Embodiment 566: The composition according to any one of embodiments 529-565, wherein said dsRNA comprises a nucleotide sequence corresponding to (encoded by) the sequence of SEQ ID NO:248.

Embodiment 567: The composition according to any one of embodiments 529-566, wherein said dsRNA comprises a nucleotide sequence corresponding to (encoded by) the sequence of SEQ ID NO:249.

Embodiment 568: The composition according to any one of embodiments 529-567, wherein said dsRNA comprises a nucleotide sequence corresponding to (encoded by) the sequence of SEQ ID NO:250.

Embodiment 569: The composition according to any one of embodiments 529-568, wherein said dsRNA comprises a nucleotide sequence corresponding to (encoded by) the sequence of SEQ ID NO:251.

Embodiment 570: The composition according to any one of embodiments 529-569, wherein said dsRNA comprises a nucleotide sequence corresponding to (encoded by) the sequence of SEQ ID NO:252.

Embodiment 571: The composition according to any one of embodiments 529-570, wherein said dsRNA comprises a nucleotide sequence corresponding to (encoded by) the sequence of SEQ ID NO:253.

Embodiment 572: The composition according to any one of embodiments 529-571, wherein said dsRNA comprises a nucleotide sequence corresponding to (encoded by) the sequence of SEQ ID NO:254.

Embodiment 573: The composition according to any one of embodiments 529-572, wherein said dsRNA comprises a nucleotide sequence corresponding to (encoded by) the sequence of SEQ ID NO:255.

Embodiment 574: The composition according to any one of embodiments 314-573, wherein said composition is formulated for spray on application to a plant.

Embodiment 575: The composition according to any one of embodiments 314-574, wherein said composition is provided as a spray, a drench, granules, a seed coating, inoculation in plant growth medium, as a mist or fog, injection in stem or other plant parts or as a plant-incorporated protectant.

Embodiment 576: The composition of embodiment 575, wherein said composition is provided as a spray, a drenche, granules, a seed coating, or a plant-incorporated protectant.

Embodiment 577: The composition according to any one of embodiments 314-574, wherein said composition is formulated for use in drip irrigation.

Embodiment 578: The composition according to any one of embodiments 314-576, wherein said composition comprises said dsRNA(s) associated with a particle or nanoparticle.

Embodiment 579: The composition of embodiment 578, wherein said particle or nanoparticle comprise a clay or polymer.

Embodiment 580: The composition of embodiment 579, wherein said polymer is an inorganic or organic polymer.

Embodiment 581: The composition of embodiment 582, wherein the organic polymer is a chitosan or chitosan-derived polymer.

Embodiment 582: The composition according to any one of embodiments 314-576, wherein said composition comprises said dsRNA(s) incorporated into a lipid or liposome.

Embodiment 583: The composition according to any one of embodiments 314-576, wherein said composition comprises said dsRNA(s) are incorporated into an anucleated cell.

Embodiment 584: The composition according to any one of embodiments 314-576, wherein said composition is provided as a component of a microbial cell or microbial fermentation product.

Embodiment 585: The composition according to any one of embodiments 314-584, wherein said composition further comprises at least one additive from the group consisting of adjuvants, attractants, growth-regulating substances, insect feed, pheromones, proteins, carbohydrates, polymers, organic compounds, biologics, biostimulants, other biological or synthetic pesticidal agents, and other biological or synthetic growth-regulating agents.

Embodiment 586: The composition according to any one of embodiments 314-576, wherein said dsRNA(s) comprise a lyophilized powder.

Embodiment 587: The composition according to any one of embodiments 314-576, wherein said dsRNA(s) comprise a solution or suspension.

Embodiment 588: The composition according to any one of embodiments 314-576, wherein said dsRNA(s) is formulated for injection into a part of a host plant.

Embodiment 589: The composition according to any one of embodiments 314-590, wherein the concentration of dsRNA in said composition ranges from about 1 μg/mL, or from about 5 μg/mL, or from about 10 μg/mL, or from about 15 μg/mL, or from about 20 μg/mL, or from about 25 μg/mL up to about 500 μg/mL, or up to about 400 μg/mL, or up to about 300 μg/mL, or up to about 200 μg/mL, or up to about 100 μg/mL, or up to about 80 μg/mL.

Embodiment 590: The composition of embodiment 589, wherein the concentration of dsRNA in said composition ranges from about 10 μg/mL up to about 80 μg/mL.

Embodiment 591: A transgenic plant or a part thereof comprising a transgene, wherein the transgene comprises a DNA that expresses a double stranded RNA (dsRNA) according to any one of embodiments 1-313.

Embodiment 592: The transgenic plant or a part thereof of embodiment 591, wherein said dsRNA is expressed at a level that reduces spore count of said phytopathogenic fungus compared to a plant without said DNA.

Embodiment 593: The transgenic plant or a part thereof according to any one of embodiments 591-592, wherein said dsRNA is expressed at a level that reduces hyphal length of said phytopathogenic fungus as compared to a plant without said DNA.

Embodiment 594: The transgenic plant or a part thereof according to any one of embodiments 591-592, wherein said dsRNA is expressed at a level that reduces germination of said phytopathogenic fungus as compared to a plant without said DNA.

Embodiment 595: The transgenic plant or a part thereof according to any one of embodiments 591-592, wherein said dsRNA is expressed at a level that reduces penetration of host plant by said phytopathogenic fungus as compared to a plant without said DNA.

Embodiment 596: The transgenic plant or a part thereof according to any one of embodiments 591-595, wherein the transgene comprises an expression cassette comprising the DNA.

Embodiment 597: The transgenic plant or the part thereof of embodiment 596, wherein the transgene is stably integrated into the genome of the transgenic plant or the part thereof, or wherein the transgene is present on a vector in the transgenic plant or the part thereof.

Embodiment 598: The transgenic plant or the part thereof according to any one of embodiments 596-597, wherein the expression of the dsRNA(s) is controlled by a promoter.

Embodiment 599: The transgenic plant or the part thereof according to any one of embodiments 591-598, wherein said transgenic plant or part thereof comprises a plant or part of a plant selected from the group consisting of grape, wheat, barley, legumes, e.g. soybean, onion, artichoke, apples, peaches, pears, cucurbits, cucumbers, squashes (including pumpkins), luffas, melons, watermelons, hemp, cannabis, roses, strawberry, hops, coffee, oats, ryegrass, a cereal, rice, corn, and sugar cane.

Embodiment 600: The transgenic plant or the part thereof according to any one of embodiments 591-598, wherein said transgenic plant or part thereof comprises a plant or part of a plant selected from the group consisting of grape, wheat, barley, legumes, e.g. soybean), onion, artichoke, apples, peaches, pears, cucumbers, squashes (including pumpkins), luffas, melons, watermelons, coffee rust, oats, ryegrass, a cereal, corn, and sugar cane.

Embodiment 601: The transgenic plant or the part thereof of embodiment 599, wherein said transgenic plant or part thereof comprises a grape plant or a grape plant part.

Embodiment 602: The transgenic plant or the part thereof according to any one of embodiments 591-601, wherein said plant or part thereof comprises a plant.

Embodiment 603: The transgenic plant or the part thereof according to any one of embodiments 591-601, wherein said plant or part thereof comprise seed.

Embodiment 604: A DNA encoding double stranded RNA (dsRNA) according to any one of embodiments 1-313.

Embodiment 605: An expression cassette comprising the DNA of embodiment 604.

Embodiment 606: A vector comprising (a) the DNA of embodiment 604, or (b) an expression cassette comprising the DNA.

Embodiment 607: A method of producing the transgenic plant or the part thereof according to any one of embodiments 591-603, said method comprising:
introducing into at least a cell of the plant:
   (a) a DNA of embodiment 604; or
   (b) a dsRNA according to any one of embodiments 1-313; and regenerating the transgenic plant from the at least one cell.

Embodiment 608: A method of conferring phytopathogenic fungal resistance to a plant or the part thereof, said method comprising:
introducing into the plant or the part thereof:
   (a) a DNA of embodiment 604;
   (b) an expression cassette comprising the DNA of embodiment 604, or
   (c) a dsRNA according to any one of embodiments 1-313.

Embodiment 609: A method for controlling or preventing an infection of a plant by a phytopathogenic fungus, said method comprising:
(a) contacting said plant and/or said pathogenic fungus with an isolated double-stranded RNA according to any one of embodiments 1-313; or
(b) expressing in said plant at least one isolated double-stranded RNA according to any one of embodiments 1-313.

Embodiment 610: The method of embodiment 609, wherein said applying contacting comprises spraying, dunking, coating, watering, or irrigating said plant with a composition according to any one of embodiments 314-587.

Embodiment 611: The method according to any one of embodiments 609-610, wherein said contacting is before infection by said phytopathogenic fungus.

Embodiment 612: The method according to any one of embodiments 609-610, wherein said contacting is during infection by said phytopathogenic fungus.

Embodiment 613: The method according to any one of embodiments 609-612, wherein said contacting comprises applying said dsRNA to a plant selected from the group consisting of grape, wheat, barley, legumes, e.g. soybean, onion, artichoke, apples, peaches, pears, cucurbits, cucumbers, squashes (including pumpkins), luffas, melons, watermelons, hemp, cannabis, roses, strawberry, hops, coffee, oats, ryegrass, a cereal, rice, corn, and sugar cane.

Embodiment 614: The method according to any one of embodiments 609-612, wherein said contacting comprises applying said dsRNA to a plant selected from the group consisting of grape, wheat, barley, legumes, onion, artichoke, apples, peaches, pears, cucumbers, squashes (including pumpkins), luffas, melons, watermelons, coffee rust, oats, ryegrass, a cereal, corn, and sugar cane.

Embodiment 615: The method of embodiment 609, wherein said expressing in said plant at least one isolated double-stranded RNA comprises providing a transgenic plant according to any one of embodiments 591-603.

Embodiment 616: The method of embodiment 615, wherein said transgenic comprises a plant selected from the group consisting of grape, wheat, barley, legumes, e.g., soybean, onion, artichoke, apples, peaches, pears, cucurbits, cucumbers, squashes (including pumpkins), luffas, melons, watermelons, hemp, cannabis, roses, strawberry, hops, coffee, oats, ryegrass, a cereal, rice, corn, and sugar cane.

Embodiment 617: The method of embodiment 615, wherein said transgenic comprises a plant selected from the group consisting of grape, wheat, barley, legumes, onion, artichoke, apples, peaches, pears, cucumbers, squashes (including pumpkins), luffas, melons, watermelons, coffee, oats, ryegrass, a cereal, corn, and sugar cane.

Embodiment 618: The method of embodiment 613, wherein said plant comprises a grape plant.

Embodiment 619: The method of embodiment 616, wherein said transgenic plant comprises a grape plant.

Embodiment 620: The method according to any one of embodiments 618-619, wherein said phytopathogenic fungus comprises Erysiphe necator.

Embodiment 621: A method of identifying genes in a phytopathogen that when downregulated in said phytopathogen inhibit infection of a host plant by said phytopathogen, said method comprising:
selecting target gene(s) in the phytopathogen that have ortholog(s) in a reference powdery mildew;
designing long dsRNA or siRNA to inhibit the reference powdery mildew orthologues with minimal off-targets in the host plant;
inoculating said host plant with said reference powdery mildew;
contacting said host plant with said dsRNA and/or siRNA; and
determining growth and/or reproduction of said reference powdery mildew in said host plant, where reduced growth or reproduction of said reference powdery mildew compared to an untreated plant of the same species indicates that the target genes are phytopathogen genes whose downregulation inhibits infection of said host plant by said phytopathogen.

Embodiment 622: The method of embodiment 621, wherein said method comprises determining growth and/or reproduction of phytopathogen in said host plant, where reduced growth or reproduction of said phytopathogen compared to an untreated plant of the same species indicates that the target genes are phytopathogen genes whose downregulation inhibits infection of said host plant by said phytopathogen.

Embodiment 623: The method according to any one of embodiments 621-622, wherein said host plant comprises a grape plant.

Embodiment 624: The method according to any one of embodiments 621-623, wherein said host plant is Aripsopsis spp.

Embodiment 625: The method according to any one of embodiments 621-624, wherein said reference powdery mildew is a powdery mildew selected from the group consisting of G. orontii, Erysiphe necator (or Uncinula necator) (powdery mildew of grapes), Erysiphe pisi (powdery mildew of pea), Blumeria graminis f. sp. tritici (causes powdery mildew of wheat), f. sp. hordei (powdery mildew of barley), Microsphaera diffusa (powdery mildew of legumes, e.g. soybean), Leveillula taurica (also known as Oidiopsis taurica) (powdery mildew of onion or artichoke), Podosphaera leucotricha (powdery mildew of apples and pears), Podosphaera macularis (powdery mildew of hemp and cannabis), Podosphaera fusca and Podosphaera xanthii (powdery mildew of cucurbits: cucumbers, squashes (including pumpkins), luffas, melons, and watermelons), Podosphaera pannosa (powdery mildew of roses), and G. cichoracearum (powdery mildew of curcubits).

Embodiment 626: The method according to any one of embodiments 621-624, wherein said reference powdery mildew is a powdery mildew selected from the group consisting of Golovinomyces orontii, Erysiphe necator, Blumeria graminis f. sp. Tritici, Blumeria graminis f. sp. hordei (powdery mildew of barley), Microsphaera diffusa (powdery mildew of legumes, e.g. soybean), Leveillula taurica (also known as Oidiopsis taurica) (powdery mildew of onion or artichoke), Podosphaera leucotricha (powdery mildew of apples and pears), and Podosphaera xanthii.

Embodiment 627: The method of embodiment 625, wherein said reference powdery mildew is Golovinomyces orontii.

Embodiment 628: The method according to any one of embodiments 621-624, wherein said ortholog is an ortholog found in a powdery mildew.

Embodiment 629: The method of embodiment 628, wherein said ortholog is an ortholog found in a powdery mildew selected from the group consisting of Erysiphe necator (or Uncinula necator) (powdery mildew of grapes), Blumeria graminis f. sp. tritici (causes powdery mildew of wheat), f. sp. hordei (powdery mildew of barley), Microsphaera diffusa (powdery mildew of legumes, e.g. soybean), Leveillula taurica (also known as Oidiopsis taurica) (powdery mildew of onion or artichoke), Podosphaera leucotricha (powdery mildew of apples and pears), and Podosphaera xanthii (powdery mildew of cucurbits: cucumbers, squashes (including pumpkins), luffas, melons, and watermelons).

Embodiment 630: The method of embodiment 629, wherein said ortholog is an ortholog found in Erysiphe necator (or Uncinula necator).

Embodiment 631: The method according to any one of embodiments 621-624, wherein said ortholog is an ortholog found in a plant rust.

Embodiment 632: The method of embodiment 631, wherein said ortholog is an ortholog found in a plant rust selected from the group consisting of Cronartium ribicola (White pine blister rust), Gymnosporangium juniperi-virginianae (Cedar-apple rust), Hemileia vastatrix (Coffee rust), Phakopsora meibomiae and P. pachyrhizi (Soybean rust), Puccinia coronata (Crown Rust of Oats and Ryegrass), Puccinia graminis (Stem rust of wheat and Kentucky bluegrass, or black rust of cereals), Puccinia hemerocallidis (Daylily rust), Puccinia triticina (Brown Wheat Rust), Puccinia sorghi (Common Rust of Corn), Puccinia striiformis (Yellow Rust) of cereals, Uromyces appendiculatus (Bean Rust), Puccinia melanocephala (Brown Rust of Sugarcane), and Puccinia kuehnii (Orange rust of Sugar cane).

Embodiment 633: The method according to any one of embodiments 621-624, wherein said ortholog is an ortholog found in an ascomycete selected from the group consisting of Fusarium spp. (Fusarium wilt disease), Thielaviopsis spp. (canker rot, black root rot, Thielaviopsis root rot), Verticillium spp., Magnaporthe grisea (rice blast), Botrytis spp. (grey mold), and Sclerotinia sclerotiorum (cottony rot).

Embodiment 634: The method according to any one of embodiments 621-624, wherein said ortholog is an ortholog found in a Basidiomycete selected from the group consisting of Ustilago spp. (smuts) smut of barley, Rhizoctonia spp., and Armillaria spp. (honey fungus species, virulent pathogens of trees).

Embodiment 635: The method according to any one of embodiments 621-634, wherein said ortholog shares at least 35% sequence identify, or at least 40% sequence identity, or at least 50% sequence identity, or at least 60% sequence identity, or at least 70% sequence identity, or at least 80% sequence identity, or at least 85% sequence identity, or at least 90% sequence identity, or at least 95% sequence identity with the corresponding G. orontii gene.

Embodiment 636: The method according to any one of embodiments 621-635, wherein said ortholog encodes a protein that shares at least 50% sequence identity, or at least 60% sequence identity, or at least 70% sequence identity, or at least 80% sequence identity, or at least 85% sequence identity, or at least 90% sequence identity, or at least 95% sequence identity, or at least 98% sequence identity with a protein expressed by the corresponding G. orontii gene.

Embodiment 637: The method according to any one of embodiments 621-636, wherein said determining growth and/or reproduction comprises counting spore production and/or determining hyphal length and/or determining spore germination, and/or determining penetration of host plant, and/or visual disease symptoms, and/or quantification of pathogen DNA or RNA.

Embodiment 638: The method according to any one of embodiments 621-636, wherein said determining growth and/or reproduction comprises counting spore production and/or determining hyphal length.

Embodiment 639: The method according to any one of embodiments 621-636, wherein said determining growth and/or reproduction comprises counting spore production and/or determining hyphal length and/or visual disease symptoms.

Embodiment 640: In any of the forgoing embodiments, the target genes or groups of target genes expressly omit CYP51 (SEQ ID NO:85).

### Definitions

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. In various embodiments, the polynucleotides may comprise heterocyclic nucleosides consisting of purines and the pyrimidines bases, bonded in a 3′ to 5′ phosphodiester linkage. In certain embodiments, polynucleotides may be modified in backbone, and/or internucleotide linkages, and/or bases, e.g., as described hereinunder. Specific examples of polynucleotides include polynucleotide agents containing modified backbones or non-natural internucleoside linkages. Polynucleotide agents having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050. Other modified polynucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Alternatively, modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. Other polynucleotides include, but are not limited to those modified in both sugar and the internucleoside linkage, i.e., the backbone, of the nucleotide units are replaced with novel groups. The base units are maintained for complementation with the appropriate polynucleotide target. An example for such a polynucleotide mimetic, includes peptide nucleic acid (PNA). A PNA polynucleotide refers to a polynucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. In various embodiments, polynucleotides can include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. These include, for example, 5-substituted pyrimidines, 6-azapyrimidines and N2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and can be when combined with 2′-O-methoxyethyl sugar modifications.

By “isolated” nucleic acid molecule(s) is intended a nucleic acid molecule, DNA or RNA, that has been removed from its native environment or that has never existed in its native environment or that is a de novo novel nucleic acid molecule. For example, recombinant DNA molecules contained in a DNA construct are considered isolated. Further examples of isolated DNA molecules include recombinant DNA molecules maintained in heterologous host cells or purified (partially or substantially) DNA molecules in solution. Isolated RNA molecules can include in vitro RNA transcripts of DNA molecules. Isolated nucleic acid molecules, according to the present invention, further include such molecules produced synthetically.

A “plant”, as used herein refers to any of various photosynthetic, eukaryotic, multicellular organisms of the kingdom Plantae characteristically producing embryos, containing chloroplasts, and having cellulose cell walls. A part of a plant, i.e., a “plant tissue” may be treated according to the methods described herein to prevent phytopathogen infestation on the plant or on the part of the plant. Many suitable plant tissues can be treated according to the present invention and include, but are not limited to, somatic embryos, pollen, leaves, stems, calli, stolons, microtubers, and shoots. Thus, in various embodiments, the treatment of angiosperm and gymnosperm plants including vegetables, fruits, cereals and others such as acacia, alfalfa, apple, apricot, artichoke, ash tree, asparagus, avocado, banana, barley, beans, beet, birch, beech, blackberry, blueberry, broccoli, brussels sprouts, cabbage, cannabis, canola, cantaloupe, carrot, cassava, cauliflower, cedar, a cereal, celery, chestnut, cherry, cabbage, citrus, clementine, clover, coffee, corn, cotton, cowpea, cucumber, cucurbits, cypress, eggplant, elm, endive, eucalyptus, fennel, figs, fir, geranium, grape, grapefruit, groundnuts, ground cherry, gum hemlock, hemp, hickory, hops, jatropha, kale, kiwifruit, kohlrabi, larch, lettuce, leek, legumes, lemon, lime, locust, pine, maidenhair, maize, mango, maple, melon, millets, mushroom, mustard, nuts, oak, oats, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, soybean, spinach, spruce, squash, strawberry, sugarbeet, sugarcane, sunflower, sweet potato, sweet corn, tangerine, tea, tobacco, tomato, trees, triticale, turf grasses, turnips, a vine, walnut, watercress, watermelon, wheat, yams, yew, and zucchini is envisioned. The term “plant” encompasses all plants and plant populations such as desirable and undesirable wild plants, cultivars, and plant varieties. Cultivars and plant varieties can be plants obtained by conventional propagation and breeding methods which can be assisted or supplemented by one or more biotechnological methods such as by use of double haploids, protoplast fusion, random or directed mutagenesis, molecular or genetic markers or by bioengineering or genetic engineering methods. The term plant also encompasses modified plants, in which the plant genome is modified by any method including (e.g., by mutation, deletion, gene editing, introduction of a transgene, hybridization, etc.).

The term “plant tissue” also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendants of any of these, such as cuttings or seed.

The term “controlling an infection of a plant by a phytopathogen” indicates a reduction in the infectivity, and/growth rate, and/or proliferation rate of the phytopathogen in the host plant. Such control can comprise cessation of infection, growth, development, reproduction and/or pathogenicity and/or eventual death of the phytoathogen.

The term “fungus” refers to any of numerous spore-producing eukaryotic organisms of the kingdom Fungi, that lack chlorophyll and vascular tissue and range in form from a single cell to a mass of branched filamentous hyphae that often produce specialized fruiting bodies. The kingdom includes the yeasts, smuts, rusts, mushrooms, and many molds, excluding the slime molds and the water molds. Also included within the term “fungus” as used herein are fungus-like eukaryotic microorganisms such as the oomycota or oomycetes which include notorious pathogens of plants, causing devastating diseases such as late blight of potato and sudden oak death. Examples of oomycetes include, but are not limited to Phytophthora infestans, Phytophthora palmivora, Phytophthora sojae, and the like. oomycetes are for the purposes of the present invention, included within the term “fungal” or “fungus”.

As used herein, the term “phytopathogenic” or “pathogenic” refers to a fungus or other pathogen that causes a disease in a plant.

As used herein, “a DNA capable of expressing a dsRNA” or a “DNA that expresses a dsRNA” or “a DNA that provides a transcriptional template for inhibitory dsRNA or antisense RNA” refers to any DNA molecule that encodes an inhibitory nucleic acid molecule (e.g., a dsRNA). Upon transcription, an inhibitory nucleic acid (e.g., an RNA molecule that can comprise dsRNA), is produced as described herein. In various embodiments the RNA molecule may be an mRNA molecule, a single-stranded antisense RNA, or a dsRNA, for example an shpRNA, siRNA, tasiRNA, miRNA or other classes of inhibitory RNAs. The DNA may comprise one or more than one antisense sequence(s). Thus, the DNA may comprise one or more than one antisense sequence(s) against the target gene(s) described herein. In certain embodiments, the DNA may comprise one or more than one sense sequence(s) which is (are) substantially complementary to the antisense sequence(s). The antisense and sense sequences may be present on the same or on different DNA strands. Upon transcription, inhibitory molecule(s) comprising the antisense and possibly sense sequences are generated.

DNA capable of expressing an inhibitory nucleic acid molecule (e.g., a dsRNA) may be present within an expression cassette.

As used herein, an “expression cassette” is a nucleic acid molecule that is composed of one or more genes or genetic sequences and the sequences controlling their expression. An expression cassette may contain a promoter regulatory sequence, also designated promoter, operably linked to an open reading frame or another genetic sequence, and typically a 3′ untranslated region that may contain a polyadenylation site. The promoter directs the machinery of the cell to make RNA and/or protein. As used herein, “operably linked” means that transcription and/or expression of the linked DNA sequences is under control of the promoter and, in various embodiments, occurs in a plant transfected with the cassette. In certain embodiments, an expression cassette may be part of a vector used for cloning and introducing the DNA into a cell.

The term “downregulate expression of a gene” refers to reduction of expression of at least 20%, or at least 30%, or at least 40%, or at least 50%, or at least 60%, or at least 70%, or at least 80%, or at least 90%, or at least 95% or of 100% for a target gene as described herein as compared to expression in a non-transgenic plant of identical phenotype, or an untreated plant of identical phenotype (e.g., in either case a plant not contacted with and not comprising a dsRNA described herein). Preferably, the damages on the plant are reduced by at least 10%, 20%, 30%, 40%, 50%, 60%, 70%, 80%, 90% or by 100% as compared to a plant not contacted with and not comprising a dsRNA described herein.

The transgenic plant is produced according to methods well known to those of skill in the art. Thus, for introducing the DNA encoding the dsRNA(s) described herein molecule into a plant or a part thereof, the DNA molecule or the expression cassette harboring the DNA may be inserted into a vector. Vectors that harbor a DNA polynucleotide for effecting inhibition of gene expression in a plant cell are known to those in the art and are also useful for the transgenic plants described herein. In addition to the DNA molecule, the vector may comprise heterologous regulatory element(s) in the 5′ and optionally in the 3′ positions that are able to function in a plant. The vector may comprise a promoter regulatory sequence that is functional in plant cells, operably linked to the DNA molecule, and optionally a terminator regulatory sequence. In certain embodiments, such a vector may be a hairpin vector or a double promoter vector, as known in the art. Using this type of vector, the inserted DNA molecule is transcribed bidirectionally. An inverted double promoter allows the expression of one DNA sequence in the 3′ direction and of a second DNA sequence in the 5′ direction, whereby the resulting RNAs are substantially complementary to each other and generate dsRNA. Alternatively, two promoters or a bidirectional promoter, e.g. the mannopine synthase promoter (Guevara-Garcia et al. (1993) Plant J. (3); 495-505), may be employed such that one promoter regulates the transcription of a DNA sequence comprising an antisense sequence and the second promoter regulates the transcription of a DNA sequence comprising an antisense sequence, which, however, is present on the DNA not in complementary location to the sense sequence. Suitable binary vectors for the transformation of plants include but are not limited to the pBINPLUS vector (van Engelen et al. (1995) Transgenic Res. 4: 288-290), the pGPTV vector (Becker et al. (1992) Plant Mol. Biol., 29: 1195-1197), the p6U and p7U vector (DNA Cloning Service e. K., Hamburg, Germany; www.dna-cloning.com; U.S. Pat. No. 7,834,243), and the like.

The term “promoter regulatory sequence” or “promoter” is intended to mean any promoter of a gene that can be expressed in a plant. Such promoter may be a promoter that is naturally present in a plant or is of fungal, bacterial or viral origin. In certain embodiments, the promoter is a tissue specific promoter and/or a pathogen inducible promoter. In certain embodiments, the promoter can be a synthetic promoter. Examples of promoters of plant origin are the histone promoter (EP 0 507 698) or the rice actin promoter (U.S. Pat. No. 5,641,876). Examples of promoters of a plant virus gene are the cauliflower mosaic virus (CaMV 19S or 35S), the cassava vein mosaic virus (CsVMV: PCT Publication No: WO97/048819) or the circovirus promoter (Australian Patent AU 689 311). Examples of tissue-specific promoters are the napin (European patent No: EP 255 378), phaseolin, glutenin, helianthinin (PCT Publication No; WO 92/017580), albumin (PCT Publication No; WO 98/045460) and oleosin (PCT Publication No; WO 98/045461) promoter. Examples of inducible promoters include, but are not limited to the promoters of phenylalanine ammonia lyase (PAL), of HMG-CoA reductase (HMG), of chitinases, of glucanases, of proteinase inhibitors (PI), of genes of the PR1 family, of nopaline synthase (nos) or of the vspB gene (U.S. Pat. No. 5,670,349), the HMG2 promoter (U.S. Pat. No. 5,670,349), the apple beta-galactosidase (ABG1) promoter or the apple amino cyclopropane carboxylate synthase (ACC synthase) promoter (PCT Publication No: WO 98/045445), or chimeric pathogen inducible promoters (PCT Publication Nos WO 2000/029592; WO 2007/147395; WO 2013/091612).

The terms “ortholog” or “orthologous” genes refer to genes that encode a polypeptide or protein in one species that is the functional counterpart of a polypeptide or protein in different species. While, formally, the term “orthologous” reflects separation via a speciation event, as used herein, orthologs need not be so limited. For example, an ortholog may also refer to genes that encode a polypeptide or protein that is the functional counterpart of a polypeptide or protein in the same species. For example, an ortholog may also refer to a gene that encodes protein that is a functionally similar counterpart to the protein encoded by the “target gene” of interest. In certain embodiments orthologs can be defined by percentage identity or by percentage similarity at the protein level or at the DNA level. Percentage identity at the protein level correlates with the proportion of identical amino-acid residues shared between two protein sequences compared in an alignment. Similarly, percentage identity at the DNA level correlates with the proportion of identical nucleic acid residues shared between two nucleic acid sequences compared in an alignment. Percentage similarity at the protein level correlates with the proportion of amino-acid residues having similar structural properties that is shared between two sequences compared in an alignment. Percentages of similarity and identity can be calculated over a portion of the primary structure and/or over the entire gene/protein sequence. For example, amino-acid residues having similar structural properties can be substituted for one another, such as the substitutions of analogous hydrophilic amino-acid residues, and the substitution of analogous hydrophobic amino-acid residues. Percentages of similarity and identity can be calculated over a portion of the primary structure and not over the entire gene/protein sequence. For the present disclosure, an ortholog or an orthologous sequence at the protein or or DNA level is a protein or DNA molecule with sequence identity of at least 35%, 40%, 45%, 50%, 55%, 60%, 65%, 70%, 75%, 80%, 85%, 90%, 95%, and/or 98%. In certain embodiments an ortholog or orthologous sequence is also defined as a homologous molecule with a protein sequence identity of 20% to 34% and/or protein sequence similarity of 30% to 39% if the E value is smaller than 1e-10.

The Expect value (E) is a parameter that describes the number of hits one can “expect” to see by chance when searching a database of a particular size. The lower the E-Value or closer it is to zero, the more “significant” the match is. The E-value takes into account both the length of the query sequence (e.g., the GolorMGH1v4 sequence) and the sequence score based on local ungapped alignments. Essentially, the E value describes the random background noise. For example, an E value of 1 assigned to a hit can be interpreted as meaning that in a database of the current size one might expect to see 1 match with a similar score simply by chance. The lower the E-value, or the closer it is to zero, the more “significant” the match is. However, virtually identical short alignments can have relatively high E values. This is because the calculation of the E value takes into account the length of the query sequence. These high E values reflects the fact that shorter sequences have a higher probability of occurring in the database purely by chance. The Expect value can also be used as a convenient way to create a significance threshold for reporting results. The Expect value threshold on most BLAST search pages can be adjusted. When the Expect value is increased from the default value of 10, a larger list with more low-scoring hits can be reported.

The term “target gene” as used herein refers to a gene that is to be inhibited and/or whose expression is to be downregulated using, e.g., an inhibitory RNA as described herein. A “target gene ortholog” or “orthologous target gene” refers to a gene orthologous to the reference target gene that is to be downregulated. In certain embodiments the downregulated target is a gene in a different strain or species than the reference gene.

As used herein, the terms “identity,” “sequence identity”, e.g., “percent identity” to an amino acid sequence or to a nucleotide sequence disclosed herein refers to a relationship between two or more amino acid sequences or between two or more nucleotide sequences. When a position in one sequence is occupied by the same nucleic acid base or amino acid in the corresponding position of the comparator sequence, the sequences are said to be “identical” at that position. The percentage of “sequence identity” is calculated by determining the number of positions at which the identical nucleic acid base or amino acid occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a nucleotide or amino acid sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using, e.g., the program “BLAST” which is available from the National Center for Biotechnology Information, and which program incorporates the programs BLASTN (for nucleotide sequence comparison) and BLASTP (for amino acid sequence comparison), which programs are based on the algorithm of Karlin and Altschul ((1993). Proc. Natl. Acad. Sci. USA. 90(12); 5873-5877).

The term “percent similarly can refer to the extent to which nucleotide or protein sequences are related. Similarity between two sequences can be expressed as percent sequence identity and/or percent positive substitutions. Percent similarly can refer to percent positives in BLAST (see, e.g., //www.ncbi.nlm.nih.gov/books/NBK62051; Fassler & Cooper (2008-) BLAST Glossary. 2011 Jul. 14. In: BLAST® Help [Internet]. Bethesda (MD); National Center for Biotechnology Information (US), available from: //www.ncbi.nlm.nih.gov/books/NBK62051).

The term “substitution” refers to the presence of a non-identical amino acid at a given position in an alignment. If the aligned residues have similar physico-chemical properties or have a positive score in the governing scoring matrix the substitution is said to be conservative.

A “conserved substitution” refers to a change at a specific position of an amino acid or DNA sequence that preserves the physico-chemical properties of the original residue or achieves a positive score in the governing scoring matrix.

As used herein, the term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. When using RNA as opposed to DNA, uracil (U) rather than thymine (T) is the base that is considered to be complementary to adenosine. However, when a U is denoted in the context of the present invention, the ability to substitute a T is implied, unless otherwise stated.

The term “agrochemical” as used herein means a chemical substance, whether naturally or synthetically obtained, that is applied to a plant, to a pest or to a locus thereof to result in expressing a desired biological activity.

The term “biological activity” as used herein refers to elicitation of a stimulatory, inhibitory, regulatory, therapeutic, toxic or lethal response in a plant or in a pest such as a pathogen, parasite or feeding organism present in or on a plant or the elicitation of such a response in a locus of a plant, a pest or a structure.

A “fungicide” can be a compound or agent, whether chemical or biological, that can inhibit the growth or propagation of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and/or providing some level of control of soil-borne pathogens or providing some level control of leaf-borne fungal pathogen.

It will be noted that where a dsRNA is referenced by a nucleotide sequence that is a DNA (i.e., contains “U” rather than “T”), the corresponding RNA sequences are contemplated. Similarly, where a single strand is provided the double stranded nucleic acid is contemplated.

### BRIEF DESCRIPTION OF THE DRAWINGS

FIG. 1 shows that G. orontii MGH1 uptakes extracellular RNA. Germinated spores were incubated with RNA containing fluorescein conjugated UTPs or water. Before imaging RNases were added. Images taken with Zeiss Axio Imager using the 20× objective. DIC=Differential Interference Contrast. GFP=Green Fluorescent Protein Filter Set. Scale bar=50 μm.

FIG. 2 shows an overview of one embodiment of spray-induced gene silencing methodology. The flowchart outlines target gene selection, siRNA and long dsRNA design, spray-induced gene silencing protocol, and methods used to identify powdery mildew infection phenotypes. Noted methods of selecting gene targets and evaluating pathogen growth and reproduction are illustrative.

FIG. 3, panels a-e, shows that siRNA and long dsRNA targeting CYP51 reduces spore production and hyphal length. Panel a) Diagram of CYP51 and targeting RNAs. CYP51 is 1578 bp. Panel b) Average normalized spore production displayed from n≥3. Spore suspension concentrations were normalized to the input tissue weight (g) before being normalized to the control. Error bars±SEM. *p<0.05 by Student's T-Test (unpaired, 2-tailed compared to control. Panel c) Less powdery mildew coverage and density observed on CYP51 dsRNA-1 sprayed A. thaliana plants. Image taken 9 days post infection. Panel d) G. orontii MGH1 hyphal length is reduced by siRNAs targeting CYP51 on A. thaliana. Calcofluor-stained G. orontii imaged at 2 dpi with a Zeiss Axio Imager using a 10× objective and DAPI long pass filter set. Scale bar is 100 μm. Panel e) Average total hyphal length of colonies at 2 and 3 dpi from detached leaf experiments using 1% agar plates. Error bars±SEM. *p<0.05 by Student's T-Test (unpaired, 2-tailed) compared to control. Similar results observed in an independent experiment.

FIG. 4 shows that G. orontii MGH1 spore production is impacted by long dsRNA targeting metabolic, regulatory, and effector genes on A. thaliana whole plant and detached leaf assays. Average normalized spore production displayed from n≥3. Spore suspension concentrations were normalized to the input tissue weight (g) before being normalized to the control. Error bars±SEM. *p<0.05 by Student's T-Test (unpaired, 2-tailed compared to control. Note, this represents a subset of target categories and G. orontii powdery mildew targets similarly tested. See, for example, Tables 1 and 2 for additional targets.

FIG. 5 illustrates G. orontii MGH1 gene expression for the spray-induced gene silencing targets in FIG. 4 at 0, 6, 12, 24, 72, and 120 hours post infection. Mean normalized FPKM (fragments per kilobase of exon per million reads) with n=3 is displayed for each gene+/−SE. Golor4 annotated genome can be accessed through MycoCosm (Grigoriev et al., 2014). Black and gray box denote central metabolic functions (cell membrane biosynthesis, methionine salvage pathway). Genes involved in glycogen metabolism are in blue box. Light blue box encloses genes involved in storage lipid metabolism. Green box includes genes expected to be involved in fungal manipulation of plant processes or response to plant-induced stress. As in FIG. 4, this figure represents a subset of genes profiled.

FIG. 6, panels a-b, shows that G. orontii MGH1 spore production is reduced by siRNAs targeting EC2 and BCDO on A. thaliana. Panel a) Detached leaves sprayed with BCDO siRNA have reduced powdery mildew and less chlorosis than the control at 8 days post infection. Panel b) Average normalized spore production displayed from n≥3. Spore suspension concentrations were normalized to the input tissue weight (g) before being normalized to the control. Error bars±SEM. *p<0.05 by Student's T-Test (unpaired, 2-tailed) compared to control. † n=2, each experiment p<0.00005.

FIG. 7, panels a-b, shows that long dsRNA targeting genes in E. necator reduces spore production on V. vinifera. Panel a) Average normalized spore production displayed from n≥3. Spore suspension concentrations were normalized to the input tissue weight (g) before being normalized to the control. Error bars±SEM. *p<0.05 by Student's T-Test (unpaired, 2-tailed compared to control. † n=2, each experiment p<0.05. Panel b) Less powdery mildew density observed on CYP51 long dsRNA sprayed V. vinifera 9 days post infection.

FIG. 8 illustrates G. orontii developmental timeline. Modified from Both et al., 2005.

FIG. 9, panels a-b, illustrates carotenoid biosynthesis enzymes annotated in G. orontii MGH1 and A. thaliana. Panel a) Enzyme classes in red-colored boxes represent genes identified in G. orontii. Panel b) Enzyme classes in boxes highlighted green represent genes present in A. thaliana. Among abscisic acid biosynthesis genes, genes in red boxes are down-regulated (p<0.05) at the infection site during G. orontii infection at 5 dpi (Chandran et al., 2010). Down-regulated genes infected: uninfected expression ratios were: 0.2 for EC 1.23.5.1 (AT1G08550, NPQ4), 0.1 for EC 1.14.14.137 (AT3G19270, CYP707A4), and 0.3 for EC 3.2.1.175 (AT1G52400, BGLU18). Carotenoid Biosynthesis maps (00906) were accessed through KEGG.

FIG. 10, panels a-d, shows that powdery mildew infection assays identify A. thaliana TCP transcription factor and G. orontii effector (OEC) genes contributing to spore production. Panel a) TCP-OEC Protein interaction map and associated powdery mildew spore production phenotypes Interactions were identified by yeast-two hybrid using G. orontii MPIPZ effectors (WeBling et al., 2014). Effector homologs not identified in G. orontii MGH1 are omitted. The genetic contribution to spore production of all genes inside bolded shapes were measured in G. orontii MPIPZ infection of A. thaliana experiments. TCP ovals shaded if associated adult tcp mutant has enhanced powdery mildew spore production. tcp15 mutant seedlings have enhanced disease susceptibility (WeBling et al., 2014). Panel b) G. orontii MGH1 effector-TCP interactions inferred from G. ororntii MPIPZ interactions (WeBling et al., 2014) investigated in this study. The genetic contribution to spore production of all genes inside bolded shapes were measured in G. orontii MGH1 infection of A. thaliana adult plant experiments. TCP ovals shaded if associated tcp mutant has enhanced powdery mildew spore production. OEC circles are shaded to indicate less spore production when homologs of OECs were targeted using spray-induced gene silencing. Panel c) G. orontii MGH1 spore production on T-DNA insertion tcp mutants. Average normalized spore production of n≥6 displayed±SE. Panel d) Spore production of G. orontii MGH1 when targeting homologs of OECs using spray-induced gene silencing. CYP51 is included as a positive control gene required for fungal growth. Average normalized spore production of n≥3 displayed±SE. *p<0.05 by Student's T-Test (unpaired, 2-tailed)

FIG. 11 illustrates G. orontii MGH1 OEC60/OEC61 homologs have structural homology to other glycosyl hydrolase family 17 domain-containing proteins. RaptorX protein structure prediction (Wang et al., 2016) model for G. orontii MGH1 protein OEC60/OEC61 (blue) aligned with Barley endoglucanase (green, PDB ID:1ghs) and Rhizomucor miehei glycoside hydrolase family 17 beta-1,3-glucanosyltransferase which had the highest scored protein structure in the Protein Databank (Pink, PBD ID: 4wtp).

FIG. 12, shows that a long dsRNA targeting homolog of OEC60/OEOC61 in E. necator reduces spore production on V. vinifera leaf discs. Average normalized spore production displayed from n=4. Spore suspension concentrations were normalized to the input tissue weight (g) before being normalized to the control. CYP51 is included as a positive control gene required for growth. Error bars±SEM. *p<0.05 by Student's T-Test (unpaired, 2-tailed) compared to control.

FIG. 13 shows that G. orontii MGH1 homolog of OEC60/OEC61 colocalizes with TCP13 in plant nuclei. YFP-TCP13 and OEC60/OEC61-CFP were co-expressed in Nicotiana benthamiana leaf tissue using Agrobacterium-mediated transient expression. Before microscopy, DAPI was infiltrated into tissue to stain DNA and highlight the nuclei. Top panel: Individual fluorescence channels of coinfiltrated leaf tissue. Scale bar is 100 μm. Bottom panel: Red vector (30 μm) drawn through a nucleus of a merged image from top panel shows strong nuclear accumulation of fluorescence signal for DAPI, YFP, and CFP channels.

FIG. 14, shows that A. thaliana TCP13 interacts in planta with G. orontii MGH1. YFP-TCP13 and OEC60/OEC61-3×HA were co-expressed in Nicotiana benthamiana leaf tissue using Agrobacterium-mediated transient expression. Co-immunoprecipitation was performed to purify GFP-containing protein complexes. Western blot shows HA and GFP detection. Co-immunoprecipitations, and western blotting methods were performed using methods described in WeBling et al., 2014.

FIG. 15 illustrates G. orontii MGH1 effector expression during infection at 0, 6, 12, 24, 72, and 120 hours post infection. Mean normalized FPKM (fragments per kilobase of exon per million reads) with n=3 is displayed for each gene, ±SEM, for each gene indicated by protein ID for G. orontii MGH1 v4.0 annotated and accessed through MycoCosm (Grigoriev et al., 2014). Gene copies are omitted if not identified in RNA-Seq.

FIG. 16 shows two adjacent vines used as one replicate (larger box labeled “A”). The smaller box (labeled “B”) represents the canopy area sprayed for one treatment and data was collected from this region only.

FIG. 17 shows an overview of the powdery mildew treatments at the Kearney site (Kearney Agriculture Research and Extension Center, Parlier, CA).

FIG. 18, panels A-B, shows the disease incidence of grape berries at Kearney site. Panel A) dsRNA treated vines. Panel B) Gold Standard Control. Ttest p-value<0.05 (**); <0.01 (*). Data is average of 5 replicate sample units with standard deviation.

FIG. 19, panels A-B, shows disease severity for dsRNA treated-vines compared with the untreated control vines (t-test shown in panel A) and with Gold Standard treated vines (t-test shown in panel B). **=p-value<0.01. Data is an average of 5 replicate sample units, with standard deviation.

FIG. 20 shows berry chemistry per dsRNA treatment compared with untreated control. Mean of five samples per treatment with standard deviation is shown. “*”=ttest p<0.05 compared to untreated control. Brix (g sucrose/100 g clarified berry juice); pH units, TA (titratable acidity; g tartartic acid added per L of clarified berry juice).

FIG. 21 shows an overview of treatment regime at Armstrong field site. Red (*) boxes indicate chemical fungicide utilized. R—Rally; Q—Quintec; M—Mettel; P=Pristine; IS=Inspire Super; L=Luna; V=Vivando. Last dsRNA spray was Jun. 2, 2021.

FIG. 22, panels A-B, shows the disease incidence at the Armstrong site. Panel A) Data show mean and stdev for 5 replicates per treatment. ttest compared to untreated control; **=p<0.01, *=p<0.05. Panel B) Mean disease incidence per treatment over time.

FIG. 23 panel A, shows Powdery mildew disease severity at Armstrong site. Mean and standard deviation are shown, 5 replicates per treatment. ttest vs. untreated control p-value≤0.01 (**), ≤0.05 (*). FIG. 23, panel B, shows representative berry clusters from untreated (top) and dsRNA-treated (bottom) vines.

FIG. 24 shows that double stranded RNA targeting CYP51 reduces its expression.

### DETAILED DESCRIPTION

In various embodiments, methods and compositions for treating and/or protecting a plant from infection by a phytopathogenic fungus are provided. In particular, fungal gene targets are identified where inhibition of the gene target function (e.g., via inhibition of the expression of the gene targets) reduces the infectivity or severity of infection of a host plant by the phytopathogenic fungus. Additionally, fungal gene target process categories are identified that exhibit a high degree of success as targets that when inhibited result in decreased growth and reproduction of the phytopathogen. Methods are also provided for identifying target genes effective in controlling fungal and other plant infections. This includes both methods for prioritization of genes of interest for screening and screening methods.

RNAi-mediated gene-silencing offers a sustainable alternative approach to control of plant pathogens. Phytopathogens are in constant contact with their plant host(s) and it has been demonstrated that some plant pathogens are capable of taking up polynucleotides present in or on the host plant. Moreover, it was discovered that the obligate biotrophic powdery mildews can take up externally supplied polynucleotides, e.g., when applied to s surface of a plant. Accordingly, it is believed that RNAi-mediated gene silencing can provide an effective means for controlling or preventing an infection of a plant by a phytopathogen (e.g., a phytopathogenic fungus).

However, RNAi-mediated gene silencing requires identifying target genes in the pathogen inhibition of which provides effective control of the phytopathogen, while minimizing impact on the host plant. Because obligate biotrophs such as powdery mildews have proven difficult or impossible to culture, identification of important target genes has proven problematic.

Described herein is a novel screening system that exploits the G. orontii-A. thaliana pathosystem to rapidly identify target genes downregulation or inhibition of which can alter the pathogenesis of G. orontii (see, e.g., Example 1). Moreover, genes identified using this screening system are highly conserved and, accordingly, orthologous genes in other phytopathogenic species can be downregulated to similarly control or preventing an infection of other plant species.

Accordingly, in various embodiments, isolated double stranded RNAs (dsRNAs) are provided that are capable of reducing, controlling, or preventing an infection of a plant by a phytopathogenic fungus by downregulating one or more target G. orontii genes or orthologs of those genes as described herein. The dsRNAs include, but are not limited to long dsRNA, small inhibitory RNA (siRNA), small hairpin RNA (shRNA), miRNA, and the like. Also, in various embodiments, compositions for controlling or preventing an infection of a plant by a phytopathogenic are provided where the composition comprises one or more of the isolated dsRNA molecules described herein. In various embodiments, the composition is suitable for spray induced gene silencing (SIGS).

Having identified useful and effective target genes herein, in various embodiment a transgenic plant or a part thereof is provided where the transgenic plant comprises a transgene, where the transgene comprises a DNA that expresses (transcribes) dsRNA(s) that are capable of reducing, controlling, or preventing an infection of a plant by a phytopathogenic fungus by downregulating one or more target G. orontii genes or orthologs of those genes as described herein. Additionally, in various embodiments, a DNA encoding double stranded RNA (dsRNA) as described herein, an expression cassette comprising the DNA, and a vector comprising the expression cassette are provided. Also provided are methods of producing the transgenic plants described herein.

In various embodiments, method for controlling or preventing an infection of a plant by a phytopathogenic fungus, is provided. In certain embodiments, the method involves contacting the plant or part thereof (e.g., seed) and/or the pathogenic fungus with an isolated double-stranded RNA according as described herein. In certain embodiments, the method involves expressing in the plant to be protected at least one isolated double-stranded RNA as described herein.

Additionally methods are provided for identifying genes in a phytopathogen that when downregulated or inhibited in said phytopathogen inhibit infection of a host plant by the pathogen. In certain embodiments, the methods are implemented in a high throughput manner.

### Target Genes and Phytopathogens.

In various embodiments, target genes are identified that when inhibited (e.g., downregulated) in a phytopathogen inhibit infectivity, and/or growth, and/or reproduction of that phytopathogen on a host plant. More specifically it was discovered that inhibition of certain “target” genes (or their orthologs) implicated in certain metabolic processes can be exploited to effectively control or prevent an infection of a plant by a phytopathogen. In certain embodiments the target gene is selected from the process category: metabolism, regulation, manipulation of plant host, and/or essential cellular processes (see, e.g., Tables 1-3.

In certain embodiments, the target gene is selected from the process sub category: lipid metabolism/acquisition; carbohydrate metabolism; amino acid metabolism/salvage, nucleotide metabolism/salvage, and cofactor metabolism; essential fungal-specific metabolism; mitochondrial electron transport chain and ATP synthesis; transcriptional regulation; protein regulation; signaling; secreted proteins including effectors; plant hormone metabolism; cell cycle/DNA replication; vesicles/autophagy, and phagocytosis; ion/metal transport and osmotic homeostasis. In certain embodiments the target gene comprises a gene with a predicted important, but unknown function (see, e.g., Tables 1-3).

In certain embodiments target genes are identified that when successfully reduce phytopathogen infectivity, and/or growth, and/or reproduction. In certain embodiments these target genes fall into general categories including but not limited to: metabolism, regulation, manipulation of plant host, and essential cellular processes (see, e.g., Tables 1 and 3, below).

In certain embodiments target genes are identified that when successfully reduce phytopathogen infectivity, and/or growth, and/or reproduction. In certain embodiments these target genes fall into subcategories including but not limited to: Metabolism: lipid metabolism/acquisition, carbohydrate metabolism, amino acid metabolism/salvage, nucleotide metabolism/salvage, cofactor metabolism, essential fungal-specific metabolism, mitochondrial electron transport chain and ATP synthesis; Regulation: transcriptional regulation, protein regulation, signalling; Manipulation of plant host: secreted proteins including effectors, plant hormone metabolism; Essential cellular processes: cell cycle/DNA replication, vesicles/autophagy & phagocytosis, ion/metal transport and osmotic homeostasis (see, e.g., Tables 1 and 3, below).

Table 1 shows an illustrative, but non-liming list of powdery mildew SIGS gene targets detailed by process category. Without being bound to a particularly theory, it is believed that inhibition of those gene targets, or their orthologs can effectively control or prevent an infection of a plant by a phytopathogen.

TABLE 1
Shows an illustrative, but non-liming list of powdery mildew SIGS gene targets
detailed by process category. Subprocess categories are also illustrative, not limiting.
Bolded (black) genes were tested for efficacy in the A. thaliana-G. orontii
system. Bolded and underlined genes were also tested for efficacy in the
V. vinifera-E. necator system. Targets flagged with “**”
were tested in vineyard trials (see Example 4). Sequence ID numbers are shown are for
G. orontii sequences. Sequence ID numbers for orthologous genes in
E. necator are shown in Table 2. See Example 3 for additional detail
regarding single copy genes in G. orontii MGH1 following
recent whole genome duplication (WGD).

| SEQ | | | Putative | | Go DNA |
|---|---|---|---|---|---|
| ID | Process | Gene name or | Gene | EC | sequence |
| NO: | Category | PFAM domain | Symbol | number | (transcript) |
| | Process Category 1 - Metabolism | | | | |
| | 1A. Lipid metabolism/acquisition | | | | |
| 1** | TAG/glycerolipid | TAG lipase 1, | LIP1 | — | 4746636 |
| | | carboxyesterase | | | |
| | | domain | | | |
| 2 | TAG/glycerolipid | TAG lipase A, | LIPA | — | 4656038 |
| | | extracellular lipase | | | |
| 3 | TAG/glycerolipid | diglyceride | DGAT | 2.3.1.20 | 2922561 |
| | | acyltransferase | | | |
| 4 | TAG/glycerolipid | glycerol 2- | | 1.1.1.156 | 4323785 |
| | | dehydrogenase | | | |
| | | (NADP+) | | | |
| 5 | TAG/glycerolipid | aldehyde reductase | | 1.1.1.21 | 5367112 |
| 6 | TAG/glycerolipid | aldehyde | ALDH | 1.2.1.3 | 272360 |
| | | dehydrogenase | | | |
| | | (NAD+) | | | |
| 7 | TAG/glycerolipid | phospholipid:diacylglycerol | | 2.3.1.158 | 5327918 |
| | | acyltransferase | | | |
| 8 | TAG/glycerolipid | 2-acylglycerol 0- | | 2.3.1.22 | 5344905 |
| | | acyltransferase | | | |
| 9 | TAG/glycerolipid | 1-acylglycerol-3- | | 2.3.1.51 | 1474628 |
| | | phosphate 0- | | | |
| | | acyltransferase | | | |
| 10 | TAG/glycerolipid | glycerol kinase | | 2.7.1.30 | 5324432 |
| 11 | TAG/glycerolipid | acylglycerol lipase | | 3.1.1.23 | 4665153 |
| 12 | TAG/glycerolipid | triacylglycerol lipase; | | 3.1.1.3 | 4465299 |
| | | autophagy related | | | |
| | | lipase | | | |
| 13 | TAG/glycerolipid | triacylglycerol lipase; | | | 4402504 |
| | | Patatin-like | | | |
| | | phospholipase | | | |
| | | domain | | | |
| 14 | TAG/glycerolipid | phosphatidate | | 3.1.3.4 | 5370699 |
| | | phosphatase | | | |
| 15 | PPP pathway | transaldolase | TAL | 2.2.1.2 | 74388 |
| 16 | PPP pathway | glucose-6-phosphate | G6PD | 1.1.1.49 | 4149977 |
| | | dehydrogenase | | | |
| 17 | PPP pathway | 6- | PGLS | 3.1.1.31 | 1356707 |
| | | phosphogluconolactonase | | | |
| 18 | PPP pathway | transketolase | TKL | 2.2.1.1 | 778339 |
| 19 | PPP pathway | Glucose-6-P | GPI | 5.3.1.9 | 4423521 |
| | | isomerase | | | |
| 20** | TCA & | isocitrate lyase | ICL | 4.1.3.1 | 2918897 |
| | glyoxylate | | | | |
| | cycle | | | | |
| 21** | TCA & | malate synthase | MS | 2.3.3.9 | 5327664 |
| | glyoxylate | | | | |
| | cycle | | | | |
| 22 | TCA & | Citrate (Si) synthase | CS/MCS | 2.3.3.1 | 2948811 |
| | glyoxylate | | | | |
| | cycle | | | | |
| 23 | TCA & | Malate | | 1.1.1.37 | 4219560 |
| | glyoxylate | dehydrogenase | | | |
| | cycle | | | | |
| 24 | TCA & | Aryl formamidase | | 3.5.1.9 | 5390218 |
| | glyoxylate | | | | |
| | cycle | | | | |
| 25 | TCA & | Aconitase hydratase | | 4.2.1.3 | 2916095 |
| | glyoxylate | | | | |
| | cycle | | | | |
| 26 | TCA & | ATP citrate synthase | | 2.3.3.8 | 5327852 |
| | glyoxylate | (With CoA ligase | | | |
| | cycle | domain) | | | |
| 27 | TCA & | mt | SFC1 | | 5320942 |
| | glyoxylate | succinate/fumarate | | | |
| | cycle | antiporter | | | |
| 28 | TCA & | mt dicarboxylic | | | 5327871 |
| | glyoxylate | acid/Pi antiporter | | | |
| | cycle | | | | |
| 29 | TCA & | Pyruvate carboxylase | | 6.4.1.1 | 4650796 |
| | glyoxylate | | | | |
| | cycle | | | | |
| 30 | TCA & | Phosphoenol | | 4.1.1.49 | 5327097 |
| | glyoxylate | pyruvate kinase | | | |
| | cycle | | | | |
| 31 | TCA & | Glutamine | | 6.3.1.2 | 4727341 |
| | glyoxylate | synthetase/glutamate | | | |
| | cycle | ammonia ligase) | | | |
| 181 | TCA & | Alanine-glyoxylate | AGAT | 2.6.1.44 | 4149171 |
| | glyoxylate | aminotransferase | | | |
| | cycle | | | | |
| 32 | FA | trans-2-enoyl CoA | TER | 1.3.1.38 | 1760648 |
| | synthesis/ | reductase | | | |
| | transport | | | | |
| 33 | FA | ATP citrate | | 2.3.3.8 | 4237011 |
| | synthesis/ | lyase/synthase (with | | | |
| | transport | grasp domain) | | | |
| 34 | FA | acyl-coA synthetase | | 6.2.1.1 | 5321903 |
| | synthesis/ | | | | |
| | transport | | | | |
| 35 | FA | long chain fatty acyl- | | 6.2.1.3 | 231879 |
| | synthesis/ | coA | | | |
| | transport | synthetase/transporter | | | |
| 36 | FA | very long chain fatty | | 6.2.1._{—} | 5341277 |
| | synthesis/ | acyl-CoA | | | |
| | transport | synthetase/transporter | | | |
| 37 | FA | fatty acid elongase | | | 1366207 |
| | synthesis/ | | | | |
| | transport | | | | |
| 38 | FA | fatty acid desaturase | | 1.14.19.- | 1482059 |
| | synthesis/ | | | | |
| | transport | | | | |
| 39 | FA | stearoyl-CoA 9- | | 1.14.19.1 | 5368423 |
| | synthesis/ | desaturase | | | |
| | transport | | | | |
| 40 | FA | Acetyl-CoA | | | 5338404 |
| | synthesis/ | transporter | | | |
| | transport | | | | |
| 41 | FA | SUR7/PalI - | | | 4423742 |
| | synthesis/ | containing genes | | | |
| | transport | | | | |
| 42 | FA B- | carnitine acyl | CRAT | 2.3.1.7 | 4146576 |
| | oxidation | transferase I | | | |
| 43 | FA B- | enoyl-CoA hydratase | | 4.2.1.17 | 2910430 |
| | oxidation | | | | |
| 44 | FA B- | acyl-CoA | FadA, FadI | 2.3.1.16 | 581215 |
| | oxidation | acetyltransferase | | | |
| | | (thiolase) | | | |
| 45 | FA B- | acyl-CoA | | 1.3.99.12 | 4202856 |
| | oxidation | dehydrogenases, | | | |
| | | short chain | | | |
| 46 | FA B- | acyl-CoA | | 1.3.8.7 | 4,811,245 |
| | oxidation | dehyrogenase, | | | |
| | | medium chain | | | |
| 47 | FA B- | acyl-CoA | | 1.3.99.- | 5,328,179 |
| | oxidation | dehydrogenase, very | | | |
| | | long chain | | | |
| 48 | FA B- | acetyl-CoA C- | AtoB, | 2.3.1.9 | 1,827,800 |
| | oxidation | acetyltransferase | ACAT | | |
| 49 | FA B- | hydroxymethylglutaryl- | | 2.3.3.10 | 2922372 |
| | oxidation | CoA synthase | | | |
| 50 | FA B- | 3-hydroxybutyrate | BDH | 1.1.1.3 | 5210932 |
| | oxidation | dehydrogenase | | | |
| 51 | Phospholipid | pf01066~CDP- | | 2.7.8.1 | 5328371 |
| | metabolism | alcohol | | | |
| | | phosphatidyltransferase | | | |
| | 1B. Carbohydrate Metabolism | | | | |
| 52 | Glycogen | glycogen | GDB1 | | 5348921 |
| | metabolism | debranching | | | |
| | | enzyme | | | |
| 53 | Glycogen | glycogen | GPH1 | | 4899748 |
| | metabolism | phosphorylase | | | |
| 54 | Glycolysis | Fructose 1,6 | FBA | 4.1.2.13 | 5370817 |
| | | bisphosphate | | | |
| | | aldolase | | | |
| 55 | Glycolysis | Hexokinase | HK | 2.7.1.1 | 4537794 |
| 56 | Glycolysis | Phosphofructokinase-1 | PFK-1 | 2.7.1.11 | 4568150 |
| 57 | Glycolysis | Pyruvate kinase | PK | 2.7.1.40 | 353189 |
| 58 | Carbohydrate | pf13418~Galactose | | | 5295479 |
| | utlization | oxidase, central | | | |
| | | domain; pf00651~BT | | | |
| | | B/POZ | | | |
| | | domain; pf13854~Kelch | | | |
| | | motif; pf01344~Kelch | | | |
| | | motif | | | |
| 59 | Carbohydrate | pf04321~RmlD | | | 5322874 |
| | utlization | substrate binding | | | |
| | | domain | | | |
| 60 | Carbohydrate | pf01256~Carbohydrate | | | 5327463 |
| | utlization | kinase | | | |
| 61 | Carbohydrate | Myo-inositol, sugar, | | | 4724431 |
| | utlization | and other transporter | | | |
| | 1C. Amino acid & nucleotide metabolism & salvage; cofactor metabolism | | | | |
| 62 | Amino acid | 2,3-dioxomethio | UTR4/EN | 3.1.3.77 | 2949221 |
| | metabolism/ | pentane- 1- | OPH1 | | |
| | salvage | phosphate enolase/ | | | |
| | | phosphatase | | | |
| 63 | Amino acid | glutamate | GDH | 1.4.1.2 | 2919558 |
| | metabolism/ | dehydrogenase | | | |
| | salvage | | | | |
| 64 | Amino acid | threonine aldolase | THA | 4.1.2.5 | 2908555 |
| | metabolism/ | | | | |
| | salvage | | | | |
| 65 | Amino acid | serine o- | SATase | 2.3.1.30 | 5323679 |
| | metabolism/ | acetyltransferase | | | |
| | salvage | | | | |
| 66 | Amino acid | spermidine synthase | | 2.5.1.16 | 2912504 |
| | metabolism/ | | | | |
| | salvage | | | | |
| 67 | Amino acid | Putative 2- | | 2.3.3.14 | 4723293 |
| | metabolism/ | isopropylmalate | | | |
| | salvage | synthase | | | |
| 68 | Amino acid | Transmembrane AA | | | 5321389 |
| | metabolism/ | transporter | | | |
| | salvage | | | | |
| 69 | Amino acid | OPT oligopeptide | | | 5320262 |
| | metabolism/ | transporter | | | |
| | salvage | | | | |
| 70 | Cofactor | pf02548~Ketopantoate | KPHMT | 2.1.2.11 | 2925689 |
| | metabolism | hydroxymethyltransferase | | | |
| 71 | Cofactor | tetrahydrofolate | | 6.3.2.17 | 1288071 |
| | metabolism | synthase | | | |
| 72 | Cofactor | pf02558~Ketopantoate | | | 4442131 |
| | metabolism | reductase | | | |
| | | PanE/ApbA; | | | |
| | | pf08546~Ketopantoate | | | |
| | | reductase | | | |
| | | PanE/ApbA C | | | |
| | | terminal | | | |
| 73 | Cofactor | glycine | | 2.1.2.1 | 1010163 |
| | metabolism | hydroxymethyltransfe | | | |
| | | rase/serine aldolase | | | |
| 74 | Cofactor | dihydrofolate | DHFR | 1.5.1.3 | 327732 |
| | metabolism | reductase | | | |
| 75 | Cofactor | aminodeoxychorismate | ADC | 2.6.185 | 2912363 |
| | metabolism | synthase | synthase | | |
| 76 | Cofactor | thiamine | TPK1 | 2.7.6.2 | 2117296 |
| | metabolism | pyrophospokinase | | | |
| 77 | Cofactor | | PNC1 | 3.5.1.19 | 2645295 |
| | metabolism | | | | |
| 78 | Cofactor | pf02492~CobW/HypB/ | | | 2910885 |
| | metabolism | UreG, nucleotide- | | | |
| | | binding | | | |
| | | domain; pf07683~Cobalamin | | | |
| | | synthesis | | | |
| | | protein cobW C- | | | |
| | | terminal domain | | | |
| 79 | Nucleotide | thymidylate | TS | 2.1.1.45 | 2912202 |
| | metabolism/ | synthase | | | |
| | salvage | | | | |
| 80 | Nucleotide | adenine | | 2.4.2.7 | 4154475 |
| | metabolism/ | phosphoribosyltransferase | | | |
| | salvage | | | | |
| 81 | Nucleotide | uracil | | 2.4.2.9 | 1168816 |
| | metabolism/ | phosphoribosyltransferase | | | |
| | salvage | | | | |
| 82 | Nucleotide | uridine kinase | | 2.7.1.48 | 4200233 |
| | metabolism/ | | | | |
| | salvage | | | | |
| 83 | Nucleotide | pf00383~Cytidine | | | 5321215 |
| | metabolism/ | and deoxycytidylate | | | |
| | salvage | deaminase zinc- | | | |
| | | binding region | | | |
| 84 | Nucleotide | Nucleoside | | | 4703978 |
| | metabolism/ | transporter | | | |
| | salvage | | | | |
| | 1D. Essential fungal-specific processes | | | | |
| 85** | Sterol | Cytochrome P450 | CYP51 | — | 2918117 |
| | biosynthesis | monoxygenase 51 | | | |
| 86 | Fungal sexual | GPCR fungal | STE2 | | 454143 |
| | reproduction/ | pheromone mating | | | |
| | compatibility | factor, STE2 | | | |
| | 1E. Mitochondrial electron transport chain & ATP synthesis | | | | |
| 87 | mitochondrial | pf08238~Sel1 repeat | | | 5345424 |
| | electron | | | | |
| | transport | | | | |
| 88 | ATP synthesis | pf05405~Mitochondrial | | | 5327449 |
| | | ATP synthase B | | | |
| | | chain precursor | | | |
| | | (ATP-synt_B) | | | |
| 89 | ATP synthesis | Mitochondrial carrier | | | 4771748 |
| | | protein | | | |
| 90 | Alternative | alternative oxidase | AOX | 1.10.3.11 | 5108926 |
| | Respiratory | | | | |
| | Pathway | | | | |
| 91 | Alternative | NADH | NDE2 | 1.6.5.9 | 2918016 |
| | Respiratory | dehydrogenase | | | |
| | Pathway | | | | |
| | Process Category 2 - Regulation | | | | |
| | 2A. Transcriptional Regulation | | | | |
| 92 | Stress | apoptosis | AATF | — | 5415797 |
| | response | antagonizing | | | |
| | | transcription factor | | | |
| 93 | Stress | BZip, AP1-like | AP1-like | | 2926335 |
| | response | transcription factor | | | |
| 94 | Metabolic | glucose-induced | GID9 | | 5322369 |
| | regulation | degradation 9, | | | |
| | | negative regulator | | | |
| | | of gluconeogenesis | | | |
| 95 | Metabolic | Transcription factor, | Opi1 | | 5322885 |
| | regulation | negative regulator of | | | |
| | | phospholipid | | | |
| | | biosynthesis | | | |
| 96 | Metabolic | Transcription factor, | BrlA | | 2351034 |
| | regulation | Starvation stress, | | | |
| | | aspergillus | | | |
| 97 | Development | Transcription factor, | VelB | | 2923239 |
| | | Velvet factor (VelB), | | | |
| | | positive regulator | | | |
| 98 | Development | fungal specific | | | 5325399 |
| | | transcription factor | | | |
| | | domain, Zn2Cys6 | | | |
| | | binuclear cluster | | | |
| | | domain | | | |
| 99 | Development | Brucentaur or | BCNT | | 2718758 |
| | | craniofacial | | | |
| | | development protein | | | |
| 100 | Transcription | pf04818~CID domain | | | 4330818 |
| 101 | Transcription | pf02270~TFIIF, beta | | | 5191535 |
| | | subunit HTH domain | | | |
| 102 | RNA-binding | pf00076~RNA | | | 5400372 |
| | | recognition motif. | | | |
| | | (a.k.a. RRM, RBD or | | | |
| | | RNP domain) | | | |
| 103 | RNA-binding | RNA Recognition | | | 4204905 |
| | | Motif (RRM) | | | |
| | | containing protein | | | |
| | 2B. Protein regulation (synthesis/turnover/localization) | | | | |
| 104 | translation | pf10453~Nuclear | NUFP1 | | 2925357 |
| | | fragile X mental | | | |
| | | retardation- | | | |
| | | interacting protein 1 | | | |
| | | (NUFIP1) | | | |
| 105 | translation | pf01205~Uncharacterized | | | 4592123 |
| | | protein family | | | |
| | | UPF0029; pf05773~RWD | | | |
| | | domain | | | |
| 106 | translation | pf00642~Zinc finger | | | 5191542 |
| | | C-x8-C-x5-C-x3-H | | | |
| | | type (and similar) | | | |
| 107 | translation | pf10343~Potential | | | 5322984 |
| | | Queuosine, Q, | | | |
| | | salvage protein | | | |
| | | family | | | |
| 108 | translation | pf03914~CBF/Mak21 | | | 5345426 |
| | | family | | | |
| 109 | translation | pf01746~tRNA | | | 5252961 |
| | | (Guanine-1)- | | | |
| | | methyltransferase | | | |
| 110 | protein | pf13519~von | | | 5327452 |
| | turnover | Willebrand factor | | | |
| | | type A domain | | | |
| 111 | protein | pf02290~Signal | | | 4154136 |
| | localization/ | recognition particle | | | |
| | folding | 14kD protein | | | |
| 112 | protein | pf03810~Importin- | | | 5413492 |
| | localization/ | beta N-terminal | | | |
| | folding | domain | | | |
| 113 | protein | pf00639~PPIC-type | | | 4145655 |
| | localization/ | PPIASE domain; | | | |
| | folding | peptidyl prolyl | | | |
| | | isomerase | | | |
| | 2C. Signaling | | | | |
| 114 | G-protein | G-protein coupled | | | 4976661 |
| | coupled | glucose receptor | | | |
| | receptors | regulating Gpa2 | | | |
| 115 | G-protein | 7 Lung | | | 565343 |
| | coupled | transmembrane | | | |
| | receptors | receptor | | | |
| 116 | Mitogen- | Mitogen-activated | | | 5321860 |
| | activated | protein kinase | | | |
| | protein kinase | (MAPK) | | | |
| | and associated | | | | |
| 117 | Mitogen- | MAPK and MTOR | | | 1275796 |
| | activated | activator | | | |
| | protein kinase | | | | |
| | and associated | | | | |
| 118 | Ca2+/calmodulin- | Ca2+/Calmodulin - | CAMK1 | 2.7.11.17 | 630401 |
| | dependent | dependent protein | protein | | |
| | protein kinase | kinase | kinase | | |
| 119 | Ca2+/calmodulin- | Ca2+/Calmodulin - | CAMK1 | | 36740 |
| | dependent | dependent protein | protein | | |
| | protein kinase | kinase, EF hand | kinase | | |
| | | superfamily, | | | |
| | | Forkhead domain | | | |
| 120 | 14-3-3 | Multifunctional | | | 5323330 |
| | proteins | chaperone 14-3-3 | | | |
| 121 | 14-3-3 | 14-3-3 protein | | | 2905756 |
| | proteins | | | | |
| | Process Category 3 - Manipulation of Plant Host | | | | |
| | 3A. Secreted proteins | | | | |
| 122 | Associated | G. orontii effector | OEC60/ | — | 4531743 |
| | with TCP | candidate 60/61 | OEC61 | | |
| | network | | | | |
| 123 | Associated | G. orontii effector | OEC70 | — | 5103140 |
| | with TCP | candidate 70 | | | |
| | network | | | | |
| 124 | Associated | G. orontii effector | OEC14 | | 4149374 |
| | with TCP | candidate 14 | | | |
| | network | | | | |
| 125 | Associated | G. orontii effector | OEC21 | | 4150772 |
| | with TCP | candidate 21 | | | |
| | network | | | | |
| 126 | Associated | G. orontii effector | OEC27/ | | 4575775 |
| | with TCP | candidate 27/39 | OEC39 | | |
| | network | | | | |
| 127 | Associated | G. orontii effector | OEC59/ | | 4590467 |
| | with TCP | candidate 59/68 | OEC68 | | |
| | network | | | | |
| 128 | Associated | G. orontii effector | OEC119 | | 743938 |
| | with TCP | candidate 119 | | | |
| | network | | | | |
| 129 | Associated | G. orontii effector | OEC45 | | 4735914 |
| | with TCP | candidate 45 | | | |
| | network | | | | |
| 130 | Associated | G. orontii effector | OEC16a | | 4153770 |
| | with TCP | candidate 16a | | | |
| | network | | | | |
| 131 | Associated | G. orontii effector | OEC35 | | 4145494 |
| | with TCP | candidate 35 | | | |
| | network | | | | |
| 132 | Associated | G. orontii effector | OEC49 | | 1049062 |
| | with TCP | candidate 49 | | | |
| | network | | | | |
| 133 | Associated | G. orontii effector | OEC55 | | 5329416 |
| | with TCP | candidate 55 | | | |
| | network | | | | |
| 134 | Associated | G. orontii effector | OEC101 | | 2952104 |
| | with TCP | candidate 101 | | | |
| | network | | | | |
| 135 | Associated | G. orontii effector | OEC110 | | 2010020 |
| | with TCP | candidate 110 | | | |
| | network | | | | |
| 136 | Associated | G. orontii effector | OEC117 | | 5333593 |
| | with TCP | candidate 117 | | | |
| | network | | | | |
| 137 | Associated | G. orontii effector | OEC120 | | 4382934 |
| | with TCP | candidate 120 | | | |
| | network | | | | |
| 138 | Associated | G. orontii effector | OEC123 | | 4611944 |
| | with TCP | candidate 123 | | | |
| | network | | | | |
| 139 | Associated | G. orontii effector | OEC125 | | 5332254 |
| | with TCP | candidate 125 | | | |
| | network | | | | |
| 140 | Most highly | effector protein EC2 | EC2 | — | 5406840 |
| | expressed | | | | |
| 141 | Most highly | Heat shock protein | HSP70 | EC:4.2.1.24 | 1225464 |
| | expressed | 70 family | | | |
| 142 | Most highly | hypothetical protein | | | 5098516 |
| | expressed | | | | |
| 143 | Most highly | expressed protein | | | 492385 |
| | expressed | | | | |
| 144 | Most highly | expressed protein | | | 4653020 |
| | expressed | | | | |
| | 3B. Plant hormone metabolism | | | | |
| 145 | Abscisic acid | β-carotene 15,15′- | BCDO, | 1.13.11.51 | 2191786 |
| | (carotenoid) | dioxygenase | CarX, | | |
| | | | NCED | | |
| 146 | Abscisic acid | xanthoxin oxidase | XO | 1.1.1.288 | 4598152 |
| | (carotenoid) | | | | |
| 147 | Abscisic acid | HMG CoA | HMGCR | | 2911432 |
| | (carotenoid) | Reductase | | | |
| 148 | Abscisic acid | ABA G-protein | ABA | | 2905863 |
| | (carotenoid) | coupled receptor | GPCR | | |
| 149 | Jasmonate | acyl-coA oxidase | ACX | 1.3.3.6 | 4150159 |
| | (a-linolenate, | | | | |
| | oxylipin) | | | | |
| 150 | Jasmonate (a- | enol-CoA hydratase | MFP2 | 4.2.1.17; | 5328133 |
| | linolenate, | | | 1.1.1.35; | |
| | oxylipin) | | | 1.1.1.211 | |
| 151 | Jasmonate (a- | acetyl-coA | | 2.3.1.16 | 581215 |
| | linolenate, | acyltransferase | | | |
| | oxylipin) | | | | |
| 152 | Salicylic acid, | chorismate synthase | CS | 4.2.3.5 | 4329927 |
| | auxin | | | | |
| | (chorismate) | | | | |
| 153 | Salicylic acid, | chorismate mutase | CMU | 5.4.99,5 | 161315 |
| | auxin | | | | |
| | (chorismate) | | | | |
| 154 | Salicylic acid, | anthranilate synthase | AS | 4.1.3.27 | 5323922 |
| | auxin | | | | |
| | (chorismate) | | | | |
| | Process Category 4- Essential cellular processes | | | | |
| | 4A. Cell cycle and DNA replication | | | | |
| 155 | cell | pf00566~Rab- | | | 1449934 |
| | cycle/DNA | GTPase-TBC domain | | | |
| | replication | | | | |
| 156 | cell | pf10444~Nbl1/ | | | 5400393 |
| | cycle/DNA | Borealin N terminal | | | |
| | replication | | | | |
| 157 | cell | pf06632~DNA | | | 5400544 |
| | cycle/DNA | double-strand break | | | |
| | replication | repair and V(D)J | | | |
| | | recombination | | | |
| | | protein XRCC4 | | | |
| 158 | cell | pf00515~Tetratricopeptide | | | 242553 |
| | cycle/DNA | repeat; pf07719~Tetra | | | |
| | replication | tricopeptide | | | |
| | | repeat; pf12895~Anaphase- | | | |
| | | promoting | | | |
| | | complex, cyclosome, | | | |
| | | subunit | | | |
| | | 3; pf13424~Tetratrico | | | |
| | | peptide repeat | | | |
| 159 | cell | pf12720~Protein of | | | 2660435 |
| | cycle/DNA | unknown function | | | |
| | replication | (DUF3807) | | | |
| 160 | cell | pf00515~Tetratricopeptide | | | 2911524 |
| | cycle/DNA | repeat; pf13414~TPR | | | |
| | replication | repeat | | | |
| 161 | cell | pf00400~WD | | | 5320921 |
| | cycle/DNA | domain, G-beta | | | |
| | replication | repeat | | | |
| 162 | cell | pf13414~TPR repeat | | | 5401173 |
| | cycle/DNA | | | | |
| | replication | | | | |
| 163 | cell | Anaphase- | | | 5334394 |
| | cycle/DNA | promoting | | | |
| | replication | complex/20S | | | |
| | | cyclosome subunit | | | |
| 180 | cell | Cyclin-like protein | | | 5320204 |
| | cycle/DNA | | | | |
| | replication | | | | |
| | 4B. Vesicles, autophagy, and phagocytosis | | | | |
| 164** | vesicles, | pf04109~Autophagy | | — | 4442491 |
| | autophagy, | protein Apg9 | | | |
| | and | | | | |
| | phagocytosis | | | | |
| 165 | vesicles, | pf04727~ELMO/CED-12 | | | 5400432 |
| | autophagy, | family | | | |
| | and | | | | |
| | phagocytosis | | | | |
| 166 | vesicles, | pf00789~UBX | | | 2926434 |
| | autophagy, | domain; pf08059~SEP | | | |
| | and | | | | |
| | phagocytosis | domain; pf14555~UBA- | | | |
| | | like domain | | | |
| | 4C. Ion/Metal transport and osmotic homeostasis | | | | |
| 167 | Cell volume | pf03517~Regulator | | | 2926268 |
| | control | of volume decrease | | | |
| | | after cellular | | | |
| | | swelling | | | |
| 168 | Metal/ion | CorA-like Mg2+ | | | 5322738 |
| | transporter | transporter | | | |
| 169 | Metal/ion | Ctr family Cu2+ | | | 2945681 |
| | transporter | transporter | | | |
| | 4D. Predicted important - unknown function | | | | |
| 170 | Single copy | N/A | | | 2908248 |
| | gene | | | | |
| | following | | | | |
| | WGD; | | | | |
| | Unknown | | | | |
| 171 | Single copy | N/A | | | 2914047 |
| | gene | | | | |
| | following | | | | |
| | WGD; | | | | |
| | Unknown | | | | |
| 172 | Single copy | N/A | | | 2925115 |
| | gene | | | | |
| | following | | | | |
| | WGD; | | | | |
| | Unknown | | | | |
| 173 | Single copy | N/A | | | 4153429 |
| | gene | | | | |
| | following | | | | |
| | WGD; | | | | |
| | Unknown | | | | |
| 174 | Single copy | N/A | | | 4153434 |
| | gene | | | | |
| | following | | | | |
| | WGD; | | | | |
| | Unknown | | | | |
| 175 | Single copy | N/A | | | 5376484 |
| | gene | | | | |
| | following | | | | |
| | WGD; | | | | |
| | Unknown | | | | |
| 176 | Single copy | N/A | | | 5322922 |
| | gene | | | | |
| | following | | | | |
| | WGD; | | | | |
| | Unknown | | | | |
| 177 | Single copy | N/A | | | 5300814 |
| | gene | | | | |
| | following | | | | |
| | WGD; | | | | |
| | Unknown | | | | |
| 178 | Single copy | pf11937~Protein of | | | 5222583 |
| | gene | unknown function | | | |
| | following | (DUF3455) | | | |
| | WGD; | | | | |
| | Unknown | | | | |
| 179 | Single copy | N/A | | | 5386051 |
| | gene | | | | |
| | following | | | | |
| | WGD; | | | | |
| | Unknown | | | | |

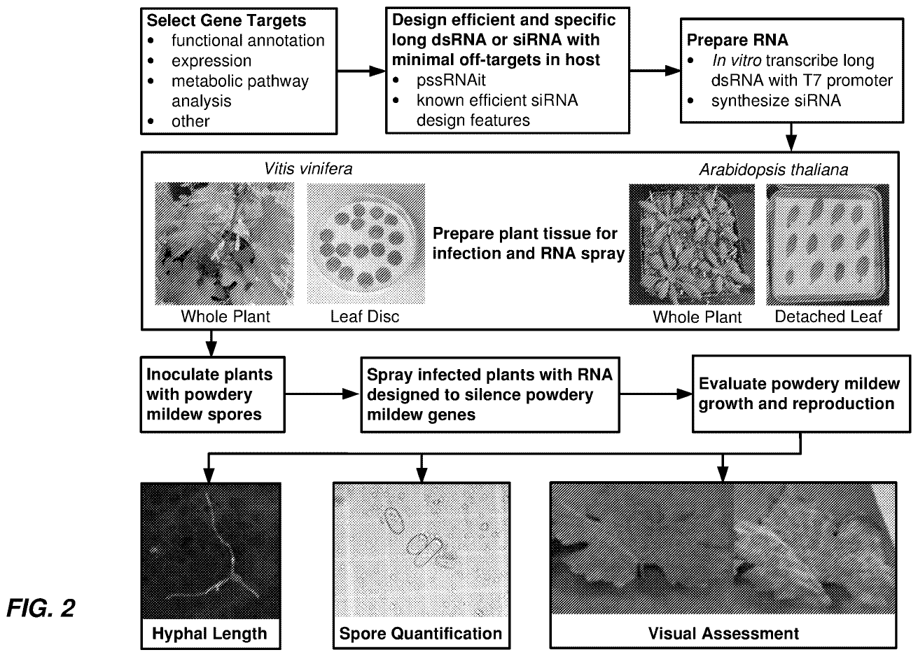

Using the G. orontii-A. thaliana pathosystem described herein (see, e.g. Example 1, and FIG. 2) target genes were readily identified that when downregulated in a phytopathogen will inhibit infectivity, and/or growth, and/or reproduction of that phytopathogen on a host plant (see, e.g., Table 3).

As demonstrated herein the selection and screening system described herein readily identifies effective powdery mildew target genes and Table 2, below, provides a list of illustrative G. orontii target genes and their orthologs in E. necator.

TABLE 2
Illustrative G. orontii genes and E. necator orthologs that, when down
regulated in their respective phytopathogen, inhibit infectivity, and/or
growth, and/or reproduction of that phytopathogen on a host plant. All
31 of these gene targets when down-regulated by SIGS in the G. orontii-
Arabidopsis system resulted in significantly reduced powdery mildew
growth and reproduction. Eleven grapevine powdery mildew orthologs of
these genes were silenced (bolded and underlined) and all eleven showed
significantly reduced grapevine powdery mildew growth and reproduction.
E. necator targets tested in vineyard trials are indicated with “**”
below the Sequence ID Number. All effectively reduced powdery mildew
proliferation (see, e.g., Examples 4 and 5). Nineteen of these individual
gene targets show reproducible mean >50% reduction in powdery mildew
growth and reproduction. Note that two E. necator orthologs of ICL were
tested and found to be effective targets. High confidence E. necator
orthologs of G. orontii effector candidate 14 (SEQ 124), single copy gene
unknown function (SEQ 179) and Alanine-glyoxylate aminotransferase
(SEQ 181) were not identified. The sequence IDs for the specific dsRNA
tested against a target are also provided.

| | | |
|---|---|---|
| | | dsRNA |
| | | SEQ |
| SEQ ID NO | Gene Name or PFAM Domain | ID NO |

G. orontii

| | | |
|---|---|---|
| 85 | Cytochrome P450 monoxygenase (CYP51) | 212 |
| 1 | TAG lipase 1, caroboxyesterase domain | 213 |
| 2 | TAG lipase A, extracellular lipase | 214 |
| 15 | Transaldolase | 215 |
| 20 | Isocitrate lyase (ICL) | 216 |
| 21 | Malate synthase (MS) | 217 |
| 22 | Citrate (Si) synthase | 218 |
| 32 | Trans-2-enoyl CoA reductase | 219 |
| 63 | Glutamate dehydrogenase | 220 |
| 64 | Threonine aldolase | 221 |
| 70 | Pf02548-Ketopantoate | 222 |
| | hydroxymethyltransferase | |
| 71 | Tetrahydrofolate synthase | 223 |
| 79 | Thymidine synthase | 224 |
| 87 | Pf8238-Sel1 repeat | 225 |
| 92 | Apoptosis antagonizing transcription factor | 226 |
| | (AATE) | |
| 104 | Pf10453- Nuclear fragile X mental retardation- | 227 |
| | interacting protein 1 (NUFIP1) | |
| 122 | G. orontii effector candidate 60/61 | 228 |
| 123 | G. orontii effector candidate 70 | 229 |
| 124 | G. orontii effector candidate 14 | 230 |
| 140 | Effector protein EC2 | 231 |
| 141 | Heat shock protein 70 family (HSP70) | 232 |
| 145 | β-carotene 15,15′-dioxygenase (BCDO) | 233 |
| 146 | xanthoxin oxidase | 234 |
| 148 | ABA G-protein coupled receptor | 235 |
| 149 | Acyl-CoA oxidase (ACX) | 236 |
| 163 | Anaphase promoting complex/20S cyclosome | 237 |
| | subunit | |
| 164 | Pf04109 - Autophagy protein Apg9 | 238 |
| 167 | Pf03517, Regulator of cell volume after swelling | 239 |
| 169 | Ctr family Cu2+ transporter | 240 |
| 179 | Single copy gene, unknown function | 241 |
| 180 | Cyclin-like protein | 242 |
| 181 | Alanine-glyoxylate aminotransferase | 243 |

E. necator orthologs

| | | |
|---|---|---|
| 182 | En Cytochrome P450 monoxygenase (CYP51) | 244 |
| ** | | |
| 183 | En TAG lipase 1, caroboxyesterase domain | 245 |
| ** | | |
| 184 | En TAG lipase A, extracellular lipase | 246 |
| 185 | Transaldolase | |
| 186, 187 | Isocitrate lyase (ICL) | 247, 248 |
| ** | | |
| 188 | Malate synthase (MS) | 249 |
| ** | | |
| 189 | Citrate (Si) synthase | |
| 190 | Trans-2-enoyl CoA reductase | |
| 191 | Glutamate dehydrogenase | |
| 192 | Threonine aldolase | |
| 193 | Pf02548- Ketopantoate hydroxymethyltransferase | |
| 194 | Tetrahydrofolate synthase | |
| 195 | Thymidine synthase | |
| 196 | Pf8238-Sell repeat | |
| 197 | Apoptosis antagonizing transcription factor | 250 |
| | (AATF) | |
| 198 | Pf10453- Nuclear fragile X mental retardation- | |
| | interacting protein 1 (NUFIP1) | |
| 199 | G. orontii effector candidate 60/61 ortholog | 251 |
| 200 | G. orontii effector candidate 70 ortholog | 252 |
| 201 | Effector protein EC2 | 253 |
| 202 | Heat shock protein 70 family (HSP70) | |
| 203 | β-carotene 15,15′-dioxygenase (BCDO) | 254 |
| 204 | xanthoxin oxidase | |
| 205 | ABA G-protein coupled receptor | |
| 206 | Acyl-CoA oxidase (ACX) | |
| 207 | Anaphase promoting complex/20S cyclosome | |
| | subunit | |
| 208 | Pf04109 - Autophagy protein Apg9 | 255 |
| ** | | |
| 209 | Pf03517, Regulator of cell volume after swelling | |
| 210 | Ctr family Cu2+ transporter | |
| 211 | Cyclin-like protein | |

All of the gene targets shown in Table 2, when down-regulated by SIGS in the G. orontii-Arabidopsis system resulted in significantly reduced powdery mildew growth and reproduction. Nineteen of these individual gene targets show reproducible mean>=50% reduction in powdery mildew growth and reproduction (e.g., see Example 5, Table 11).

Eleven of the E. necator grapevine powdery mildew orthologs of these genes were silenced (Table 2, bolded) and all eleven showed significantly reduced grapevine powdery mildew growth and reproduction. Eight of the eleven E. necator gene targets tested showed reproducible mean≥50% reduction in powdery mildew growth and reproduction (e.g. see Example 5, Table 11). Note that two orthologs of the JCL gene were tested; each gave significantly reduced powdery mildew growth and reproduction.

TABLE 3
Provides a summary of powdery mildew SIGS targets tested by process category. Sequence IDs are given
for G. orontii. See Table 2 for Sequence ID numbers for tested E. necator orthologs.

| | G. orontii | | | E. nectar | |
|---|---|---|---|---|---|
| | | | # Tested with | | # Tested with |
| | SEQ ID | # | reduced pathogen | # | reduced pathogen |
| Process Category | NOs | Tested | growth/reproduction | Tested | growth/reproduction |

Process Category 1 - Metabolism

| 1A. Lipid metabolism/acquisition | | 8 | 7 | 4 | 4 |
|---|---|---|---|---|---|
| TAG/glycerolipid | 1-14 | 3 | 2 | 2 | 2 |
| PPP pathway | 15-19 | 1 | 1 | | |
| TCA and glyoxylate | 20-31, 181 | 4 | 4 | 2 | 2 |
| cycle | | | | | |
| FA synthesis/transport | 32-41 | 1 | 1 | | |
| FA B-oxidation | 42-50 | 0 | 0 | | |
| Phospholipid | 51 | 0 | 0 | | |
| metabolism | | | | | |
| 1B. Carbohydrate metabolism | | 3 | 0 | | |
| glycogen metabolism | 52-53 | 2 | 0 | | |
| glycolysis | 54-57 | 1 | 0 | | |
| Carbohydrate utlization | 58-61 | 0 | 0 | | |
| 1C. Amino acid & nucleotide | | 6 | 5 | | |
| metabolism & salvage; cofactor | | | | | |
| metabolism | | | | | |
| amino acid | 62-69 | 3 | 2 | | |
| metabolism/salvage | | | | | |
| cofactor metabolism | 70-78 | 2 | 2 | | |
| nucleotide | 79-84 | 1 | 1 | | |
| metabolism/salvage | | | | | |
| 1D. Essential fungal-specific | | 1 | 1 | 1 | 1 |
| processes | | | | | |
| sterol biosynthesis | 85 | 1 | 1 | 1 | 1 |
| fungal sexual | 86 | 0 | 0 | | |
| reproduction/compatibility | | | | | |
| 1E. Mitochondrial electron | | 1 | 1 | | |
| transport chain & ATP synthesis | | | | | |
| mitochondrial electron | 87 | 1 | 1 | | |
| transport chain | | | | | |
| ATP synthesis | 88-89 | 0 | 0 | | |
| Alternative Respiratory | 90-91 | 0 | 0 | | |
| Pathway | | | | | |

Process Category 2 - Regulation

| 2A. Transcriptional Regulation | | 2 | 1 | 1 | 1 |
|---|---|---|---|---|---|
| stress response | 92-93 | 1 | 1 | 1 | 1 |
| metabolic regulation | 94-96 | 1 | 0 | | |
| development | 97-99 | 0 | 0 | | |
| transcription | 100-101 | 0 | 0 | | |
| RNA-binding | 102-103 | 0 | 0 | | |
| 2B. Protein regulation | | 1 | 1 | | |
| (synthesis/turnover/localization) | | | | | |
| translation | 104-109 | 1 | 1 | | |
| protein turnover | 110 | 0 | 0 | | |
| protein | 111-113 | 0 | 0 | | |
| localization/folding | | | | | |
| 2C. Signaling | | 0 | 0 | | |
| G-protein coupled | 114-115 | 0 | 0 | | |
| receptors | | | | | |
| Mitogen-activated | 116-117 | 0 | 0 | | |
| protein kinase and | | | | | |
| associated | | | | | |
| Ca2+/Calmodulin- | 118-119 | 0 | 0 | | |
| dependent protein | | | | | |
| kinases | | | | | |
| 14-3-3 proteins | 120-121 | 0 | 0 | | |

Process Category 3 - Manipulation of plant host

| 3A. Secreted proteins | | 9 | 5 | 3 | 3 |
|---|---|---|---|---|---|
| Associated with TCP | 122-139 | 7 | 3 | 2 | 2 |
| network | | | | | |
| Most highly expressed | 140-144 | 2 | 2 | 1 | 1 |
| 3B. Plant hormone metabolism | | 6 | 4 | 1 | 1 |
| abscisic acid | 145-148 | 4 | 3 | 1 | 1 |
| (carotenoid) | | | | | |
| jasmonate (a- | 149-151 | 1 | 1 | | |
| linolenate) | | | | | |
| salicylic acid, auxin | 152-154 | 1 | 0 | | |
| (chorismate) | | | | | |

Process Category 4 - Essential cellular processes

| 4A. Cell cycle and DNA | | 1 | 1 | | |
|---|---|---|---|---|---|
| replication | | | | | |
| cell cycle/DNA | 155-163, 180 | 1 | 1 | | |
| replication | | | | | |
| 4B. Vesicles, autophagy, and | | 1 | 1 | 1 | 1 |
| phagocytosis | | | | | |
| vesicles, autophagy, | 164-166 | 1 | 1 | 1 | 1 |
| and phagocytosis | | | | | |
| 4C. Ion/Metal transport and | | 2 | 2 | | |
| osmotic homeostasis | | | | | |
| Cell volume control | 167 | 1 | 1 | | |
| Metal/ion transporter | 168-169 | 1 | 1 | | |
| 4D. Predicted important - | | 1 | 1 | | |
| unknown function | | | | | |
| Single copy gene | 170-179 | 1 | 1 | | |
| following WGD; | | | | | |
| Unknown | | | | | |
| Total targets tested | | 42 | 31 | 11 | 11 |

Novel fungal targets identified in the powdery mildew-host plant (G. orontii-A. thaliana pathosystem) system described herein can be validated for similar roles in development and/or virulence in other fungi or other phytopathogens. Moreover, it is noted that the genomes of obligate biotrophs of plants have few genes compared to other similar fungi, but the genes and pathways that are retained are similar as are the genes and pathways that are lost (e.g., Spanu (2012) Ann. Rev. Phytopath. 50: 91-109).

It has previously proven difficult to assess the function and importance of genes of obligate biotrophs as most are not culturable or genetically tractable. However, in view of the genes and pathways retained by obligate biotrophs of plants, important targets identified using SIGS against powdery mildew genes identified using the G. orontii-A. thaliana pathosystem described herein are believed to also be important for the growth and interactions of other obligate biotrophs with their host.

Accordingly, translation of identified target genes from the powdery mildew system is not limited to obligate biotrophs but may also pertain to diverse pathogens including non-obligate biotrophs, hemibiotrophs, necrotrophs and saprotroph plant pathogens. Accordingly, in various embodiments, target genes as described herein also include orthologs of the G. orontii genes identified herein (see, e.g., Table 4).

TABLE 4
Illustrative, but non-limiting examples of G. orontii genes and orthologs that,
when down regulated in their respective phytopathogen, inhibit infectivity, and/or
growth, and/or reproduction of that phytopathogen on a host plant. See Definition of
orthologs. Orthologs sharing <35% protein identify to G. orontii sequence and an
E-value of ≤E−10 are flagged with the label “**”. Note that topical dsRNA
against E. necator orthologs of ten G. orontii targets (bolded) included below
were tested and shown to effectively reduce powdery mildew of grapevine.

| Golovinomyces orontii MGH1 | | Powdery mildew: Erysiphe necator | | |
|---|---|---|---|---|
| | SEQ | | Protein | |
| Gene | ID NO | E Value | Identity | Accession |
| CYP51 | 85 | 0.00E+00 | 74.43% | AAD55135.1 |
| MS | 21 | 0.00E+00 | 76.01% | KHJ34436.1 |
| ICL | 20 | 0.00E+00 | 78.47% | KHJ31678.1 |
| LIP1 | 1 | 0.00E+00 | 76.42% | KHJ32463.1 |
| LIPA | 2 | 3.00E−146 | 51.53% | KHJ34018.1 |
| BCDO | 145 | 0.00E+00 | 67.53% | KHJ33569.1 |
| ACX | 149 | 0.00E+00 | 80.40% | KHJ31624.1 |
| AATF | 92 | 4.00E−167 | 52.66% | KHJ33437.1 |
| EC2 | 140 | 3.00E−46 | 73.91% | KHJ33901.1 |
| OEC60/OEC61 | 122 | 0.00E+00 | 60.09% | KHJ32462.1 |
| OEC70 | 123 | 1.00E−21 | 53.40% | KHJ35927.1 |
| OEC14 | 124 | — | — | no hit |
| HSP70 | 141 | 0.00E+00 | 89.58% | KHJ35398.1 |
| XO | 146 | 0.00E+00 | 79.55% | KHJ32774.1 |
| APG9 | 164 | 0.00E+00 | 62.36% | KHJ32900.1 |

| | | Powdery mildew: | | |
|---|---|---|---|---|
| Golovinomyces orontii MGH1 | | Golovinomyces chicoracearum | | |
| | SEQ | | Protein | |
| Gene | ID NO | E Value | Identity | Accession |
| CYP51 | 85 | 0E+00 | 88.95% | RKF57889.1 |
| MS | 21 | 0E+00 | 89.85% | RKF62510.1 |
| ICL | 20 | 0E+00 | 92.65% | RKF59197.1 |
| LIP1 | 1 | 0E+00 | 96.58% | RKF56564.1 |
| LIPA | 2 | 0E+00 | 89.93% | RKF53624.1 |
| BCDO | 145 | 0E+00 | 88.23% | RKF53159.1 |
| AATF | 92 | 0E+00 | 86.27% | RKF77188.1 |
| EC2 | 140 | 8E−79 | 89.05% | RKF60824.1 |
| OEC60/OEC61 | 122 | 0E+00 | 87.75% | RKF80766.1 |
| OEC70 | 123 | 2E−46 | 85.29% | RKF78082.1 |
| HSP70 | 141 | 0E+00 | 96.75% | RKF75625.1 |
| XO | 146 | 0E+00 | 93.11% | RKF78744.1 |
| APG9 | 164 | 0E+00 | 77.32% | RKF78781.1 |

| | | Powdery Mildew: | | |
|---|---|---|---|---|
| Golovinomyces orontii MGH1 | | Erysiphe pisi Palampur-1 | | |
| | SEQ | | Protein | |
| Gene | ID NO | E Value | Identity | Accession |
| CYP51 | 85 | 0E+00 | 77.1% | 2309251 |
| MS | 21 | 0E+00 | 79.3% | 1335422 |
| ICL | 20 | 0E+00 | 79.0% | 2512998 |
| LIP1 | 1 | 0E+00 | 78.1% | 2804836 |
| LIPA | 2 | 0E+00 | 78.3% | 2804836 |
| BCDO | 145 | 0E+00 | 69.5% | 2319553 |
| AATF | 92 | 7E−144 | 62.8% | 2599204 |
| EC2 | 140 | 7E−53 | 76.3% | 2549560 |
| OEC60/OEC61 | 122 | 1E−152 | 65.2% | 2315721 |
| OEC70 | 123 | 2E−13 | 67.5% | 2526873 |
| HSP70 | 141 | 0E+00 | 91.4% | 2643551 |
| XO | 146 | 2E−155 | 81.8% | 2321156 |
| APG9 | 164 | 0E+00 | 59.5% | 2690623 |

| | | Powdery Mildew: Blumeria graminis | | |
|---|---|---|---|---|
| Golovinomyces orontii MGH1 | | f. sp. hordei DH14 | | |
| | SEQ | | Protein | |
| Gene | ID NO | E Value | Identity | Accession |
| CYP51 | 85 | 0E+00 | 77.89% | CCU76296.1 |
| MS | 21 | 0E+00 | 71.56% | CCU83094.1 |
| ICL | 20 | 0E+00 | 74.54% | CCU76011.1 |
| LIP1 | 1 | 0E+00 | 70.19% | CCU76452.1 |
| LIPA | 2 | 9E−118 | 42.53% | CCU76779.1 |
| BCDO | 145 | | | no hit |
| AATF | 92 | 5E−128 | 53.42% | CCU74232.1 |
| EC2 | 140 | 2E−34 | 60.67% | CCU81481.1 |
| OEC60/OEC61 | 122 | 5E−127 | 49.49% | CCU76102.1 |
| OEC70 | 123 | 1E−05 | 42.11% | CCU76753.1 |
| HSP70 | 141 | 0E+00 | 88.26% | CCU75862.1 |
| XO | 146 | 3E−171 | 78.47% | CCU82271.1 |
| APG9 | 164 | 0E+00 | 54.73% | CCU75759.1 |

| Golovinomyces orontii MGH1 | | Grey Mold: Botrytis cinerea | | |
|---|---|---|---|---|
| | SEQ | | Protein | |
| Gene | ID NO | E Value | Identity | Accession |
| CYP51 | 85 | 0.00E+00 | 72.14% | XP_001549961.1 |
| MS | 21 | 0.00E+00 | 76.01% | XP_001551737.1 |
| ICL | 20 | 0.00E+00 | 75.57% | EMR88386.1 |
| LIP1 | 1 | 0.00E+00 | 48.60% | XP_001553224.2 |
| LIPA | 2 | 6.00E−102 | 44.69% | XP_001561332.1 |
| BCDO | 145 | 0.00E+00 | 63.38% | EMR86130.1 |
| ACX | 149 | 0.00E+00 | 74.82% | XP_001553115.1 |
| AATF | 92 | 7.00E−22 | 55.21% | XP_001546894.1 |
| EC2 | 140 | — | — | Below cutoff |
| OEC60/OEC61 | 122 | 1.00E−104 | 59.38% | EMR90602.1 |
| OEC70 | 123 | — | — | no hit |
| OEC14 | 124 | — | — | no hit |
| HSP70 | 141 | 0.00E+00 | 88.13% | XP_001557140.1 |
| XO | 146 | 1.00E−176 | 79.00% | XP_024550660.1 |
| APG9 | 164 | 0.00E+00 | 49.26% | XP_024553178.1 |
| Golovinomyces orontii MGH1 | | Rust: Fusarium graminearum | | |
| | SEQ | | Protein | |
| Gene | ID NO | E Value | Identity | Accession |
| CYP51 | 85 | 0.00E+00 | 63.89% | XP_011316750.1 |
| MS | 21 | 0.00E+00 | 73.06% | XP_011320034.1 |
| ICL | 20 | 0.00E+00 | 72.07% | XP_011315789.1 |
| LIP1 | 1 | 1.00E−177 | 48.42% | XP_011317424.1 |
| LIPA | 2 | 2.00E−96 | 41.90% | XP_011317015.1 |
| BCDO | 145 | 0.00E+00 | 53.87% | SCB65744.1 |
| ACX | 149 | 0.00E+00 | 66.13% | EYB30953.1 |
| AATF | 92 | 1.00E−67 | 41.90% | XP_011320396.1 |
| EC2 | 140 | — | — | no hit |
| OEC60/OEC61 | 122 | 5.00E−81 | 36.54% | EYB23888.1 |
| OEC70 | 123 | — | — | no hit |
| OEC14 | 124 | — | — | no hit |
| HSP70 | 141 | 0.00E+00 | 82.53% | XP_011328252.1 |
| XO | 146 | 2.00E−145 | 71.79% | XP_011315790.1 |
| APG9 | 164 | 0.00E+00 | 43.45% | PCD18080.1 |

| Golovinomyces orontii MGH1 | | Smut Fungi: Ustilago maydis | | |
|---|---|---|---|---|
| | SEQ | | Protein | |
| Gene | ID NO | E Value | Identity | Accession |
| CYP51 | 85 | 1.00E−165 | 50.81% | XP_011390148.1 |
| MS | 21 | 0.00E+00 | 55.06% | XP_011389866.1 |
| ICL | 20 | 0.00E+00 | 55.84% | XP_011387825.1 |
| LIP1 | 1 | 2.00E−76 ** | 33.68% ** | XP_011386124.1 ** |
| LIPA | 2 | — | — | no hit |
| BCDO | 145 | — | — | no hit |
| ACX | 149 | 0.00E+00 | 46.61% | XP_011388551.1 |
| AATF | 92 | 3.00E−15 ** | 25.34% ** | XP_011390839.1 ** |
| EC2 | 140 | — | — | no hit |
| OEC60/OEC61 | 122 | — | — | no hit |
| OEC70 | 123 | — | — | no hit |
| OEC14 | 124 | — | — | no hit |
| HSP70 | 141 | 0.00E+00 | 64.84% | XP_011387505.1 |
| XO | 146 | 4.00E−94 | 53.21% | XP_011392305.1 |
| APG9 | 164 | 4.00E−142 | 43.40% | XP_011391082.1 |
| Golovinomyces orontii MGH1 | | Oomycete: Phytophthora infestans | | |
| | SEQ | | Protein | |
| Gene | ID NO | E Value | Identity | Accession |
| CYP51 | 85 | 1.00E−10 | 25.37% | KAF4039312.1 |
| MS | 21 | 0.00E+00 | 54.39% | XP_002906508.1 |
| ICL2 | 20 | 7.00E−150 | 42.83% | XP_002904993.1 |
| LIP1 | 1 | 5.00E−55 ** | 30.87% ** | KAF4042355.1 ** |
| LIPA | 2 | 4.00E−17 | 35.88% | XP_002902375.1 |
| BCDO | 145 | — | — | no hit |
| ACX | 149 | 4.00E−125 | 35.37% | KAF4034308.1 |
| AATF | 92 | 9.00E−14 ** | 25.49% ** | XP_002905548.1 ** |
| EC2 | 140 | — | — | no hit |
| OEC60/OEC61 | 122 | 2.00E−13 ** | 30.85% ** | KAF4038576.1 ** |
| OEC70 | 123 | — | — | no hit |
| OEC14 | 124 | — | — | no hit |
| HSP70 | 141 | 0.00E+00 | 65.47% | XP_002909118.1 |
| XO | 146 | 2.00E−82 | 48.76% | XP_002906531.1 |
| APG9 | 164 | 5.00E−46 | 27.84% | XP_002906147.1 |

Thus, in certain embodiments the target gene(s) include the target genes shown in Tables 1 and 2, as well as the orthologs shown in Table 4, as well as other orthologs.

In certain embodiments, the target gene is selected from the group consisting of G. orontii lipase A, G. orontii lipase 1, G. orontii 03-carotene 15,15′-dioxygenase, G. orontii apoptosis-antagonizing transcription factor (AATF), effector candidate 2, OEC14, OEC60/OEC61, and OEC70. In certain embodiments, the target gene is a homolog/ortholog of a gene selected from the group consisting of G. orontii lipase a, G. orontii lipase 1, G. orontii 03-carotene 15,15′-dioxygenase, G. orontii apoptosis-antagonizing transcription factor (AATF), effector candidate 2, OEC14, OEC60/OEC61, and OEC70.

In certain embodiments, the target gene is selected from genes identified as single copy following recent whole genome duplication in G. orontii MGH1 (see, e.g., Table 1 and Example 3) as well as orthologs thereof.

In certain embodiments, the target gene is selected from genes that encode identified as potentially secreted by the powdery mildew, including effectors (see, e.g., Table 1) and Examples 1 and 2).

In certain embodiments, the target gene is selected genes encoding proteins identified as interacting with plant TCP transcription factors or their interactors (see, e.g., Table 1, and Example 2).

In certain embodiments, the target gene is a gene identified using comparative genomics, transcriptomics, evolutionary analyses, and integrated metabolic pathway analysis where the gene is:
1. Predicted to function in primary metabolism including energy generation (see, e.g., Tables 1 and 3, Category 1; and Example 1);
2. Predicted to function in fungal-specific metabolism (see, e.g., Tables 1 and 3; Category 1D, and Example 1);
3. Predicted to function in acquiring or modifying an essential nutrient not made de novo in the powdery mildew based on incomplete or missing metabolic pathways (see, e.g., Tables 1 and 3, Category 1C, Amino acid & nucleotide metabolism & salvage; cofactor metabolism, and Example 1);
4. Predicted to function in acquiring or modifying an energy-rich or costly-to-make precursor or compound from the plant and utilize it (see, e.g., Table 1, Category 1A. Lipid metabolism/acquisition, and Example 1);
5. Predicted to function in manipulating a plant hormone or other plant metabolite (see, e.g., Tables 1 and 3, Category 3B, Plant hormone metabolism, and Example 1);
6. Predicted to function in the regulation of phytopathogen growth, development, homeostasis, and response to the environment (see, eg., Tables 1 and 3, Category 2);
7. Predicted to function in other essential cellular processes (see eg., Tables 1 and 3, Category 4); and/or
8. Predicted to function in other processes of added import to an obligate biotroph (see e.g., Table 1, Category 4B vesicles, autophagy and phagocytosis and 4C ion/metal transport and osmotic homeostasis).

In certain embodiments, the ortholog is an ortholog found in an obligate biotroph. In certain embodiments, the ortholog is an ortholog found in a powdery mildew. In certain embodiments, the ortholog is an ortholog found in a powdery mildew selected from the group consisting of Erysiphe necator (or Uncinula necator) (powdery mildew of grapes), Blumeria graminis (or Erysiphe graminis) f. sp. tritici (causes powdery mildew of wheat), f. sp. hordei (powdery mildew of barley), f. sp. avenae (powdery mildew of oats), f. sp. secalis (powdery mildew of rye), Microsphaera diffusa (powdery mildew of legumes, e.g. soybean), Erysiphe pisi (powdery mildew of pea and other legumes) Leveillula taurica (also known as Oidiopsis taurica) (powdery mildew of onion, tomato or artichoke), Psuedoidium neolycopersici (or Oidium neolycopersici) (powdery mildew of tomato), Oidium sp. (powdery mildew of citrus), Podosphaera leucotricha (powdery mildew of apples and pears), Golovinomyces sp. Including G. cichoracearum, G. orontii, G. ambrosiae, G. spadiceus (powdery mildew of cucurbits, cannabis, hemp and sunflower) and Podosphaera sp. including P. macularis, P. xanthii, P. fusca, and P. aphanis, (powdery mildew of hops, cannabis, hemp, sunflower, cucurbits, melons and gourds), Sphaerotheca humuli (ornamentals), Sphaerotheca aphanis (ornamentals), Sphaerotheca mors-uvae (Black currant), Sphaerotheca pannosa (rose), Erysiphe polygoni (Cowpeas), Oidium begonia (Begonia), Podosphaera leucotricha (Fruit trees) and Podosphaera xanthii (or Sphaerotheca fuligenea, Sphaerotheca cucurbiteae) (powdery mildew of cucurbits: cucumbers, squashes (including pumpkins), luffas, melons, and watermelons). In certain embodiments, the ortholog is an ortholog found in Erysiphe necator (or Uncinula necator). In certain embodiments the ortholog comprises Arthraocladiella on goji berries, Brasiliomyces malachrae on cotton, Cystotheca wrightii on oaks, Erysiphe alphitoides on Oaks, Neoerysiphe galeopsidis on herbs, Oidium heveae on rubber plant, Parauncinula polyspora on oaks, Phyllactinia guttata on Hazel and others, Pleochaeta shiraiana on berries and trees, Sawadaea tulasnei on maple, and the like.

In certain embodiments, the target gene ortholog is an ortholog found in an ascomycete (e.g., Fusarium spp. (Fusarium wilt, stem rot and root rot disease), Thielaviopsis spp. (canker rot, black root rot, Thielaviopsis root rot), Verticillium spp., Magnaporthe grisea (rice blast), Botrytis spp. (Botrytis rots, grey mold) and Sclerotinia spp. (stem rot or white mold)). In certain embodiments, target gene ortholog an ortholog found in a Basidiomycete (e.g., Smut of cereals Ustilago spp., Sphacelotheca spp., Urocystis spp., Tilletia spp., etc.), Rhizoctonia spp., and Armillaria spp. (honey fungus species, virulent pathogens of trees), and plant rusts).

In certain embodiments, the target gene ortholog is an ortholog found in a plant rust. In certain embodiments, the ortholog is an ortholog found in a plant rust selected from the group consisting of Cronartium ribicola (White pine blister rust), Gymnosporangium juniperi-virginianae (Cedar-apple rust), Hemileia vastatrix (Coffee rust), Phakopsora meibomiae and P. pachyrhizi (Soybean rust), Puccinia spp. on many plants e.g. Puccinia coronata (Crown Rust of Oats and Ryegrass), Puccinia graminis (Stem rust of wheat and Kentucky bluegrass, or black rust of cereals), Puccinia hemerocallidis (Daylily rust), Puccinia triticina (Brown Wheat Rust), Puccinia sorghi (Common Rust of Corn), Puccinia striiformis (Yellow Rust) of cereals, Uromyces appendiculatus (Bean Rust), Puccinia melanocephala (Brown Rust of Sugarcane), and Puccinia kuehnii (Orange rust of Sugar cane), and the like).

In certain embodiments, the target gene ortholog is an ortholog found in an oomycete. E.g. Phytophthora spp. including but not limited to Phytophthora infestans (Late blight), Phytophthora ramorum (Sudden oak death and Ramorum disease), Phytophthora sojae (Stem and root rot) Phytophthora capsici (Blight; stem and fruit rot; various others), Phytophthora cinnamomi (Root rot and dieback), Phytophthora parasitica (Root and stem rot and various others), Phytophthora palmivora, Phytophthora alni, Phytophthora brassicae, Phytophthora cactorum (formerly P. citricola), Phytophthora meadii, Phytophthora phaseoli, Phytophthora plurivora; White rust pathogens Albugo candida and Albugo laibachii; Downy mildew pathogens Bremia lactucae, Hyaloperonospora arabidopsidis, Pseudoperonospora cubensis, Plasmopara halstedii, Plasmopara obducens, Plasmopara viticola, Plasmopara halstedii, Peronophythora litchi, Peronospora manshurica, Peronosclerospora sorghi, Plasmodiophora brassicae, Peronospora belbahrii, Pseudoperonospora humuli, Hyaloperonospora brassicae; root rot pathogen Aphanomyces euteiches; Damping off and root rot pathogens Pythium ultimum, Pythium aphanidermatum, Pythium oligandrum, Brown stripe downy mildew pathogen of Barley and Maize Sclerophthora rayssiae.

The above-identified fungal diseases (and corresponding orthologous target genes) are illustrative and non-limiting.

Thus, other illustrative fungal diseases herein include, for example, Sclerotinia rots (S. sclerotiorum and S. minor), Sclerotium rots (Sclerotium rolfsii and S. cepivorum), Fusarium wilts and rots (various Fusarium species including F. solani and F. oxysporum), Botrytis rots (e.g., Grey mould (Botrytis cinerea), Anthracnose (Colletotrichum spp. except for in lettuce—Microdochium panattonianum), Rhizoctonia rots (e.g., Rhizoctonia solani)—range of common names, e.g. Bottom rot (lettuce) and Wire stem (Brassicas), Damping-off (Pythium, Rhizoctonia, Phytophthora, Fusarium or Aphanomyces), Cavity spot (e.g., Pythium sulcatum), Tuber diseases, Black root rot, Target spot, Aphanomyces root rot (e.g., Aphanomyces euteiches pv. phaseoli (beans), Aschochyta collar rot (peas), Gummy stem blight (e.g., Didymella bryoniae (cucurbits)), alternaria leaf spot (e.g., Alternaria cucumerina, A. alternata), black leg (e.g., Leptosphaeria maculans (brassicas)), ring spot (e.g., Mycosphaerella brassicicola (brassicas)), late blight (e.g., Septoria apiicola (celery)), cercospora leaf spot (e.g. Cercospora beticola (beets)), leaf blight (e.g., Septoria petroelini (parsley)), septoria spot (e.g., Septoria lactucae (lettuce)), leaf blight (e.g., Stemphylium vesicarium (spring onions)), leaf blight (e.g., Alternaria dauci (carrots)), and the like).

In certain embodiments, the target gene ortholog is a gene found in a fungus selected from the genus Alternaria, Aphanomyces, Arthraocladiella, Ashbya, Aspergillus, Beauveria, Bipolaris, Blumeria, Botryotinia, Botrytis, Brasiliomyces, Ceratocystis, Cercospora, Chaetomium, Cladosporium, Claviceps, Coccidioides, Cochiobolus, Colletotrichum, Cordyceps, Corticum, Corynespora, Cryptococcus, Cycloconium, Cystotheca, Dematophora, Diaporthe, Diplocarpon, Diplodia, Drecshlera, Elsinoe, Endocarpon, Erysiphe, Esca, Eutypa, Exophiala, Exserophilum, Fusarium, Gaeumannomyces, Geotrichum, Gibberella, Glomerella, Guignardia, Golovinomyces, Helminthosporium, Isariopsis, Lachancea, Leptosphaeria, Leveillula, Macrophomina, Magnaporthe, Marssonina, Metarhizium, Microsphaera, Monographella, Myceliophthora, Mycosphaerella, Neoerysiphe, Neurospora, Oidium, Paracoccidioides, Parauncinula, Penicillium, Phaeosphaeria, Phakospora, Phomopsis, Phyllactinia, Pleochaeta, Podosphaera, Podospora, Pseudocercospora., Pseudopeziza, Psuedoidium, Puccinia, Pyrenophora, Pyricularia, Ramularia, Rhizoctonia, Rhizopus, Saccharomyces, Sawadaea, Schizsaccharomyces, Sclerotinia, Sclerotium, Sordaria, Stagonospora, Talaromyces, Taphrina, Thielavia, Trichoderma, Ustilago, Venturia, Verticillium, Zygosaccharomyces, Zymoseptoria, and the like.

In certain embodiments, the target gene ortholog is a gene found in selected from the group consisting of Alternaria alternata, Alternaria solani, Aphanomyces cochioides, Ashbya gossypii, Aspergillus flavus, Beauveria bassiana, Bipolaris maydis, Bipolaris sorokiniana, Blumeria graminis, Blumeria graminis, Botryotinia fuckeliana, Botrytis cinerea, Bremia lactucae, Cercospora beticola, Cercospora kikuchii, Cercospora sojina, Cercospora zea maydis, Chaetomium globosum, Claviceps purpurea, Coccidioides posadasi, Cochliobolus heterostrophus, Colletotrichum gloeosporioides, Colletotrichum graminicola, Colletotrichum higginsianum, Colletotrichum orbiculare, Cordyceps militaris, Diplodia maydis, Drechsiera oryzae, Drechslera glycines, Endocarpon pusillum, Erysiphe betae, Erysiphe necator, Erysiphe pisi, Exophiala dermatitidis, Exserohilum turcicum, Fusariumfujikuroi, Fusarium graminearum, Fusarium moniliforme, Fusarium oxysporum, Fusarium solani, Gaeumannomyces graminis, Golovinomyces cichoracearum, Golovinomyces cichoracearum, Lachancea thermotoleran, Leptosphaeria maculans, Leveillula taurica, Macrophomina phaseolina, Magnaporthe grisea, Magnaporthe oryzae, Marssonina brunnea, Metarhizium acridum, Metarhizium anisopliae, Microsphaera diffusa, Monographella albescens, Myceliophthora thermophile, Paracoccidioides brasiliensis, Penicillium spp., Phaeoshaeria maydis, Phaeosphaeria nodorum, Phakopsora pachyrhizi, Phytophthora infestans, Podosphaera xanthii, Pseudocercospora fijiensis, Puccinia graminis, Puccinia polysora, Puccinia striiformis, Puccinia triticina, Pyrenophora teres, Pyrenophora tritici-repentis, Pyricularia oryzae, Pythium ultimum, Ramularia beticola, Rhizoctonia oryzae, Rhizoctonia solani, Saccharomyces arboricola, Schizsacchaormyces japonicus, Sclerotinia sclerotiorum, Sclerotium rolfsii, Sordaria macrospora, Sphaerotheca pannosa, Talaromyces marneffei, Talaromyces stipitatus, Thielavia terrestris, Trichoderma vireos, Ustilago hordei, Ustilago maydis, Venturia inaequalis, Verticillium albo-atrum, Verticillium alfalfa, Verticillium dahlia, Verticillium dahliae, Zygosaccharomyces bailii, Zygosaccharomyces rouxii, Zymoseptoria tritici, and the like. In certain embodiments, the target gene may be a gene in a fungus selected from the genus Candida such as Candida albicans, Candida glabrata, Candida orthopsilosis, Candida parapsilosis, or Candida tropicalis.

In certain embodiments, the fungal genes described herein may serve in the finding of not yet identified gene(s) of phytopathogenic fungi, e.g., via conserved sequences.

In certain embodiments the phytopathogen comprise a bacterial phytopathogen, e.g., Erwinia sp., Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasma., streptomyces, Bacillus, Clostridium, Candidatus, Pectobacterium.

In certain embodiments, the target gene ortholog is an ortholog found in a nematod phytopathogen. In certain embodiments the nematode phytopathogen is from the group of e.g., Root-knot nematodes (Meloidogyne spp.); Cyst nematodes (Heterodera and Globodera spp.); Root lesion nematodes (Pratylenchus spp.); Burrowing nematode Radopholus similis; Ditylenchus dipsaci; The pine wilt nematode Bursaphelenchus xylophilus; The reniform nematode Rotylenchulus reniformis; Xiphinema index; Nacobbus aberrans; Aphelenchoides besseyi; Hirschmaniella spp.; Ancylostoma; Anisakid; Aphelenchoides spp.; a nematode which is a plant pathogenic nematode, such as but not limited to Meloidogyne spp. (e.g., M. incognita, M. javanica, M. graminicola, M. arenaria, M. chitwoodi, M. hapla or M. paranaensis); Heterodera spp. (e.g., H. oryzae, H. glycines, H. zeae or H. schachtii); Globodera spp. (e.g., G. pallida or G. rostochiensis); Roty-lenchulus spp. (e.g., R. reniformis); Pratylenchus spp. (e.g., P. coffeae, P. Zeae or P. goodeyi); Radopholus spp. (e.g., R. similis); Hirschmaniella spp. (e.g., H. oryzae), Ancylostoma spp. (e.g., A. caninum, A. ceylanicum, A. duodenale or A. tubaeforme); Anisakid; Aphelenchoides spp. (e.g., A. Besseyi); Ascarids; Ascaris spp., (e.g., A. suum or A. lum-bridoides); Belonolaimus spp.; Brugia spp. (e.g., B. malayi or B. pahangi); Bursaphelenchus spp.; Caenorhabditis spp. (e.g., C. elegans, C. briggsae or C. remand); Clostridium spp. (e.g., C. acetobutylicum); Cooperia spp. (e.g., C. onco-phora)′, Criconemoides spp.; Cyathostomum spp. (e.g., C. catinatum, C. coronation or C. pateratum); Cylicocyclus spp. (e.g., C. insigne, C. nassatus or C. radiatus)′, Cyli-costephanus spp. (e.g., C. goldi or C. longibursatus); Diphyllobothrium; Dirofilaria spp. (e.g., D. immitis); Ditylenchus spp. (e.g., I), dipsaci, I), destructor or D. augustus); Entero-bius spp. (e.g., E. vermicularis); Haemonchus spp. (e.g., H. contortus); Hirschmanniella spp., Helicotylenchus spp.; Hoplolaimus spp.; Lito-mosoides spp. (e.g., L. sigmodontis); Longidorus spp. (e.g., L. macrosoma); Necator spp. (e.g., N. americanus)′, Nip-postrongylus spp. (e.g., N. brasiliensis)′, Onchocerca spp. (e.g., O. volvulus)′, Ostertagia spp. (e.g., O. ostertagi); Paras-trongyloides spp. (e.g., P. trichosuri)′, Paratrichodorus spp. (e.g., P. minor or P. teres); Parelaphostrongylus spp. (e.g., P. tenuis); Radophulus spp.; Rotylenchulus spp.; Scutellonerna. spp.; Strongyloides spp. (e.g., S′. ratti or S. stercoralis); Teladorsagia spp. (e.g., T. circumcincta); Toxascaris spp. (e.g., T. leonina); Toxocara spp. (e.g., T. canis or T. cati); Trichinella spp. (e.g., T. britovi, T. spiralis or T. spirae); Trichodorus spp. (e.g., T. similis); Trichuris spp. (e.g., T. muris, T. vulpis or T. trichiura); Tylenchulus spp.; Tylenchorhynchus spp.; Uncinaria spp. (e.g., U. stenocephala); Wuchereria spp. (e.g., W. Bancrofti) Xiphinema spp. (e.g., X. index or X. americanum), and the like.

In certain embodiments, the target gene ortholog is an ortholog found in an insect pest. In another embodiment, the insect pests is from the Subphyla Chelicerata, Myriapoda, and/or Hexapoda. In yet another embodiment, insect pest belongs to the Classes of Arachnida, Symphyla, and/or Insecta. In another embodiment, the ortholog is selected from the insect from Order Homoptera.

In another embodiment, the insect pest is from the Order Anoplura. Non-limiting examples of genera may include, but are not limited to, Haematopinus spp., Hoplopleura spp., Linognathus spp., Pediculus spp., and Polyplax spp. Non-limiting examples of species may include, but are not limited to, Haematopinus asini, Haematopinus suis, Linognathus setosus, Linognathus ovillus, Pediculus humanus capitis, Pediculus humanus humanus, and Pthirus pubis.

In yet another embodiment, the insect pest is from the Order Coleoptera. Non-limiting examples of genera may include, but are not limited to, Acanthoscelides spp., Agriotes spp., Anthonomus spp., Apion spp., Apogonia spp., Aulacophora spp., Bruchus spp., Cerosterna spp., Cerotoma spp., Ceutorhynchus spp., Chaetocnema spp., Colaspis spp., Ctenicera spp., Curculio spp., Cyclocephala spp., Diabrotica spp., Hypera spp., Ips spp., Lyctus spp., Megascelis spp., Meligethes spp., Otiorhynchus spp., Pantomorus spp., Phyllophaga spp., Phyllotreta spp., Rhizotrogus spp., Rhynchites spp., Rhynchophorus spp., Scolytus spp., Sphenophorus spp., Sitophilus spp., and Tribolium spp. Non-limiting examples of species may include, but are not limited to, Acanthoscelides obtectus, Agrilus planipennis, Anoplophora glabripennis, Anthonomus grandis, Ataenius spretulus, Atomaria linearis, Bothynoderes punctiventris, Bruchus pisorum, Callosobruchus maculatus, Carpophilus hemipterus, Cassida vittata, Cerotoma trifurcata, Ceutorhynchus assimilis, Ceutorhynchus napi, Conoderus scalaris, Conoderus stigmosus, Conotrachelus nenuphar, Cotinis nitida, Crioceris asparagi, Cryptolestes ferrugineus, Cryptolestes pusillus, Cryptolestes turcicus, Cylindrocopturus adspersus, Deporaus marginatus, Dermestes lardarius, Dermestes maculatus, Epilachna varivestis, Faustinus cubae, Hylobius pales, Hypera postica, Hypothenemus hampei, Lasioderma serricorne, Leptinotarsa decemlineata, Liogenys fuscus, Liogenys suturalis, Lissorhoptrus oryzophilus, Maecolaspis joliveti, Melanotus communis, Meligethes aeneus, Melolontha melolontha, Oberea brevis, Oberea linearis, Oryctes rhinoceros, Oryzaephilus mercator, Oryzaephilus surinamensis, Oulema melanopus, Oulema oryzae, Phyllophaga cuyabana, Popillia japonica, Prostephanus truncatus, Rhyzopertha dominica, Sitona lineatus, Sitophilus granarius, Sitophilus oryzae, Sitophilus zeamais, Stegobium paniceum, Tribolium castaneum, Tribolium confusum, Trogoderma variabile, and Zabrus tenebrioides.

In an alternative embodiment, the insect pest is from the Order Dermaptera. In another embodiment, the insect pest is from the Order Blattaria. Non-limiting examples of species may include, but are not limited to, Blattella germanica, Blatta orientalis, Parcoblatta pennsylvanica, Periplaneta americana, Periplaneta australasiae, Periplaneta brunnea, Periplaneta fuliginosa, Pycnoscelus surinamensis, and Supella longipalpa.

In yet another embodiment, the insect pest is from the Order Diptera. Non-limiting examples of genera may include, but are not limited to, Aedes spp., Agromyza spp., Anastrepha spp., Anopheles spp., Bactrocera spp., Ceratitis spp., Chrysops spp., Cochliomyia spp., Contarinia spp., Culex spp., Dasineura spp., Delia spp., Drosophila spp., Fannia spp., Hylemyia spp., Liriomyza spp., Musca spp., Phorbia spp., Tabanus spp., and Tipula spp. Non-limiting examples of species may include, but are not limited to, Agromyza frontella, Anastrepha suspensa, Anastrepha ludens, Anastrepha obliqa, Bactrocera cucurbitae, Bactrocera dorsalis, Bactrocera invadens, Bactrocera zonata, Ceratitis capitata, Dasineura brassicae, Delia platura, Fannia canicularis, Fannia scalaris, Gasterophilus intestinalis, Gracillia perseae, Haematobia irritans, Hypoderma lineatum, Liriomyza brassicae, Melophagus ovinus, Musca autumnalis, Musca domestica, Oestrus ovis, Oscinella frit, Pegomya betae, Psila rosae, Rhagoletis cerasi, Rhagoletis pomonella, Rhagoletis mendax, Sitodiplosis mosellana, and Stomoxys calcitrans.

In a particular embodiment, the insect pest is from the Order Hemiptera. Non-limiting examples of genera may include, but are not limited to, Adelges spp., Aulacaspis spp., Aphrophora spp., Aphis spp., Bemisia spp., Ceroplastes spp., Chionaspis spp., Chrysomphalus spp., Coccus spp., Empoasca spp., Lepidosaphes spp., Lagynotomus spp., Lygus spp., Macrosiphum spp., Nephotettix spp., Nezara spp., Philaenus spp., Phytocoris spp., Piezodorus spp., Planococcus spp., Pseudococcus spp., Rhopalosiphum spp., Saissetia spp., Therioaphis spp., Toumeyella spp., Toxoptera spp., Trialeurodes spp., Triatoma spp. and Unaspis spp. Non-limiting examples of species may include, but are not limited to, Acrosternum hilare, Acyrthosiphon pisum, Aleyrodes proletella, Aleurodicus dispersus, Aleurothrixus floccosus, Amrasca biguttula biguttula, Aonidiella aurantii, Aphis gossypii, Aphis glycines, Aphis pomi, Aulacorthum solani, Bemisia argentifolii, Bemisia tabaci, Blissus leucopterus, Brachycorynella asparagi, Brevennia rehi, Brevicoryne brassicae, Calocoris norvegicus, Ceroplastes rubens, Cimex hemipterus, Cimex lectularius, Dagbertus fasciatus, Dichelops furcatus, Diuraphis noxia, Diaphorina citri, Dysaphis plantaginea, Dysdercus suturellus, Edessa meditabunda, Eriosoma lanigerum, Eurygaster maura, Euschistus heros, Euschistus servus, Helopeltis antonii, Helopeltis theivora, Icerya purchasi, Idioscopus nitidulus, Laodelphax striatellus, Leptocorisa oratorius, Leptocorisa varicomis, Lygus hesperus, Maconellicoccus hirsutus, Macrosiphum euphorbiae, Macrosiphum granarium, Macrosiphum rosae, Macrosteles quadrilineatus, Mahanarva frimbiolata, Metopolophium dirhodum, Mictis longicomis, Myzus persicae, Nephotettix cinctipes, Neurocolpus longirostris, Nezara viridula, Nilaparvata lugens, Parlatoria pergandii, Parlatoria ziziphi, Peregrinus maidis, Phylloxera vitifoliae, Physokermes piceae, Phytocoris californicus, Phytocoris relativus, Piezodorus guildinii, Poecilocapsus lineatus, P. sallus vaccinicola, Pseudacysta perseae, Pseudococcus brevipes, Quadraspidiotus perniciosus, Rhopalosiphum maidis, Rhopalosiphum padi, Saissetia oleae, Scaptocoris castanea, Schizaphis graminum, Sitobion avenae, Sogatella furcifera, Trialeurodes vaporariorum, Trialeurodes abutiloneus, Unaspis yanonensis, and Zulia entrerriana.

In another embodiment, the insect pest is from the Order Hymenoptera. Non-limiting examples of genera may include, but are not limited to, Acromyrmex spp., Atta spp., Camponotus spp., Diprion spp., Formica spp., Monomorium spp., Neodiprion spp., Pogonomyrmex spp., Polistes spp., Solenopsis spp., Vespula spp., and Xylocopa spp. Non-limiting examples of species may include, but are not limited to, Athalia rosae, Atta texana, Iridomyrmex humilis, Monomorium minimum, Monomorium pharaonis, Solenopsis invicta, Solenopsis geminata, Solenopsis molesta, Solenopsis richtery, Solenopsis xyloni, and Tapinoma sessile.

In an alternative embodiment, the insect pest is from the Order Isoptera. Non-limiting examples of genera may include, but are not limited to, Coptotermes spp., Cornitermes spp., Cryptotermes spp., Heterotermes spp., Kalotermes spp., Incisitermes spp., Macrotermes spp., Marginitermes spp., Microcerotermes spp., Procomitermes spp., Reticulitermes spp., Schedorhinotermes spp., and Zootermopsis spp. Non-limiting examples of species may include, but are not limited to, Coptotermes curvignathus, Coptotermes frenchi, Coptotermes formosanus, Heterotermes aureus, Microtermes obesi, Reticulitermes banyulensis, Reticulitermes grassei, Reticulitermes flavipes, Reticulitermes hageni, Reticulitermes hesperus, Reticulitermes santonensis, Reticulitermes speratus, Reticulitermes tibialis, and Reticulitermes virginicus.

In another embodiment, the insect pest is from the Order Lepidoptera. Non-limiting examples of genera may include, but are not limited to, Adoxophyes spp., Agrotis spp., Argyrotaenia spp., Cacoecia spp., Caloptilia spp., Chilo spp., Chrysodeixis spp., Colias spp., Crambus spp., Diaphania spp., Diatraea spp., Earias spp., Ephestia spp., Epimecis spp., Feltia spp., Gortyna spp., Helicoverpa spp., Heliothis spp., Indarbela spp., Lithocolletis spp., Loxagrotis spp., Malacosoma spp., Peridroma spp., Phyllonorycter spp., Pseudaletia spp., Sesamia spp., Spodoptera spp., Synanthedon spp., and Yponomeuta spp. Non-limiting examples of species may include, but are not limited to, Achaea janata, Adoxophyes orana, Agrotis ipsilon, Alabama argillacea, Amorbia cuneana, Amyelois transitella, Anacamptodes defectaria, Anarsia lineatella, Anomis sabulifera, Anticarsia gemmatalis, Archips argyrospila, Archips rosana, Argyrotaenia citrana, Autographa gamma, Bonagota cranaodes, Borbo cinnara, Bucculatrix thurberiella, Capua reticulana, Carposina niponensis, Chlumetia transversa, Choristoneura rosaceana, Cnaphalocrocis medinalis, Conopomorpha cramerella, Cossus cossus, Cydia caryana, Cydia funebrana, Cydia molesta, Cydia nigricana, Cydia pomonella, Darna diducta, Diatraea saccharalis, Diatraea grandiosella, Earias insulana, Earias vittella, Ecdytolopha aurantianum, Elasmopalpus lignosellus, Ephestia cautella, Ephestia elutella, Ephestia kuehniella, Epinotia aporema, Epiphyas postvittana, Erionota thrax, Eupoecilia ambiguella, Euxoa auxiliaris, Grapholita molesta, Hedylepta indicata, Helicoverpa armigera, Helicoverpa zea, Heliothis virescens, Hellula undalis, Keiferia lycopersicella, Leucinodes orbonalis, Leucoptera coffeella, Leucoptera malifoliella, Lobesia botrana, Loxagrotis albicosta, Lymantria dispar, Lyonetia clerkella, Mahasena corbetti, Mamestra brassicae, Maruca testulalis, Metisa plana, Mythimna unipuncta, Neoleucinodes elegantalis, Nymphula depunctalis, Operophtera brumata, Ostrinia nubilalis, Oxydia vesulia, Pandemis cerasana, Pandemis heparana, Papilio demodocus, Pectinophora gossypiella, Peridroma saucia, Perileucoptera coffeella, Phthorimaea operculella, Phyllocnistis citrella, Pieris rapae, Plathypena scabra, Plodia interpunctella, Plutella xylostella, Polychrosis viteana, Prays endocarpa, Prays oleae, Pseudaletia umpuncta, Pseudoplusia includens, Rachiplusia nu, Scirpophaga incertulas, Sesamia inferens, Sesamia nonagrioides, Setora nitens, Sitotroga cerealella, Sparganothis pilleriana, Spodoptera exigua, Spodoptera frugiperda, Spodoptera eridania, Thecla basilides, Tineola bisselliella, Trichoplusia ni, Tuta absoluta, Zeuzera coffeae, and Zeuzera pyrina.

In a particular embodiment, the insect pest is from the Order Mallophaga. Non-limiting examples of genera may include, but are not limited to, Anaticola spp., Bovicola spp., Chelopistes spp., Goniodes spp., Menacanthus spp., and Trichodectes spp. Non-limiting examples of species may include, but are not limited to, Bovicola bovis, Bovicola caprae, Bovicola ovis, Chelopistes meleagridis, Goniodes dissimilis, Goniodes gigas, Menacanthus stramineus, Menopon gallinae, and Trichodectes canis.

In another embodiment, the insect pest is from the Order Orthoptera. Non-limiting examples of genera may include, but are not limited to, Melanoplus spp., and Pterophylla spp. Non-limiting examples of species may include, but are not limited to, Anabrus simplex, Gryllotalpa africana, Gryllotalpa australis, Gryllotalpa brachyptera, Gryllotalpa hexadactyla, Locusta migratoria, Microcentrum retinerve, Schistocerca gregaria, and Scudderia furcata.

In yet another embodiment, the insect pest is from the Order Siphonaptera. Non-limiting examples of species may include, but are not limited to, Ceratophyllus gallinae, Ceratophyllus niger, Ctenocephalides canis, Ctenocephalides felis, and Pulex irritans.

In an alternative embodiment, the insect pest is from the Order Thysanoptera. Non-limiting examples of genera may include, but are not limited to, Caliothrips spp., Frankliniella spp., Scirtothrips spp., and Thrips spp. Non-limiting examples of species may include, but are not limited to, Frankliniella fusca, Frankliniella occidentalis, Frankliniella schultzei, Frankliniella williamsi, Heliothnps haemorrhoidalis, Rhipiphorothnps cruentatus, Scirtothrips citri, Scirtothrips dorsalis, and Taeniothrips rhopalantennalis, Thrips hawaiiensis, Thrips nigropilosus, Thrips orientalis, Thrips tabaci.

In another embodiment, the insect pest is from the Order Thysanura. Non-limiting examples of genera may include, but are not limited to, Lepisma spp. and Thermobia spp.

In yet another embodiment, the insect pest is from the Order Acarina. Non-limiting examples of genera may include, but are not limited to, Acarus spp., Aculops spp., Boophilus spp., Demodex spp., Dermacentor spp., Epitrimerus spp., Eriophyes spp., Ixodes spp., Oligonychus spp., Panonychus spp., Rhizoglyphus spp., and Tetranychus spp. Non-limiting examples of species may include, but are not limited to, Acarapis woodi, Acarus siro, Aceria mangiferae, Aculops lycopersici, Aculus pelekassi, Aculus schlechtendali, Amblyomma americanum, Brevipalpus obovatus, Brevipalpus phoenicis, Dermacentor variabilis, Dermatophagoides pteronyssinus, Eotetranychus carpini, Notoedres cati, Oligonychus coffeae, Oligonychus ilicis, Panonychus citri, Panonychus ulmi, Phyllocoptruta oleivora, Polyphagotarsonemus latus, Rhipicephalus sanguineus, Sarcoptes scabiei, Tegolophus perseaflorae, Tetranychus urticae, and Varroa destructor.

In a particular embodiment, the insect pest is from the Order Symphyla. Non-limiting examples of species may include, but are not limited to, Scutigerella immaculata.

In certain embodiments, the target gene ortholog is an ortholog that shares at least 35%, or at least 50% sequence identity, or at least 60% sequence identity, or at least 70% sequence identity, or at least 80% sequence identity, or at least 85% sequence identity, or at least 90% sequence identity, or at least 95% sequence identity with the corresponding reference gene e.g., a gene listed in Table 1 and/or Table 2). In certain embodiments the target gene orgholog can show less than 35% sequence when the when the E value is <E-10. identity In certain embodiments, the target gene ortholog encodes a protein that shares at least 70% sequence identity, or at least 75% sequence identity, or at least 80% sequence identity, or at least 85% sequence identity, or at least 90% sequence identity, or at least 95% sequence identity, or at least 98% sequence identity with a protein expressed by the corresponding reference gene e.g., a gene listed in Table 1 and/or Table 2).

The foregoing target genes are illustrative and non-limiting. Using the teaching provided herein, numerous other target genes downregulation of which can inhibit a phytopathogen can readily be identified using the methods described herein.

### Gene Silencing—Inhibitory Polynucleotides.

In various embodiments, the agent that down-regulates expression of one or more genes described herein comprises an agent that induces gene silencing of the target gene(s). “Gene silencing” is a general term used to describe the regulation of gene expression. In particular, this term refers to the ability of a cell to prevent the expression of a gene in the cell. Gene silencing can occur during either transcription or translation of a gene. The application of gene silencing methods in plants is well known in the art. Reference is made to Plant Gene Silencing—Methods and Protocols, Mysore K. S and Senthil-Kumar M. (eds.), Springer Protocols, Humana Press, 2015, which provides a comprehensive review of various gene silencing methodologies and its applications. In various embodiments, the skilled person may apply any methods described in this document or elsewhere that are suitable to achieve gene silencing in a plant and in a fungus.

In certain embodiments, RNA silencing is used to down regulate the target gene(s) described herein. “RNA silencing” refers to a group of regulatory mechanisms (e.g. RNA interference (RNAi), transcriptional gene silencing (TGS), post-transcriptional gene silencing (PTGS), quelling, co-suppression, and translational repression] mediated by RNA molecules that result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Illustrative RNA silencing agents include, but are not limited to dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily conserved cellular defense mechanism used to prevent the expression of foreign genes and is commonly shared by diverse flora and phyla.

The presence of long double stranded RNAs (dsRNAs) in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly in various embodiment nucleic acids and/or nucleic acid constructs for silencing one or more of the target genes identified herein (see, e.g., Table 2) or homologs and/or orthologs thereof as described herein are provided.

Accordingly, in certain embodiments, an isolated double stranded RNA (dsRNA) for controlling or preventing an infection of a plant by a phytopathogen (e.g., a phytopathogenic fungus) is provided. In certain embodiments, the dsRNA downregulates (is capable of downregulating) expression of one or more target G. orontii genes identified in Table 2 or homologs/orthologs thereof.

In certain embodiments, the isolated dsRNA molecule comprises two annealed complementary RNA strands. In certain embodiments, the double-stranded RNA molecule comprises a nucleotide sequence that is complementary to at least 17, 19 or 21 contiguous nucleotides of one or more of said target G. orontii genes (e.g., the genes shown in Table 1 and/or Table 2) or homologs/orthologs thereof, or an RNA transcribed therefrom. In certain embodiments, the double-stranded RNA molecule comprises a nucleotide sequence that is complementary to at least 17, or at least about 18, or at least about 19, or at least about 20, or at least about 21 to up to about 2000, or up to about 1000, or up to about 500, or up to about 300, or up to about 200, or up to about 100 contiguous nucleotides of the target G. orontii genes or target G. orontii gene orthologs, or an RNA transcribed therefrom. In certain embodiments the double-stranded RNA molecule comprises a nucleotide sequence that is complementary to 17 up to 500, or 17 up to 2000, or 21 up to 2000, or 17 up to 100, or 21 up to 200, or 50 up to 300, or 100 up to 1000, or 100 up to 1500, or 200 up to 2000 contiguous nucleotides of the target G. orontii genes or target G. orontii gene orthologs, or an RNA transcribed therefrom. In certain embodiments, the isolated dsRNA molecule comprises a nucleic acid sequence complementary to about 15 to 100, 19 to 100, 20 to 200, 17 to 500, 100 to 1000 or complementary to about 200 to 1,000, or complementary to about 200 to 650 contiguous nucleotides of the protein coding region of the target G. orontii genes or target G. orontii gene orthologs or an RNA transcribed therefrom. In certain embodiments, the dsRNA comprises a nucleic acid sequence complementary to about 17 to 1000 or complementary to about 200 to 1,000, or complementary to about 100 to 650 contiguous nucleotides of the 5′ UTR region or the 3′ UTR region said target G. orontii genes or target G. orontii gene orthologs.

In certain embodiments, the dsRNA comprises a nucleic acid sequence complementary to a contiguous region comprising at least about 0.1%, 0.5%, 1%, 2%, 3%, 5%, 10%, 15%, 20%, 25%, 50%, 60%, 70%, 80%, 90%, 95%, 98%, 99%, or 100% of the length of the target gene sequence protein coding region, 5′ UTR region, or 3′ UTR region of said target G. orontii genes or target G. orontii gene orthologs.

In certain embodiments, the dsRNA molecule comprises a single RNA strand comprising an inversely repeated sequence with a spacer in between and wherein the single RNA strand can anneal to itself to form a hairpin loop structure (shRNA). In certain embodiments, the shRNA is about 30 nucleotides or shorter in length. In certain embodiments, the shRNA is longer in length, e.g., up to 1000 nt. In certain embodiments, the shRNA ranges in length from about 25-29 nucleotides in length. In certain embodiments, the shRNA comprises a duplex length ranging from bout 17-21 nucleotides or from about 18-23 nucleotides, or from about 19-21 nucleotides. In certain embodiments, the shRNA comprises a loop sequence ranging from 3 to 9 nucleotides in length or has a loop length of 5, 7, or 9 nucleotides.

In certain embodiments, the dsRNA comprises an miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target). The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (see, e.g., Parizotto et al. (2004) Genes Dev. 18: 2237-2242; Guo et al. (2005) Plant Cell 17: 1376-1386; and the like).

Unlike, siRNAs, miRNAs bind to target gene or to target gene transcript sequences with only partial complementarity (Zeng et al. (2002) Molec. Cell, 9: 1327-1333) and repress translation without affecting steady-state RNA levels (see, e.g., Lee et al. (1993) Cell, 75: 843-854; Wightman et al. (1993) Cell 75: 855-862). Both miRNAs and siRNAs are processed by Dicer and associate with components of the RNA-induced silencing complex (Hutvagner et al. (2001) Science 293: 834-838; Grishok et al. (2001) Cell, 106: 23-34; Ketting et al. (2001) Genes Dev. 15: 2654-2659; Williams et al. (2002) Proc. Natl. Acad. Sci. USA 99: 6889-6894; Hammond et al. (2001) Science, 293: 1146-1150; Mourlatos et al. (2002) Genes Dev. 16: 720-728).

Target sites are recognized by an miRNA primarily by sequence pairing with the “seed” region, typically comprising positions 2-7 of miRNA's 5′ end. In various embodiments 6-8 ntd complete complementarity in the miRNA is desirable to to provide effective translation inhibition.

In various embodiments, the dsRNA is capable of reducing infection, growth, and/or spore production of a phytopathogen comprising a gene that the dsRNA downregulates. In certain embodiments, the dsRNA capable of reducing hyphal length of a phytopathogen comprising a gene that the dsRNA downregulates.

The dsRNAs described herein and/or DNAs encoding the dsRNAs are readily produced using routine methods known to those of skill in the art. Thus, for example, in various embodiments, the dsRNAs described herein can be generated according to any polynucleotide synthesis method known in the art such as enzymatic synthesis or solid phase synthesis. Equipment and reagents for executing solid-phase synthesis are commercially available from, for example, Applied Biosystems. Any other means for such synthesis may also be employed. The synthesis of the polynucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

In certain embodiments, following synthesis, the dsRNAs or DNAs encoding the dsRNAs described herein may optionally be purified. For example, dsRNAs or DNAs can be purified from a mixture by extraction with a solvent or resin, precipitation, electrophoresis, chromatography, or a combination thereof. Alternatively, polynucleotides may be used with no, or a minimum of, purification to avoid losses due to sample processing. In various embodiments, the dsRNAs may be dried for storage or dissolved in an aqueous solution. The solution may contain buffers or salts to promote annealing, and/or stabilization of the duplex strands.

It will be appreciated that in various embodiments, a dsRNA described herein can be provided per se, or as a nucleic acid construct comprising a nucleic acid sequence encoding the dsRNA. Typically, the nucleic acid construct comprises a promoter sequence which is functional in a host cell, as detailed herein below. The DNAs encoding the dsRNAs under the control of an operably linked promoter sequence, may further be flanked by additional sequences that advantageously affect its transcription and/or the stability of a resulting transcript. Such sequences are generally located upstream of the promoter and/or downstream of the 3′ end of the expression construct. Regulatory sequences for expression (transcription) of the dsRNA may include promoters, translation leader sequences, introns, enhancers, stem-loop structures, repressor binding sequences, termination sequences, pausing sequences, polyadenylation recognition sequences, and the like.

The foregoing dsRNAs are illustrative and non-limiting. Using the teachings provided herein numerous dsRNAs that can downregulate the target genes and/or target gene orthologs identified herein will be available to one of skill in the art.

### Compositions for Controlling or Preventing an Infection of a Plant by a Phytopathogen

In various embodiments, compositions for controlling or preventing an infection of a plant by a phytopathogen (e.g., a phytopathogenic fungus) are provided. Typically, in various embodiments, such compositions are applied to a plant at risk for infection by a phytopathogen, or after identification of infection by a phytopathogen.

In various embodiments, such compositions contain one or more of the dsRNAs described herein (e.g., a dsRNA that downregulates a target gene shown in Table 1 or Table 2 or that downregulates a homolog/ortholog of a gene shown in Table 1 or Table 2). In certain embodiments, the composition comprises a dsRNA or multiple dsRNAs that downregulates a single target gene. In certain embodiments, the composition comprises a dsRNA or dsRNAs that downregulates a target gene and orthologous target genes in the same species or other related species or strains. In certain embodiments, the composition comprises a single dsRNA has a plurality of domains that each downregulate a different target gene. In certain embodiments, the composition comprises a plurality of dsRNAs that each downregulate a different target gene. In certain embodiments, the composition comprises dsRNA(s) that downregulate 2, or 3, or 4, or 5, or 6, or 7, or 8, or 9, or 10 target gene(s).

In certain embodiments, the composition is formulated for spray on application to a plant to provide spray-induced gene silencing (SIGS). In certain embodiments, the composition is provided as a spray, a dust, drenches, drip application, granules, injecting into a plant and plant part, aerosol application, particle bombardment, a seed coating, or a plant-incorporated protectant.

In certain embodiments, the composition comprises said dsRNA(s) associated with particulate of any desirable size. In certain embodiments the particulate range in size from about 1 nanometer to about 100 micrometers. In certain embodiments the particulate is supported by, attached to, or embedded in a matrix.

In certain embodiments, the composition comprises said dsRNA(s) associated with a particle or nanoparticle (e.g., a clay or a polymer particle or nanoparticle). In certain embodiments, the particles or nanoparticles are of a size and composition selected to abrade the plant surface and facilitate entry of the dsRNA(s) into the plant. In certain embodiments the particle or nanoparticle control the release of said dsRNA overtime. In certain embodiments the particle or nanoparticle increases the availability and/or stability. In certain embodiments, the particle or nanoparticle increases the attachment of dsRNA on pathogen surface and/or increase the absorption of said dsRNA in pathogen.

In certain embodiments the particulate is selected from the group consisting of a mineral abrasive, a metal abrasive, a synthetic abrasive, and an organic abrasive. In certain embodiments the particulate is selected from the group consisting of aluminum oxide, silicon carbide, silicon dioxide, soda lime glass, diatomaceous silica (diatomaceous earth), flint, quartz, garnet, silicon dioxide, pumice, sand, feldspar, calcite, steel, tungsten, ceramic, boron carbide, tungsten carbide.

In certain embodiments, the particle or nanoparticle comprises a metal, metal oxides, metalloids, polymeric nanoparticles, carbon- or carbon-based material, silicon or silicon-based material, silica vesicles, dendrimers, quantum dots etc. In certain embodiments, the particle or nanoparticle comprises calcium phosphate or calcium carbonate. In certain embodiments, the particle or nanoparticle comprises clay minerals or clay complexes. In certain embodiments the particle or nanoparticle comprises the dsRNA loaded or adsorbed onto Layered Double Hydroxide (LDH) particles.

In certain embodiments the particles or nanoparticles are biopolymers. In some embodiments, the polymer is selected from the group consisting of but not limited to poly(acrylic acid), poly(methacrylic acid), poly (styrene sulfonate), chitosan, poly (dimethyldiallylammonium chloride), poly (allylamine hydrochloride), or copolymers or graft polymers thereof and combinations thereof. In certain embodiments the biopolymers are chitin or chitosan derivatives. In certain embodiments the dsRNA can be attached to chitosan particles or encapsulated with chitosan polymers. In certain embodiments the chitosan polymers can be allosperse. In some embodiments, at least a portion of the active ingredient (dsRNA) in the composition is in the interior of the polymer nanoparticle. In some embodiments, at least a portion of it is on the surface of the polymer nanoparticle.

In certain embodiments other nanomaterials used in compositions for application of the dsRNAs described herein can be oil-based, polymer-based, lipid-based, and porous inorganic nanomaterials. In certain embodiments the composition may comprise of cellulose and its modified forms e.g., microfibrillated cellulose. In certain embodiments, the composition comprises dsRNA(s) incorporated into a lipid or liposome, a functionalized liposome, protein matrix, or other biologically derived delivery platforms.

In certain embodiments, the compositions for delivery of the dsRNAs described herein composition comprise the inhibitory dsRNA molecule(s) described herein and a synthetic carrier or microbial conduit that can be a microorganism that has a natural capacity or is engineered to produce and/or deliver dsRNA to increase its bioavailability and/or biostability for causing RNA interference. In certain embodiments, dsRNA is encapsulated and delivered using an anucleated cell-based platform. In certain embodiments, RNA is produced and/or delivered in a microbial host where the microbial host can be a prokaryotic cell, a gram-negative bacterial cell, a gram-positive bacterial cell, or an eukaryotic cell. In certain embodiments, the microbes belong to the category of GRAS (Generally Recognized As Safe) microbes. In certain embodiments, the GRAS microbes are selected from the group of Bacillus, Corynebacterium, and Lactobacillus. In certain embodiments, dsRNA can be applied mixed with GRAS chemicals. In certain embodiments, applications of the dsRNAs described herein may be made as RNA mixed with fertilizers.

In certain embodiments, the composition applied on plant for controlling pathogen(s) comprises one or more dsRNAs described herein associated with a particulate of any size further applied with (a) at least one selected from the group consisting of water, a biocide, a chelator, a buffer, an ionic or nonionic surfactant, a zwitterionic surfactant, a defoamer, a rainfastness agent, and a photoprotectant, and/or (b) at least one selected from the group consisting of agricultural carrier agent, a surfactant, an organosilicone, and/or (c) at least one selected from the group consisting of adhesion agent, cmpatability agent, conditioning agent, wetting agent, spreading agent, extenders, stabilizer, preservative, a dessicant, dispersants, tackifiers, and emulsifiers; and/or (d) at least one selected from the group consisting of fertilizers, plant nutrients, plant growth regulators (PGRs) and biostimulants; and/or e) at least one pesticidal agent from a group of fungicides, antibacterials, insecticides, herbicides, and nematicides. In certain embodiments, the dsRNA(s) comprise a lyophilized powder or granules.

In certain embodiments, the compositions for application/delivery of one or more dsRNAs described herein comprises dsRNA(s) as a solution or suspension. In certain embodiments, applications of RNAs may be applied in a way wherein the particulate, the RNA, or both, are further applied with an osmolyte, and whereby the target gene is silenced. In certain embodiments, the composition comprising the dsRNA(s) described herein associated with any adjuvants that meets the standards of the Chemical Producers and Distributors Association (CPDA) adjuvant certification program.

In certain embodiments a composition for application/delivery of one or more dsRNA(s) described herein may comprise dsRNA(s) mixed with an adhesion agent such as a non-naturally occurring adhesion agent. A non-naturally occurring adhesion agent can be, for example, a polymer, copolymer, or synthetic wax. For example, any of the coated seeds, seedlings, or plants described herein can contain such an agriculturally acceptable carrier in the seed coating.

In certain embodiments, a composition for application/delivery of one or more dsRNA(s) described herein comprises dsRNA(s) mixed with other biological pesticides, plant growth regulators, and/robiocontrol or biostimulants. In certain embodiments, the biostimulant compound is a peptide, a polypeptide, another RNA molecule, a fermentation product, a metabolite, an antibody, a semiochemical, or a micronutrient. In certain embodiments, the dsRNA composition(s) are applied with biostimulants including hormones and biochemical growth agents. These include but are not limited to abscisic acid, auxins, cytokinins, ACC Deaminase, gibberellins, brassinosteroids, salicylic acid, jasmonates, plant peptide hormones, polyamines, nitric oxide, strigolactones, karrikins, and triacontanol, which are used to both positively and negatively regulate the growth of plants. In some embodiments, the biologically active compounds are pheromones. In certain embodiments, biostimulants comprises microbial properties such as rhizobium (PGPRs) properties. In certain embodiments, biostimulants comprises acids (such as humic substances, humin, fulvic acids, B vitamins, amino acids, fatty acids/lipids), extracts (such as carboxyls, botanicals, allelochemicals, betaines, polyamines, polyphenols, chitosan and other biopolymers), phosphites, phosphate solubilizers, nitrogenous compounds, inorganic salts, protein hydrolysates, and beneficial elements. In certain embodiments the biocontrol product is a peptide, a polypeptide, another RNA molecule, a fermentation product, a metabolite, an antibody, a semiochemical, or a micronutrient.

dsRNA Compositions Comprising Growth Regulators.

In certain embodiments, a composition for application/delivery of one or more dsRNA(s) described herein may include one or more growth regulators. Illustrative growth regulators include, but are not limited to Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoalexins (e.g., glyceollin), and phytoalexin-inducing oligosaccharides (e.g., pectin, chitin, chitosan, polygalacuronic acid, and oligogalacturonic acid), and gibellerins.

dsRNA Compositions Comprising Fertilizers, Plant Micronutrients, and/or Plant Macronutrients.

In certain embodiments, a composition for application/delivery of one or more dsRNA(s) described herein comprises dsRNA(s) mixed with fertilizers, plant micronutrients and plant macro-nutrients, which can include, but are not limited to, nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH4H2PO4, (NH4)2S04, glycerol, valine, L-leucine, lactic acid, propionic acid, succinic acid, malic acid, citric acid, KH tartrate, xylose, lyxose, and lecithin. Further examples of fertilizers which may be useful in embodiments include ammonium nitrate, ammonium sulfate, anhydrous ammonia, calcium nitrate/urea, oxamide, potassium nitrate, urea, urea sulfate, ammoniated superphosphate, diammonium phosphate, nitric phosphate, potassium carbonate, potassium metaphosphate, calcium chloride, magnesium ammonium phosphate, magnesium sulfate, ammonium sulfate, potassium sulfate, and others disclosed herein. Some examples of nutrients can be selected from the group consisting of a nitrogen fertilizer including, but not limited to urea, ammonium nitrate, ammonium sulfate, non-pressure nitrogen solutions, aqua ammonia, anhydrous ammonia, ammonium thiosulfate, sulfur-coated urea, urea-formaldehydes, IBDU, polymer-coated urea, calcium nitrate, ureaform, and methylene urea, phosphorous fertilizers such as diammonium phosphate, monoammonium phosphate, ammonium polyphosphate, concentrated superphosphate and triple superphosphate, and potassium fertilizers such as potassium chloride, potassium sulfate, potassium-magnesium sulfate, potassium nitrate. Such compositions can exist as free salts or ions within the seed coat composition. Alternatively, nutrients/fertilizers can be complexed or chelated to provide sustained release over time.

dsRNA Compositions Comprising Agrochemicals.

In certain embodiments, compositions comprising the dsRNAs described herein comprises a mixture of one or more dsRNA molecules described herein and at least one of a variety of agricultural chemicals or agrochemicals. Examples of agrochemical substances include, but are not limited to, chemical pesticides (such as herbicides, algicides, fungicides, bactericides, viricides, insecticides, acaricides, miticides, nematicides and molluscicides), herbicide safeners, plant growth regulators (such as hormones and chemical growth agents), fertilizers and nutrients, gametocides, defoliants, desiccants, mixtures thereof and the like, and any combination thereof.

Non-limiting examples of agrochemical herbicides, insecticides, miticides, fungicides, nematicide, bactericide, pesticidal agents and/or biopesticidal (e.g., microbial, plant-incorporated-protectant (PIP), and/or biochemical) agents include, but are not limited to Spiromesifen, Spirodiclofen, Spirotetramat, Pyridaben, Tebufenpyrad, Tolfenpyrad, Fenpyroximate, Flufenerim, Pyrimidifen, Fenazaquin, Rotenone, Cyenopyrafen, Hydramethylnon, Acequinocyl, Fluacrypyrim, Aluminium phosphide, Calcium phosphide, Phosphine, Zinc phosphide, Cyanide, Diafenthiuron, Azocyclotin, Cyhexatin, Fenbutatin oxide, Propargite, Tetradifon, Bensultap, Thiocyclam, Thiosultap-sodium, Flonicamid, Etoxazole, Clofentezine, Diflovidazin, Hexythiazox, Chlorfluazuron, Bistrifluron, Diflubenzuron, Flucycloxuron, Flufenoxuron, Hexaflumuron, Fufenuron, Novaluron, Noviflumuron, Teflubenzuron, Triflumuron, Buprofezin, Cyromazine, Hydroprene, Kinoprene, Methoprene, Fenoxycarb, Pyriproxyfen, Pymetrozine, Pyrifluquinazon, Chlorfenapyr, Tralopyril, methyl bromide and/or other alkyl halides, Chloropicrin, Sulfuryl fluoride, Benclothiaz, Chinomethionat, Cryolite, Methylneodecanamide, Benzoximate, Cymiazole, Fluensulfone, Azadirachtin, Bifenazate, Amidoflumet, Dicofol, Plifenate, Cyflumetofen, Pyridalyl, Beauveria bassiana GHA, Sulfoxaflor, Spinetoram, Spinosad, Spinosad, Emamectin benzoate, Fepimectin, Milbemectin, Abamectin, Methoxyfenozide, Chromafenozide, Halofenozide, Tebufenozide, Amitraz, Chlorantraniliprole, Cyantraniliprole, Flubendiamide, alpha-endosulfan, Chlordane, Endosulfan, Fipronil, Acetoprole, Ethiprole, Pyrafluprole, Pyriprole, Indoxacarb, Metaflumizone, Acrinathrin, Allethrin, Allethrin-cis-trans, Allethrin-trans, beta-Cyfluthrin, beta-Cypermethrin, Bifenthrin, Bioallethrin, Bioallethrin S-cyclopentenyl, Bioresmethrin, Cycloprothrin, Cyfluthrin, Cyhalothrin, Cypermethrin, Cyphenothrin [(1R)-trans-isomers], Dimefluthrin, Empenthrin [(EZ)-(1R)-isomers], Esfenvalerate, Etofenprox, Fenpropathrin, Fenvalerate, Flucythrinate, Flumethrin, Gamma-cyhalothrin, lambda-Cyhalothrin, Meperfluthrin, Metofluthrin, Permethrin, Phenothrin [(1R)-trans-isomer], Prallethrin, Profluthrin, Protrifenbute, Resmethrin, Silafluofen, tau-Fluvalinate, Tefluthrin, Tetramethrin, Tetramethrin [(1R)-isomers], Tetramethylfluthrin, theta-Cypermethrin, Tralomethrin, Transfluthrin, zeta-Cypermethrin, alpha-Cypermethrin, Deltamethrin, DDT, Methoxychlor, Thiodicarb, Alanycarb, Aldicarb, Bendiocarb, Benfuracarb, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiofanox, Triazamate, Trimethacarb, XMC, Xylylcarb, Chlorpyrifos, Malathion, Acephate, Azamethiphos, Azinphos-ethyl, Azinphos-methyl, Cadusafos, Chlorethoxyfos, Chlorfenvinphos, Chlormephos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fonofos, Fosthiazate, Imicyafos, Isofenphos-methyl, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-ethyl, Profenofos, Propaphos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, Vamidothion Imidacloprid, Thiamethoxam, Acetamiprid, Clothianidin, Dinotefuran, Nitenpyram, Nithiozine, Nicotine, Thiacloprid, cyantraniliprole, carbamates, organophosphates, cyclodiene organochlorines, phenylpyrazoles (fiproles), pyrethroids, pyrethins, DDT Methoxychlor, Neonicotinoids, Nicotine, Sulfoximines, Butenolides, Mesoionics, Spinosyns, Avermectins, Milbernycins, Juvenile hormone analogues, Fenoxycarb, Pyriproxyfen, Alkyl halides, Chloropicrin, Fluorides, Borates, Tarter emetic, Methyl isothiocyanate generators, Pyridine azomethine derivatives, Pyropenes, Clofentezine, Diflovidazin, Hexythiazox, Etoxazole, Diafenthiuron, Organotin miticides, Propargite, Tetradifon, Pyrroles, Dinitrophenols, Sulfuramid, Nereistoxin analogues, Benzoylureas, Buprofezin, Cyromazine, Diacylhydrazines, Amitraz, Hydramethylnon, Acequinocyl, Fluacrypyrim, Bifenazate, METI acaricides and insecticides, Rotenone, Oxadiazines, Semicarbazones, Tetronic and Tetramic acid derivatives, Phosphides, Cyanides, Beta-ketonitrile derivatives, Carboxanilides, Diamides, Flonicamid, Meta-diamides Isoxazolines, Granuloviruses (GVs), Nucleopolyhedrovimses (NPVs), GS-omega/kappa HXTX-Hvla peptide, Azadirachtin, Benzoximate, Bromopropylate, Chinomethionat, Dicofol, Lime sulfur, Mancozeb, Pyridalyl, Sulfur, Benzimidazoles, Dicarboximides, Pyridines, Pyrimidines, Triazoles, Acylalanines, Pyridine carboxamides, Anilino-pyrimidines, Quinone outside Inhibitors (Qol-fungicides), Phenylpyrroles, Quinolines, Hydroxyanilides, Toluamides, Cyanoacetamide-oximes, Dinitrophenyl crotonates, Phosphonates, Carboxylic Acid Amides (CAA-fungicides), M1 inorganic, M2 inorganic, M3 dithiocarbamates, M4 phthalimides, paraffinic oil, petroleum-based horticultural oils, palmitic oil, steric oil, linoleic oil, oleic oils, canola oil, soybean oil, oregano oil, tagetes oil, balsam fir oil, thyme oil, black pepper oil, mint oil, cedarwood oil, fish oil, jojoba oil, lavadin oil, castor oil, eucalyptus oil, ocimum oil, patchouli oil, citrus oil, artemisia oil, camphor oil, wintergreen oil, methyl eugenol oil, thymol oil, geranium oil, sesame oil, linseed oil, cottonseed oil, lemongrass oil, bergamot oil, mustard oil, orange oil, citronella oil, tea tree oil, neem oil, garlic oil, Bacillus sphaericus, Bacillus thuringiensis (e.g., Bacillus thuringiensis var. aizawai, Bacillus thuringiensis var. israelensis, Bacillus thuringiensis var. kurstaki, Bacillus thuringiensis var. sphaericus, Bacillus thuringiensis var. tenebrionensis) and the insecticidal proteins they produce (e.g., Cry1Ab, Cry 1 Ac, CrylFa, Cry 1 A.105, Cry2Ab, Vip3A, mCry3A, Cry3Ab, Cry3Bb, Cry34Abl/Cr35Abl, and as further exemplified in Crickmore, N., Baum, J., Bravo, A., Lereclus, D., Narva, K., Sampson, K., Schnepf, E., Sun, M. and Zeigler, D. R. “Bacillus thuringiensis toxin nomenclature” (2018)), Paenibacillus popilliae, Serratia entomophila, nuclear polyhedrosis viruses, granulosis viruses, non-occluded baculoviruses, Beauveria spp, Metarhizium, Entomophaga, Zoopthora, Paecilomyces fumosoroseus, Normuraea, Lecanicillium lecanii, Nosema, Thelohania, Vairimorpha, Steinernema spp, Heterorhabditis spp or any combination thereof, which may further comprise an active ingredient selected from the group consisting of azinphos-methyl, acephate, isoxathion, isofenphos, ethion, etrimfos, oxydemeton-methyl, oxydeprofos, quinalphos, chlorpyrifos, chlorpyrifos-methyl, chlorfenvin phos, cyanophos, dioxabenzofos, dichlorvos, disulfoton, dimethylvinphos, dimethoate, sulprofos, diazinon, thiometon, tetrachlorvinphos, temephos, tebupirimfos, terbufos, naled, vamidothion, pyraclofos, pyridafen thion, pirimiphos-methyl, fenitrothion, fenthion, phenthoate, flupyrazophos, prothiofos, propaphos, profenofos, phoxime, phosalone, phosmet, formothion, phorate, malathion, mecarbam, mesulfenfos, methamidophos, methidathion, parathion, methyl parathion, monocrotophos, trichlorphon, EPN, isazophos, isamidofos, cadusafos, diamidaphos, dichlofenthion, thionazin, fenamiphos, fosthiazate, fosthietan, phosphocarb, DSP, ethoprophos, alanycarb, aldicarb, isoprocarb, ethiofen carb, carbaryl, carbosulfan, xylylcarb, thiodicarb, pirimicarb, fenobucarb, furathiocarb, propoxur, ben diocarb, benfuracarb, methomyl, metolcarb, XMC, carbofuran, aldoxycarb, oxamyl, acrin athrin, allethrin, esfenvalerate, empenthrin, cycloprothrin, cyhalothrin, gamma-cyhalothrin, lambda-cyhalothrin, cyfluthrin, beta-cyfluthrin, cypermethrin, alpha-cypermethrin, zeta-cyper-methrin, silafluofen, tetramethrin, tefluthrin, deltamethrin, tralomethrin, bifenthrin, phenothrin, fenvalerate, fenpropathrin, furamethrin, prallethrin, flucythrinate, fluvalinate, flubrocythrinate, permethrin, resmethrin, ethofenprox, cartap, thiocyclam, ben sultap, acetamiprid, imidacloprid, clothianidin, dinotefuran, thiacloprid, thiamethoxam, nitenpyram, chlorfluazuron, difluben zuron, teflubenzuron, triflumuron, novaluron, noviflumuron, bistrifluoron, fluazuron, flucycloxuron, flufenoxuron, hexaflumuron, lufenuron, chromafen ozide, tebufenozide, halofen ozide, methoxyfen ozide, diofen olan, cyromazin e, pyriproxyfen, buprofezin, methop-rene, hydroprene, kinoprene, triazamate, endosulfan, chlorfenson, chlorobenzilate, dicofol, bromopropylate, acetoprole, flpronil, ethiprole, pyrethrin, rotenone, nicotinesulphate, spinosad, finpronil, spirotetramat abamectin, acequinocyl, amidoflumet, amitraz, etoxazole, chinomethionat, clofentezine, fenbutatin oxide, dienochlor, cyhexatin, spirodiclofen, spiromesifen, tetradifon, tebufenpyrad, binapacryl, bifenazate, pyridaben, pyrimidifen, fenazaquin, fenothiocarb, fenpyroximate, fluacrypyrim, flu-azinam, flufenzin, hexythiazox, propargite, polynactin complex, milbemectin, lufenuron, mecarbam, methiocarb, mevinphos, halfenprox, azadirachtin, diafenthiuron, indoxacarb, emamectin benzoate, potassium oleate, sodium oleate, chlorfenapyr, tolfenpyrad, pymetrozine, fenoxycarb, hydramethylnon, hydroxy propyl starch, pyridalyl, flufenerim, flubendiamide, flonicamid, metaflumizole, lepimectin, TPIC, albendazole, oxibendazole, oxfendazole, trichlamide, fensulfothion, fenbendazole, levamisole hydrochloride, morantel tartrate, dazomet, metam-sodium, tri-adimefon, hexaconazole, propiconazole, ipconazole, prochloraz, triflumizole, tebuconazole, epoxiconazole, difenoconazole, flusilazole, triadimenol, cyproconazole, metconazole, fluquinconazole, bitertanol, tetraconazole, triticonazole, flutriafol, penconazole, diniconazole, fenbuconazole, bromuconazole, imibenconazole, simeconazole, myclobutanil, hymexazole, imazalil, furametpyr, thifluzamide, etridiazole, oxpoconazole, oxpoconazole fumarate, pefurazoate, prothioconazole, pyrifenox, fenarimol, nuari-mol, bupirimate, mepanipyrim, cyprodinil, pyrimethanil, metalaxyl, mefenoxam, oxadixyl, benalaxyl, thiophanate, thiophanate-methyl, benomyl, carbendazim, fuberidazole, thiabendazole, manzeb, propineb, zineb, metiram, maneb, ziram, thiuram, chlorothalonil, ethaboxam, oxycarboxin, carboxin, flutolanil, silthiofam, mepronil, dimethomorph, fenpropidin, fenpropimorph, spiroxamine, tridemorph, dodemorph, flumorph, azoxystrobin, kresoxim-methyl, metominostrobin, orysastrobin, fluoxastrobin, trifloxystrobin, dimoxystrobin, pyraclostrobin, picoxystrobin, iprodione, procymidone, vinclozolin, chlozolinate, flusulfamide, dazomet, methyl isothiocyanate, chloropicrin, methasulfocarb, hydroxyisoxazole, potassium hydroxyisoxazole, echlomezol, D-D, carbarn, basic copper chloride, basic copper sulfate, copper nonylphenolsulfonate, oxine copper, DBEDC, anhydrous copper sulfate, copper sulfate pentahydrate, cupric hydroxide, inorganic sulfur, wettable sulfur, lime sulfur, zinc sulfate, fentin, sodium hydrogen carbonate, potassium hydrogen carbonate, potassium bicarbonate, sodium hypochlorite, silver, edifenphos, tolclofos-methyl, fosetyl, iprobenfos, dinocap, pyrazophos, carpropamid, fthalide, tricyclazole, pyroquilon, diclocymet, fenoxanil, kasugamycin, validamycin, polyoxins, blasticiden S, oxytetracycline, mildiomycin, streptomycin, rape seed oil, machine oil, benthiavalicarbisopropyl, iprovalicarb, propamocarb, diethofencarb, fluoroimide, fludioxanil, fenpiclonil, quinoxyfen, oxolinic acid, chlorothalonil, captan, folpet, probenazole, acibenzolar-S-methyl, tia-dinil, cyflufenamid, fenhexamid, diflumetorim, metrafenone, picobenzamide, proquinazid, famoxadone, cyazofamid, fenamidone, zoxamide, boscalid, cymoxanil, dithianon, fluazinam, dichlofluanide, triforine, isoprothiolane, ferimzone, diclomezine, tecloftalam, pencycuron, chinomethionat, iminoctadine acetate, iminoctadine albesilate, ambam, polycarbamate, thiadiazine, chloroneb, nickel dimethyldithiocarbamate, guazatine, dodecylguanidine acetate, quintozene, tolylfluanid, anilazine, nitrothalisopropyl, fenitropan, dimethirimol, benthiazole, flumetover, mandipropamide, and penthiopyrad, or any combinations thereof.

dsRNA Compositions Comprising Additional Fungicides.

In certain embodiments, compositions comprising the dsRNAs described herein may include an additional fungicide—a fungicide other than the dsRNA(s) described herein. Illustrated fungicides include, but are not limited to: AIGA compounds, 2-(thiocyanatomethylthio)-benzothiazole, 2-phenylphenol, 8-hydroxyquinoline sulfate, Ampelomyces quisqualis, azaconazole, azoxystrobin, Bacillus subtilis, benalaxyl, benomyl, benthiavalicarb-isopropyl, benzylaminobenzene-sulfonate (BABS) salt, bicarbonates, biphenyl, bismerthiazol, bitertanol, blasticidin-S, borax, Bordeaux mixture, boscalid, bromuconazole, bupirimate, calcium polysulfide, captafol, captan, carbendazim, carboxin, carpropamid, carvone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquation, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fentin, fentin acetate, fentin hydroxide, ferbam, ferimzone, fluazinam, fludioxonil, flumorph, fluopicolide, fluoroimide, fluoxastrobin, fluquinconazole, flusilazole, flusulfamide, flutolanil, flutriafol, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), ipconazole, iprobenfos, iprodione, iprovalicarb, isoprothiolane, kasugamycin, kasugamycin hydrochloride hydrate, kresoxim-methyl, mancopper, mancozeb, maneb, mepanipyrim, mepronil, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrazophos, pyributicarb, pyrifenox, pyrimethanil, pyroquilon, quinoclamine, quinoxyfen, quintozene, Reynoutria sachalinensis extract, silthiofam, simeconazole, sodium 2-phenylphenoxide, sodium bicarbonate, sodium pentachlorophenoxide, spiroxamine, sulfur, SYP-Z071, tar oils, tebuconazole, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, vinclozolin, zineb, ziram, zoxamide, Candida oleophila, Fusarium oxysporum, Gliocladium spp., Phlebiopsis gigantean, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme: ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, mefenoxam, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluene sulfonanilide, nickel bis(dimethyldithiocarbamate), OCH, phenylmercury dimethyldithiocarbamate, phenylmercury nitrate, phosdiphen, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, XRD-563, and zarilamid, and any combinations thereof.

In certain embodiments, composition are provided that comprise a mixture of one or more dsRNA molecule(s) described herein and at least one of the fungicides belonging to a certain FRAC groups developed by The Fungicide Resistance Action Committee (FRAC). In certain embodiments, the added fungicide acts on Nucleic acid metabolism and belong to FRAC groups 4, 8, 31 and 32. In certain embodiments, the added fungicide acts on the cytoskeletal and motor proteins of the cell of pathogen and belong to FRAC groups 1, 10, 20, 22, 43, 47, 50. In certain embodiments, the added fungicide acts on respiration and belong to FRAC groups 7, 11, 21, 29, 30, 38, 39, 45. In certain embodiments, the added fungicide acts on Amino acid and protein synthesis and belong to FRAC groups 9, 23, 24, 25, 41, In certain embodiments, the added fungicide acts on Signal transduction and belong to FRAC groups 13, 12 and 2. In certain embodiments, the added fungicide acts on Lipid synthesis and belong to FRAC groups 6, 14, 28, 46, 48 and 49. In certain embodiments, the added fungicide acts on sterol biosynthesis in membranes and belong to FRAC groups 3, 5, 17, 18. In certain embodiments, the added fungicide acts on cell wall biosynthesis and belong to FRAC groups 19 and 40. In certain embodiments, the added fungicide acts on melanin synthesis in cell wall and belong to FRAC groups 16.1, 16.2, 16.3. In certain embodiments, the added fungicide acts as a host plant defence induction and belong to FRAC groups P01 to P07. In certain embodiments, the added fungicide acts by an unknown mechanism and belong to FRAC groups 27, 34, 35, 36, 37, U06, U12, U13, U14, U16, U17 and U18. In certain embodiments, the added fungicide is not classified. In certain embodiments, the added fungicide is a chemical with multi-site activity and belong to FRAC groups M01 to M12. In certain embodiments, the added fungicide is a biological and belong to FRAC groups BM01, BM02 and other groups added to this category.

dsRNA Compositions Comprising Insecticides.

In certain embodiments, compositions comprising the dsRNAs described herein may include an additional insectide—a insecticide other than the dsRNA(s) described herein. Illustrative insecticides include but are not limited to AIGA compounds, antibiotic insecticides such as allosamidin and thuringiensin; macrocyclic lactone insecticides such as spinosad, spinetoram, and other spinosyns including the 21-butenyl spinosyns and their derivatives; avermectin insecticides such as abamectin, doramectin, emamectin, eprinomectin, ivermectin and selamectin; milbemycin insecticides such as lepimectin, milbemectin, milbemycin oxime and moxidectin; arsenical insecticides such as calcium arsenate, copper acetoarsenite, copper arsenate, lead arsenate, potassium arsenite and sodium arsenite; biological insecticides such as Bacillus popilliae, B. sphaericius, B. thurinigiensis subsp. aizqwai, B. thuringiensis subsp. kurstaki, B. thuriugiensis subsp. tenebrionis, Beauveria bassiana, Cydia pomonella granulosis virus, Douglas fir tussock moth nuclear polyhedrosis virus NPV, gypsy moth NPV, Helicoverpa zea NPV, Indian meal moth granulosis virus, Metarhizium anisopliae, Nosema locustae, Paecilomyces fumosoroseus, P. lilacinus, Photorhabdus luminescens, Spodoptera exigua NPV, trypsin modulating oostatic factor, Xenorhabdus nematophilus, and X. bovienii; plant incorporated protectant insecticides such as Cry1Ab, Cry1Ac, Cry1F, Cry1A.105, Cry2Ab2, Cry3A, mir Cry3A, Cry3Bb1, Cry34, Cry35, and VIP3A; botanical insecticides such as anabasine, azadirachtin, d-limonene, nicotine, pyrethrins, cinerins, cinerin I, cinerin II, jasmolin I, jasmolin II, pyrethrin I, pyrethrin II, quassia, rotenone, ryania and sabadilla; carbamate insecticides such as bendiocarb and carbaryl; benzofuranyl methylcarbamate insecticides such as benfuracarb, carbofuran, carbosulfan, decarbofuran and furathiocarb; dimethylcarbamate insecticides dimitan, dimetilan, hyquincarb and pirimicarb; oxime carbamate insecticides such as alanycarb, aldicarb, aldoxycarb, butocarboxim, butoxycarboxim, methomyl, nitrilacarb, oxamyl, tazimcarb, thiocarboxime, thiodicarb and thiofanox; phenyl methylcarbamate insecticides such as allyxycarb, aminocarb, bufencarb, butacarb, carbanolate, cloethocarb, dicresyl, dioxacarb, EMPC, ethiofencarb, fenethacarb, fenobucarb, isoprocarb, methiocarb, metolcarb, mexacarbate, promacyl, promecarb, propoxur, trimethacarb, XMC and xylylcarb; dinitrophenol insecticides such as dinex, dinoprop, dinosam and DNOC; fluorine insecticides such as barium hexafluorosilicate, cryolite, sodium fluoride, sodium hexafluorosilicate and sulfluramid; formamidine insecticides such as amitraz, chlordimeform, formetanate and formparanate; fumigant insecticides such as acrylonitrile, carbon di sulfide, carbon tetrachloride, chloroform, chloropicrin, para-dichlorobenzene, 1,2-dichloropropane, ethyl formate, ethylene dibromide, ethylene dichloride, ethylene oxide, hydrogen cyanide, iodomethane, methyl bromide, methylchloroform, methylene chloride, naphthalene, phosphine, sulfuryl fluoride and tetrachloroethane; inorganic insecticides such as borax, calcium polysulfide, copper oleate, mercurous chloride, potassium thiocyanate and sodium thiocyanate; chitin synthesis inhibitors such as bistrifluoron, buprofezin, chlorfluazuron, cyromazine, diflubenzuron, flucycloxuron, flufenoxuron, hexaflumuron, lufenuron, novaluron, noviflumuron, penfluoron, teflubenzuron and triflumuron; juvenile hormone mimics such as epofenonane, fenoxycarb, hydroprene, kinoprene, methoprene, pyriproxyfen and triprene; juvenile hormones such as juvenile hormone I, juvenile hormone II and juvenile hormone III; moulting hormone agonists such as chromafenozide, halofenozide, methoxyfenozide and tebufenozide; moulting hormones such as alpha-ecdysone and ecdysterone; moulting inhibitors such as diofenolan; precocenes such as precocene I, precocene II and precocene III; unclassified insect growth regulators such as dicyclanil; nereistoxin analogue insecticides such as bensultap, cartap, thiocyclam and thiosultap; nicotinoid insecticides such as flonicamid; nitroguanidine insecticides such as clothianidin, dinotefuran, imidacloprid and thiamethoxam; nitromethylene insecticides such as nitenpyram and nithiazine; pyridylmethylamine insecticides such as acetamiprid, imidacloprid, nitenpyram and thiacloprid; organochlorine insecticides such as bromo-DDT, camphechlor, DDT, pp′-DDT, ethyl-DDD, HCH, gamma-HCH, lindane, methoxychlor, pentachlorophenol and TDE; cyclodiene insecticides such as aldrin, bromocyclen, chlorbicyclen, chlordane, chlordecone, dieldrin, dilor, endosulfan, endrin, HEOD, heptachlor, HHDN, isobenzan, isodrin, kelevan and mirex; organophosphate insecticides such as bromfenvinfos, chlorfenvinphos, crotoxyphos, dichlorvos, dicrotophos, dimethylvinphos, fospirate, heptenophos, methocrotophos, mevinphos, monocrotophos, naled, naftalofos, phosphamidon, propaphos, TEPP and tetrachlorvinphos; organothiophosphate insecticides such as dioxabenzofos, fosmethilan and phenthoate; aliphatic organothiophosphate insecticides such as acethion, amiton, cadusafos, chlorethoxyfos, chlormephos, demephion, demephion-O, demephion-S, demeton, demeton-O, demeton-S, demeton-methyl, demeton-O-methyl, demeton-S-methyl, demeton-S-methyl sulphon, disulfoton, ethion, ethoprophos, PSP, isothioate, malathion, methacrifos, oxydemeton-methyl, oxydeprofos, oxydisulfoton, phorate, sulfotep, terbufos and thiometon; aliphatic amide organothiophosphate insecticides such as amidithion, cyanthoate, dimethoate, ethoate-methyl, formothion, mecarbam, omethoate, prothoate, sophamide and vamidothion; oxime organothiophosphate insecticides such as chlorphoxim, phoxim and phoxim-methyl; heterocyclic organothiophosphate insecticides such as azamethiphos, coumaphos, coumithoate, dioxathion, endothion, menazon, morphothion, phosalone, pyraclofos, pyridaphenthion and quinothion; benzothiopyran organothiophosphate insecticides such as dithicrofos and thicrofos; benzotriazine organothiophosphate insecticides such as azinphos-ethyl and azinphos-methyl; isoindole organothiophosphate insecticides such as dialifos and phosmet; isoxazole organothiophosphate insecticides such as isoxathion and zolaprofos; pyrazolopyrimidine organothiophosphate insecticides such as chlorprazophos and pyrazophos; pyridine organothiophosphate insecticides such as chlorpyrifos and chlorpyrifos-methyl; pyrimidine organothiophosphate insecticides such as butathiofos, diazinon, etrimfos, lirimfos, pirimiphos-ethyl, pirimiphos-methyl, primidophos, pyrimitate and tebupirimfos; quinoxaline organothiophosphate insecticides such as quinalphos and quinalphos-methyl; thiadiazole organothiophosphate insecticides such as athidathion, lythidathion, methidathion and prothidathion; triazole organothiophosphate insecticides such as isazofos and triazophos; phenyl organothiophosphate insecticides such as azothoate, bromophos, bromophos-ethyl, carbophenothion, chlorthiophos, cyanophos, cythioate, dicapthon, dichlofenthion, etaphos, famphur, fenchlorphos, fenitrothion, fensulfothion, fenthion, fenthion-ethyl, heterophos, jodfenphos, mesulfenfos, parathion, parathion-methyl, phenkapton, phosnichlor, profenofos, prothiofos, sulprofos, temnephos, trichlormetaphos-3 and trifenofos; phosphonate insecticides such as butonate and trichlorfon; phosphonothioate insecticides such as mecarphon; phenyl ethylphosphonothioate insecticides such as fonofos and trichloronat; phenyl phenylphosphonothioate insecticides such as cyanofenphos, EPN and leptophos; phosphoramidate insecticides such as crufomate, fenamiphos, fosthietan, mephosfolan, phosfolan and pirimetaphos; phosphoramidothioate insecticides such as acephate, isocarbophos, isofenphos, methamidophos and propetamphos; phosphorodiamide insecticides such as dimefox, mazidox, mipafox and schradan; oxadiazine insecticides such as indoxacarb; phthalimide insecticides such as dialifos, phosmet and tetramethrin; pyrazole insecticides such as acetoprole, ethiprole, fipronil, pyrafluprole, pyriprole, tebufenpyrad, tolfenpyrad and vaniliprole; pyrethroid ester insecticides such as acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin and transfluthrin; pyrethroid ether insecticides such as etofenprox, flufenprox, halfenprox, protrifenbute and silafluofen; pyrimidinamine insecticides such as flufenerim and pyrimidifen; pyrrole insecticides such as chlorfenapyr; tetronic acid insecticides such as spirodiclofen, spiromesifen and spirotetramat; thiourea insecticides such as diafenthiuron; urea insecticides such as flucofuron and sulcofuron; and unclassified insecticides such as AKD-3088, closantel, crotamiton, cyflumetofen, E2Y45, EXD, fenazaflor, fenazaquin, fenoxacrim, fenpyroximate, FKI-1033, flubendiamide, HGW86, hydramethylnon, isoprothiolane, malonoben, metaflumizone, metoxadiazone, nifluridide, NNI-9850, NNI-0101, pymetrozine, pyridaben, pyridalyl, Qcide, rafoxanide, rynaxypyr, SYJ-159, triarathene and triazamate and any combinations thereof.

dsRNA Compositions Comprising a Herbicide.

In certain embodiments, compositions comprising the dsRNAs described herein may include one or more herbicide(s) (e.g., a herbicide other than a dsRNA described herein. Illustrative herbicides include but are not limited to: amide herbicides such as allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid and tebutam; anilide herbicides such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen and propanil; arylalanine herbicides such as benzoylprop, flamprop and flamprop-M; chloroacetanilide herbicides such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchior, thenylchlor and xylachlor; sulfonanilide herbicides such as benzofluor, perfluidone, pyrimisulfan and profluazol; sulfonamide herbicides such as asulam, carbasulam, fenasulam and oryzalin; antibiotic herbicides such as bilanafos; benzoic acid herbicides such as chloramben, dicamba, 2,3,6-TBA and tricamba; pyrimidinyloxybenzoic acid herbicides such as bispyribac and pyriminobac; pyrimidinylthiobenzoic acid herbicides such as pyrithiobac; phthalic acid herbicides such as chlorthal; picolinic acid herbicides such as aminopyralid, clopyralid and picloram; quinolinecarboxylic acid herbicides such as quinclorac and quinmerac; arsenical herbicides such as cacodylic acid, CMA, DSMA, hexaflurate, MAA, MAMA, MSMA, potassium arsenite and sodium arsenite; benzoylcyclohexanedione herbicides such as mesotrione, sulcotrione, tefuryltrione and tembotrione; benzofuranyl alkylsulfonate herbicides such as benfuresate and ethofumesate; carbamate herbicides such as asulam, carboxazole chlorprocarb, dichlormate, fenasulam, karbutilate and terbucarb; carbanilate herbicides such as barban, BCPC, carbasulam, carbetamide, CEPC, chlorbufam, chlorpropham, CPPC, desmedipham, phenisopham, phenmedipham, phenmedipham-ethyl, propham and swep; cyclohexene oxime herbicides such as alloxydim, butroxydim, clethodim, cloproxydim, cycloxydim, profoxydim, sethoxydim, tepraloxydim and tralkoxydim; cyclopropylisoxazole herbicides such as isoxachlortole and isoxaflutole; dicarboximide herbicides such as benzfendizone, cinidon-ethyl, flumezin, flumiclorac, flumioxazin and flumipropyn; dinitroaniline herbicides such as benfluralin, butralin, dinitramine, ethalfluralin, fluchloralin, isopropalin, methalpropalin, nitralin, oryzalin, pendimethalin, prodiamine, profluralin and trifluralin; dinitrophenol herbicides such as dinofenate, dinoprop, dinosam, dinoseb, dinoterb, DNOC, etinofen and medinoterb; diphenyl ether herbicides such as ethoxyfen; nitrophenyl ether herbicides such as acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen and oxyfluorfen; dithiocarbamate herbicides such as dazomet and metam; halogenated aliphatic herbicides such as alorac, chloropon, dalapon, flupropanate, hexachloroacetone, iodomethane, methyl bromide, monochloroacetic acid, SMA and TCA; imidazolinone herbicides such as imazamethabenz, imazamox, imazapic, imazapyr, imazaquin and imazethapyr; inorganic herbicides such as ammonium sulfamate, borax, calcium chlorate, copper sulfate, ferrous sulfate, potassium azide, potassium cyanate, sodium azide, sodium chlorate and sulfuric acid; nitrile herbicides such as bromobonil, bromoxynil, chloroxynil, dichlobenil, iodobonil, ioxynil and pyraclonil; organophosphorus herbicides such as amiprofos-methyl, anilofos, bensulide, bilanafos, butamifos, 2,4-DEP, DMPA, EBEP, fosamine, glufosinate, glyphosate and piperophos; phenoxy herbicides such as bromofenoxim, clomeprop, 2,4-DEB, 2,4-DEP, difenopenten, disul, erbon, etnipromid, fenteracol and trifopsime; phenoxyacetic herbicides such as 4-CPA, 2,4-D, 3,4-DA, MCPA, MCPA-thioethyl and 2,4,5-T; phenoxybutyric herbicides such as 4-CPB, 2,4-DB, 3,4-DB, MCPB and 2,4,5-TB; phenoxypropionic herbicides such as cloprop, 4-CPP, dichlorprop, dichlorprop-P, 3,4-DP, fenoprop, mecoprop and mecoprop-P; aryloxyphenoxypropionic herbicides such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; phenylenediamine herbicides such as dinitramine and prodiamine; pyrazolyl herbicides such as benzofenap, pyrazolynate, pyrasulfotole, pyrazoxyfen, pyroxasulfone and topramezone; pyrazolyiplpiethyl herbicides such as fluazolate and pyraflufen; pyridamine herbicides such as credazine, pyridafol and pyridate; pyridazitiotte herbicides such as brompyrazon, chloridazon, dimidazon, flufenpyr, metflurazon, norflurazon, oxapyrazon and pydanon; pyridine herbicides such as aminopyralid, cliodinate, clopyralid, dithiopyr, fluoroxypyr, haloxydine, picloram, picolinafen, pyriclor, thiazopyr and triclopyr; pyrimidinediamine herbicides such as iprymidam and tioclorim; quaternary ammonium herbicides such as cyperquat, diethamquat, difenzoquat, diquat, morfamquat and paraquat; thiocarbamate herbicides such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate and vernolate; thiocarbonate herbicides such as dimexano, EXD and proxan; thiourea herbicides such as methiuron; triazine herbicides such as dipropetryn, triaziflam and trihydroxytriazine; chlorotriazine herbicides such as atrazine, chlorazine, cyanazine, cyprazine, eglinazine, ipazine, mesoprazine, procyazine, proglinazine, propazine, sebuthylazine, simazine, terbuthylazine and trietazine; methoxytriazine herbicides such as atraton, methometon, prometon, secbumeton, simeton and terbumeton; methylthiotriazine herbicides such as ametryn, aziprotryne, cyanatryn, desmetryn, dimethametryn, methoprotryne, prometryn, simetryn and terbutryn; triazinone herbicides such as ametridione, amibuzin, hexazinone, isomethiozin, metamitron and metribuzin; triazole herbicides such as amitrole, cafenstrole, epronaz and flupoxam; triazolone herbicides such as amicarbazone, bencarbazone, carfentrazone, flucarbazone, propoxycarbazone, sulfentrazone and thiencarbazone-methyl; triazolopyrimidine herbicides such as cloransulam, diclosulam, florasulam, flumetsulam, metosulam, penoxsulam and pyroxsulam; uracil herbicides such as butafenacil, bromacil, flupropacil, isocil, lenacil and terbacil; 3-phenyluracils; urea herbicides such as benzthiazuron, cumyluron, cycluron, dichloralurea, diflufenzopyr, isonoruron, isouron, methabenzthiazuron, monisouron and noruron; phenylurea herbicides such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluoron, phenobenzuron, siduron, tetrafluoron and thidiazuron; pyrimidinylsulfonylurea herbicides such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron and trifloxysulfuron; triazinylsulfonylurea herbicides such as chlorsulfuron, cinosulfuron, ethametsulfuron, iodosulfuron, metsulfuron, prosulfuron, thifensulfuron, triasulfuron, tribenuron, triflusulfuron and tritosulfuron; thiadiazolylurea herbicides such as buthiuron, ethidimuron, tebuthiuron, thiazafluoron and thidiazuron; and unclassified herbicides such as acrolein, allyl alcohol, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, fluorochloridone, flurtamone, fluthiacet, indanofan, methazole, methyl isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan and tritac.

dsRNA Compositions Comprising Other Agricultural Compounds

In certain embodiments, compositions comprising the dsRNAs described herein comprise a rodenticide. Illustrative rodenticides include but are not limited to 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pynnuron, scilliroside, sodium arsenite, sodium cyanide, sodium fluoroacetate, strychnine, thallium sulfate, warfarin and zinc phosphide.

In certain embodiments, compositions comprising the dsRNAs described herein may include other antibacterial agents. Illustrative antibacterial agents include, but are not limited to Streptomycin, oxytetracycline, oxolinic acid, or gentamicin. Other examples of antibacterial compounds include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie). In certain embodiments, compositions comprising the dsRNAs described herein mixed with other group of chemicals including for example, hormones, nutrients, crop beneficials and crop protection products are provided.

In certain embodiments, application of RNAs may be made as seed treatment, foliar application, stem application, drench or drip application (chemigation) to the seed, the plant or the fruit, to the soil or to inert substrate (e.g., inorganic substrates like sand, rockwool, glasswool; expanded minerals like perlite, vermiculite, zeolite or expanded clay), Pumice, Pyroclastic materials or stuff, synthetic organic substrates (e.g., polyurethane) organic substrates (e.g. peat, composts, tree waste products like coir, wood fiber or chips, tree bark) or to a liquid substrate (e.g. floating hydroponic systems, Nutrient Film Technique, Aeroponics) wherein the plant is growing or wherein it is desired to be grown.

It will be recognized that in various embodiments where compositions comprising one or more dsRNA(s) described herein and additional agent as described above are provided, methods that involve administration of the dsRNA(s) and the additional agents separately are also contemplated.

In certain embodiments, application of RNAs may be made as seed treatment, foliar application, stem application, drench or drip application (chemigation) to the seed, the plant or the fruit, to the soil or to inert substrate (e.g., inorganic substrates like sand, rockwool, glasswool; expanded minerals like perlite, vermiculite, zeolite or expanded clay), Pumice, Pyroclastic materials or stuff, synthetic organic substrates (e.g., polyurethane) organic substrates (e.g. peat, composts, tree waste products like coir, wood fibre or chips, tree bark) or to a liquid substrate (e.g. floating hydroponic systems, Nutrient Film Technique, Aeroponics) wherein the plant is growing or wherein it is desired to be grown.

In certain embodiments the compositions facilitate methods for controlling a phytopathogen (e.g., a phtopathogenic fungus) characterized in that an effective and non-phytotoxic amount of dsRNA molecules described herein, or of compositions described herein are applied to the soil where plants grow or are capable of growing, to the leaves and/or the fruit of plants or to the seeds of such plants.

In certain embodiments the dsRNAs can be formulated for and applied by the plants by means of various methods of treatment such as: 1) Spraying onto the aerial parts of the said plants a liquid comprising one of the said compositions; 2) Dusting, incorporating granules or powders into the soil, spraying, around the said plants and in the case of trees injection or daubing; 3) Coating or film-coating the seeds of the plants with the aid of a plant-protection mixture comprising one of the said compositions; and the like.

In certain embodiments the methods provided herein can be a curing, preventing or eradicating method. In certain embodiments, the dose of active dsRNA compound usually applied in the methods of treatment described herein is generally and advantageously:
1) For foliar treatments: from 0.0001 to 10,000 g/ha, preferably from 0,0001 to 1000 g/ha, more preferably from 0.001 to 300 g/ha;
2) In case of drench or drip application, the dose can even be reduced, especially while using inert substrates like rockwool or perlite;
3) For seed treatment: from 0.0001 to 200 g per 100 kilogram of seed, preferably from 0.001 to 150 g per 100 kilogram of seed;
4) For soil treatment: from 0.0001 to 10,000 g/ha, preferably from 0.001 to 5,000 g/ha.

Optimal application methods and dosages can readily be determined by one of skill in the art.

The former is illustrative and non-limiting. Using the teaching provided herein, numerous compositions/formulations of the dsRNAs suitable for application to a plant or to a plant part (e.g., a seed, leaf, or fruit) will be available to one of skill in the art.

### Recombinant Constructs

In certain embodiments, a recombinant nucleic acid construct, such as a DNA, comprising and/or encoding a nucleic acid molecule disclosed anywhere herein for silencing a target gene, including long dsRNA, hpRNA, miRNA, and siRNA is provided. Certain aspects provide for recombinant nucleic acid constructs comprising and/or encoding an RNAi precursor of a nucleic acid molecule disclosed anywhere herein for silencing a target gene, including long dsRNA, hpRNA, miRNA, tasiRNA, and siRNA.

In certain embodiments, a recombinant nucleic acid construct, such as a DNA vector, encoding and capable of expressing a target gene silencing sequence for silencing a target gene described anywhere herein is provided. In certain aspects, a recombinant DNA construct comprises a gene silencing sequence comprising about any of 17, 18, 19, 20, 21, 22, 23, 24, 25, 30, 40, 50, 60, 100, 120, 200, 240, 300, 400, 500, 600, 650, 750, 1000 to about any of 21, 22, 23, 24, 25, 30, 40, 50, 100, 200, 300, 400, 500, 600, 650, 750, 1000, or 2000 contiguous nucleotides of a target gene or target gene transcript disclosed anywhere herein. In certain aspects, a recombinant DNA construct comprises or encodes a gene silencing sequence complementary to about 17 to 21 contiguous nucleotides, or complementary to about 17 to 50 contiguous nucleotides, or complementary to about 50 to 100 contiguous nucleotides, or complementary to about 50 to 250 contiguous nucleotides, or complementary to about 250-600 contiguous nucleotides, or complementary to about 500-1,000 contiguous nucleotides of a target gene sequence or transcript thereof. In certain aspects, the gene silencing sequence comprises about contiguous nucleotides of the protein coding region of the target gene sequence. In certain aspects, the gene silencing sequence comprises about 21 to 500 or 100 to 1000 or about 200 to 1000 contiguous nucleotides of the 5′ UTR region or the 3′ UTR region of the target gene sequence. In certain aspects, the gene silencing sequence comprises at least 0.1%, 0.4%, 0.6%, 1%, 2%, 3%, 5%, 7%, 10%, 20%, 30%, 40%, 50%, 60%, 70%, 80%, 90%, 95%, 98%, 99%, or 100% contiguously of the length of target gene sequence protein coding region, the target gene sequence 5′ UTR region, target gene sequence 3′ UTR region and/or any combination thereof.

In certain embodiments, the recombinant DNA construct comprises a sequence coding a gene silencing sequence (e.g., dsRNA as described herein) operably linked to one or more promoters for the expression of a dsRNA molecule that silences the target gene when transcribed into a host plant. Thus, in certain aspects, the construct comprises an expression vector.

Representative promoters for use in expressing a dsRNA molecule include, but are not limited to promoters that are operable in plants. Such promoters include constitutive promoters, inducible promoters, and tissue-specific promoters.

Constitutive promoters facilitate expression of the gene in all tissues regardless of the surrounding environment and development stage of the organism. Such promoters can turn on the gene in every living cell of the organism, all the time, throughout the organism's lifetime. These promoters can often be utilized across species. Examples of constitutive promoters that are commonly used for plants include Cauliflower mosaic virus (CaMV) 355, opine promoters, plant ubiquitin (Ubi), rice actin 1 (Act-1) and maize alcohol dehydrogenase 1 (Adh-1). CaMV 35S is the most commonly used constitutive promoter for high levels of gene expression in dicot plants. Maize Ubi and rice Act-1 are currently the most commonly used constitutive promoters for monocots.

Tissue-specific or development-stage-specific promoters facilitate expression of a gene in specific tissue(s) or at certain stages of development while leaving the rest of the organism unmodified. In the case of plants, such promoters might specifically influence expression of genes in the roots, fruits, or seeds, or during the vegetative, flowering, or seed-setting stage. If the developer wants a gene of interest to be expressed in more than one tissue type for example the root, anthers and egg sac, then multiple tissue-specific promoters may have to be included in the gene construct.

Effective gene expression in specific plant parts or development stages often has been observed when promoters from closely related species are used. There are many promoters in this category because they have different tissue and developmental specificities. An example of a tissue-specific promoter is the phosphoenolpyruvate (PEP) carboxylase promoter which induces gene expression only in cells that are actively involved in photosynthesis. In plant genetic engineering, this promoter is used for traits desired in the shoot, leaves and sometimes the stem. Expression of genes controlled by this promoter is reduced later in the growing season as the plant approaches senescence.

Inducible promoters are activated by exogenous (i.e., external) factors. Exogenous factors may be abiotic such as heat, water, salinity, chemical, or biotic like pathogen or insect attack. Promoters that react to abiotic factors are the most commonly used in plant genetic engineering because these can easily be manipulated. Such promoters respond to chemical compounds such as antibiotics, herbicides or changes in temperature or light. Inducible promoters can also be tissue or development stage specific.

Promoters can be derived directly from naturally occurring genes, or may be synthesized to combine regulatory sequences from different promoter regions. The promoters interact with other regulatory sequences (enhancers or silencers) and regulatory proteins (transcription factors) to influence the amount of gene transcription/expression.

### Transgenic Plants and Host Induced Gene Silencing

In certain embodiments, transgenic plants are provided that provide host induced gene silencing (HIGS). Host-induced gene silencing (HIGS) which is a method where a plant controls a pathogen by RNA interference (RNAi) and exhibits, for example, an enhanced resistance when carrying suitable gene silencing constructs.

In various embodiments for the production of transgenic phytopathogen resistant plants in the present invention, a DNA capable of expressing one or more dsRNA as described herein is provided selected and introduced into the plant or a part thereof.

The DNA capable of expressing the dsRNA(s) may be present within an expression cassette. As used herein, an “expression cassette” is a nucleic acid molecule that is composed of one or more genes or genetic sequences and the sequences controlling their expression. An expression cassette may contain a promoter regulatory sequence, also designated promoter, operably linked to an open reading frame or another genetic sequence, and a 3′ untranslated region that may contain a polyadenylation site. The promoter directs the machinery of the cell to make RNA and/or protein. As used herein, “operably linked” means that expression of the linked DNA is under control of the operably linked promoter. In certain embodiments, an expression cassette may be part of a vector used for cloning and introducing the DNA into a cell.

A DNA capable of expressing a dsRNA described herein may comprise one or more DNA sequence capable of expressing one or more dsRNAs against one target gene or target gene ortholog described herein or more than one target gene or target gene ortholog described herein such as 2, 3, 4, 5, etc. Consequently, in certain embodiments, the transgenic plants contemplated herein may comprise in a cell one DNA molecule expressing one or more dsRNA(s) against one target gene or target gene ortholog described herein, or against more than one target gene or target gene ortholog described herein. Alternatively, in certain embodiments, the transgenic plant may comprise in a cell more than one DNA expressing more than one dsRNA described herein, one dsRNA molecules, each DNA or each dsRNA molecule being against one or more than one specific target gene(s) or target gene analogs described herein. Also, combinations of DNA encoding dsRNA against one gene and DNA encoding dsRNA against more than one gene are contemplated herein.

In certain embodiments, the DNA introduced into the transgenic plants is capable of expressing an inhibitory nucleic acid molecule results, upon transcription in the plant cell in an mRNA molecule that may function as an antisense or other inhibitory RNA and inhibit mRNA(s) involved expression of the gene product(s) of the target gene(s) described herein.

In certain embodiments, the DNA introduced into the transgenic plants is results upon expression (transcription) in the plant cell in an mRNA molecule or in mRNA molecules that may form a dsRNA which may activate the RNAi machinery. Within this embodiment, the DNA encodes an RNA molecule in sense direction and an RNA molecule in antisense direction. The sense sequence and the antisense sequence may be present on one DNA strand. This may result in an mRNA molecule which comprises the sense sequence and antisense sequence on the same mRNA molecule. Due to the reverse substantial complementarity of the sense sequence and antisense sequence, a hairpin dsRNA will form. Alternatively, if e.g., the sense and antisense sequences being present on the same DNA strand are under the control of different promoters or of one bidirectional promoter, different mRNA molecules are transcribed, one harboring the sense sequence and the other harboring the antisense sequence. Different mRNA molecules, one harboring the sense sequence and the other harboring the antisense sequence, may also form if the sense sequence and the antisense sequence are present on different strands of the DNA. Due to the reverse substantial complementarity of the sense and antisense sequences, the mRNAs form a dsRNA. The dsRNA may be recognized as foreign by the plant cell and may activate the RNAi machinery. The dsRNA transcribed from the DNA may be an siRNA, miRNA or other inhibitory RNA molecules or may be processed to an siRNA, miRNA or other inhibitory RNA molecules. After infection of the plant with a phytopathogen (e.g., a phytopathogenic fungus), an exchange of the RNA generated in the plant may occur between the plant and the phytopathogen. The RNA incorporated in the phytopathogen may lead to a sequence-specific gene silencing of the target gene(s) or target gene ortholog(s). It is known that the siRNA effect can be continued in plants if the RNA dependent RNA polymerase synthesizes new siRNAs from the degraded mRNA fragments. These secondary or transitive RNAis can enhance the silencing.

For introducing the DNA molecule into a plant or a part thereof, numerous methods are known in the art. One illustrative, but non-limiting method is transformation of the DNA molecule, expression cassette or vector harboring the DNA molecule by the use of bacteria of the Agrobacterium genus, e.g., by infection of the cells or tissues of plants with A. tumefaciens (see, e.g., Knopf (1979) Subcell. Biochem. 6: 143-173; Shaw et al. (1983) Gene, 23(3); 315-330) or A. rhizogenes (see, e.g., Bevan & Chilton (1982) Annu. Rev. Genet. 16: 357-384; Tepfer & Casse-Delbart (1987) Microbiol. Sci. 4(1); 24-28). For example, the transformation of plant cells or tissues with Agrobacterium tumefaciens can be carried out according to the protocol described by Hiei et al. (1994) Plant J. 6(2); 271-282).

Other illustrative, but non limiting methods for introducing DNA molecules into the plant genome include engineered nucleases such as zinc finger nucleases (ZFNs), Transcription Activator Like Effector Nucleases (TALENs), engineered homing endonucleases, and RNA or DNA guided endonucleases, such as CRISPR gene editing technology to carry out site specific integration (see, e.g., Wada et al. (2020) BMC Plant Biol 20: 234). Additionally, or alternatively, RNA targeting systems can use used, such as CRISPR/Cas systems have RNA targeting nucleases.

Another method is the biolistic transformation method, wherein cells or tissues are bombarded with particles onto which the vectors of the invention are adsorbed (see, e.g., Bruce et al. (1989) Proc. Natl. Acad. Sci. USA 86(24); 9692-9696; Klein et al. (1992) Biotechnology, 10(3); 286-291; U.S. Pat. No. 4,945,050, and the like). A further method is the widely used protoplast transformation where the plant cells are separated by pectinases and subsequently, the cell wall is degraded to generate protoplasts. For transformation, polyethylene glycol is added or electroporation is applied. Other methods can involve bringing the plant cells or tissues into contact with polyethylene glycol (PEG) and the vectors (see, e.g., Chang & Cohen (1979) Mol. Gen. Genet. 168(1); 111-115; Mercenier & Chassy (1988) Biochimie 70(4); 503-517; and the like). Electroporation is another method, that consists in subjecting the cells or tissues to be transformed and the vectors described herein to an electric field (see, e.g., Andreason & Evans (1988) Biotechniques 6(7); 650-660; Shigekawa & Dower (1989) Aust. J. Biotechnol. 3(1); 56-62; and the like). Another method consists in directly injecting the vectors into the cells or the tissues by microinjection (see, e.g., Gordon & Ruddle (1985) Gene 33(2); 121-136). Those skilled in the art will choose the appropriate method according to the nature of the plant to be transformed and the phytopathogen which the plant is to be rendered resistant.

In various embodiments, the selection step for identifying a transformed plant or a part thereof comprising the DNA molecule, or a processed construct can be carried out via a selectable gene present in the vector, as referred to above. In certain embodiments, the selectable gene may comprise an operably linked promoter regulatory sequence and terminator regulatory sequence that are functional in plant cells.

Among the selectable markers that can be used reference is made to genes for resistance against antibiotics, such as the hygromycin phosphotransferase gene, the neomycin phosphotransferase II gene inducing resistance against kanamycin, or the aminoglycoside 3-adenyltransferase gene, genes for tolerance to herbicides such as the bar gene (White et al. (1990) Nucl. Acids Res., 18: 1062) for tolerance to bialaphos, the EPSPS gene (U.S. Pat. No. 5,188,642) for tolerance to glyphosate or else the HPPD gene (PCT Pub No: WO 96/038567) for tolerance to isoxazoles, genes encoding identifiable enzymes, such as the GUS enzyme, GFP protein or genes encoding pigments or enzymes regulating pigment production in the transformed cells. Such selectable marker genes are described in PCT Publication Nos: WO 91/002071, WO 95/006128, WO 96/038567, and WO 97/004103.

Cells or tissues of plants, e.g., root cells grown in culture, can be transformed with the desired gene (encoding a dsRNA described herein) and grown into mature plants. When transformation is effective, the transgene will be incorporated into the pollen and eggs and passed on to the next generation.

In one embodiment, the DNA molecule or expression cassette is stably integrated into the genome of the transgenic plant, preferably into a chromosome of the plant. Integration can, however, also occur into an extrachromosomal element. By stable integration into the genome of a plant, the DNA sequences can be passed to subsequent generations of the transgenic plant. Alternatively, the DNA molecule or expression cassette is present within the plant cell on the vector used to introduce the DNA molecule and is not stably integrated into the genome of the plant. Therefore, the DNA sequences may not be passed to subsequent generations of the plant.

In certain embodiments, all plants and all parts of a plant can be treated according to the methods described herein. By plants is meant all plants and plant populations such as desirable and undesirable wild plants, cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random or directed mutagenesis, molecular or genetic markers or by bioengineering or genetic engineering methods.

The term “part of a plant” refers to any parts or organs of a plant such shoot vegetative organs/structures, e.g., leaves, stems or tubers; roots, flowers or floral organs/structures, e.g. bracts, sepals, petals, stamens, carpels, anthers or ovules; seed, including embryo, endosperm or seed coat; fruit or the mature ovary; plant tissue, e.g. vascular tissue or ground tissue; or cells, e.g. guard cells, egg cells or trichomes; or progeny of the same. The term “cell” refers to a cell or cell accumulation within the plant as well as to an isolated cell or isolated cell accumulation. A cell may have a cell wall or may be a protoplast. In particular, in certain embodiments a seed which comprises the DNA, expression cassette or vector is provided. Preferably, the seeds of a transgenic plant retain the DNA, expression cassette or vector described herein, so that the new plants generated from a seed continues to comprise the DNA, expression cassette or vector

Plants that can be protected by the method according to the invention comprise all plants, preferably plants of economic interest. In certain embodiments, the plant is a plant selected from the group consisting of barley (Hordeum vulgare), sorghum (Sorghum bicolor), rye (Secale cereale), Triticale, sugar cane (Saccharum officinarium), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), sugar beet (Beta vulgaris), sunflower (Helianthus annuus), strawberry (Fragaria ananassa), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), potato (Solanum tuberosum), pepper (Capsicum annum), coffee (Coffea canephora), grape vine (Vitis vinifera and other Vitis species), cucumber (Cucumis sativus), mulberry (Morus notabilis), thale cress (Arabidopsis thaliana), Arabidopsis lyrata, sand rock-cress (Arabidopsis arenosa), rose (Rosa sp.), hops (Humulus lupulus), cannabis and hemp (Cannabis sativa), Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, cotton (Gossipium sp.), soybean (Glycine max), and black cottonwood (Populus trichocarpa). Particularly preferred, the plant is selected from the group consisting of barley (Hordeum vulgare), sorghum (Sorghum bicolor), rye (Secale cereale), Triticale, sugar cane (Saccharum officinarium), maize (Zea mays), rice (Oryza sativa), wheat (Triticum aestivum), Triticum durum, Avena sativa, Hordeum bulbosum, sugar beet (Beta vulgaris), sunflower (Helianthus annuus), carrot (Daucus carota), tobacco (Nicotiana tabacum), tomato (Solanum lycopersicum), potato (Solanum tuberosum), coffee (Coffea canephora), grape vine (Vitis sp. Including vinifera, labrusca, girdiana, arizonica, berlandieri, other vitis species and hybrids), cucumber (Cucumis sativus), thale cress (Arabidopsis thaliana), rape (Brassica napus), broccoli (Brassica oleracea), Brassica rapa, Brassica juncacea, black mustard (Brassica nigra), radish (Raphanus sativus), cotton (Gossipium sp.) and soy-bean (Glycine max). In certain embodiment, the plant is a plant that is a legume, a cereal, millets, berry plant, a fruit tree, an ornamental, a melon or gourd, a cucurbit, or other tree or plant infected by powdery mildew and other plant pathogens.

In certain embodiments, the plant is a modified plant, in which the plant genome is modified (e.g., by mutation, deletion, gene editing, introduction of a transgene, hybridization, etc.).

In various embodiments, a method of producing a transgenic plant or a part thereof is provided where the method comprises introducing into at least a cell of the plant the DNA or the expression cassette or the vector encoding a dsRNA described herein and regenerating the transgenic plant from the at least one cell.

As used herein “regenerating” means a process of growing an entire plant from a single cell, a group of cells, a part of the plant or a tissue of the plant. The skilled person knows methods of introducing DNA into at least a cell of the plant and growing a plant therefrom. “At least a cell” means a single cell, a group of cells, a part of the plant or a tissue of the plant.

In certain embodiments, a method of conferring phytopathogen (e.g., fungal) resistance to a plant or a part thereof is provided where the method comprises introducing into the plant or the part thereof the DNA or the expression cassette or the vector encoding a dsRNA described herein, and causing expression of the DNA or the expression cassette.

“As used herein, the term “causing expression” means that under the conditions, under which the plant is kept and/or cultivated, transcription of the DNA having been introduced into the plant is induced. For example, if the promoter is an inducible promoter, the activity of such promoter can be induced by the presence or absence of specific biotic or abiotic factors, according to the choice of the user of the present invention. If the promoter is a constitutive promoter, expression continuously occurs.

### Methods of Use.

In various embodiments, method of use of the dsRNAs described herein and/or DNAs encoding the dsRNAs are provided. In certain embodiments, the methods include a method for controlling or preventing an infection of a plant by a phytopathogen (e.g., a phytopathogenic fungus). In certain embodiments, the method comprises (a) contacting the plant or part thereof (e.g., a seed) and/or phytopathogen with an isolated double-stranded RNA (dsRNA) described herein or expressing in the plant at least one dsRNA as described herein.

In certain embodiments, the contacting comprises spraying, dunking, or coating the plant said plant with a composition for controlling or preventing an infection of a plant by a phytopathogen as described herein. In certain embodiments, the contacting is before infection by said phytopathogenic fungus. In certain embodiments, the contacting is during infection by said phytopathogenic fungus. In certain embodiments, the contacting comprises applying one or more dsRNAs described herein to a plant in need thereof. In certain embodiments, the plant comprises a plant of economic interest. In certain embodiments, the plant is a plant selected from the group consisting of barley (Hordeum vulgare), sorghum (Sorghum bicolor), rye (Secale cereale), Triticale, sugar cane (Saccharum officinarium), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), strawberry, sugar beet (Beta vulgaris), sunflower (Helianthus annuus), strawberry (Fragaria ananassa), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), pepper (Capsicum annum), potato (Solanum tuberosum), coffee (Coffea canephora), grape vine (Vitis vinifera and other Vitis species), cucumber (Cucumis sativus), mulberry (Morus notabilis), thale cress (Arabidopsis thaliana), Arabidopsis lyrata, sand rock-cress (Arabidopsis arenosa), rose (Rosa sp.), hops (Humulus lupulus), hemp (Cannabis sativa) and other Cannabis spp., Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, cotton (Gossipium sp.), soybean (Glycine max), and black cottonwood (Populus trichocarpa). Particularly preferred, the plant is selected from the group consisting of barley (Hordeum vulgare), sorghum (Sorghum bicolor), rye (Secale cereale), Triticale, sugar cane (Saccharum officinarium), maize (Zea mays), rice (Oryza sativa), wheat (Triticum aestivum), Triticum durum, Avena sativa, Hordeum bulbosum, sugar beet (Beta vulgaris), sunflower (Helianthus annuus), carrot (Daucus carota), tobacco (Nicotiana tabacum), tomato (Solanum lycopersicum), potato (Solanum tuberosum), coffee (Coffea canephora), grape vine (Vitis sp. Including, vinifera, labrusca, girdiana, arizonica, berlandieri other Vitis species and hybrids), cucumber (Cucumis sativus), thale cress (Arabidopsis thaliana), rape (Brassica napus), broccoli (Brassica oleracea), Brassica rapa, Brassica juncacea, black mustard (Brassica nigra), radish (Raphanus sativus), cotton (Gossipium sp.) and soy-bean (Glycine max). In certain embodiment, the plant is a plant that is a legume, a cereal, millets, berry plant, a fruit tree, an ornamental, a melon or gourd, a cucurbit, or other tree or plant infected by powdery mildew and other plant pathogens.

In certain embodiments, the contacting comprises applying one or more dsRNAs described herein to a plant selected from the group consisting of grape, wheat, barley, hops, hemp, cannabis, legumes, e.g., soybean), onion, artichoke, apples, cherry, peaches, pears, carrot, citrus, sunflower, cucumbers, strawberry, tomato, peppers, squashes (including pumpkins), luffas, melons, watermelons, all other cucurbits, coffee rust, oats, ryegrass, a cereal, corn, cotton, sugar cane, rose and other ornamentals. In certain embodiments, the contacting comprises applying one or more dsRNAs described herein to a grapevine.

In certain embodiments, the said expressing in the plant at least one isolated double-stranded RNA described herein comprises providing a transgenic plant as described herein.

### Screening Systems and Methods.

In various embodiments, methods of identifying genes in a phytopathogen that when downregulated in the phytopathogen control infection of a host plant by the phytopathogen are provided. In certain embodiments the methods involve i) selecting target gene(s) in the phytopathogen that have ortholog(s) in a reference powdery mildew; ii) designing long dsRNA or siRNA to inhibit the reference powdery mildew orthologues with minimal off-targets in the host plant; iii) inoculating (and/or contacting) said host plant with said reference powdery mildew; contacting said host plant with said dsRNA and/or siRNA; and iv) determining growth and/or reproduction of said reference powdery mildew in said host plant, where reduced growth or reproduction of said reference powdery mildew compared to an untreated plant of the same species indicates that the target genes are phytopathogen genes whose downregulation inhibits infection of said host plant by said phytopathogen. In certain embodiments the method comprises determining growth and/or reproduction of phytopathogen in said host plant, where reduced growth or reproduction of said phytopathogen compared to an untreated plant of the same species indicates that the target genes are phytopathogen genes whose downregulation inhibits infection of said host plant by said phytopathogen.

In certain embodiments, the method involves: i) selecting target gene(s) in the phytopathogen that have ortholog(s) in Golovinomyces orontii (G. orontii); 2) designing long dsRNA or siRNA to inhibit the G. orontii homologs or orthologues with minimal off-targets in the host plant; 3) inoculating an Arabidopsis spp. plant with the G. orontii; 4) contacting the Arabidopsis spp. plant with the dsRNA and/or siRNA; and 5) determining growth and/or reproduction of the G. orontii, where reduced growth or reproduction of the G. orontii compared to an untreated plant of the same species indicates that the target genes are phytopathogen genes whose downregulation inhibits infection of the host plant by the phytopathogen. One embodiment of such methods is illustrated in Example 1 (see also FIG. 2). In certain embodiments, the ortholog is an ortholog found in a powdery mildew. In certain embodiments, the ortholog is an ortholog found in a powdery mildew selected from the group consisting of powdery mildew genus Golovinomyces including G. orontii and G. cichorachearum (of curcubits), Erysiphe including E. necator (or Uncinula necator) (powdery mildew of grapes), Microsphaera including M. diffusa (powdery mildew of legumes, e.g. soybean), Leveillula including L. taurica (also known as Oidiopsis taurica) (powdery mildew of onion or artichoke), Oidium including O. neolycopersici (powdery mildew of tomato), Podosphaera including P. leucotricha (powdery mildew of apples and pears), P. macularis (powdery mildew of hemp and cannabis), P. xanthii (powdery mildew of cucurbits: cucumbers, squashes (including pumpkins), luffas, melons, and watermelons), and P. pannosa (also known as Sphaerotheca pannosa, powdery mildew of roses).

In certain embodiments, the ortholog is an ortholog as described above, with respect to target genes and ortholog target genes.

In certain embodiments, determining growth and/or reproduction comprises counting spore germination and/or differentiation, penetration of cell wall, haustoria formation, hyphal length and area, hyphal transect analysis colony size, conidiophores per colony, spore counts, phytopathogen DNA/RNA quantity, microscopic and visual estimation by eye, thermal imaging, fluorescent dyes/reporters, etc. In certain embodiments the determining growth and/or reproduction comprises counting spore production and/or determining hyphal length. In certain embodiments the determining growth and/or reproduction comprises counting spore production and/or determining hyphal length and/or visual disease symptoms.

### EXAMPLES

The following examples are offered to illustrate, but not to limit the claimed invention.

### Example 1

### Spray-Induced Gene Silencing Reduces Powdery Mildew Disease

### Summary of Example 1

Spray-induced gene silencing (SIGS) is an emerging tool being developed to protect crops from disease. It utilizes exogenously applied RNA designed to reduce gene expression of critical genes in plant pests using endogenous RNA interference machinery. The widespread obligate biotrophic pathogen, powdery mildew, infects agriculturally important crops like wheat, barley, cucurbits, and grapevine which can require numerous fungicide treatments to control. For grape powdery mildew, dominant varieties are susceptible to powdery mildew and extensive resistance to fungicides has caused a need for new disease management strategies. As described herein, we developed SIGS methods for powdery mildews and identified novel gene targets that significantly contribute to fungal growth. Golovinomyces orontii, a powdery mildew that infects Arabidopsis thaliana, was found to uptake RNA directly from its environment, independent of the host. SIGS methods using long double-stranded (dsRNA) and small interfering RNA (siRNA) were developed using whole plant and detached leaf assays. Method optimization utilized the azole-fungicide target CYP51. Ten additional powdery mildew targets were screened identifying 5 novel genes that contribute to fungal proliferation: lipase a, lipase 1, β-carotene 15,15′-dioxygenase, apoptosis-antagonizing transcription factor (AATF), and effector candidate 2. Translation of these methods to the Erysiphe necator-Vitis vinifera pathosystem showed similar reduction in disease with SIGS against CYP51. Furthermore, SIGS against the five effective targets, including AATF, identified in the G. orontii-A. thaliana pathosystem also showed reduced fungal proliferation in grapevine suggesting high throughput screening of targets in the G. orontii-A. thaliana pathosystem could prioritize targets for testing in the grapevine powdery mildew system. SIGS may be the future of controlling powdery mildew growth on crops because of its flexibility, resilience to resistance development, reduced environmental and health risks, and rapid transition from the bench to the field.

### Introduction

Powdery mildews are widespread obligate biotrophic pathogens that infect a variety of agriculturally important crops (Agrios, 2004). Hosts like grapevine require fungicides to control infection, though there are increasing needs for other options to manage disease due to fungicide resistance and increasing human health and environmental concerns associated with fungicide application (Calvert et al., 2008; Frenkel et al., 2015; Jones et al., 2014; Zubrod et al., 2014). Erysiphe necator, which causes grape powdery mildew, is especially troublesome for California grape growers as dominant cultivars are susceptible.

Powdery mildew disease management accounts for 74% of pesticide use amounting to 9% of table and raisin grape production costs and up to 20% of wine grape production costs of highly susceptible cultivars like chardonnay wine grapes in the Central Coast of California (Fuller et al., 2014). Sulfur is heavily used in the grape industry to prevent fungal disease. Although cheap and effective, sulfur is routinely applied during high disease pressure every 7-10 days (Jones et al., 2014) which can cause respiratory symptoms in workers and people living in the area (Raanan et al., 2017). In addition, some wine producers are concerned about a potential impact of sulfur on taste when applied repeatedly later in the growing season. Fungicides including azole-fungicides (demethylase inhibitors) are also used to control powdery mildew disease. These fungicides inhibit CYP51 function, which is required for sterol biosynthesis, a component of fungal cell membranes (Frenkel et al., 2015). Powdery mildew isolates that have increased resistance to azole-fungicides have increased expression of CYP51 and acquire mutations that decrease azole-fungicide sensitivity (Frenkel et al., 2015; Jones et al., 2014).

RNA interference (RNAi) strategies are being developed to offer additional solutions to combat plant disease. These tools utilize conserved eukaryotic host and/or pathogen RNAi machinery to mediate silencing of pathogen genes by degrading messenger RNA (Majumdar et al., 2017). Double-stranded RNA (dsRNA) can be provided by the host plant using host-induced gene silencing (HIGS) and has been demonstrated to be effective in reducing barley powdery mildew infection (Pliego et al., 2013) and other fungal diseases like Fusarium head blight (Machado et al., 2018), wheat stripe rust (Zhu et al., 2017), and grey mold (Xiong et al., 2019) suggesting plant-mediated RNA uptake is widespread. HIGS, however, requires the use of transgenic crops which can require years of engineering to create the transgenic host contributing to high production costs. Despite years of advancement, there are limited RNAi based plant products in the marketplace (Bramlett et al., 2020). Spray-induced gene silencing (SIGS) offers a higher throughput and adaptable strategy that is not transgenic. Much like HIGS, it relies on endogenous RNAi machinery to mediate gene silencing of critical pathogen genes. The necrotrophic fungus Botrytis cinerea has been shown to directly uptake RNA from the environment. Both long dsRNA and small RNAs (sRNAs) applied onto plants limited infection of B. cinerea (Wang et al., 2016). This strategy has the potential to be readily adaptable to more than one crop or pest. It requires that the pest is able to take up dsRNA and/or siRNA, has endogenous RNAi machinery, and has sequenced transcripts to target. SIGS has been shown to be effective in reducing disease in many fungal and oomycete pathogens with various lifestyles including obligate biotrophs, Hyaloperonospora arabidopsidis (Bilir et al., 2019) and Phakopsora pachyrhizi (Hu et al., 2020), the hemibiotroph, Fusarium graminearum (Koch et al., 2016), and the necrotroph, B. cinerea (Wang et al., 2016).

In this study, we developed SIGS methods in the Golovinomyces orontii-Arabidopsis thaliana model system using CYP51 as the positive control gene target (Koch et al., 2016) and translated these methods to the agriculturally relevant system, Vitis vinifera-Erysiphe necator. SIGS phenotypes can be detected at early time points in infection using microscopic hyphal length measurements or at later stages by quantifying fungal spore production. Both siRNAs (small interfering RNAs) and long dsRNAs were shown to be effective in reducing powdery mildew growth and reproduction. After screening methods were optimized, ten additional conserved powdery mildew genes were selected for testing. SIGS against five of these targets significantly reduce spore production and represent diverse target genes that function as metabolic enzymes, regulatory genes, and secreted protein effectors. These five, including AATF, were silenced in grapevine, with results similar to Arabidopsis, showing findings in Arabidopsis translates to other agronomic species. Through this research we can rapidly identify important powdery mildew gene targets suitable for RNAi-based control of powdery mildew disease.

### Results

G. orontii Spores can Take Up Environmental RNA

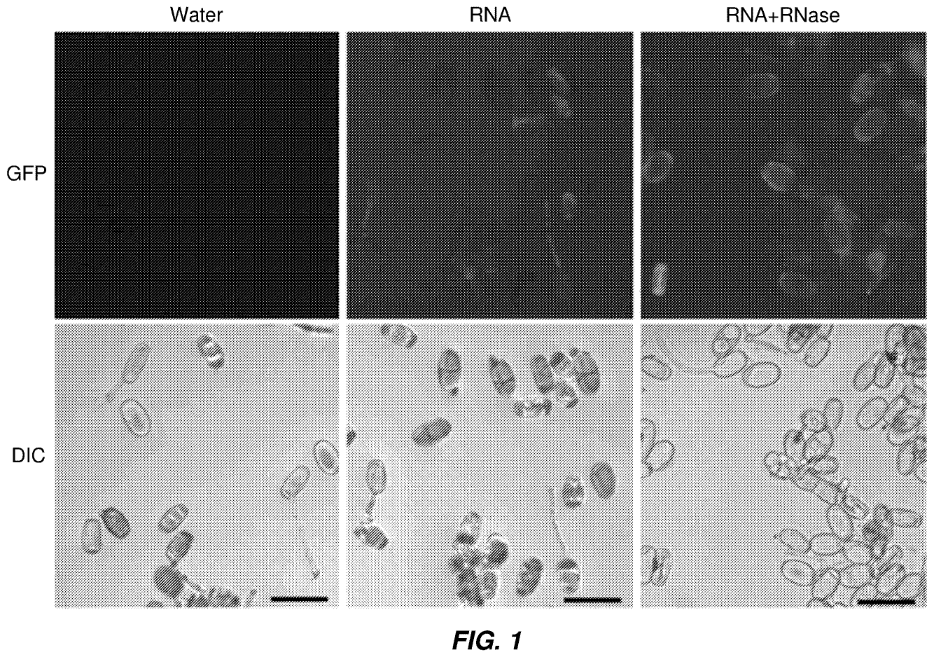

To determine if G. orontii can uptake RNA directly from its environment, germinated conidia were incubated with in vitro transcribed Fluorescein-labeled P-actin-Mouse RNA (300 bp) using methods adapted from (Wang et al., 2016). To ensure RNA signal was intracellular, RNases were added before imaging. Powdery mildew uptake of RNA is rapid and observed within 1.5 hours of application demonstrating powdery mildews can uptake RNA independent from the host plant. Fluorescein was observed in all tissues present: germinated conidia, germ tubes of germinated conidia, and ungerminated conidia (FIG. 1). Not all spores observed had fluorescein signal, possibly indicating more time would be required to observe uptake in all cells or not all spores were at the appropriate stage for uptake.

siRNA and Long dsRNA Design

siRNA and/or long dsRNA was designed for each gene target selected (FIG. 2, Table 5). Analysis was performed to minimize off-target impacts and incorporate known features of efficient siRNA design. The online resource pssRNAit was used to minimize off-target silencing in the host (A. thaliana or V. vinifera) and identify efficient siRNAs and dsRNAs in silico (FIG. 2). Known features of efficient siRNAs were incorporated into siRNA design (Mittal, 2004; Reynolds et al., 2004; Ui-Tei et al., 2004). Long dsRNAs in this study were 199-449 bps and siRNAs were composed of 21 base long sense and antisense strands that anneal over 19 bases with 2 base overhangs at the 3′ ends. Where applicable, dsRNAs were also designed to anneal to multiple copies of the gene where possible as there are varying degrees of sequence conservation among copies.

TABLE 5
Gene information for G. orontii MGH1 targets selected for spray-induced silencing.

| | Putative | Putative | | | |
|---|---|---|---|---|---|
| Gene | Gene | Gene | Copy | | |
| Category | Symbol | Name | Number¹ | Annotated Domains¹ | Rationale |
| Metabolic | CYP51 | cytochrome | 2 | cytochrome P450 | Encodes the azole-fungicide |
| | | P450 51 | | | target, sterol 14a-demethylase. |
| | | | | | It is critical for ergosterol |
| | | | | | synthesis which is required for |
| | | | | | fungal cell membranes. |
| | | | | | Fusarium graminearum |
| | | | | | infection of barley is reduced |
| | | | | | by spray-induced gene |
| | | | | | silencing of CYP51.2 |
| | GDB1 | glycogen | 2 | amylo-alpha-1,6- | Glycogen is broken down in |
| | | debranching | | glucosidase, glycogen | germinating and developing |
| | | enzyme | | debranching enzyme | powdery mildew conidia.³ It is |
| | | | | (glucanotransferase | thought to fuel development |
| | | | | domain), N-terminal | from germination to |
| | | | | and central domain | penetration. Glycogen |
| | | | | from the human | breakdown can be visualized |
| | | | | glycogen debranching | with Lugol staining, showing |
| | | | | enzyme | reduced glycogen content in |
| | GPH1 | glycogen | 2 | carbohydrate | germinated spores.³ |
| | | phosphorylase | | phosphorylase | |
| | LIP1 | lipase 1 | 2 | carboxylesterase | Lipid droplets are components |
| | | | | family | of powdery mildew conidia |
| | LIPA | lipase A | 2 | lipase (class 3) | that are consumed and |
| | DGAT | diglyceride | 2 | MBOAT (membrane- | mobilized during germination |
| | | acyltransferase | | bound O- | of powdery mildew conidia.³ |
| | | | | acyltransferase family) | |
| | BCDO | P-carotene | 1 | retinal pigment | Abscisic acid (ABA) |
| | | 15,15- | | epithelial membrane | promotes biotic fungal |
| | | dioxygenase | | protein (carotenoid | interactions with plants and |
| | | | | oxygenase) | decreases plant immunity. |
| | | | | | Many pathogens are known to |
| | | | | | produce or modulate ABA |
| | | | | | biosynthesis in host plants.⁴ G. |
| | | | | | orontii may require host ABA |
| | | | | | precursor as most genes are |
| | | | | | lost in carotenoid |
| | | | | | biosynthesis.¹ A. thaliana |
| | | | | | ABA biosynthetic gene |
| | | | | | expression is modulated at the |
| | | | | | powdery mildew infection |
| | | | | | site.⁵ |
| | UTR4 | unidentified | 2 | haloacid | UTR4 is a 2,3- |
| | | transcript 4 | | dehalogenase-like | dioxomethiopentane-1- |
| | | | | hydrolase | phosphate enolase/ |
| | | | | | phosphatase involved in the |
| | | | | | methionine salvage pathway |
| | | | | | which allows cells to |
| | | | | | regenerate methionine and |
| | | | | | reuse sulfur. Saccharomyces |
| | | | | | cerevisiae utr4 mutants grow |
| | | | | | poorly on media lacking |
| | | | | | methionine and have a |
| | | | | | significantly lower growth |
| | | | | | rate.⁶ |
| Regulatory | GID9 | glucose- | 2 | LisH, CTLH/CRA C- | Involved in proteasome- |
| | | induced | | terminal to LisH motif | dependent catabolite |
| | | degradation | | domain | inactivation of gluconeogenic |
| | | 9 | | | enzymes fructose-1,6- |
| | | | | | bisphosphatase, |
| | | | | | phosphoenolpyruvate |
| | | | | | carboxykinase, and |
| | | | | | cytoplasmic malate |
| | | | | | dehydrogenase.^{7,8} Glucose and |
| | | | | | sucrose concentrations are |
| | | | | | enhanced in powdery mildew |
| | | | | | infected leaf apoplast.⁹ |
| | AATF/ | apoptosis- | 2 | apoptosis-antagonizing | Required for proliferation in |
| | Che-1 | antagonizing | | transcription factor (C- | mammalian systems and |
| | | transcription | | terminal), apoptosis | involved in many cellular |
| | | factor | | antagonizing | processes including cell stress |
| | | | | transcription factor | response and cell cycle |
| | | | | | control.¹⁰ Pathogens |
| | | | | | manipulate the expression of |
| | | | | | AATF.^{11,12} |
| Effector | EC2 | effector | 2 | CFEM domain | Transgenic A. thaliana |
| | | candidate 2 | | | expressing EC2 have |
| | | | | | increased entry by nonadapted |
| | | | | | powdery mildew.¹³ EC2 is |
| | | | | | among the most highly |
| | | | | | expressed genes in G. orontii |
| | | | | | MGH1 and is widely |
| | | | | | conserved in powdery |
| | | | | | mildews¹. |

¹G. orontii MGH1 (v4.0) genome annotated and accessed through MycoCosm (Grigoriev et al., 2014),
2(Koch et al., 2016),
³(Both et al., 2005),
⁴(Lievens et al., 2017),
⁵(Chandran et al., 2010),
⁶(Pirkov et al., 2008),
⁷(Braun et al., 2011),
⁸(Regelmann et al., 2003),
⁹(Aked and Hall, 1993),
¹⁰(Iezzi and Fanciulli, 2015)
¹¹(Benakanakere et al., 2019),
¹²(Kaul et al., 2009),
¹³(Schmidt et al., 2014).

siRNA and dsRNA Designed to Target G. orontii CYP51 Significantly Reduces Spore Production in A. Thaliana Whole Plant and Detached Leaf Assays

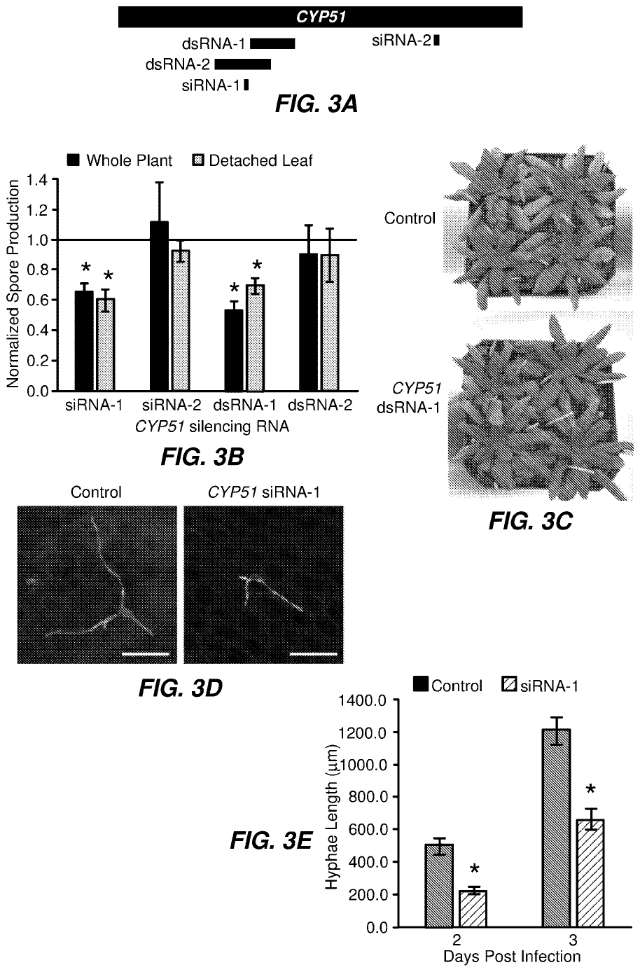

SIGS methods were optimized (FIG. 2) using the G. orontii MGH1 gene target, CYP51, which has been successfully silenced using SIGS to reduce growth of the fungal pathogen, F. graminearum (Koch et al., 2016). CYP51 function is required for sterol biosynthesis, a component of fungal cell membranes, and an azole-fungicide target (Frenkel et al., 2015). In G. orontii, there are two copies of CYP51 with 98.1% sequence identity. Two 21 bp siRNAs, siRNA-1 and siRNA-2, and two long dsRNAs, dsRNA-1 and dsRNA-2, targeting different regions of CYP51 were designed (FIG. 3, panel a).

The spraying methods, amount of RNA, number of RNA applications, timing of RNA application, RNA purification methods, and detached leaf or whole plant protocols were troubleshooted as part of SIGS protocol development to improve the efficiency and reproducibility of results (FIG. 2). Concentrations of dsRNA tested ranged from 10-80 ug/ml. The impact of SIGS on growth was measured by determining the asexual reproductive output as measured by harvesting infected tissue, liberating spores in 0.01% Tween-80, and determining the concentration of spores in suspensions (e.g., as adapted from WeBling et al., 2014). We focused on phenotyping fungal growth by measuring spore production as spores are what propagate the fungus and it can be rapidly assessed. For all experiments, results from control treatments with buffer alone were compared with the RNA-treated samples.

In whole plant and detached leaf experiments using CYP51 dsRNA-1, spore production decreased by an average of 46% and 30% respectively (FIG. 3, panel b) and visually less powdery mildew coverage and density was observed on dsRNA-sprayed plants than buffer control-sprayed plants (FIG. 3, panel c). Spore production was similarly reduced on average by 35% and 40% in whole plant and detached leaf CYP51 siRNA-1 treated samples, respectively (FIG. 3, panel b). These results show that SIGS against the G. orontii CYP51 target can reproducibly limit powdery mildew disease on Arabidopsis.

By contrast, the CYP51 dsRNA-2 and siRNA-2 treated samples did not affect fungal growth as spore production was similar to the control samples (FIG. 3, panel b). This is in accordance with previous findings that a negative result does not mean the targeted transcript is not important to the phenotype assessed; instead, the negative result can be due to poor silencing efficiency of a particular siRNA or long dsRNA for reasons not yet understood (Wu et al., 2004). The lack of impact of CYP51 dsRNA-2 and siRNA-2 on G. orontii spore production also indicates that the addition of dsRNA or siRNA alone does not impact powdery mildew growth.

Targeting CYP51 Significantly Reduces G. orontii Hyphal Length on A. Thaliana Detached Leaves

To see the impact of SIGS on early G. orontii colony development, we measured the impact of CYP51 siRNA-1 on hyphal length at 2 and 3 dpi in A. thaliana detached leaf assays. Observing the hyphal length of colonies is more time consuming compared to measuring spore production, requiring fixing and staining of harvested leaves, fluorescence microscopy, and image analysis, but may reveal early infection phenotypes masked at later points in infection. The average total hyphal length of CYP51 siRNA-1 colonies was significantly reduced at 2 and 3 dpi by 56% and 45% respectively compared to control samples (FIG. 3, panel e). Although the length was reduced, the branching architecture was not altered (FIG. 3, panel d). Coupled with the similar decrease in spore production with CYP51 siRNA-1 treatment (FIG. 3, panel b), this suggests the SIGS treatment against the cell membrane sterol biosynthetic enzyme CYP51 target was reproducibly effective in limiting powdery mildew growth, but was unable to fully prevent it. This could be due to a number of factors including RNA stability, design, uptake efficiency, and mobility that can be further optimized. In summary, the optimized SIGS protocol provides a high throughput means of screening for key powdery mildew targets of import to its proliferation. The extent of growth limitation for given target can be subsequently optimized. Finally, those targets without a powdery mildew disease phenotype are not necessarily unimportant; additional dsRNA and/or siRNAs would need to be designed and tested.

dsRNA Designed to Target Metabolic, Regulatory, and Effector Genes Identifies Novel Powdery Mildew Targets that Contribute to G. orontii Spore Production on A. Thaliana

To identify novel powdery mildew genes that contribute to its growth and development, metabolic, regulatory, and effector genes were targeted using SIGS to determine their impact on proliferation (spore production). Spore production was chosen for screening as it results in the propagation of the infection in the field and therefore is most relevant as an agricultural disease outcome assessment. dsRNA was chosen for initial screening as multiple siRNAs are generated from each dsRNA increasing the likelihood of a successful silencing outcome.

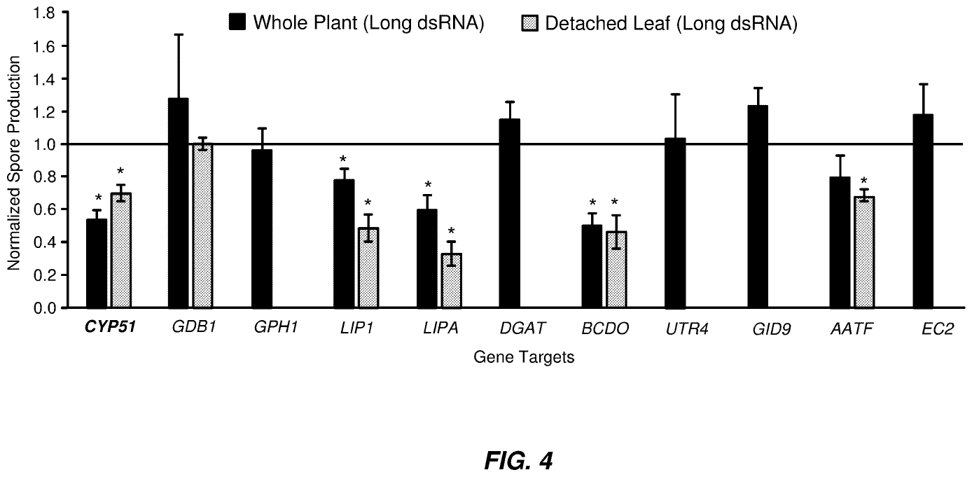

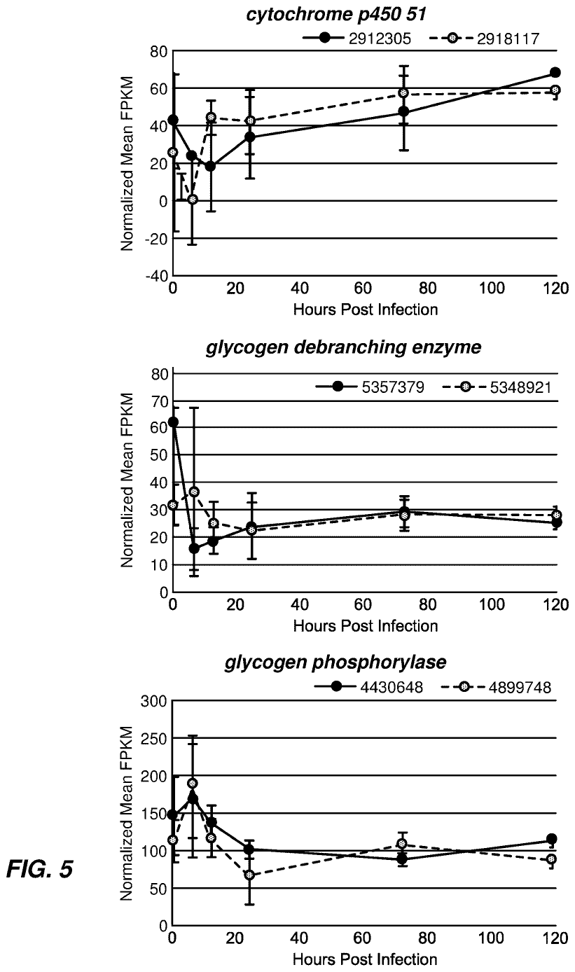

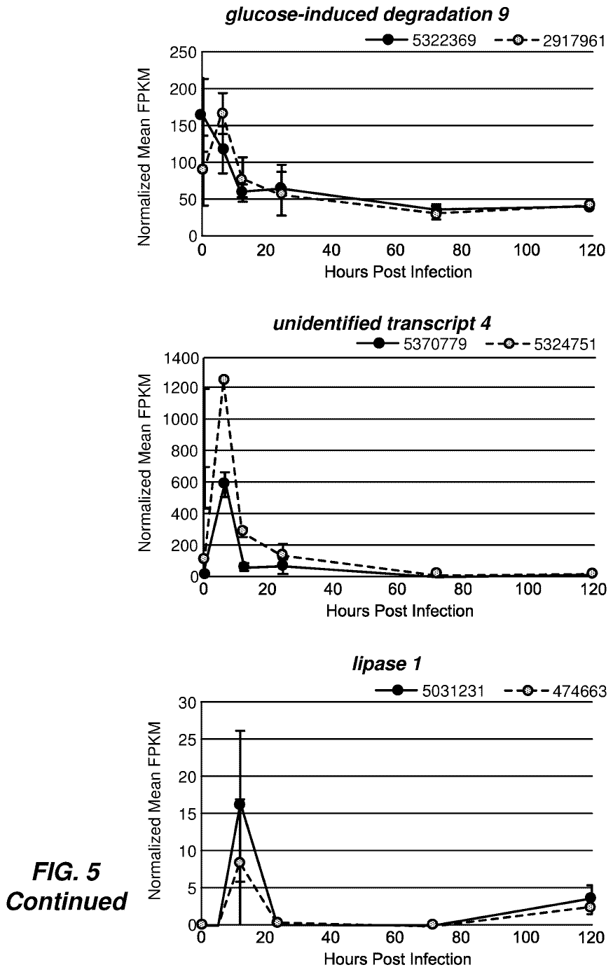

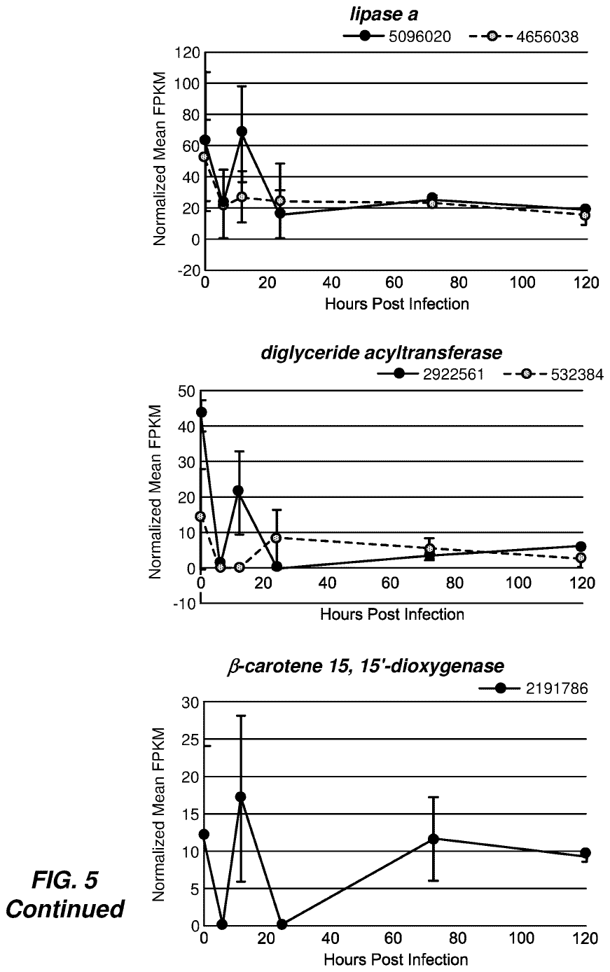

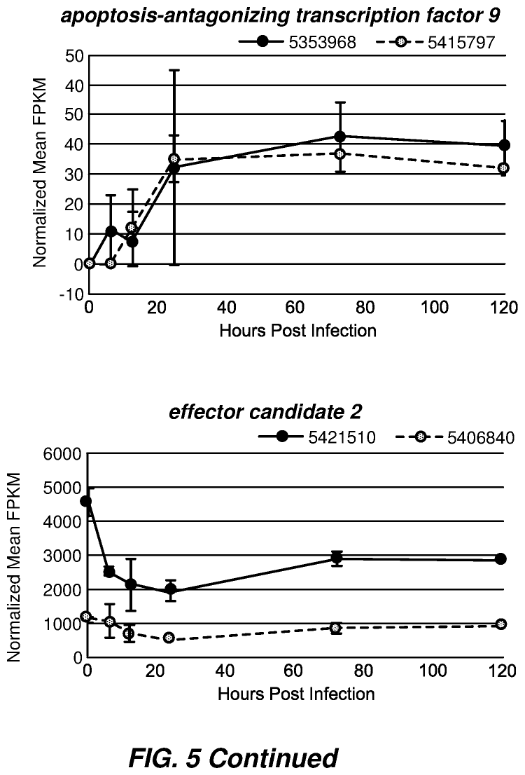

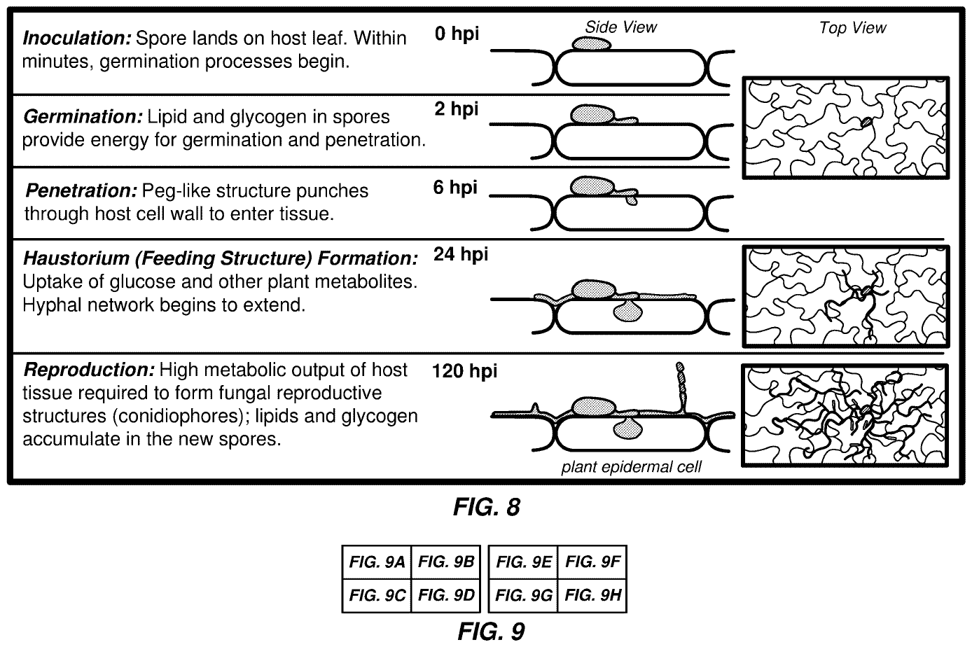

The gene targets were chosen based on their annotation and potential function such as maintaining essential cell function, mobilizing spore energy reserves early in powdery mildew development and/or manipulating the plant host. In addition, pattern and magnitude of expression of the genes through development, number of highly similar genes, and putative positioning in a metabolic or regulatory pathway was considered (Table 5, FIG. 5, FIG. 8). In addition to CYP51, powdery mildew gene targets tested consist of G. orontii MGH1 homologs of unidentified transcript 4 (UTR4), glycogen debranching enzyme 1 (GDB1), glycogen phosphatase 1 (GPH1), lipase 1 (LIP1), lipase a (LIPA), diglyceride acyltransferase (DGAT), β-carotene 15,15′-dioxygenase (BCDO), glucose-induced degradation 9 (GID9), apoptosis-antagonizing transcription factor (AATF), and effector candidate 2 (EC2) (Table 5, FIG. 4). A brief rationale on why these targets were selected is presented in Table 5. All targets were tested using A. thaliana whole plant assays. A portion of those genes were also tested in the detached leaf assay to compare the outcome based on the assay type. SIGS against four out of these ten targets tested reproducibly and significantly reduced fungal proliferation (FIG. 4). For initial screening, this gives a 40% success rate in identifying single powdery mildew targets important to fungal proliferation.

As previously shown, SIGS against CYP51 involved in maintaining cell function (membrane component generation) resulted in an average spore reduction of 46% when compared to control treatment (FIG. 4). Consistent with its essential cellular function, normalized CYP51 expression (to total RNA) is essentially stable across the infection timeline (Black box, FIG. 5). The metabolic gene, UTR4, an enzyme in the methionine salvage pathway (Pirkov et al., 2008), allows cells to regenerate methionine and reuse sulfur (Table 5). UTR4 expression levels are very high and unlike CYP51, UTR4 expression dramatically peaked at 6 hpi (Grey box, FIG. 5). This suggests it is utilized early in development before the haustorium is developed and plant nutrients are acquired. No SIGS phenotype was observed with the tested dsRNA against UTR4 (FIG. 4). However, SIGS using additional UTR4 dsRNAs and dsRNAs against other powdery mildew genes in the methionine salvage pathway should be explored.

A number of targets were chosen because they could play a role in breaking down and mobilizing/metabolizing spore glycogen or storage lipids to provide energy and metabolic precursors for early development and growth of the fungus before the haustorium has fully developed (FIG. 2). As shown in FIG. 5 (blue box), the genes encoding biosynthetic enzymes GDB1 and GPH1 and the regulatory enzyme GID9 all exhibit peaks of expression before the haustorium is fully developed. These enzymes are postulated to play a role in mobilizing energy and precursors from spore glycogen (Table 5). SIGS against these targets did not impact spore production (FIG. 4). The impact of other dsRNAs targeting these genes and this process remains to be explored. However, the similar findings for all three targets tested suggests that spore glycogen mobilization is not the prime contributor to spore germination and early colonization events.

Three metabolic genes involved in storage lipid metabolism were targeted with SIGS: lipases LIP1 and LIPA and DGAT. Triacylglycerol lipases hydrolyze triacylglycerols producing fatty acids and glycerol while diglyceride acyltransferases form triglycerides from diacylglycerol and acyl-CoA. Triacylglycerols are stored in lipid droplets in spores and are degraded to build membranes and fuel cellular metabolism (Subramoni et al., 2010). SIGS of LIP1 reduced growth an average of 22% and 51% in whole plant and detached leaf experiments, respectively (FIG. 4). SIGS of LIPA reduced growth an average of 41% and 67% in whole plant and detached leaf experiments, respectively (FIG. 4). There are 2 copies of LIPA and 2 copies of LIP1; the dsRNA was designed to silence both copies. All copies of these lipases are most highly expressed within 12 hpi (Light blue box, FIG. 5). This suggests these lipases may be especially important for obligate biotrophs before the haustorium is fully developed. In contrast to the glycogen mobilization targets, initial screening of storage lipid mobilization targets resulted in 2 of 3 tested with a reproducible significant reduction in powdery mildew spore production. This suggests spore storage lipid breakdown/mobilization may contribute more to initial powdery mildew growth and development than glycogen breakdown/mobilization.

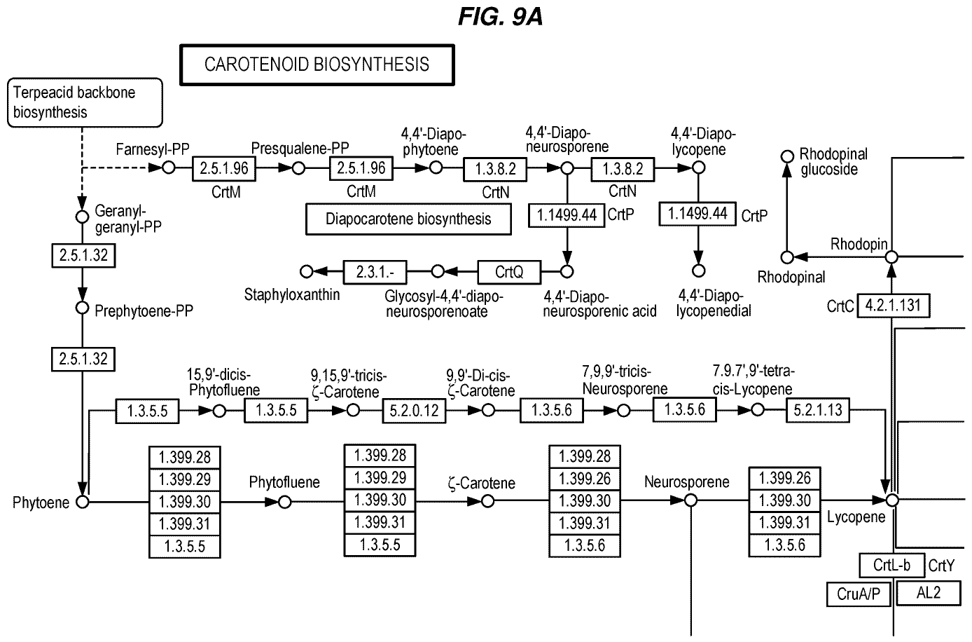

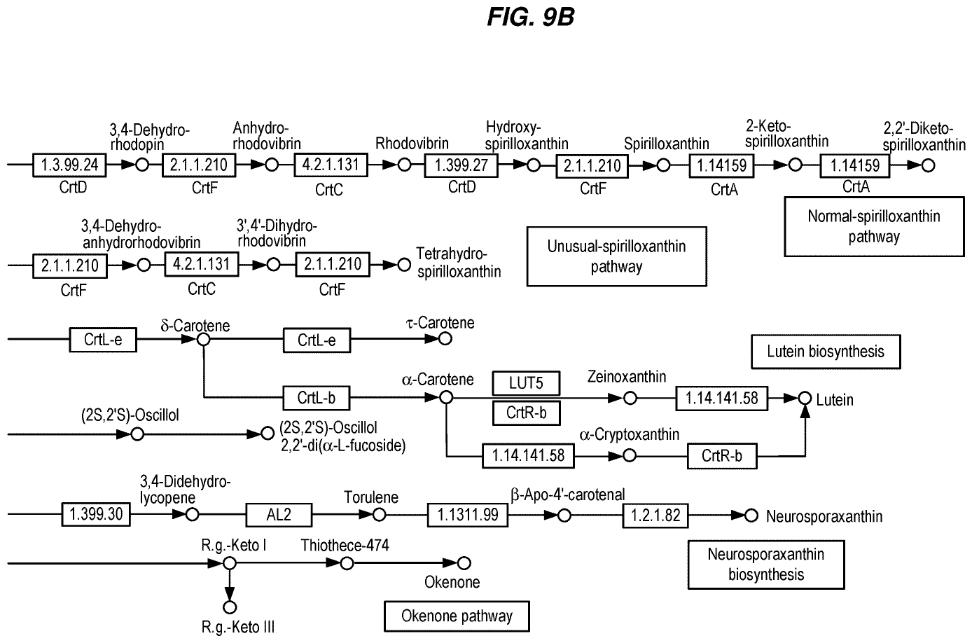

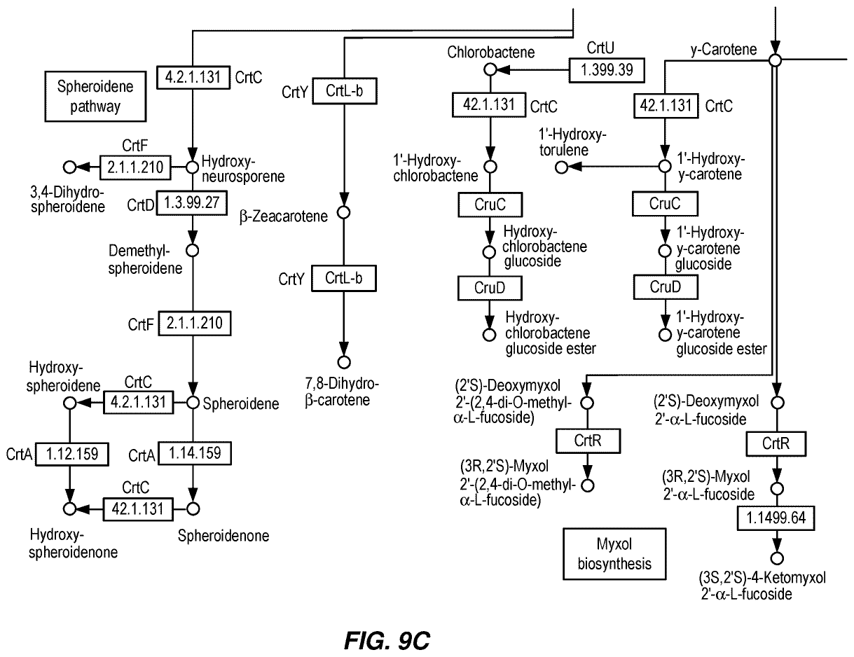

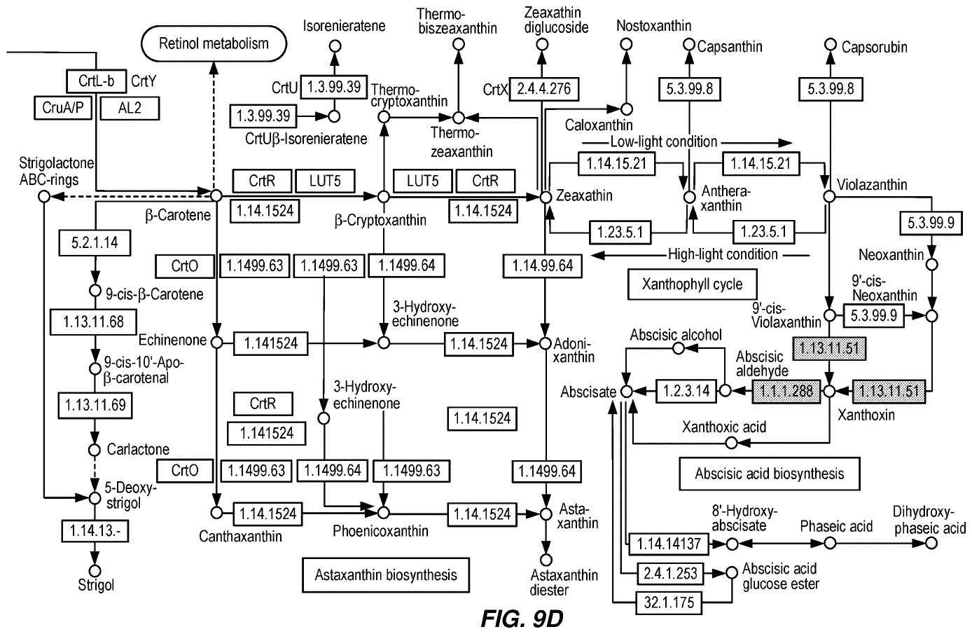

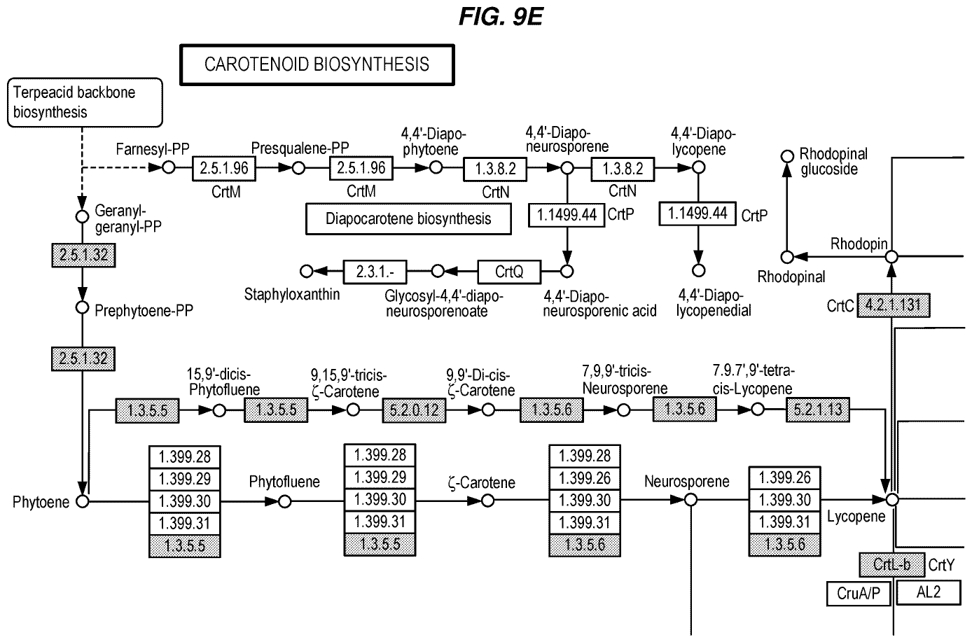

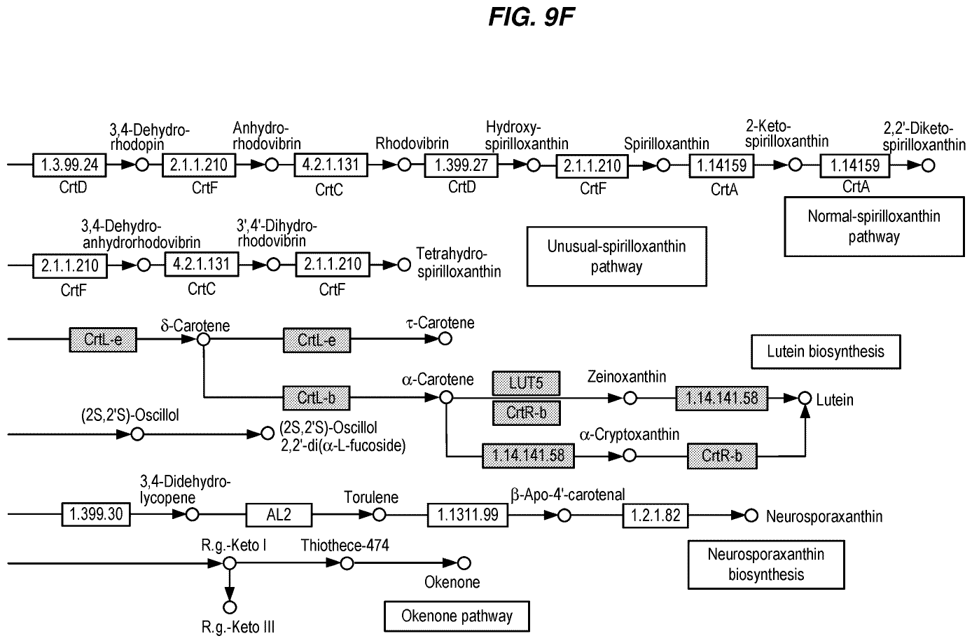

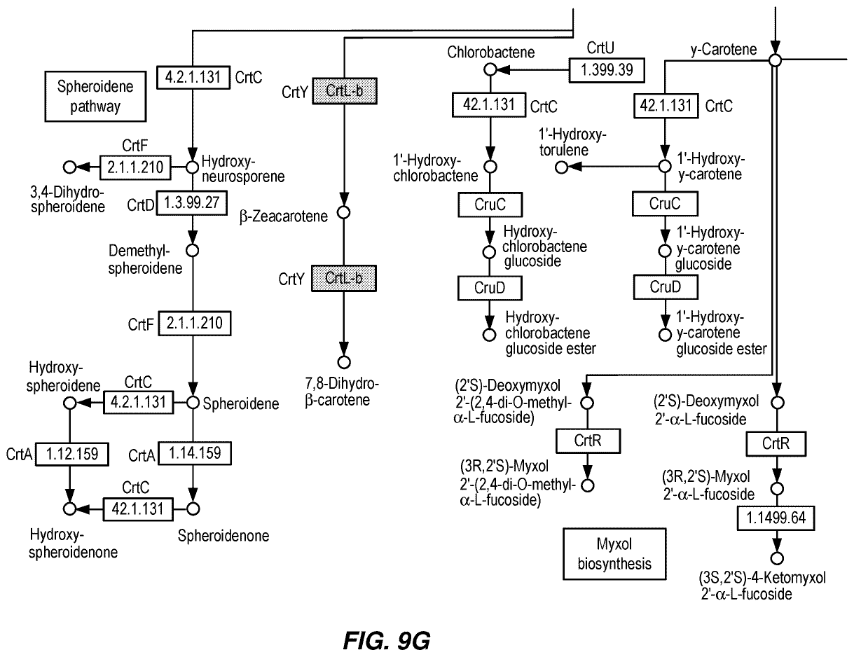

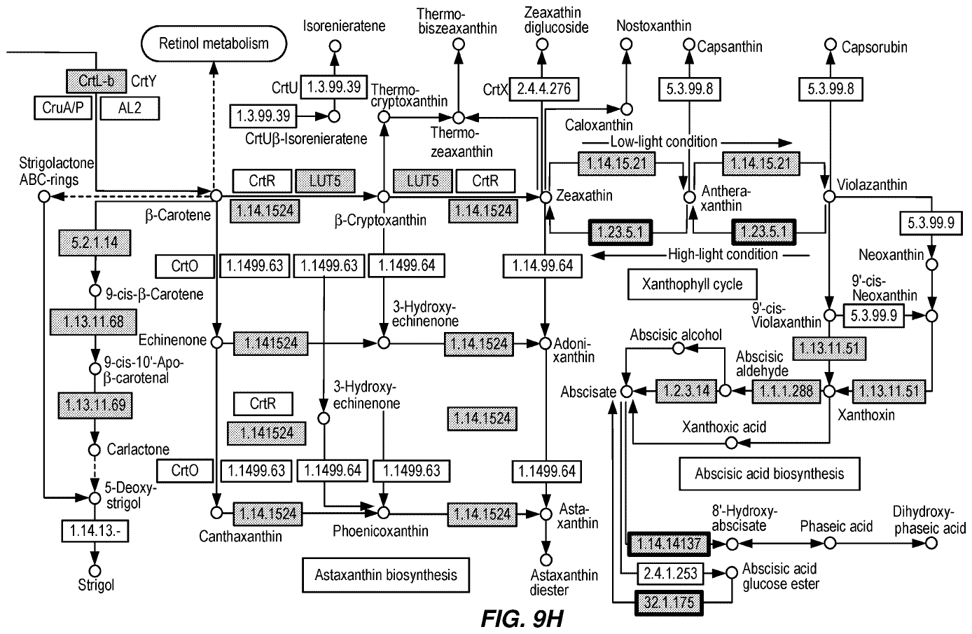

Manipulation of the plant host by pathogens can take many forms and is a key feature of the evolved interaction of an obligate biotroph with its host. Carotenoid oxygenases (members of the BCDO family) are enzymes responsible for cleaving carotenoids and have been identified in plants, fungi, animals, and bacteria (Ahrazem et al., 2016; Tan et al., 2003). They can function in diverse roles including signaling in response to environmental cues, regulation, and biosynthesis. In G. orontii MGH1, the targeted BCDO is further specified as a 9-cis-epoxycarotenoid dioxygenase (EC 1.1.3.11.51) involved in ABA biosynthesis (FIG. 9). In addition, another ABA biosynthetic gene encoding xanthoxin dehydrogenase (EC 1.1.1.288) is also annotated as being present in G. orontii MGH1. The low and variable expression of this target gene as shown in FIG. 5 (green box) does not allow us to further postulate on its involvement in a specific phase of infection. However, SIGS using long dsRNA targeting G. orontii MGH1 BCDO had a significant impact on growth, reducing spore production by 50% in whole plant experiments, the highest average reduction observed among the whole plant experiments (FIG. 4). Reduction was also seen in detached leaf assays with long dsRNA reducing spore production by 54% (FIG. 4).

The powdery mildew apoptosis antagonizing transcription factor, AATF, could mediate fungal response to plant-induced cell stress (Table 5). Alternately, it could be secreted into the plant by a yet unknown mechanism to limit cell death. AATF expression increases with infection through 24 hpi and then remains elevated (FIG. 5, green box). SIGS of AATF was found to reduce spore production an average of 21% and 32% in whole plant and detached leaf assays, respectively (FIG. 4). Only the detached leaf experiment showed the reduced growth to be statistically significant (FIG. 4). This reduction in spore production is close to the resolution of spore counting. However, based on its potential functions, it remains an interesting target for further analysis.

Effectors are secreted proteins that manipulate plant functions. EC2 is a widely conserved powdery mildew effector protein first described in B. graminis f. sp. hordei as BEC2 (CSEP0214; (Schmidt et al., 2014). Both copies of EC2 in G. orontii MGH1 are extremely highly expressed, with one copy being the most highly expressed gene in the fungal transcriptome (FIG. 5, green box). SIGS using long dsRNA against EC2 did not impact spore production (FIG. 4). However, given the fact that expression of EC2 in transgenic A. thaliana increased non-host powdery mildew penetration (Schmidt et al., 2014), we decided to further test it using siRNA (below).

siRNA Targeting BCDO and EC2 Significantly Reduces G. orontii Spore Production on A. Thaliana Detached Leaves

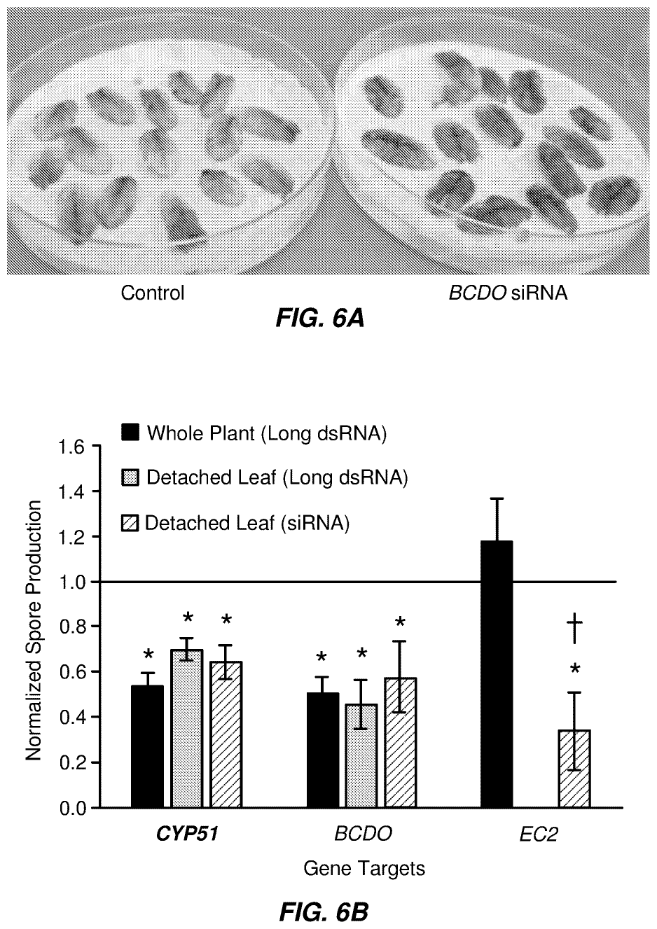

The efficacy of siRNAs was further tested using the gene targets BCDO and EC2 in A. thaliana detached leaf assays (FIG. 6). siRNA targeting BCDO was tested because the long dsRNA SIGS experiments consistently showed very strong suppression of spore production (FIG. 4). SIGS of EC2 did not show reduced spore production (FIG. 4) using dsRNA, but given its demonstrated role in powdery mildew pathogenesis (Schmidt et al., 2014), it was worth testing another silencing RNA (FIG. 4). BCDO siRNA sprayed leaves visually had less powdery mildew growth and chlorosis (FIG. 6, panel a). Spore production was reduced an average of 42% (FIG. 6, panel b), providing further confirmation for the role of BCDO in promoting powdery mildew proliferation. SIGS using siRNA-1 EC2 reduced growth an average of 66% (FIG. 6, panel b), showing EC2 does promote powdery mildew proliferation and the EC2 dsRNA previously tested (FIG. 4) was not effective. This result shows that known powdery mildew effectors, even those that are very highly expressed, can be targeted via SIGS to reduce powdery mildew growth and development.

SIGS Methods and Gene Target AATF Translate to the Erysiphe Necator-Vitis vinifera Pathosystem

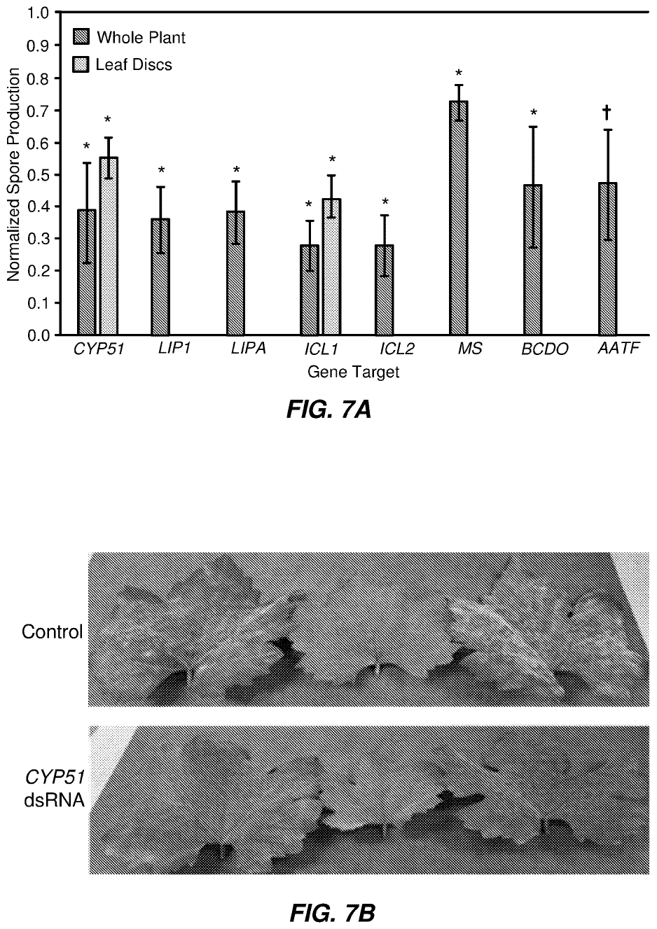

With SIGS methods optimized and gene targets identified, we sought to translate these methods and targets to grape powdery mildew (FIG. 2). New long dsRNAs were designed for each target tested as there is significant sequence divergence between G. orontii MGH1 and E. necator c. First, procedures were optimized for the grapevine system by silencing CYP51. Spore production was reduced by 62% and 45% in grapevine whole plant and leaf disc assays, respectively when treated with long dsRNA against the same region of CYP51 as dsRNA-1 against G. orontii MGH1 CYP51 (FIG. 2; FIG. 7, panel a). In addition, visual inspection showed infected grape leaves had less powdery mildew coverage and density when sprayed with dsRNA targeting CYP51 than with buffer alone (FIG. 7, panel b). We tested SIGS against the effective targets including AATF in the grapevine system, as we were interested in ascertaining whether they would show a statistically significant difference in grape powdery mildew spore production. The SIGS target, AATF, exhibited on average of 53% reduced spore production in E. necator when silenced with long dsRNA (FIG. 7). The average reduction of powdery mildew spore production for SIGS against CYP51 and AATF was greater in E. necator compared to G. orontii (FIG. 4 and FIG. 7). One notable difference is G. orontii has 2 copies each of CYP51 and AATF while E. necator has 1 copy of each of these genes. SIGS against the other effective targets also were effective in reducing powdery mildew of grapevine (FIG. 7). These results indicate we can successfully translate work performed using the Arabidopsis powdery mildew system to the grapevine powdery mildew system.

### Discussion

We developed a high-throughput assay to silence powdery mildew genes and determine their contribution to disease (FIG. 2). Our success in SIGS of powdery mildew targets is consistent with HIGS working in the powdery mildew, B. graminis (Pliego et al., 2013), and genes encoding the required endogenous RNAi machinery being annotated in G. orontii MGH1 v4.0 genome. Screening gene targets in Arabidopsis powdery mildew is rapid (FIG. 2) and provides insight into genes important for powdery mildews, as about 86% of dicot powdery mildew and 77% of dicot and monocot powdery mildew genes are conserved (Wu et al., 2018). Furthermore, as obligate biotrophs, powdery mildews have few genes relative to other Ascomycota (Wu et al., 2018) making target testing using the G. orontii-A. thaliana pathosystem an efficient means of prioritizing targets to test in other powdery mildew-plant host interactions. A. thaliana plants have a quick generation time, can be highly controlled in growth chambers, and require little space for multiple replicates compared to many powdery mildew-impacted crops making this system valuable in quickly identifying novel gene targets.

In this study we also demonstrated SIGS methods and conserved targets in G. orontii can translate to the grape powdery mildew E. necator and we anticipate they can translate to other powdery mildews. All five genes targets identified in G. orontii that were tested in E. necator with SIGS also impacted growth and reproduction (see, e.g., FIG. 7). For example, the gene target AATF was found to contribute to G. orontii spore production (FIG. 4) with an even greater impact on E. necator (FIG. 7). AATF expression is modulated upon pathogen infection (Benakanakere et al., 2019; Kaul et al., 2009) and in cancer cells (Iezzi and Fanciulli, 2015) to prolong the life of a cell. If the powdery mildew AATF is secreted into the plant, by a yet undefined means, it could potentially prolong plant cell life and inhibit cell death, features of critical import to obligate biotrophy. If it functions in the fungi, it could act to maintain normal cellular function in the presence of plant-induced stress. Other regulators of cell fate should be tested via SIGS to identify additional important targets.

Using SIGS we probed the importance of ten metabolic, regulatory, and effector genes and identified five powdery mildew targets that impact G. orontii MGH1 spore production (FIGS. 4, 6). When screening targets, multiple long dsRNAs or siRNAs may need to be tested in order to determine the contribution of a gene. The efficiency of siRNA mediated silencing depends on several factors including the accessibility of target region, structural features of the siRNA leading to efficient loading of antisense strand of siRNA into RISC complex (Reynolds et al., 2004; Ui-Tei et al., 2004). Our preliminary screen also highlights the importance of screening multiple genes involved in a process in an initial screen to increase the likelihood of identifying at least one with a powdery mildew phenotype when screened with an initial dsRNA. For example, 2/3 genes tested involved in storage lipid metabolism exhibited reduced spore production when sprayed with long dsRNA (FIG. 4). By contrast, 3/3 genes involved in glycogen metabolism did not show reductions in powdery mildew proliferation when sprayed with long dsRNA suggesting this process is not as critical to the success of the infection.

Homologs of lipases lipase a and lipase 1 proved to be significant contributors to powdery mildew growth on A. thaliana. In the powdery mildew, Blumeria graminis f. sp. hordei, lipases fall into 3 distinct expression patterns. Group III lipase expression remains constant throughout infection while group I lipase expression is elevated in ungerminated spores and at 4 hpi, decreasing as the infection progresses and group II expression is suppressed in haustoria tissue (Both et al., 2005). Lipid staining of developing powdery mildew spores reflects the activity of class I lipases suggesting lipids are consumed to fuel development before nutrient acquisition from the host begins (Both, et al. 2005). Later in infection these lipases or others can potentially be used for lipid remodeling for triacylglycerol storage in newly formed spores (Laureano et al., 2018), suggesting lipases have various functions and are regulated by development. The early expression of LIPA and LIP1 (FIG. 5) indicates they behave like group I lipases which are presumably required early in development to facilitate mobilization of energy. Extracellular lipase, LIP1, in the necrotrophic pathogen B. cinerea is induced by exposure to grape cuticle, but lip1 mutants do not have reduced lesion formation (Subramoni et al., 2010). Similarly, lip1 mutants of the hemibiotrophic pathogen, F. graminearum, does not impact virulence (Subramoni et al., 2010). For a biotrophic pathogen such as powdery mildew, dependent initially on its own spore reserves for early growth and development, it appears that LIPA and LIP1 may play more critical roles.

SIGS against the powdery mildew BCDO-family gene provided the most dramatic reduction in powdery mildew proliferation of the targets tested (FIGS. 4, 6). Carotenoid-cleaving enzymes can have diverse functions in plants, fungi, animals, and bacteria (Ahrazem et al., 2016; Tan et al., 2003). In G. orontii MGH1, the targeted BCDO is further specified as a 9-cis-epoxycarotenoid dioxygenase (EC 1.1.3.11.51) involved in ABA biosynthesis (FIG. 9). In addition, another ABA biosynthetic gene encoding xanthoxin dehydrogenase (EC 1.1.1.288) is also annotated as being present in G. orontii MGH1 supporting this putative function. Diverse fungi have been described to produce ABA, though this trait appears restricted to plant-associated fungi (Asselbergh et al., 2008). Expression of A. thaliana genes in this pathway is modulated in response to powdery mildew infection (Chandran et al., 2010). These changes may promote the formation of violaxanthin from zeaxanthin (upstream of ABA formation) via NPQ4 which is down-regulated in high-light conditions. Not only is this shift providing precursors for ABA biosynthesis but also provides additional support for the source to sink transition imposed by infection. In the rice-Magnaporthe oryzae interaction, exogenous application of ABA promotes germination and fungal ABA biosynthesis mutants have impaired lesion formation on rice (Spence et al., 2015).

Powdery mildews and other fungal pathogens can have hundreds of putative effectors identified through genomics with few having any annotated domains (de Jonge et al., 2011). These effector repertoires expand and contract in fungal pathogens allowing new adaptations to emerge at the host-pathogen interface (Fouche et al., 2018). The effector EC2 is a highly expressed powdery mildew effector (FIG. 5) with a cysteine-rich fungal extracellular membrane (CFEM) domain. CFEM domains have proposed roles in fungal pathogenesis (Kulkarni et al., 2003). Transgenic A. thaliana expressing EC2 have increased entry by non-adapted powdery mildew (Schmidt et al., 2014), demonstrating its contribution to pathogenesis of powdery mildews as well. EC2 also significantly contributes to G. orontii growth and reproduction on A. thaliana (FIG. 6). In G. orontii, EC2 is expressed 1-2 orders of magnitude higher than the other genes targeted in this study (FIG. 5). Despite being highly expressed, SIGS against EC2 still had a significant impact on powdery mildew proliferation. EC2 is highly expressed by many fungal pathogens suggesting it may represent a broad fungal target for an RNA-based pesticide.

SIGS has the potential to revolutionize agriculture if RNA pesticides prove to be efficacious in controlling pathogen growth. SIGS methods were translated to identify agriculturally important targets to be used for pest management. Using SIGS we can rapidly identify novel targets and adapt RNA constructs to the strain-specific sequences of target genes present in the field. We anticipate these RNA pesticides are more evolutionarily robust to resistance compared to chemical fungicides. Pathogens challenged with RNA pesticides would have to accumulate many mutations in the targeted gene sequence to avoid silencing, while pathogens challenged with azole-fungicides, for example, may only require a mutation in the enzyme binding site to avoid inhibition. If resistance does develop to an RNA pesticide, other dsRNAs targeting other parts of the gene can be developed to effectively silence the same gene or dsRNAs targeting other genes in the same pathway or cellular process can be designed and tested. Additionally, pathogens could downregulate RNAi processes to develop resistance to RNA pesticides, however, silencing RNAi machinery during fungal infection also impairs growth (Werner et al., 2020). Multiple required cellular processes could potentially be targeted at once by multiplexing dsRNAs. Or dsRNAs against multiple genes in a given process or pathway could be multiplexed to further increase efficacy. Finally, the efficiency of uptake and duration of gene silencing may be improved, for example by encapsulating dsRNAs in liposomes or complexing dsRNAs to clay nanosheets (Cagliari et al., 2019).

The optimization of SIGS in powdery mildews provides a novel and versatile approach to study genetics in the obligate biotroph, powdery mildew. To our knowledge, this is the first report of SIGS in powdery mildew fungi. Using this high-throughput SIGS and disease assay pipeline, we can more rapidly assess the importance of a gene on powdery mildew growth and development, removing the plasmid construction steps required for virus-induced gene silencing (VIGS) and the transformation steps required for HIGS. SIGS provides a rapid methodology to study gene function in powdery mildews which are not culturable apart from the host and not genetically tractable. Given the conservation of retained genes and essential processes in obligate biotrophs (Spanu 2012), targets identified in the powdery mildew-plant host system may translate to other plant (obligate) biotrophs as well as human intracellular pathogens.

### REFERENCES

Agrios, G., 2004. Plant Pathology, 5th ed. Elsevier Inc.
Ahrazem, O., Gomez-Gomez, L., Rodrigo, M. J., Avalos, J., Limon, M. C., 2016. Carotenoid cleavage oxygenases from microbes and photosynthetic organisms: Features and functions. International Journal of Molecular Sciences 17. doi.org/10.3390/ijms17111781
Aked, J., Hall, J. L., 1993. Effect of powdery mildew infection on concentrations of apoplastic sugars in pea leaves. New Phytologist 123, 283-288. doi.org/10.1111Zj.1469-8137.1993.tb03737.
Asselbergh, B., de Vleesschauwer, D., Hofte, M., 2008. Global switches and fine-tuning-ABA modulates plant pathogen defense. Molecular Plant-Microbe Interactions 21, 709-719. doi.org/10.1094/MPMI-21-6-0709.
Benakanakere, M. R., Zhao, J., Finoti, L., Schattner, R., Odabas-Yigit, M., Kinane, D. F., 2019. MicroRNA-663 antagonizes apoptosis antagonizing transcription factor to induce apoptosis in epithelial cells. Apoptosis 24, 108-118. doi.org/10.1007/s10495-018-01513-9.
Bilir, O., Telli, O., Norman, C., Budak, H., Hong, Y., Tor, M., 2019. Small RNA inhibits infection by downy mildew pathogen Hyaloperonospora arabidopsidis. Molecular Plant Pathology 20, 1523-1534. doi.org/10.1111/mpp.12863.
Both, M., Csukai, M., Stumpf, M. P. H., Spanu, P. D., 2005. Gene Expression Profiles of Blumeria graminis Indicate Dynamic Changes to Primary Metabolism during Development of an Obligate Biotrophic Pathogen 17, 2107-2122. doi.Org/10.1105/tpc.105.032631.1.
Bramlett, M., Plaetinck, G., Maienfisch, P., 2020. RNA-Based Biocontrols-A New Paradigm in Crop Protection. Engineering 6, 522-527. doi.org/10.1016/j.eng.2019.09.008.
Braun, B., Pfirrmann, T., Menssen, R., Hofmann, K., Scheel, H., Wolf, D. H., 2011. Gid9, a second RING finger protein contributes to the ubiquitin ligase activity of the Gid complex required for catabolite degradation. FEBS Letters 585, 3856-3861. doi.org/10.1016/j.febslet.2011.10.038.
Cagliari, D., Dias, N. P., Galdeano, D. M., dos Santos, E. A., Smagghe, G., Zotti, M. J., 2019. Management of Pest Insects and Plant Diseases by Non-Transformative RNAi. Frontiers in Plant Science 10. doi.org/10.3389/fpls.2019.01319.
Calvert, G. M., Karnik, J., Mehler, L., Beckman, J., Morrissey, B., Sievert, J., Barrett, R., Lackovic, M., Mabee, L., Schwartz, A., Mitchell, Y., Moraga-McHaley, S., 2008. Acute pesticide poisoning among agricultural workers in the United States, 1998-2005. American Journal of Industrial Medicine 51, 883-898. doi.org/10.1002/ajim.20623.
Chandran, D., Inada, N., Hather, G., Kleindt, C. K., Wildermuth, M. C., 2010. Laser microdissection of Arabidopsis cells at the powdery mildew infection site reveals site-specific processes and regulators. Proceedings of the National Academy of Sciences of the United States of America 107, 460-465. doi.org/10.1073/pnas.0912492107.
de Jonge, R., Bolton, M. D., Thomma, B. P. H. J., 2011. How filamentous pathogens co-opt plants: The ins and outs of fungal effectors. Current Opinion in Plant Biology 14, 400-406. doi.org/10.1016/j.pbi.2011.03.005.
Fouche, S., Plissonneau, C., Croll, D., 2018. The birth and death of effectors in rapidly evolving filamentous pathogen genomes. Current Opinion in Microbiology 46, 34-42. doi.org/10.1016/j.mib.2018.01.020.
Frenkel, O., Cadle-Davidson, L., Wilcox, W. F., Milgroom, M. G., 2015. Mechanisms of resistance to an azole fungicide in the grapevine powdery mildew fungus, Erysiphe necator. Phytopathology 105, 370-377. doi.org/10.1094/PHYTO-07-14-0202-R.
Fuller, K. B., Alston, J. M., Sambucci, O. S., 2014. The value of powdery mildew resistance in grapes: Evidence from California. Wine Economics and Policy 3, 90-107. doi.org/10.1016/j.wep.2014.09.001.
Grigoriev, I. v., Nikitin, R., Haridas, S., Kuo, A., Ohm, R., Otillar, R., Riley, R., Salamov, A., Zhao, X., Korzeniewski, F., Smirnova, T., Nordberg, H., Dubchak, I., Shabalov, I., 2014. MycoCosm portal: Gearing up for 1000 fungal genomes. Nucleic Acids Research 42, 699704. doi.org/10.1093/nar/gkt1183.
Hu, D., Chen, Z. Y., Zhang, C., Ganiger, M., 2020. Reduction of Phakopsora pachyrhizi infection on soybean through host- and spray-induced gene silencing. Molecular Plant Pathology 21, 794-807. doi.org/10.1111/mpp.12931.
Iezzi, S., Fanciulli, M., 2015. Discovering Che-1/AATF: A new attractive target for cancer therapy. Frontiers in Genetics 6, 1-7. doi.org/10.3389/fgene.2015.00141.
Jones, L., Riaz, S., Morales-Cruz, A., Amrine, K. C., McGuire, B., Gubler, W., Walker, M., Cantu, D., 2014. Adaptive genomic structural variation in the grape powdery mildew pathogen, Erysiphe necator. BMC Genomics 15, 1081. doi.org/10.1186/1471-2164-15-1081.
Kaul, D., Ahlawat, A., Gupta, S. D., 2009. HIV-1 genome-encoded hiv1-mir-H1 impairs cellular responses to infection. Molecular and Cellular Biochemistry 323, 143-148. doi.org/10.1007/s11010-008-9973-4.
Koch, A., Biedenkopf, D., Furch, A., Weber, L., Rossbach, O., Abdellatef, E., Linicus, L., Johannsmeier, J., Jelonek, L., Goesmann, A., Cardoza, V., McMillan, J., Mentzel, T., Kogel, K. H., 2016. An RNAi-Based Control of Fusarium graminearum Infections Through Spraying of Long dsRNAs Involves a Plant Passage and Is Controlled by the Fungal Silencing Machinery. PLoS Pathogens 12, 1-22. doi.org/10.1371/journal.ppat.1005901.
Kulkarni, R. D., Kelkar, H. S., Dean, R. A., 2003. An eight-cysteine-containing CFEM domain unique to a group of fungal membrane proteins. Trends in Biochemical Sciences 28, 118121. doi.org/10.1016/SO968-0004(03)00025-2.
Laureano, G., Figueiredo, J., Cavaco, A. R., Duarte, B., Cagador, I., Malho, R., Sousa Silva, M., Matos, A. R., Figueiredo, A., 2018. The interplay between membrane lipids and phospholipase A family members in grapevine resistance against Plasmopara viticola. Scientific Reports 8, 1-15. doi.org/10.1038/s41598-018-32559-z.
Lievens, L., Pollier, J., Goossens, A., Beyaert, R., Staal, J., 2017. Abscisic acid as pathogen effector and immune regulator. Frontiers in Plant Science 8, 1-15. doi.org/10.3389/fpls.2017.00587.
Machado, A. K., Brown, N. A., Urban, M., Kanyuka, K., Hammond-Kosack, K. E., 2018. RNAi as an emerging approach to control Fusarium head blight disease and mycotoxin contamination in cereals. Pest Management Science 74, 790-799. doi.org/10.1002/ps.4748.
Majumdar, R., Rajasekaran, K., Cary, J. W., 2017. RNA interference (RNAi) as a potential tool for control of mycotoxin contamination in crop plants: Concepts and considerations. Frontiers in Plant Science 8. doi.org/10.3389/fpls.2017.00200.
Martinez-Cruz, J., Romero, D., de Vicente, A., Perez-Garda, A., 2018. Transformation by growth onto agro-infiltrated tissues (TGAT), a simple and efficient alternative for transient transformation of the cucurbit powdery mildew pathogen Podosphaera xanthii. Molecular Plant Pathology 19, 2502-2515. doi.org/10.1111/mpp.12722.
Mittal, V., 2004. Improving the efficiency of RNA interference in mammals. Nature Reviews Genetics 5, 355-365. doi.org/10.1038/nrgl323.
Pirkov, I., Norbeck, J., Gustafsson, L., Albers, E., 2008. A complete inventory of all enzymes in the eukaryotic methionine salvage pathway. FEBS Journal 275, 4111-4120. doi.org/10.1111/j.1742-4658.2008.06552.x.
Pliego, C., Nowara, D., Bonciani, G., Gheorghe, D. M., Xu, R., Surana, P., Whigham, E., Nettleton, D., Bogdanove, A. J., Wise, R. P., Schweizer, P., Bindschedler, L. v., Spanu, P. D., 2013. Host-induced gene silencing in barley powdery mildew reveals a class of ribonuclease-like effectors. Molecular Plant-Microbe Interactions 26, 633-642. doi.org/10.1094/MPMI-01-13-0005-R.
Raanan, R., Gunier, R. B., Balmes, J. R., Beltran, A. J., Harley, K. G., Bradman, A., Eskenazi, B., 2017. Elemental sulfur use and associations with pediatric lung function and respiratory symptoms in an agricultural community (California, USA). Environmental Health Perspectives 125, 1-8. doi.org/10.1289/EHP528.
Ramanujam, R., Calvert, M. E., Selvaraj, P., Naqvi, N. I., 2013. The Late Endosomal HOPS Complex Anchors Active G-Protein Signaling Essential for Pathogenesis in Magnaporthe oryzae. PLoS Pathogens 9. doi.org/10.1371/journal.ppat.1003527.
Regelmann, J., Schule, T., Josupeit, F. S., Horak, J., Rose, M., Entian, K.-D., Thumm, M., Wolf, D. H., 2003. Catabolite Degradation of Fructose-1,6-bisphosphatase in the Yeast Saccharomyces cerevisiae: A Genome-wide Screen Identifies Eight Novel GID Genes and Indicates the Existence of Two Degradation Pathways. Molecular Biology of the Cell 14, 1652-1663. doi.org/10.1091/mbc.e02-08-0456.
Reuber, T. L., Plotnikova, J. M., Dewdney, J., Rogers, E. E., William Wood, B., Ausubel, F. M., 1998. Correlation of defense gene induction defects with powdery mildew susceptibility in Arabidopsis enhanced disease susceptibility mutants. Plant Journal 16, 473-485. doi.org/10.1046/j.1365-313X.1998.00319.x.
Reynolds, A., Leake, D., Boese, Q., Scaringe, S., Marshall, W. S., Khvorova, A., 2004. Rational siRNA design for RNA interference. Nature Biotechnology 22, 326-330. doi.org/10.1038/nbt936.
Schmidt, S. M., Kuhn, H., Micali, C., Liller, C., Kwaaitaal, M., Panstruga, R., 2014. Interaction of a Blumeria graminis f. sp. hordei effector candidate with a barley ARF-GAP suggests that host vesicle trafficking is a fungal pathogenicity target. Molecular Plant Pathology 15, 535-549. doi.org/10.1111/mpp.12110.
Spence, C. A., Lakshmanan, V., Donofrio, N., Bais, H. P., 2015. Crucial roles of abscisic acid biogenesis in virulence of rice blast fungus Magnaporthe oryzae. Frontiers in Plant Science 6, 1-13. doi.org/10.3389/fpls.2015.01082.
Subramoni, S., Suarez-Moreno, Z. R., Venturi, V., 2010. Lipases as Pathogenicity Factors of Plant Pathogens, in: Handbook of Hydrocarbon and Lipid Microbiology. Springer Berlin Heidelberg, Berlin, Heidelberg, pp. 3269-3277. doi.org/10.1007/978-3-540-77587-4_248.
Tan, B. C., Joseph, L. M., Deng, W. T., Liu, L., Li, Q. B., Cline, K., McCarty, D. R., 2003. Molecular characterization of the Arabidopsis 9-cis epoxycarotenoid dioxygenase gene family. Plant Journal 35, 44-56. doi.org/10.1046/j.1365-313X.2003.01786.x.
Ui-Tei, K., Naito, Y., Takahashi, F., Haraguchi, T., Ohki-Hamazaki, H., Juni, A., Ueda, R., Saigo, K., 2004. Guidelines for the selection of highly effective siRNA sequences for mammalian and chick RNA interference. Nucleic Acids Research 32, 936-948. doi.org/10.1093/nar/gkh247.
Wang, M., Weiberg, A., Lin, F. M., Thomma, B. P. H. J., Huang, H. da, Jin, H., 2016. Bidirectional cross-kingdom RNAi and fungal uptake of external RNAs confer plant protection. Nature Plants 2, 1-10. doi.org/10.1038/nplants.2016.151.
Werner, B. T., Gaffar, F. Y., Schuemann, J., Biedenkopf, D., Koch, A. M., 2020. RNA-Spray-Mediated Silencing of Fusarium graminearum AGO and DCL Genes Improve Barley Disease Resistance. Frontiers in Plant Science 11, 1-11. doi.org/10.3389/fpls.2020.00476.
WeBling, R., Panstruga, R., 2012. Rapid quantification of plant-powdery mildew interactions by qPCR and conidiospore counts. Plant Methods 8, 35. doi.org/10.1186/1746-4811-8-35.
Wu, W., Hodges, E., Redelius, J., Hoog, C., 2004. A novel approach for evaluating the efficiency of siRNAs on protein levels in cultured cells. Nucleic acids research 32, 1-6. doi.org/10.1093/nar/gnh010.
Wu, Y., Ma, X., Pan, Z., Kale, S. D., Song, Y., King, H., Zhang, Q., Presley, C., Deng, X., Wei, C. I., Xiao, S., 2018. Comparative genome analyses reveal sequence features reflecting distinct modes of host-adaptation between dicot and monocot powdery mildew 06 Biological Sciences 0604 Genetics. BMC Genomics 19, 1-20. doi.org/10.1186/s12864-018-5069-z
Xiong, F., Liu, M., Zhuo, F., Yin, H., Deng, K., Feng, S., Liu, Y., Luo, X., Feng, L., Zhang, S., Li, Z., Ren, M., 2019. Host-induced gene silencing of BcTOR in Botrytis cinerea enhances plant resistance to grey mould. Molecular Plant Pathology 20, 1722-1739. doi.org/10.1111/mpp.12873.
Zhu, X., Qi, T., Yang, Q., He, F., Tan, C., Ma, W., Voegele, R. T., Kang, Z., Guo, J., 2017. Host-induced gene silencing of the MAPKK gene PsFUZ7 confers stable resistance to wheat stripe rust. Plant Physiology 175, 1853-1863. doi.org/10.1104/pp. 17.01223.
Zubrod, J. P., Baudy, P., Schulz, R., Bundschuh, M., 2014. Effects of current-use fungicides and their mixtures on the feeding and survival of the key shredder Gammarus fossarum. Aquatic Toxicology 150, 133-143. doi.org/10.1016/j.aquatox.2014.03.00

### Example 2

### Powdery Mildew Effectors Targeting Arabidopsis TCP Transcription Factors Contribute to Disease

### Summary of Example 2

Plant TCP transcription factors that regulate growth and development are heavily targeted by effectors of diverse plant pathogens. In a yeast-two hybrid screen between Arabidopsis thaliana proteins and Golovinomyces orontii MPIPZ effectors, 34% of the interactions identified involved TCP proteins. In this study the contribution of TCPs and their targeting effectors during infection is measured. Four of the five TCPs targeted by effectors were found to suppress G. orontii MGH1 growth. Adult A. thaliana T-DNA mutants tcp13, tcp14, tcp15, and tcp20 were more susceptible to G. orontii MGH1. Using spray-induced gene silencing (SIGS), homologs of OECs in G. orontii MGH1 were targeted. When OEC14, OEC60/OEC61, and OEC70 were targeted via SIGS, significantly less spores were produced. Homolog OEC60/OEC61 was investigated further. It encodes a short N-terminal domain originally annotated in G. orontii MPIPZ OEC60 and OEC61 coupled to a larger glycosyl hydrolase family 17 domain (GH17). To date, OEC60 and OEC61 homologs across all sequenced powdery mildews include the GH17 domain, suggesting the lack of GH17 domain in the MPIPZ isolate may be due to the incomplete nature of that genome. GH17 domain-containing proteins are found widely in plants and fungi. When homolog of OEC60/OEC61 is silenced in the Erysiphe necator-grapevine system, significantly less spores are also produced, providing evidence this is a conserved powdery mildew virulence factor. Using Agrobacterium-mediated transient expression assays in Nicotiana benthamiana, G. orontii MGH1 homolog of OEC60/OEC61 was shown to be nuclear-targeted and co-localize with TCP13 and TCP14. Furthermore, co-immunoprecipitation shows OEC60/OEC61 interaction with TCP13 in planta. An analysis of available TCP13 and TCP14 Arabidopsis protein interaction data reveals a majority of targets interact with both TCP13 and 14; these targets are highly enriched in development and gene expression ontology. tcp14 mutants have enhanced endoreduplication at the site of powdery mildew infection which is known to correlate with enhanced powdery mildew asexual reproduction. Together, these results support a role for OEC60/OEC61 in limiting TCP13/14 function to promote endoreduplication at the infection site. To further elucidate this interaction node, proteomic analysis (2-D LC MS/MS) of the MYC-purified protein complex from G. orontii-infected and uninfected plants expressing G. orontii MGH1 OEC60/OEC61-MYC was performed. TCP13 and TCP14 protein interactions suggest OEC60/OEC61 interacts with TCP13 and TCP14 to alter gene expression associated with endoreduplication including endocycle reentry, inhibition of cell division and cytokinesis, and endocycle progression. Manipulation of the endocycle is important for the proliferation of powdery mildew and the growth and development of diverse biotrophs. These findings provide fundamental insights on regulation of the eukaryotic cell cycle.

### Introduction

Plant TCP transcription factors regulate growth and development (Lopez et al., 2015). The TCP gene family is named after the first 3 genes identified in this family: TEOSINTE BRANCHED1 in maize (Zea mays), CYCLOIDEA in snapdragon (Antirrhinum majus), and PCF (proliferating cell nuclear antigen factor) in rice (Oryza sativa) (Lopez et al., 2015). TCPs emerged 650-800 million years ago, predating land plants, and have been identified in moss and algae in addition to angiosperms which are more well studied (Navaud et al., 2007). Arabidopsis thaliana has 24 TCPs containing the 59 amino acid TCP domain (basic helix-loop-helix domain) which mediates DNA-binding (Lopez et al., 2015). TCPs are divided into two classes; class I TCPs are known to promote cell proliferation and class II TCPs act antagonistically to suppress growth (Li, 2015). They are highly interactive proteins within and outside their protein family (BioGRID; Stark et al., 2006). They interact within their protein family to form homo- and hetero-dimers which controls affinity and specificity in DNA-binding (Li, 2015). Together these transcription factors act redundantly and antagonistically to regulate leaf development, plant hormone biosynthesis, and plant immunity (Lopez et al., 2015). This regulation may be critical in balancing growth when stressed to protect the fitness of the plant.

TCPs are emerging as plant pathogen immunity modulators heavily acted upon by secreted pathogen effectors from diverse pathogens. In a yeast-two hybrid screen performed by (WeBling et al., 2014), effectors from Hyaloperonospora arabidopsidis (downy mildew, oomycete), Pseudomonas syringae DC3000 (bacterium), and Golovinomyces orontii MPIPZ (powdery mildew fungus) were screened against a library of A. thaliana proteins. Among 9 proteins that were targeted by effectors from all three pathogens, 3 of them were TCPs (TCP13, TCP14, and TCP15). TCP14 was the most highly targeted host protein with 52 effectors targeting it, primarily effectors from the obligate biotrophic pathogens downy and powdery mildews (WeBling et al., 2014). Furthermore, tcp13 and tcp14 mutants challenged by P. syringae DC3000 had opposite susceptibility phenotypes compared with challenge by obligate biotrophs (WeBling et al., 2014). Together this suggests TCPs function in lifestyle specific manners to impact plant immunity and/or pathogen virulence.

Within the 42 G. orontii MPIPZ effector candidate (OEC)-TCP interactions, several effectors were found to interact with multiple TCPs by yeast two-hybrid and all 5 targeted TCPs interacted with multiple effectors (WeBling et al., 2014). This represents 34% of all A. thaliana protein-OEC interactions. Suffice to say, there is much to uncover about the mechanism and function of these interactions. In this study we measure the genetic contribution of TCPs and OECs in the context of powdery mildew infection utilizing the G. orontii MGH1-Arabidopsis thaliana pathosystem.

Homologs of OECs in the related strain G. orontii MGH1 were identified for investigation in the annotated G. orontii MGH1 v4.0 genome accessed through MycoCosm (Grigoriev et al., 2014).

Spray-induced gene silencing is used to rapidly assess the contribution of OECs to powdery mildew spore production, prioritizing highly connected OECs that interact with TCPs that control powdery mildew susceptibility. Mutants in the five targeted TCP genes are also challenged with powdery mildew to identify novel TCP genes that contribute to spore production. The results of these disease assays along with bioinformatic analysis prioritizes G. orontii MGH1 homolog of OEC60 and OEC61 for further investigation.

The reproductive success of the powdery mildew fungus, G. orontii, is also tied to the ploidy state of the surrounding host tissues being co-opted to provide nutrients. Enhanced endoreduplication, or DNA replication without cell division, is observed in A. thaliana mesophyll cells underlying the infection site (Chandran et al., 2010). Additionally, several A. thaliana mutants with reduced or abrogated endoreduplication at the site of infection support less growth of the powdery mildew, with the extent of powdery mildew asexual reproduction highly correlated with infection site plant cell DNA ploidy levels (Chandran et al., 2013). Endoreduplication is proposed to increase the cells ability to provide energy rich metabolites to the powdery mildew during the metabolically demanding process of spore formation. It may also be associated with a metabolic shift from a source tissue to a sink tissue. TCPs, particularly class I TCPs, have many described roles in regulating the cell cycle. Powdery mildew infection assays are performed on four class I tcp mutants: tcp9, 14, 15, and tcp20. Of the 5 TCPs targeted by effectors, TCP14 and TCP15 have the most well described role in regulating endoreduplication (Peng et al., 2015). TCP14 and TCP15 redundantly suppress endoreduplication; single mutants have wildtype ploidy but double mutants have enhanced ploidy. DA1, DAR1, and DAR2 modulate the stability of TCP14 and TCP15 which represses the gene expression of RBR and CYCA3;2 among others whose expression promotes endoreduplication (Peng et al., 2015). Effectors targeting these TCPs could interfere with TCP mediated suppression of these genes. TCP20 is also a known regulator of the cell cycle and binds the CYCB1;1 promoter to regulate G2/M transition (Gutie et al., 2005) and TCP13, a class II TCP, suppresses cell expansion via suppression of ATHB12 expression (Hur et al., 2019). ATHB12 promotes expression of EXPANSIN (EXPA) a regulator of leaf expansion and CELL-CYCLE SWITCH 52A (CCS52A), an activator of the anaphase-promoting complex/cyclosome that promotes endoreduplication and has been implicated in biotroph-induced plant cell endoreduplication (Vercruysse et al., 2020; Wildermuth et al., 2017). The prevalence of effectors acting upon TCP nodes regulating growth and immunity indicates the import and complexity of their role in plant-pathogen interactions. Herein we provide evidence for a model by which powdery mildew effectors act on the TCP13-TCP14 node to promote localized host cell endoreduplication.

### Results

G. orontii Effectors Converge on A. Thaliana TCP Transcription Factors

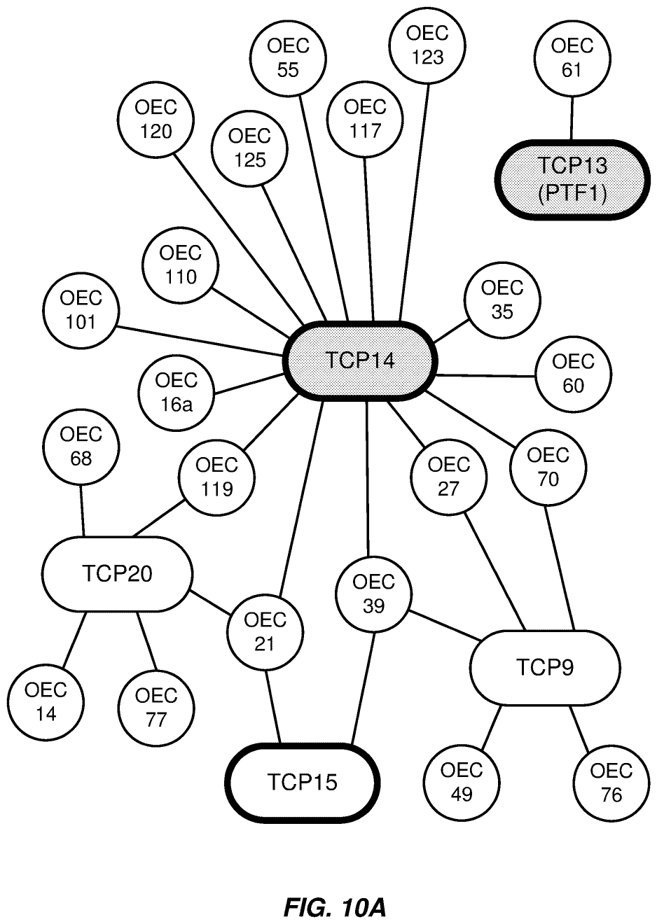

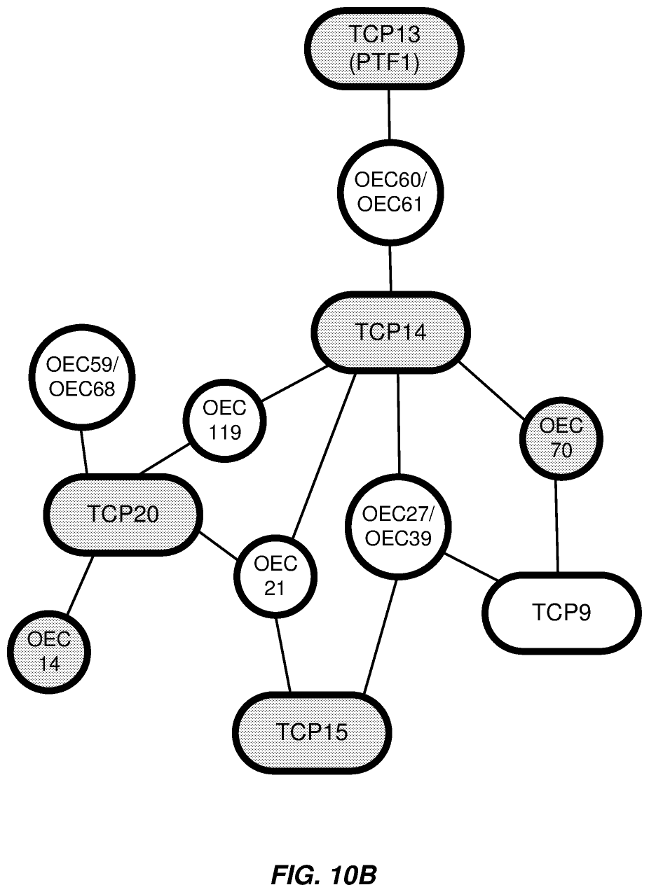

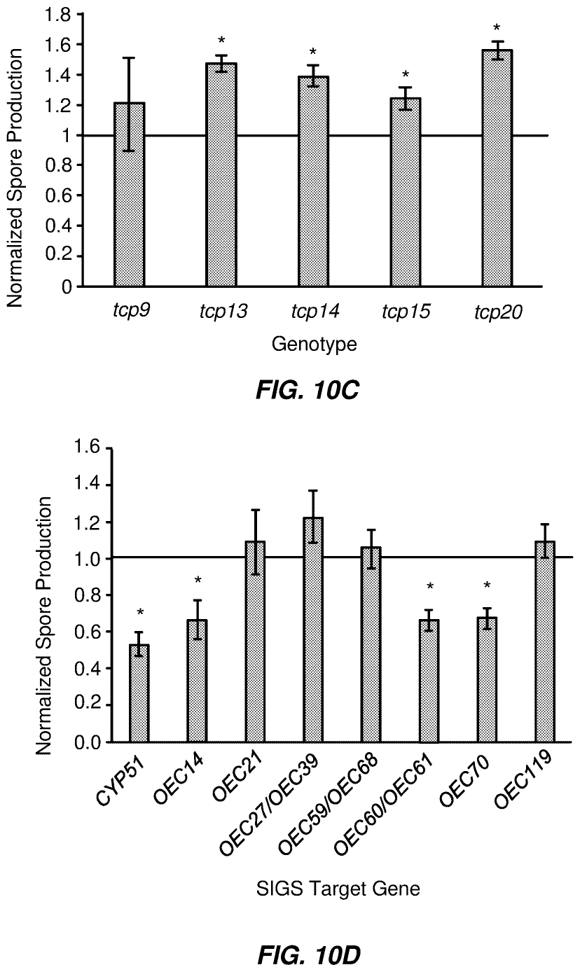

G. orontii MGH1 effector homologs of G. orontii MPIPZ effector candidates (OECs) that interact with A. thaliana TCPs via yeast 2-hybrid (WeBling et al., 2014) were identified using G. orontii MGH1 v4.0 genome (See Methods) accessed through MyCocosm (Grigoriev et al., 2014). The TCP-OEC interaction map (FIG. 10, panel a) contains all G. orontii MPIPZ effectors identified in G. orontii MGH1 and their interacting TCPs (WeBling et al., 2014). Some OECs of interest have distinct OEC names but are homologs. When naming OEC homologs in G, orontii MGH1, OEC names are grouped together to indicate the homologs identified are related to multiple OECs (Table 6). For example, OEC60 and OEC61 are homologs. In G. orontii MGH1, two homologs were identified and are referred to as homologs of OEC60/OEC61. The OEC60/OEC61 G. orontii MGH1 homologs of OEC60/OEC61 also contain a large glycosyl hydrolase family 17 (GH17) domain belonging to the enzyme class 3.2.1.58 (EC 3.2.1.58). OEC60/OEC61 homologs in other powdery mildews also include the GH17 domain; therefore, G. orontii MPIPZ OEC60 and OEC61 homologs may be missing this domain due to the incomplete nature of the G. orontii MPIPZ genome sequence (Spanu et al., 2010).

TABLE 6
G. orontii TCP-targeting effectors selected for spray-induced gene silencing.

| | | | G. orontii MPIPZ | |
|---|---|---|---|---|
| | | | OEC-TCP | |
| | Copy | | interactions identified | OEC homologs in other |
| Effector | number¹ | Annotation^{1,2} | via yeast-two hybrid² | species^{1,3} |
| OEC14 | 2 | RNAse-like | TCP20 | Powdery mildews including |
| | | effector, nuclear | | G. cichoracearum, E. pisi, E. |
| | | localization | | necator |
| | | signal (RIKRR) | | |
| OEC21 | 2 | RNAse-like | TCP14, TCP15, | Powdery mildews including |
| | | effector | TCP20, HB21, CSN5A | G. cichoracearum, E. pisi |
| OEC27/ | 7 | RNAse-like | TCP9, TCP14, TCP15 | Powdery mildews including |
| OEC39 | | effector | | G. cichoracearum |
| OEC59/ | 2 | | TCP20, TPR8 | Powdery mildews including |
| OEC68 | | | | G. cichoracearum, E. pisi, E. |
| | | | | necator |
| OEC60/ | 2 | glycosyl | TCP13, TCP14, | Powdery mildews including |
| OEC61 | | hydrolase | CSN5A, SBP | G. cichoracearum, E. |
| | | family 17 | | necator, E. pisi, B. graminis |
| | | (GH17) | | f. sp. hordei, and B. graminis |
| | | | | f. sp. triticum (BEC1005); |
| | | | | Other fungal pathogens, |
| | | | | GH17 domain-containing |
| | | | | proteins are widely present |
| | | | | and secreted in fungi |
| | | | | however the region upstream |
| | | | | of the GH17 domain |
| | | | | (containing G. orontii |
| | | | | MPIPZ OEC60 or OEC61 |
| | | | | sequence) has only been |
| | | | | identified in powdery |
| | | | | mildews |
| OEC70 | 2 | | TCP9, TCP14, CSN5A, | Powdery mildews including |
| | | | ASIL1, RCN1, UBQ- | G. cichoracearum, E. pisi, E. |
| | | | like gene | necator |
| OEC119 | 2 | | TCP14, TCP20, RCN1, | Powdery mildews including |
| | | | PP2AA2 | G. cichoracearum |

¹G. orontii MGH1 genome accessed through MycoCosm (Grigoriev et al., 2014).
²(Bernhofer et al., 2018) (Weβling et al., 2014).

A. Thaliana Tcp13, Tcp14, Tcp15, and Tcp20 have Enhanced Susceptibility to G. orontii MGH1

Of the five TCPs found to interact with G. orontii MPIPZ effectors via yeast 2-hybrid (FIG. 10, panel a; Table 7; WeBling et al., 2014), A. thaliana T-DNA insertion mutants in three exhibited a powdery mildew phenotype in seedling assays. tcp13, tcp14, and tcp15 exhibited enhanced disease susceptibility (eds) to G. orontii MPIPZ. However, only tcp13 and tcp14 exhibited eds using adult plants (WeBling et al., 2014), the focus of our study.

TABLE 7
TCP transcription factor gene information and targeting effectors.

| Arabidopsis | Gene | |
|---|---|---|
| thaliana TCP¹ | Locus | |
| TCP9 (Class I) | AT2G45680 | OEC27, OEC39, OEC49, OEC70, |
| | | OEC76 |
| TCP13 (Class II) | AT3G02150 | OEC45, OEC61 |
| TCP14 (Class I) | ATCG47620 | OEC16a, OEC21, OEC27, OEC35, |
| | | OEC39, OEC45, OEC55, OEC60, |
| | | OEC70, OEC101, OEC110, OEC117, |
| | | OEC119, OEC120, OEC123, OEC125 |
| TCP15 (Class I) | AT1G69690 | OEC21, OEC39 |
| TCP20 (Class I) | AT3G27010 | OEC14, , OEC21, OEC68, OEC77, |
| | | OEC119 |

¹(Lopez et al., 2015).
²(Weβling et al., 2014).

Using a modified spore quantification protocol developed for G. orontii MGH1 (WeBling et al., 2014), powdery mildew disease was quantified on tcp9, tcp13, tcp14, tcp15, and tcp20 adult plants compared with wild type plants (FIG. 10, panels b-c). A. thaliana tcp13, tcp14, tcp15, and tcp20 have enhanced susceptibility to G. orontii MGH1 (FIG. 10, panel c), meaning they support higher fungal proliferation. tcp15 exhibits the smallest statistically significant increase in powdery mildew spore production of the tested TCPs, with 24% more spores formed per gram fresh weight on average than wild type plants. This suggests our assay is able to resolve small differences in powdery mildew disease not previously identified. tcp13 and 14 exhibit similar increases in spore production of 48% and 39%, respectively, compared to wild type plants, and the previously untested tcp20 mutant allows the greatest increase in spore production (56%).

G. orontii MGH1 Effector Homologs OEC14, OEC60/OEC61, and OEC70 Contribute to Spore Production

OEC homologs in G. orontii MGH1 were selected for spray-induced silencing assays (SIGS) based on their ability to target multiple TCPs (FIG. 10, panels a-b) or target TCPs that contribute to spore production (FIG. 10, panel c). For each of the seven OEC homologs tested, a single long dsRNA was designed and synthesized for SIGS in A. thaliana whole plant powdery mildew disease assays. As a control, we show the reduction in spore production with SIGS against the known azole-fungicide target, CYP51 (FIG. 10, panel d), a gene required for membrane synthesis (Zhang et al., 2019). SIGS targeting G. orontii MGH1 homologs of OEC14, OEC60/OEC61, and OEC70 significantly reduced spore production in G. orontii MGH1 infection of A. thaliana compared to buffer only controls (FIG. 10, panel d).

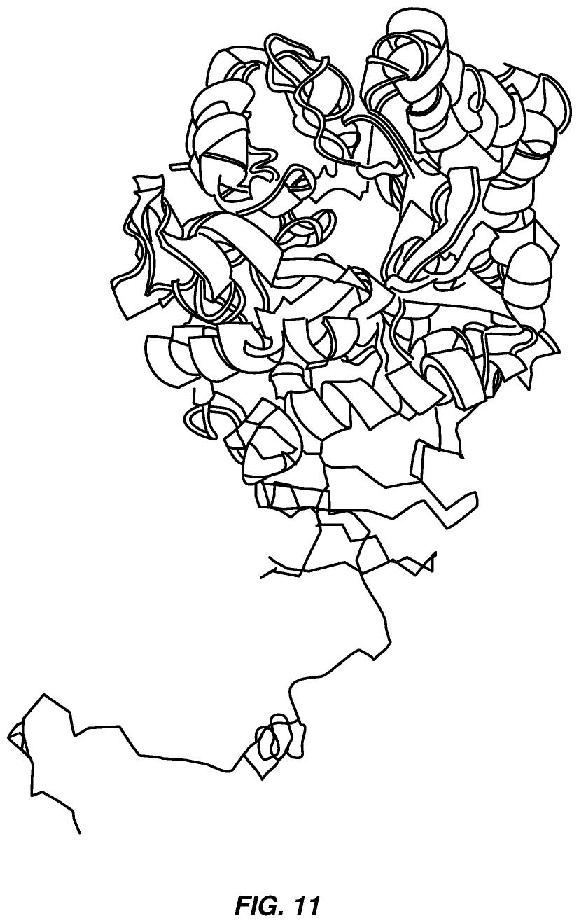

All OECs were originally identified based on their secretion signal and not having homology outside powdery mildews (WeBling et al., 2014), among other criteria. As such the effectors silenced are specific to powdery mildews except for the glycosyl hydrolase family 17 (GH17) domain-containing protein G. orontii MGH1 OEC60/OEC61. The GH17 protein domain has been identified in plants and fungi (Gaudioso-Pedraza and Benitez-Alfonso, 2014). Among other fungi accessed via MycoCosm, we found GH17 domain-containing proteins to be often secreted and have multiple copies (Grigoriev et al., 2014). The predicted protein structure of G. orontii MGH1 OEC60/OEC61 is similar to a glycoside hydrolase family 17 beta-1,3-glucanosyltransferase in Rhizomucor miehei (FIG. 11). Furthermore, it contains the catalytic amino acid identified in barley P-glucan endohydrolases that is also present in Saccharomyces cerevisiae (Chen et al., 1993).

### Spray-Induced Gene Silencing of Homolog of OEC60OEC61 in Erysiphe Necator Reduces Grape Powdery Mildew Spore Production

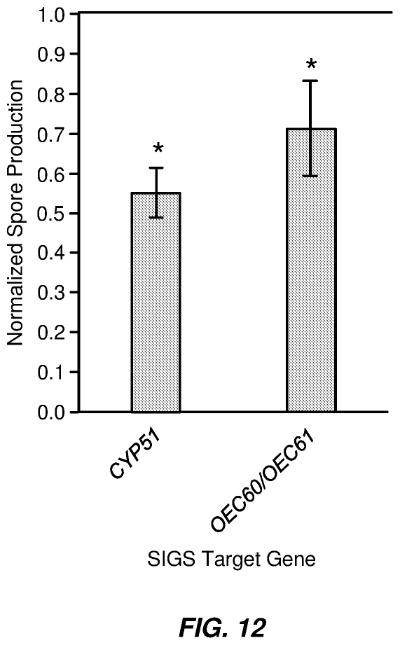

As the full G. orontii MGH1 OEC60/OEC61 is conserved in other powdery mildews (Table 6) and has the potential to interact with both TCP13 and TCP14, we tested whether findings in the G. orontii MGH1-Arabidopsis pathosystem can translate to an agriculturally important pathosystem, powdery mildew of grapevine. To do so, we identified an OEC60/OEC61 homolog in E. necator c (Jones et al., 2014) and designed a long dsRNA to target this gene in the same region effective for G. orontii. Targeting of OEC60/OEC61 in Erysiphe necator by SIGS reduces grape powdery mildew production on V. vinifera leaves to 71% of control treated plants (FIG. 12), similar to the reduction seen with the Arabidopsis powdery mildew (FIG. 10, panel d).

G. orontii MGH1 OEC60/OEC61 is Nuclear-Targeted Effector that Colocalizes and Interacts with with TCP13

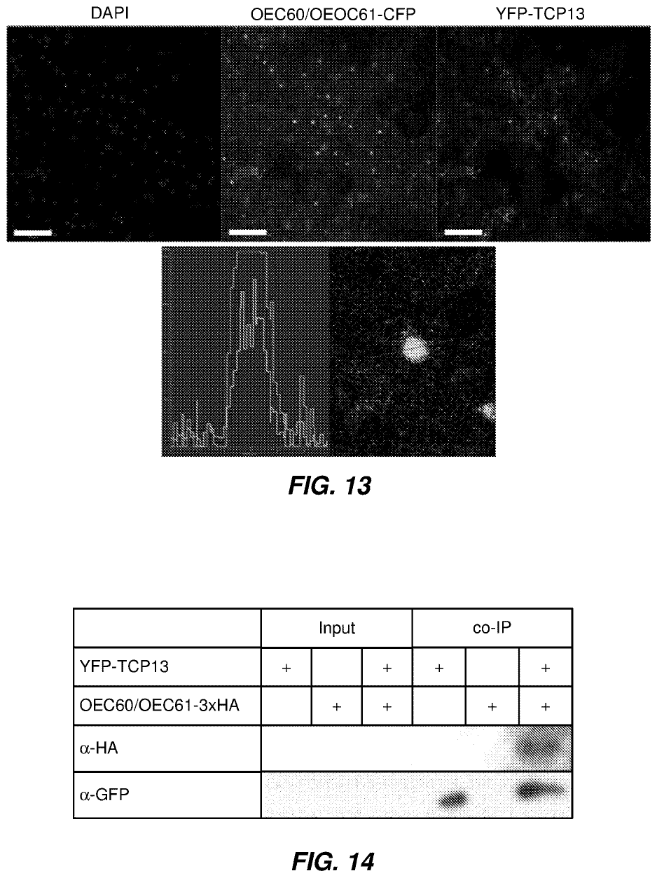

Agrobacterium-mediated transient expression experiments were performed in Nicotiana benthamiana to investigate the localization of G. orontii MGH1 homolog of OEC60/OEC61. OEC60/OEC61-CFP was strongly localized to the nucleus of plant cells (FIG. 13). Co-infiltrations experiments showed OEC60/OEC61-CFP colocalized in the nucleus with YFP-TCP13 (FIG. 13). Subnuclear foci could not be resolved to indicate specific co-localization of YFP-TCP13 and OEC60/OEC61-CFP. Although TCPs are transcription factors known to localize to the nucleus, TCP13 was found to primarily localize to chloroplasts in tobacco plants and to function as a regulator of chloroplast encoded genes like psbD (Baba et al., 2001). Co-localization should be further explored in mesophyll cells where chloroplasts are prevalent and in response to infection, which could alter localization. Additional co-infiltrations were performed to confirm a physical biochemical interaction in planta via co-immunoprecipitation (co-IP). A western blot shows YFP-TCP13 and OEC60/OEC61-3×HA interact in planta (FIG. 14). G. orontii MPIPZ OEC60 was previously shown to co-localize in sub-nuclear foci and interact in planta, confirmed by co-IP, with TCP14 (WeBling et al., 2014). In addition, TCP13 and TCP14 have been found to interact with each other via yeast-two hybrid (Danisman et al., 2013; Dreze et al., 2011) suggesting they could both function in the nucleus.

Transgenic A. Thaliana Expressing G. orontii MGH1 OEC60/OEC61-MYC Created to Identify Protein Interaction Network

To identify novel protein interactions with G. orontii MGH1 OEC60/OEC61, we transformed A. thaliana pGWB17-OEC60/OEOC61. pGWB17 contains a T-DNA that expresses genes of interest constitutively under the control of the CaMV35s and adds a C-terminal MYC epitope. Transformants do not have any noticeable or severed developmental phenotype. Using stable transgenic lines, we performed co-IP of OEC60/OEC61 from the host plant in uninfected and powdery mildew-infected leaf tissue. Multiple proteins were detected in infected and uninfected MYC co-immunoprecipitation (co-IP) protein purifications by Coomassie staining of protein run on an SDS-PAGE gel cut and sent for proteomic analysis (2-D LC MS/MS). Identified protein are in Table 8. SIGS against one of these targets, G. orontii APC1 involved in cell fate modulation was tested and resulted in 48% reduction in spore production.

TABLE 8
G. orontii MGH1 protein identified mass spectrometry from
co-immunoprecipitation of OEC60/OEC61-MYC from infected transgenic
A. thaliana. 2D (MudPIT) cation exchange/RP LC-MS/MS analysis
was performed with reference to the G. orontii MGH1 (Golor4) JGI
database “Mycocosm” //mycocosm.jgi.doe.gov/Golor4/Golor4.home.html).
Proteins identified in wildtype A. thaliana co-IP were excluded
from A. thaliana 35S::OEC60/61-MYC transgenic co-IP proteins
identified.

| | | Putative | | Protein | |
|---|---|---|---|---|---|
| | Gene Name or PFAM | Gene | | ID | Copy |
| Process | Domain | Symbol | EC ID | (Golor4) | Number |
| Cell fate | Anaphase-promoting | APC1 | | 5334394 | 3 |
| modulation | complex/20S cyclosome | | | | |
| | subunit | | | | |
| Mitochrondrial | Mitochondrial carrier (MC) | | | 4771748 | 4 |
| ADP/ATP | protein | | | | |
| transporter | | | | | |
| RNA | RNA recognition motif | | | 4204905 | 2 |
| | (RRM) containing protein | | | | |
| PPP pathway | 6-phosphogluconate | PGD | 1.1.1.44 | 549579 | 2 |
| | dehydrogenase | | | | |
| Amino acid | Glycine | SHMT | 2.1.2.1 | 1010163 | 4 |
| metabolism, | hydroxymethyltransferase/ | | | | |
| cofactor | serine aldolase/serine | | | | |
| metabolism | hydroxymethyltransferase | | | | |

### Discussion

TCPs and their targeting effectors are important modulators of susceptibility in A. thaliana-G. ororntii interactions. Our findings suggest these interactions promote infection site reprogramming to support powdery mildew proliferation. The adapted spore quantification assay and ploidy determination experiments proved to be quite sensitive to resolve the impact of single cell cycle component genes and effectors despite the prevalence of redundancy. The tcp14 mutant, for example, supported more powdery mildew growth and had enhanced ploidy at the site of infection (FIG. 10 panel c) despite being functionally redundant in developmentally-associated ploidy. tcp14 and tcp15 single mutants do not impact ploidy in leaves, but double mutants have enhanced ploidy (Peng et al., 2015). Similarly, a single mutant in MYB3R4 was abrogated in powdery mildew-induced ploidy and supported less powdery mildew asexual reproduction whereas a double mutant was required to observe a leaf ploidy phenotype (Chandran et al., 2010). These findings highlight the ability of the powdery mildew system to identify individual components of the complex cell machinery governing cell fate.

TCP15, TCP20, OEC14, OEC60/OEC61, and OEC70 were identified as novel targets regulating powdery mildew asexual reproduction in adult plants. Interestingly, most of the TCPs investigated here are not upregulated at the site of infection (Chandran et al., 2013). This suggests effector-targeted TCPs are modulated at the protein level—and not transcriptionally. This is supported by the finding that the yeast 2-hybrid screen of G. orontii MPIPZ OEC60/OEC61 and OEC70 identified eight interacting Arabidopsis proteins in total, including three TCPs as shown in FIG. 10, panel a, and three proteins involved in protein modification (CSN5A, RCN1, and UBQ-like) (WeBling et al., 2014).

Prioritizing effectors targeting TCPs via SIGS has been extremely fruitful with 43% of targets contributing to spore production. Furthermore, those that did not have a phenotype via SIGS could be tested with long dsRNAs or siRNAs designed against different regions of the transcript or in combination with another dsRNA against a similarly interacting OEC. G. orontii MGH1 homologs of ten other OEC proteins that interact with TCPs by yeast 2-hybrid remain to be tested (FIG. 10 panel a). Three additional untested OECs belong to the same subclade (OEC16a, OEC123, and OEC125) and encode RNAse-like effectors. There are 12 copies of OEC16a/OEC123/0EC125 homologs in G. orontii MGH1 and 5 copies in the closely related powdery mildew G. cichoracearum (Grigoriev et al., 2014).

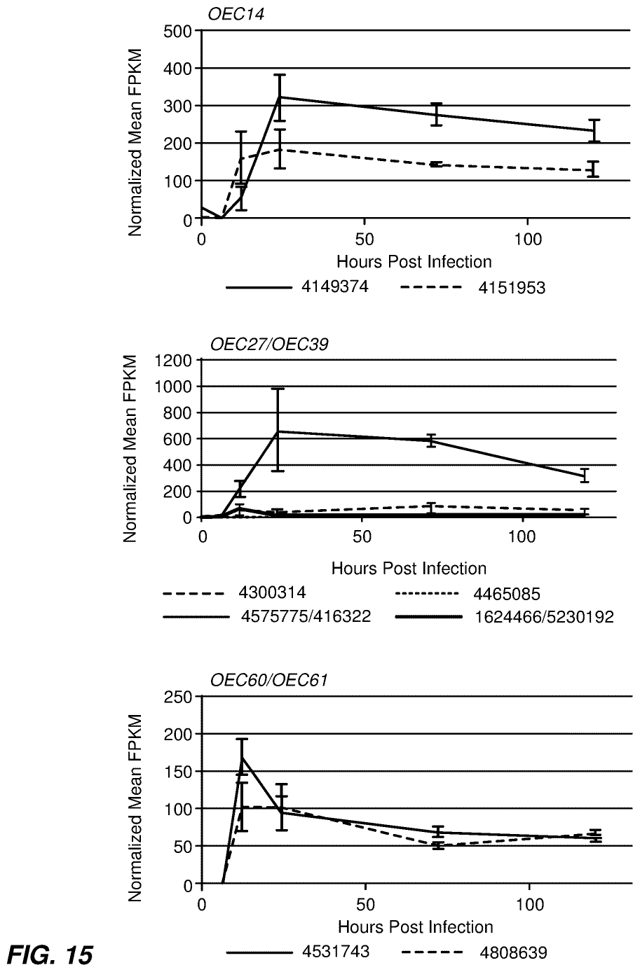

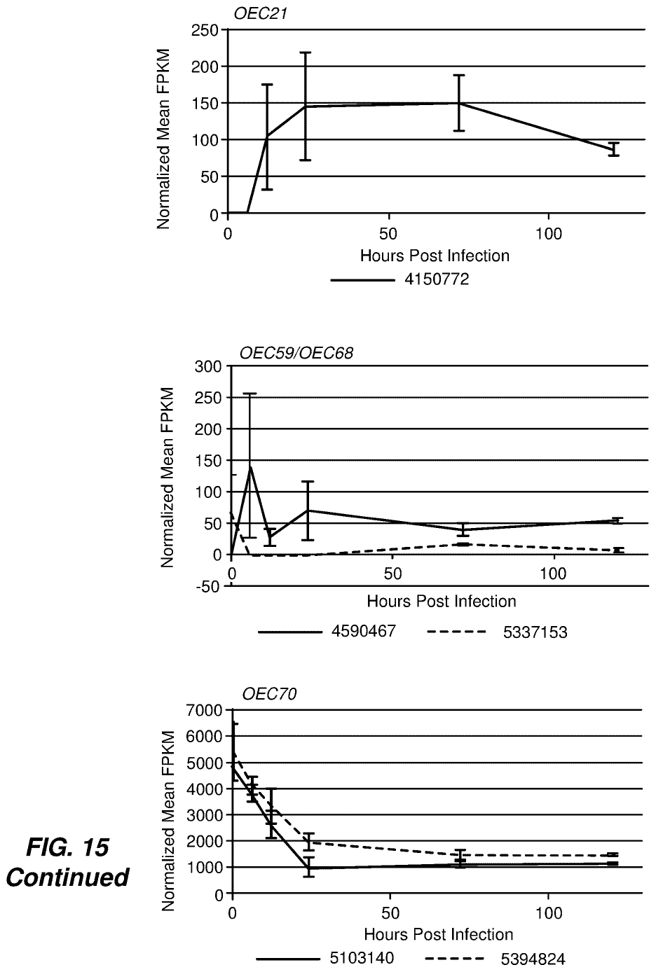

G. orontii MGH1 OEC70 which did contribute to spore production (FIG. 10 panel d) is very highly expressed (FIG. 15). As such, it may be an important conserved powdery mildew virulence factor. Future work will explore the role of OEC70 in the grape powdery mildew as was done with OEC60/OEC61. OEC14 which has a nuclear localization signal and is annotated as an RNAse-like effector (Table 6) also impacts spore production. Unlike the other effectors investigated, it only has one known target, TCP20. The impact of SIGS targeting OEC14 on grapevine powdery mildew proliferation will be assessed on its own and in combinations with OEC70 and/or OEC60/OEC61 targets.

OEC60/OEC61 emerged as an important effector in both the Arabidopsis and grapevine powdery mildew systems. Co-immunoprecipitation of this effector in planta can uncover additional protein interactions that influence this TCP 13-14 node. It can identify both host and pathogen effector proteins not originally included in the yeast-two hybrid screen (WeBling et al., 2014). Indeed, it identified five additional interacting G. orontii proteins (Table 8) including the cell fate modulator APC1 which when silenced resulted in 48% reduction in powdery mildew spore production. OEC60/OEC61 gene structure requires further investigation. The OEC60/OEC61 sequence that is responsible for TCP interaction via yeast 2-hybrid is completely independent of the GH17 domain, but both have implications in pathogenicity. In both plants and fungi, GH17 domain containing proteins are involved in immunity (Minic, 2008). One reported activity of this enzyme class is to break down callose which is deposited at plasmodesmata controlling the size exclusion limit of plasmodesmata and impacting pathogen susceptibility (Gaudioso-Pedraza and Benitez-Alfonso, 2014). However, the substrate of glycans can be hard to predict and would need to be experimentally validated. It is tempting to believe GH17 could be involved in post translational modification of TCPs. For example, TCP15 can be glycosylated to promote cytokinin responses (Steiner et al., 2016, 2012).

TCP13 and TCP14 are highly interactive proteins known to each target hundreds of proteins. These interactions modify their activity in regulating transcription. In the context of powdery mildew infection, these proteins are heavily targeted which may impact their protein and DNA binding capacities fine-tuning their role in this specific context. Several proteins that co-interact with both TCP13 and TCP14 are implicated in modulating the cell cycle. KNAT1 is highly expressed in hypocotyl tissue (Klepikova et al., 2016) which undergoes endoreduplication to support the growth of the plant before photosynthesis can take place (Jakoby and Schnittger, 2004). DA1 directly regulates endoreduplication by controlling the stability of TCP14 and TCP15 (Peng et al., 2015). CUC2 binds the DA1 promoter to promote its expression (Li et al., 2020). As our findings suggest, effectors could play a role in transiently modulating these interactions to guide TCP network outcomes. There are many examples of TCPs being targeted by pathogen effectors to promote infection. The P. syringae effector HopBB1 has been identified as a jasmonic acid (JA) regulator, facilitating the degradation of two negative regulators of JA, JAZ3 and TCP14, to promote virulence (Yang et al., 2017). Aster Yellows phytoplasma strain Witches' Broom (AY-WB) also encode effectors known to degrade TCPs. Specifically, effector SAP11 degrades class II sub group CIN-TCPs like TCP13 (Sugio et al., 2014). Expression of SAP11 in A. thaliana severely impacts development, phenocopying disease symptoms for which the disease is named after, Witches' Broom (Sugio et al., 2011). However, we found OEC60/OEC61 expression in plants does not lead to severe defects in plant development. SAP11 and OEC60/OEC61 are unrelated effectors originating in different domains of life but still converge on these TCP nodes.

### REFERENCES FOR EXAMPLE 2

Baba, K., Nakano, T., Yamagishi, K., Yoshida, S., 2001. Involvement of a nuclear-encoded basic helix-loop-helix protein in transcription of the light-responsive promoter of psbD. Plant Physiology 125, 595-603. https://doi.org/10.1104/pp. 125.2.595.
Bernhofer, M., Goldberg, T., Wolf, S., Ahmed, M., Zaugg, J., Boden, M., Rost, B., 2018. NLSdb-major update for database of nuclear localization signals and nuclear export signals. Nucleic Acids Research 46, D503-D508. https://doi.org/10.1093/nar/gkx1021.
Chandran, D., Inada, N., Hather, G., Kleindt, C. K., Wildermuth, M. C., 2010. Laser microdissection of Arabidopsis cells at the powdery mildew infection site reveals site-specific processes and regulators. Proceedings of the National Academy of Sciences of the United States of America 107, 460-465. https://doi.org/10.1073/pnas.0912492107.
Chandran, D., Rickert, J., Cherk, C., Dotson, B. R., Wildermuth, M. C., 2013. Host Cell Ploidy Underlying the Fungal Feeding Site Is a Determinant of Powdery Mildew Growth and Reproduction. Molecular Plant-Microbe Interactions 26, 537-545. doi.org/10.1094/MPMI-10-12-0254-R.
Chandran, D., Wildermuth, M. C., 2016. Modulation of Host Endocycle During Plant-Biotroph Interactions, Enzymes. https://doi.org/10.1016/bs.enz.2016.09.001.
Chen, L., Fincher, G. B., Hoj, P. B., 1993. Evolution of polysaccharide hydrolase substrate specificity. Catalytic amino acids are conserved in barley 1,3-1,4- and 1,3-P-glucanases. Journal of Biological Chemistry 268, 13318-13326.
Clough, S. J., Bent, A. F., 1998. Floral dip: A simplified method for Agrobacterium-mediated transformation of Arabidopsis thaliana. Plant Journal 16,735-743. doi.org/10.1046/j.1365-313X.1998.00343.x.
Danisman, S., van Dijk, A. D. J., Bimbo, A., van der Wal, F., Hennig, L., de Folter, S., Angenent, G. C., Immink, R. G. H., 2013. Analysis of functional red undancies within the Arabidopsis TCP transcription factor family. Journal of Experimental Botany 64, 5673-5685. https://doi.org/10.1093/jxb/ert337.
Dreze, M., Carvunis, A.-R., Charloteaux, B., Galli, M., Pevzner, S. J., Tasan, M., Ahn, Y.-Y., Balumuri, P., Barabasi, A.-L., Bautista, V., Braun, P., Byrdsong, D., Chen, H., Chesnut, J. D., Cusick, M. E., Dangl, J. L., de los Reyes, C., Dricot, A., Duarte, M., Ecker, J. R., Fan, C., Gai, L., Gebreab, F., Ghoshal, G., Gilles, P., Gutierrez, B. J., Hao, T., Hill, D. E., Kim, C. J., Kim, R. C., Lurin, C., MacWilliams, A., Matrubutham, U., Milenkovic, T., Mirchandani, J., Monachello, D., Moore, J., Mukhtar, M. S., Olivares, E., Patnaik, S., Poulin, M. M., Przulj, N., Quan, R., Rabello, S., Ramaswamy, G., Reichert, P., Rietman, E. A., Rolland, T., Romero, V., Roth, F. P., Santhanam, B., Schmitz, R. J., Shinn, P., Spooner, W., Stein, J., Swamilingiah, G. M., Tam, S., Vandenhaute, J., Vidal, M., Waaijers, S., Ware, D., Weiner, E. M., Wu, S., Yazaki, J., 2011. Evidence for Network Evolution in an Arabidopsis Interactome Map. Science 333, 601-607. doi.org/10.1126/science.1203877.
Earley, K. W., Haag, J. R., Pontes, O., Opper, K., Juehne, T., Song, K., Pikaard, C. S., 2006. Gateway-compatible vectors for plant functional genomics and proteomics. Plant Journal 45, 616-629. doi.org/10.1111/j.1365-313X.2005.02617.x.
Gaudioso-Pedraza, R., Benitez-Alfonso, Y., 2014. A phylogenetic approach to study the origin and evolution of plasmodesmata-localized glycosyl hydrolases family 17. Frontiers in Plant Science 5, 1-14. doi.org/10.3389/fpls.2014.00212.
Grigoriev, I. v., Nikitin, R., Haridas, S., Kuo, A., Ohm, R., Otillar, R., Riley, R., Salamov, A., Zhao, X., Korzeniewski, F., Smirnova, T., Nordberg, H., Dubchak, I., Shabalov, I., 2014. MycoCosm portal: Gearing up for 1000 fungal genomes. Nucleic Acids Research 42, 699-704. doi.org/10.1093/nar/gkt1183.
Gutie, R. A., Doerner, P., Li, C., Colon-Carmona, A., Potuschak, T., 2005. Arabidopsis TCP20 links regulation of growth and cell division control pathways. Proceedings of the National Academy of Sciences of the United States of America 102, 12978-12983.
Hur, Y. S., Kim, J., Kim, S., Son, O., Kim, W. Y., Kim, G. T., Ohme-Takagi, M., Cheon, C. I., 2019. Identification of TCP13 as an upstream regulator of ATHB12 during leaf development. Genes 10. doi.org/10.3390/genes10090644.
Jakoby, M., Schnittger, A., 2004. Cell cycle and differentiation. Current Opinion in Plant Biology 7, 661-669. doi.org/10.1016/j.pbi.2004.09.015.
Jones, L., Riaz, S., Morales-Cruz, A., Amrine, K. C., McGuire, B., Gubler, W., Walker, M., Cantu, D., 2014. Adaptive genomic structural variation in the grape powdery mildew pathogen, Erysiphe necator. BMC Genomics 15, 1081. doi.org/10.1186/1471-2164-15-1081.
Klepikova, A. v., Kasianov, A. S., Gerasimov, E. S., Logacheva, M. D., Penin, A. A., 2016. A high resolution map of the Arabidopsis thaliana developmental transcriptome based on RNA-seq profiling. Plant Journal 88, 1058-1070. doi.org/10.1111/tpj.13312.
Koyama, T., Mitsuda, N., Seki, M., Shinozaki, K., Ohme-takagi, M., 2010. TCP Transcription Factors Regulate the Activities of ASYMMETRIC LEAVES1 and miR164, as Well as the Auxin Response, during Differentiation of Leaves in Arabidopsis 22, 3574-3588. doi.org/10.1105/tpc.110.075598.
Li, S., 2015. The Arabidopsis thaliana TCP transcription factors: A broadening horizon beyond development. Plant Signaling and Behavior 10, 1-12. doi.org/10.1080/15592324.2015.1044192.
Li, Yu, Xia, T., Gao, F., Li, Yunhai, 2020. Control of Plant Branching by the CUC2/CUC3-DA1—UBP15 Regulatory Module. The Plant Cell 32, 1919-1932. doi.org/10.1105/tpc.20.00012.
Lopez, J. A., Sun, Y., Blair, P. B., Mukhtar, M. S., 2015. TCP three-way handshake: linking developmental processes with plant immunity. Trends in Plant Science. doi.org/10.1016/j.tplants.2015.01.005.
Minic, Z., 2008. Physiological roles of plant glycoside hydrolases. Planta 227, 723-740. doi.org/10.1007/s00425-007-0668-y.
Navaud, O., Dabos, P., Carnus, E., Tremousaygue, D., Herve, C., 2007. TCP transcription factors predate the emergence of land plants. Journal of Molecular Evolution 65, 23-33. doi.org/10.1007/s00239-006-0174-z.
Peng, Y., Chen, L., Lu, Y., Wu, Y., Dumenil, J., Zhu, Z., 2015. The Ubiquitin Receptors DA1, DAR1, and DAR2 Redundantly Regulate Endoreduplication by Modulating the Stability of TCP14/15 in Arabidopsis 27, 649-662. doi.org/10.1105/tpc.114.132274.
Reuber, T. L., Plotnikova, J. M., Dewdney, J., Rogers, E. E., William Wood, B., Ausubel, F. M., 1998. Correlation of defense gene induction defects with powdery mildew susceptibility in Arabidopsis enhanced disease susceptibility mutants. Plant Journal 16, 473-485. doi.org/10.1046/j.1365-313X.1998.00319.x.
Spanu, P. D., Abbott, J. C., Amselem, J., Burgis, T. A., Soanes, D. M., Stuber, K., van Themaat, E. V. L., Brown, J. K. M., Butcher, S. A., Gurr, S. J., Lebrun, M. H., Ridout, C. J., Schulze-Lefert, P., Talbot, N. J., Ahmadinejad, N., Ametz, C., Barton, G. R., Benjdia, M., Bidzinski, P., Bindschedler, L. v., Both, M., Brewer, M. T., Cadle-Davidson, L., Cadle-Davidson, M. M., Collemare, J., Cramer, R., Frenkel, O., Godfrey, D., Harriman, J., Hoede, C., King, B. C., Klages, S., Kleemann, J., Knoll, D., Koti, P. S., Kreplak, J., Lopez-Ruiz, F. J., Lu, X., Maekawa, T., Mahanil, S., Micali, C., Milgroom, M. G., Montana, G., Noir, S., O'Connell, R. J., Oberhaensli, S., Parlange, F., Pedersen, C., Quesneville, H., Reinhardt, R., Rott, M., Sacristan, S., Schmidt, S. M., Schon, M., Skamnioti, P., Sommer, H., Stephens, A., Takahara, H., Thordal-Christensen, H., Vigouroux, M., WeBling, R., Wicker, T., Panstruga, R., 2010. Genome expansion and gene loss in powdery mildew fungi reveal tradeoffs in extreme parasitism. Science 330, 1543-1546. doi.org/10.1126/science.1194573.
Stark, C., Breitkreutz, B. J., Reguly, T., Boucher, L., Breitkreutz, A., Tyers, M., 2006. BioGRID: a general repository for interaction datasets. Nucleic acids research 34, 535-539. doi.org/10.1093/nar/gkj109.
Steiner, E., Efroni, I., Gopalraj, M., Saathoff, K., Tseng, T., Kieffer, M., Eshed, Y., Olszewski, N., Weiss, D., 2012. The Arabidopsis O-Linked N-Acetylglucosamine Transferase SPINDLY Interacts with Class I TCPs to Facilitate Cytokinin Responses in Leaves and Flowers 24, 96-108. doi.org/10.1105/tpc.111.093518.
Steiner, E., Livne, S., Kobinson-Katz, T., Tal, L., Pri-Tal, O., Mosquna, A., Tarkowska, D., Mueller, B., Tarkowski, P., Weiss, D., 2016. The putative O-linked N-acetylglucosamine transferase SPINDLY inhibits class I TCP proteolysis to promote sensitivity to cytokinin. Plant Physiology 171, 1485-1494. doi.org/10.1104/pp. 16.00343.
Sugio, A., Kingdom, H. N., MacLean, A. M., Grieve, V. M., Hogenhout, S. A., 2011. Phytoplasma protein effector SAP11 enhances insect vector reproduction by manipulating plant development and defense hormone biosynthesis. Proceedings of the National Academy of Sciences of the United States of America 108, 1-10. doi.org/10.1073/pnas.1105664108.
Sugio, A., Maclean, A. M., Hogenhout, S. A., 2014. The small phytoplasma virulence effector SAP11 contains distinct domains required for nuclear targeting and CIN-TCP binding and destabilization. New Phytologist 202, 838-848. doi.org/10.1111/nph.12721.
Tanaka, Y., Kimura, T., Hikino, K., Goto, S., Nishimura, M., Mano, S., Nakagaw, T., 2012. Gateway Vectors for Plant Genetic Engineering: Overview of Plant Vectors, Application for Bimolecular Fluorescence Complementation (BiFC) and Multigene Construction. Genetic Engineering—Basics, New Applications and Responsibilities. doi.org/10.5772/32009.
Vercruysse, J., Baekelandt, A., Gonzalez, N., Inze, D., 2020. Molecular networks regulating cell division during Arabidopsis leaf growth. Journal of Experimental Botany 71, 2365-2378. doi.org/10.1093/jxb/erz522.
Vlieghe, K., Boudolf, V., Beemster, G. T. S., Maes, S., Magyar, Z., Atanassova, A., de Almeida Engler, J., de Groodt, R., Inze, D., de Veylder, L., 2005. The DP-E2F-like Gene DEL1 Controls the Endocycle in Arabidopsis thaliana. Current Biology 15, 59-63. doi.org/10.1016/j.cub.2004.12.038.
W, D. A. C., Danisman, S., Wal, F. van der, Dhondt, S., Waites, R., Folter, S. de, Bimbo, A., Dijk, A. D. J. van, Muino, J. M., Cutri, L., Dornelas, M. C., Angenent, G. C., Immink, R. G. H., 2012. Arabidopsis Class I and Class II TCP Transcription Factors Regulate Jasmonic Acid Metabolism and Leaf 159, 1511-1523. doi.org/10.1104/pp. 112.200303.
Wang, S., Li, W., Liu, S., Xu, J., 2016. RaptorX-Property: a web server for protein structure property prediction. Nucleic acids research 44, W430-W435. doi.org/10.1093/nar/gkw306.
WeBling, R., Epple, P., Altmann, S., He, Y., Yang, L., Henz, S. R., McDonald, N., Wiley, K., Bader, K. C., GlaBer, C., Mukhtar, M. S., Haigis, S., Ghamsari, L., Stephens, A. E., Ecker, J. R., Vidal, M., Jones, J. D. G., Mayer, K. F. X., ver Loren Van Themaat, E., Weigel, D., Schulze-Lefert, P., Dangl, J. L., Panstruga, R., Braun, P., 2014. Convergent targeting of a common host protein-network by pathogen effectors from three kingdoms of life. Cell Host and Microbe 16, 364-375. doi.org/10.1016/j.chom.2014.08.004.
WeBling, R., Panstruga, R., 2012. Rapid quantification of plant-powdery mildew interactions by qPCR and conidiospore counts. Plant Methods 8, 35. doi.org/10.1186/1746-4811-8-35.
Wildermuth, M. C., 2010. Modulation of host nuclear ploidy: A common plant biotroph mechanism. Current Opinion in Plant Biology 13, 449-458. doi.org/10.1016/j.pbi.2010.05.005.
Wildermuth, M. C., Steinwand, M. A., McRae, A. G., Jaenisch, J., Chandran, D., 2017. Adapted Biotroph Manipulation of Plant Cell Ploidy. Annual Review of Phytopathology 55, 537-564. doi.org/10.1146/annurev-phyto-080516-035458.
Yang, L., Teixeira, P. J. P. L., Biswas, S., Finkel, O. M., He, Y., Salas-Gonzalez, I., English, M. E., Epple, P., Mieczkowski, P., Dangl, J. L., 2017. Pseudomonas syringae Type III Effector.
HopBB1 Promotes Host Transcriptional Repressor Degradation to Regulate Phytohormone Responses and Virulence. Cell Host and Microbe 21, 156-168. doi.org/10.1016/j.chom.2017.01.003.
Zhang, J., Li, L., Lv, Q., Yan, L., Wang, Y., Jiang, Y., 2019. The fungal CYP51s: Their functions, structures, related drug resistance, and inhibitors. Frontiers in Microbiology 10. doi.org/10.3389/fmicb.2019.00691.

### Example 3

Single Copy Genes in G. orontii MGH1 Following Recent Whole Genome Duplication.

Powdery mildew genomes are large and are full of transposable elements that makes it difficult to assemble and analyze (Spanu et al. 2010; Frantzeskakis et al., 2018; Wicker et al 2013). With the DOE Joint Genome Institute, Walnut Creek, CA, we have led sequencing of the genomes of several powdery mildew species using long read PacBio sequencing including isolates of G. orontii (Arabidopsis), G. cichoracearum (Cucurbits), P. neolycopersici (Tomato), E. pisi (Pea) and B. graminis fsp hordei (Barley). The genome of G. orontii MGH1 is one of the best assembled powdery mildew genomes to-date and includes HiC data in its assembly.

Powdery mildew genomes are haploid. However, the genome assembly of G. orontii MGH1 (Gor) indicates a whole genome duplication (WDG) event supported by the larger genome size, twice the number of genes as compared to other powdery mildew species, and large segmental duplications. Evolutionary analyses indicate that this is a recent event, and duplicated genes have more than 95% sequence similarity in duplicate gene pairs. Despite this nearly universal duplication of genes, a small set of genes are single copy in Gor, suggesting there was strong pressure to maintain these genes as single copy.

The polyploidization or whole genome duplication is a phenomenon that happens in nature and plays a critical role in evolution of species (Duarte et al. 2010, Tan et al. 2016). Although, the duplication events provide the additional genetic material that helps the species to diversify and evolve, it is critical for cells to maintain a gene dosage balance for some genes to maintain normal developmental processes. To maintain the gene dosage balance, duplicated genes can undergo different processes including maintaining the dosage dependent expression of genes, neo-functionalization, sub-functionalization or gene loss (Shi et al. 2015, Lloyd et al. 2014). Several studies analyzing genome evolution following the genome duplication events suggest that gene loss is not a random process and that specific functional classes of genes exist that are under strong selective pressures to return to single copy. Some of these genes are involved in cellular and developmental processes e.g., transcription and translation machinery, cell organization and biogenesis e.g., genes involved in meiotic cell division, and genes involved in organelle-nuclear communication (Lloyd et al. 2014, Ha et al. 2007, Sriswasdi et al. 2016).

We analyzed the G. orontii MGH1 genome to identify single copy genes, e.g., those that have lost the duplicated copy following the WGD (see, e.g., Table 9). There are >2500 single copy genes conserved in all powdery mildews except for G. orontii MGH1 which, due to WGD, now has two copies for most of these genes. However, for 213 genes, G. orontii MGH1 has lost one copy of the duplicated gene pair. Pfam domain analysis and Gene Ontology analysis of this gene set identified enriched functional categories that overlap with those previously observed (Lloyd et al. 2014, Ha et al. 2007, Sriswasdi et al. 2016.) and that are distinct. For example, genes involved in cellular and development processes including transcription and translation, genes involved in cell organization and biogenesis, e.g., cell cycle/cell division, and genes involved in vesicular and transport functions. In addition, genes playing metabolic roles, particularly in lipid metabolism, carbohydrate utilization, and amino acid and nucleotide metabolism were enriched. Finally, there were a number of single copy genes with unknown function. Single copy genes serve as ideal targets for SIGS as they tend to be essential genes, there is only one copy of the gene to be silenced, and chances of resistance development through increased copy number are dramatically reduced or absent. For metabolic genes, single copy genes are likely to represent highly regulated enzymes in a pathway or to produce a regulatory compound. For example, Gor BCDO encodes an NCED enzyme that serves as the rate-limiting enzyme in abscisic acid synthesis in plants and plays a critical regulatory function (Xiong and Zhu, 2003).

We have prioritized 57 of these single copy gene targets for initial target testing (Table 9, below). Sequences for the additional 156 single copy gene targets are provided (see, e.g., SEQ ID NOs:255-411). In Table 1, they are distributed in distinct process categories when their predicted function is known and in Category 4 (Essential Cellular Processes) when unknown. We have tested SIGS against 13 of these single copy gene targets, and 12 of 13 resulted in significant reduction in G. orontii growth and reproduction on Arabidopsis (see, e.g., Table 2 and Table 9). This results in a 92% success rate in the identification of successful target genes to-date. It also suggests that additional dsRNA sequences against the one target that showed no phenotype in disease reduction be tested to ensure the negative result is not merely the result of an inefficient dsRNA. E. necator orthologs of BCDO and APG9 have been tested in the E. necator-grapevine system and showed dramatic reduction (>50%) in powdery mildew growth and reproduction (Table 2). Furthermore, topical dsRNA against E. necator APG9 was effective in vineyard field trials at reducing powdery mildew disease (see Example 4).

For process categories already of particular interest (see Tables 1 and 3, supra), we prioritize the single copy genes to target. In addition, categories enriched for these single copy genes point us to new process categories of importance for target prioritization as well as genes with unknown function we may not have otherwise prioritized. While some of these genes are associated with enriched process categories for single copy genes in other systems, there are also distinct genes in our set, that may be shared with other plant biotrophs and particularly plant obligate biotrophs that share an evolved dependency on plant nutrients and metabolites.

TABLE 9
Illustrative genes that are single copy following WGD in G. orontii - by process
category. Bolded genes show reduction in powdery mildew growth & reproduction when
silenced. Of 13 single copy genes targets tested, 12 showed a SIGS pathogen reduction
phenotype. The two of these orthologous E. necator targets tested, also showed
reduction in powdery mildew growth and reproduction (flagged by “**”).

| | | | |
|---|---|---|---|
| SEQ | | | |
| ID NO | Gene Name/PFAM domain | Process category | Process subcategories |

Tested Gene Targets

| | | | |
|---|---|---|---|
| 15 | Transaldolase | Metabolism | Lipid metabolism |
| | | | PPP pathway |
| 54 | Fructose 1,6-BP aldolase | Metabolism | Carbohydrate Metabolism |
| | | | Glycolysis |
| 63 | Glutamate dehydrogenase | Metabolism | Amino Acid & nucleotide |
| | | | metabolism |
| | | | Amino |
| | | | metabolism/salvage |
| 64 | Threonine aldolase | Metabolism | Amino Acid & nucleotide |
| | | | metabolism |
| | | | Amino |
| | | | metabolism/salvage |
| 70 | pf02548~Ketopantoate | Metabolism | Amino Acid & nucleotide |
| | hydroxymethyltransferase | | metabolism |
| | | | Cofactor metabolism |
| 87 | pf08238~Sell repeat | Metabolism | Mitochondrial electron |
| | | | transport chain & ATP |
| | | | synthesis |
| | | | Mitochondrial |
| | | | electron transport |
| 104 | pf10453~Nuclear fragile X | Regulation | Protein regulation |
| | mental retardation- | | Translation |
| | interacting protein 1 | | |
| | (NUFIP1) | | |
| 145 ** | β-carotene 15,15′- | Manipulation of | Plant Hormone Metabolism |
| | dioxygenase (BCDO) | Plant Host | Abscisic acid |
| | | | (carotenoid) |
| 164 ** | pf04109~Autophagy protein | Essential Cellular | Vesicle, Autophagy, |
| | Apg9 | Processes | Phagocytosis |
| | | | Vesicle Formation |
| 167 | pf03517~Regulator of | Essential Cellular | Ion/Metal transport and |
| | volume decrease after | Processes | osmotic homeostasis |
| | cellular swelling | | Cell volume control |
| 169 | Ctr family Cu2+ | Essential Cellular | Ion/Metal transport and |
| | transporter | Processes | osmotic homeostasis |
| | | | Metal/ion transporter |
| 179 | NA | Essential Cellular | Unknown Function |
| | | Processes | |
| 180 | Cyclin-like protein | Essential Cellular | Cell cycle and DNA |
| | | Processes | replication |

Additional identified targets

| | | | |
|---|---|---|---|
| 40 | Acetyl-CoA transporter | Metabolism | Lipid Metabolism |
| | | | Acetyl CoA transport |
| 51 | pf01066~CDP-alcohol | Metabolism | Lipid metabolism |
| | phosphatidyltransferase | | Phospholipid synthesis |
| 58 | pf13418~Galactose oxidase, | Metabolism | Carbohydrate Metabolism |
| | central domain; | | Carbohydrate |
| | pf00651~BTB/POZ domain; | | utilization |
| | pf13854~Kelch motif; | | |
| | pf01344~Kelch motif | | |
| 59 | pf04321~RmlD substrate | Metabolism | Carbohydrate Metabolism |
| | binding domain | | Carbohydrate |
| | | | utilization |
| 60 | pf01256~Carbohydrate | Metabolism | Carbohydrate Metabolism |
| | kinase | | Carbohydrate |
| | | | utilization |
| 61 | Myo-inositol, sugar, and | Metabolism | Carbohydrate metabolism |
| | other transporter | | Metabolite transporter |
| 65 | serine o-acetyltransferase | Metabolism | Amino Acid & nucleotide |
| | | | metabolism |
| | | | cys/met metabolism |
| 66 | spermidine synthase | Metabolism | Amino Acid & nucleotide |
| | | | metabolism |
| | | | cys/met metabolism |
| 68 | Transmembrane AA | Metabolism | Amino Acid & nucleotide |
| | transporter | | metabolism |
| | | | Amino acid transport |
| 72 | pf02558~Ketopantoate | Metabolism | Amino Acid & nucleotide |
| | reductase PanE/ApbA; | | metabolism |
| | pf08546~Ketopantoate | | Cofactor metabolism |
| | reductase PanE/ApbA C | | |
| | terminal | | |
| 78 | pf02492~CobW/HypB/UreG, | Metabolism | Amino Acid & nucleotide |
| | nucleotide-binding domain; | | metabolism |
| | pf07683~Cobalamin | | Cofactor metabolism |
| | synthesis protein cobW C- | | |
| | terminal domain | | |
| 83 | pf00383~Cytidine and | Metabolism | Amino Acid & nucleotide |
| | deoxycytidylate deaminase | | metabolism |
| | zinc-binding region | | Nucleotide metabolism |
| 88 | pf05405~Mitochondrial ATP | Metabolism | Mitochondrial electron |
| | synthase B chain precursor | | transport chain & ATP |
| | (ATP-synt_B) | | synthesis |
| | | | ATP synthesis |
| 93 | BZip, AP1-like transcription | Regulation | Transcription regulation |
| | factor | | Stress response |
| 98 | fungal specific transcription | Regulation | Transcription regulation |
| | factor domain, Zn2Cys6 | | Development |
| | binuclear cluster domain | | |
| 99 | Brucentaur or craniofacial | Regulation | Transcription regulation |
| | development protein | | Development |
| 105 | pf01205~Uncharacterized | Regulation | Protein regulation |
| | protein family UPF0029; | | |
| | pf05773~RWD domain | | Translation |
| 106 | pf00642~Zinc finger C-x8-C- | Regulation | Protein regulation |
| | x5-C-x3-H type (and similar) | | Translation |
| 107 | pf10343~Potential | Regulation | Protein regulation |
| | Queuosine, Q, salvage | | Translation |
| | protein family | | |
| 108 | pf03914~CBF/Mak21 family | Regulation | Protein regulation |
| | | | Translation |
| 109 | pf01746~tRNA (Guanine-1)- | Regulation | Protein regulation |
| | methyltransferase | | Translation |
| 110 | pf13519~von Willebrand | Regulation | Protein regulation |
| | factor type A domain | | Protein turnover |
| 111 | pf02290~Signal recognition | Regulation | Protein regulation |
| | particle 14 kD protein | | Protein turnover |
| 112 | pf03810~Importin-beta N- | Regulation | Protein regulation |
| | terminal domain | | Protein turnover |
| 113 | pf00639~PPIC-type PPIASE | Regulation | Protein regulation |
| | domain; peptidyl prolyl | | Protein turnover |
| | isomerase | | |
| 154 | anthranilate synthase | Manipulation of | Plant Hormone Metabolism |
| | | Plant Host | Salicylic acid, auxin |
| | | | (chorismate) |
| 155 | pf00566~Rab-GTPase-TBC | Essential Cellular | Essential cellular processes |
| | domain | Processes | Cell cycle/DNA |
| | | | replication |
| 156 | pf10444~Nbl1/Borealin N | Essential Cellular | Essential cellular processes |
| | terminal | Processes | Cell cycle/DNA |
| | | | replication |
| 157 | pf06632~DNA double-strand | Essential Cellular | Essential cellular processes |
| | break repair and V(D)J | Processes | Cell cycle/DNA |
| | recombination protein | | replication |
| | XRCC4 | | |
| 158 | pf00515~Tetratricopeptide | Essential Cellular | Essential cellular processes |
| | repeat; pf07719~Tetratricopeptide | Processes | Cell cycle/DNA |
| | repeat; pf12895Anaphase- | | replication |
| | promoting complex, cyclosome, | | |
| | subunit3; pf13424~ | | |
| | Tetratricopeptide repeat | | |
| 159 | pf12720~Protein of unknown | Essential Cellular | Essential cellular processes |
| | function (DUF3807) | Processes | Cell cycle/DNA |
| | | | replication |
| 160 | pf00515~Tetratricopeptide | Essential Cellular | Essential cellular processes |
| | repeat; pf13414~TPR repeat | Processes | Cell cycle/DNA |
| | | | replication |
| 161 | pf00400~WD domain, G-beta | Essential Cellular | Essential cellular processes |
| | repeat | Processes | Cell cycle/DNA |
| | | | replication |
| 162 | pf13414~TPR repeat | Essential Cellular | Essential cellular processes |
| | | Processes | Cell cycle/DNA |
| | | | replication |
| 165 | pf04727~ELMO/CED-12 | Essential Cellular | Vesicle, Autophagy, |
| | family | Processes | Phagocytosis |
| | | | Phagocytosis |
| 166 | pf00789~UBX domain; | Essential Cellular | Vesicle, Autophagy, |
| | pf08059~SEP domain; | Processes | Phagocytosis |
| | pf14555~UBA-like domain | | Vesicle membrane |
| | | | fusion |
| 168 | CorA-like Mg2+ transporter | Essential Cellular | Ion/Metal transport and |
| | | Processes | osmotic homeostasis |
| | | | Metal/ion transporter |
| 170 | N/A | Essential Cellular | Unknown function |
| | | Processes | |
| 171 | N/A | Essential Cellular | Unknown function |
| | | Processes | |
| 172 | N/A | Essential Cellular | Unknown function |
| | | Processes | |
| 173 | N/A | Essential Cellular | Unknown function |
| | | Processes | |
| 174 | N/A | Essential Cellular | Unknown function |
| | | Processes | |
| 175 | N/A | Essential Cellular | Unknown function |
| | | Processes | |
| 176 | N/A | Essential Cellular | Unknown function |
| | | Processes | |
| 177 | N/A | Essential Cellular | Unknown function |
| | | Processes | |
| 178 | pf11937~Protein of unknown | Essential Cellular | Unknown function |
| | function (DUF3455) | Processes | |

### REFERENCES FOR EXAMPLE 3

Spanu P D, Abbott J C, Amselem J et al. Genome expansion and gene loss in powdery mildew fungi reveal tradeoffs in extreme parasitism. Science. 2010 Dec. 10; 330(6010); 1543-6. http://dx.doi.org/10.1126/science.1194573
Wicker T, Oberhaensli S, Parlange F, Buchmann J P, Shatalina M, Roffler S, et al. (2013). The wheat powdery mildew genome shows the unique evolution of an obligate biotroph. Nat. Genet. 45 1092-1096. http://dx.doi.org/10.1038/ng.2704
Frantzeskakis L, Kracher B, Kusch S, Yoshikawa-Maekawa M, Bauer S, Pedersen C, et al. Signatures of host specialization and a recent transposable element burst in the dynamic one-speed genome of the fungal barley powdery mildew pathogen. BMC Genomics. 2018; 19(1); 381. https://doi.org/10.1186/s12864-018-4750-6
Tan C, Pan Q, Cui C, Xiang Y, Ge X, Li Z. Genome-Wide Gene/Genome Dosage Imbalance Regulates Gene Expressions in Synthetic Brassica napus and Derivatives (AC, AAC, CCA, CCAA). Front Plant Sci. 2016 Sep. 23; 7:1432. doi: 10.3389/fpls.2016.01432.
Shi T, Rahmani R S, Gugger P F, Wang M, Li H, Zhang Y, Li Z, Wang Q, Van de Peer Y, Marchal K, Chen J. Distinct Expression and Methylation Patterns for Genes with Different Fates following a Single Whole-Genome Duplication in Flowering Plants. Mol Biol Evol. 2020 Aug. 1; 37(8); 2394-2413. doi: 10.1093/molbev/msaa105.
Lloyd A H, Ranoux M, Vautrin S, Glover N, Fourment J, Charif D, Choulet F, Lassalle G, Marande W, Tran J, Granier F, Pingault L, Remay A, Marquis C, Belcram H, Chalhoub B, Feuillet C, Berges H, Sourdille P, Jenczewski E. Meiotic gene evolution: can you teach a new dog new tricks?Mol Biol Evol. 2014 July; 31(7); 1724-7. doi: 10.1093/molbev/msu119.
Ha M, Li W H, Chen Z J. External factors accelerate expression divergence between duplicate genes. Trends Genet. 2007 April; 23(4); 162-6. doi: 10.1016/j.tig.2007.02.005.
Sriswasdi S, Takashima M, Manabe R, Ohkuma M, Sugita T, Iwasaki W. Global deceleration of gene evolution following recent genome hybridizations in fungi. Genome Res. 2016 August; 26(8); 1081-90. doi: 10.1101/gr.205948.116.

### Example 4

Topical dsRNA to Control Powdery Mildew in Vineyards

### Summary of Example 4

To assess the effect of topical dsRNA treatments to control powdery mildew in field conditions, we conducted vineyard field trials in the 2021 growing season at 2 different locations in California. Topical dsRNA treatment against five unique powdery mildew targets was tested and compared to untreated and/or gold standard chemical treatment controls. Berry disease incidence and severity, canopy development, berry development, and phytotoxicity were assessed at both sites. Berry chemistry analysis was performed for grapes harvested at one site. The topical spraying of dsRNA against all five gene targets (Seq ID Nos:182, 183, 186, 188, 208) tested in this trial dramatically reduced powdery mildew infection at both trial sites in California. Disease severity was kept at or below 5% and the dsRNA treatments exerted their inhibitory effects beyond 3 weeks. In addition, there was no negative impact of the dsRNA treatments on canopy or berry development, or evidence of phytotoxicity.

### Methods

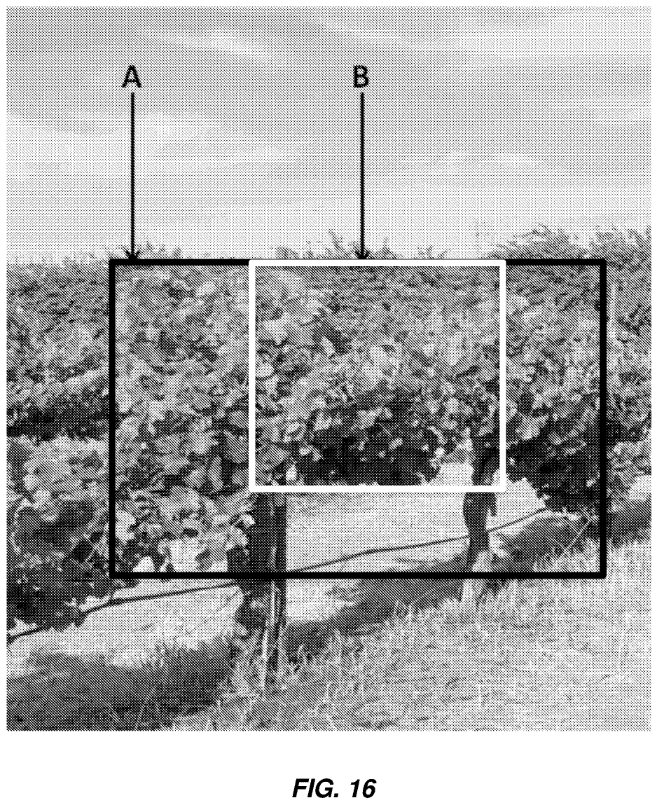

The tests were done at Kearney Agriculture Research and Extension Center, Parlier, CA (Fresno County) and Armstrong Plant Pathology Field Station at University of California, Davis, CA. Susceptible grapevine cultivars were tested—Sauvignon Blanc at Kearney and Chenin Blanc at Davis. In both cases, the Stihl backpack sprayer SR450 was used and sprayer calibration, and normalization was performed to ensure equal doses of treatment applied to each replicate. During the trial period, vines were irrigated by drip irrigation and the regular maintenance of weeding, herbicide and insecticide treatments were done by the Kearney or Davis site field staff. In early season, 2.0 gallons/5 replicates and in mid to late season 2.5 to 3.0 gallons/5 replicates were sprayed. All appropriate personal protective equipment (PPE) was used and required training was completed including pesticide application training and heat exhaustion training. A randomized block design was used with 5 replicates for each treatment. Two adjacent vines were used as one experimental unit and the area between two trunks (yellow box) was sprayed and assessed (FIG. 16).

### Details of Field Site, Experimental Setup and Treatment Timings:

The trial was conducted at Kearney Agricultural Research and Extension Center, Parlier, CA on blocks of the susceptible white wine variety Sauvignon Blanc. Total of 5 different gene targets from grapevine powdery mildew E. necator, EnCYP51 (SEQ ID NO:182), EnLIP1 (SEQ ID NO:183), EnICL2 (SEQ ID NO:186), EnMS (SEQ ID NO:188) and EnAPG9 (SEQ ID NO:208) were tested at this site by backpack spraying of the experimental units with dsRNA designed specifically against these target genes. The treatments were placed in a randomized block design with 5 replicates of each (dsRNA treatments 1-5), 5 replicates of Gold Standard (GS) and 5 replicates of untreated control (UT Control). Gold Standard refers to the pesticides normally used by growers to control powdery mildew: Sulfur and other chemical fungicides such as Rally, Quintec, and Luna. Between each treatment replicate, consisting of two vines, there was a spacer vine to avoid the spray drift from one treatment another.

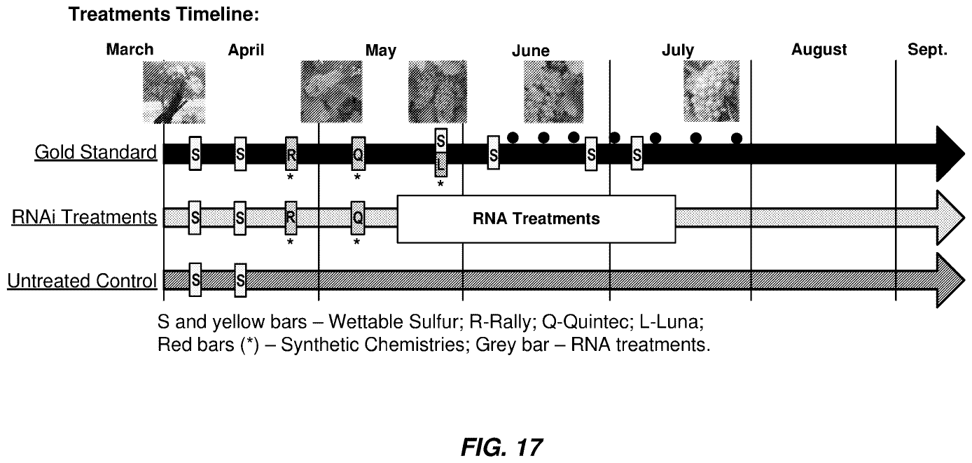

FIG. 17 provides an overview of the treatment schedule at Kearney. A total of 4 treatments of dsRNA were performed between May and July, replacing the 4 final treatments of the GS. Data were collected at the times marked by the gray dots in FIG. 17. The monitoring of powdery mildew index data and manual scouting of disease in the field was used to adjust timing schedule per standard protocol (www2.ipm.ucanr.edu/weather/grape-powdery-mildew-risk-assessment-index/?src=redirect2refresh).

### Data Collection and Analysis:

1. Disease Incidence (DI); Percent of Grape Bunches with Powdery Mildew.

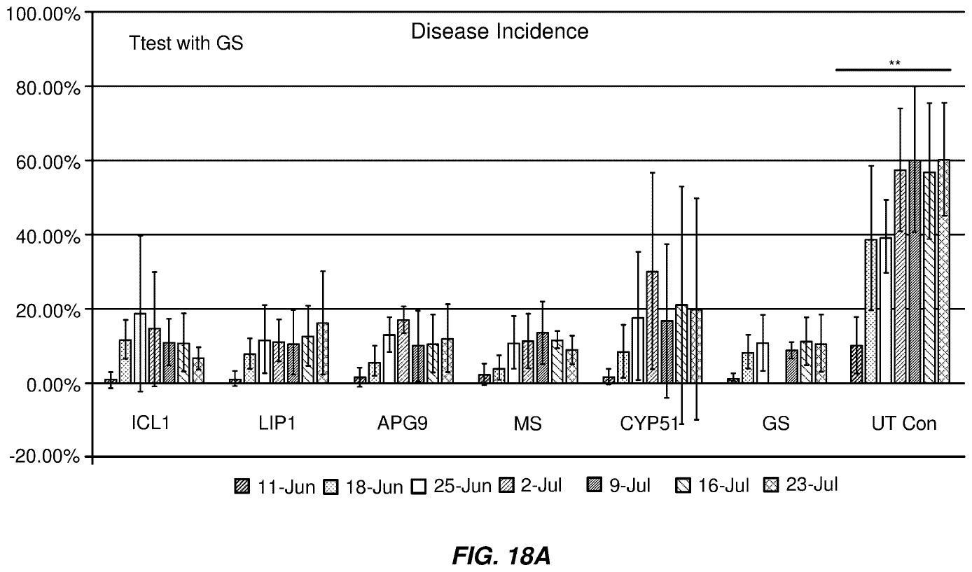

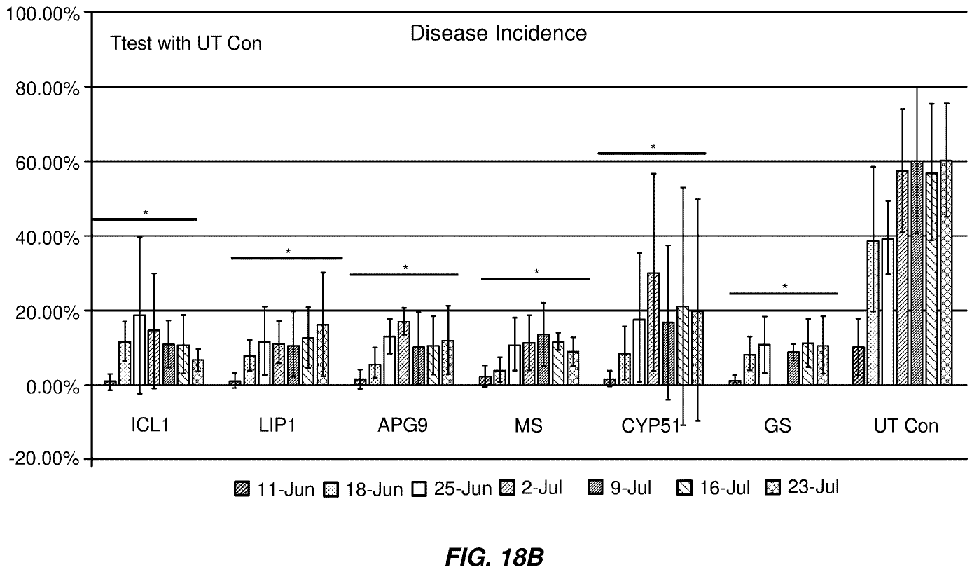

The total number of bunches per replicate (mean of 130) and the bunches with powdery mildew growth were counted and percent of infected bunches per replicate was calculated. Assessment occurred at regular intervals from June 11 through July 23 (veraison) as shown in FIG. 18. The Disease Incidence analysis clearly shows the powdery mildew in the untreated control was significantly higher in comparison to dsRNA treated vines (FIG. 18, panel A) and not different statistically from the Gold Standard Control (FIG. 18, panel B). This indicates that the dsRNA mid-season treatment was effective in reducing powdery mildew disease and as effective as the Gold Standard it replaced (see FIG. 17).

### 2. Disease Severity: Percent of Infected Berries in a Bunch.

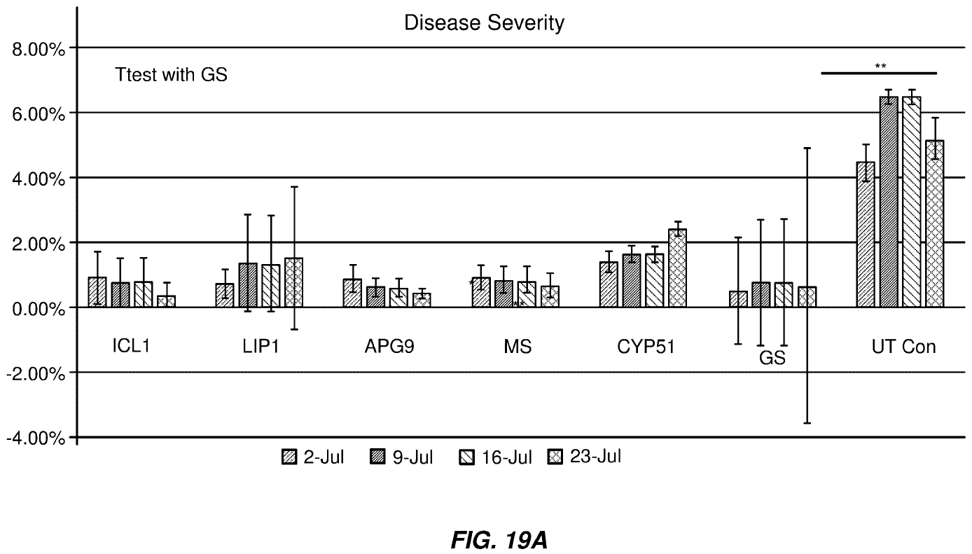

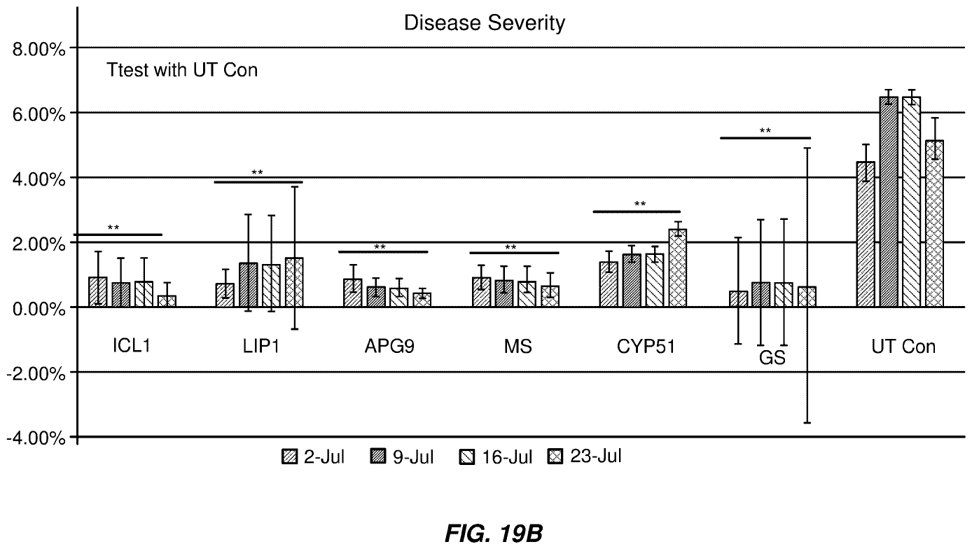

Disease severity was calculated by counting the number of infected berries compared to total berries in a bunch. 25 bunches from both sides of the canopy were assessed per replicate. As shown in FIG. 19, the disease severity for all dsRNA-treatments is well below 5%, is much reduced compared to the Untreated control (FIG. 19, panel A) and not statistically different from the gold standard treatment (FIG. 19, panel B).

### 3. Plant Characteristics: Canopy Size, Berry Development, Phytotoxicity

Canopy size visual estimation: The canopy size was visually estimated and a total of 6 readings from May 7th to July 9th were recorded. Canopy size was determined using a scale from 1 to 10 with 0.5 increments for each replicate, with 10 representing the largest canopy in the pool. Five replicates per treatment were assessed. There was no statistical difference between the canopy size growth over time in treated vs untreated vines suggesting that dsRNA treatment does not impact the canopy growth.

Phytotoxicity: Visual assessment occurred throughout the growing season and no visible signs of necrosis, aberrant leaf development, blistering, or leaf rollup were observed for any treatment. Note that minimal localized chlorosis was associated with powdery mildew infection sites.

Berry development: Qualitative visual assessment occurred throughout the growing season and no difference associated with a particular dsRNA treatment was noted compared with controls.

### 4. Berry Chemistry Analysis: Brix, pH and TA

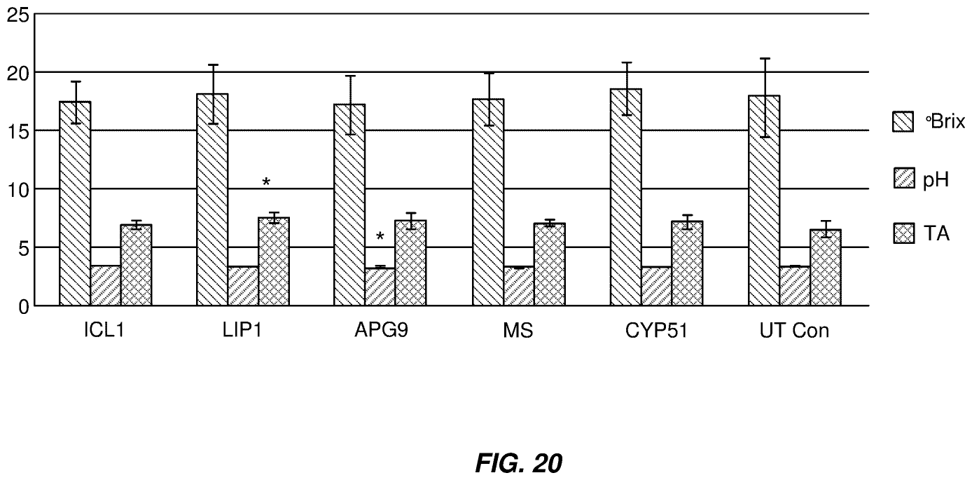

Total of 10 bunches of approximately similar size and without obvious powdery mildew infection from each replicate (5 from each side of the canopy) were collected in the 3rd week of August. The berries were crushed, and the berry juice was cleared by centrifugation. 50 ml of clear berry juice was given to Dr. Qun Sun from Fresno State University for Brix, pH and TA analysis. There was no pattern of impact of dsRNA treatments on Brix, TA or pH. One TA (treatment 2) and one pH (treatment 3) showed a very slight difference compared to the untreated control, with p-values>0.01 (FIG. 20).

### Trial 2. Armstrong Plant Pathology Fieldhouse Facility, University of California Davis, CA

### Details of Field Site, Experimental Setup and Treatment Timings:

This trial was conducted at University of California Davis Plant Pathology Armstrong Facility on white wine variety Chenin Blanc. A total of 4 different gene targets from grapevine powdery mildew E. necator, EnLIP1 (Seq ID No:183), EnICL2 (Seq ID 186), EnMS (Seq ID No:188) and EnAPG9 (Seq ID No:208) were tested at this site by treating the vines with dsRNA designed against these target genes. A randomized block design with 5 replicates of each (treatments 1-5) and 5 replicates of untreated control (UT Control) was utilized. Spray protocols and spray volumes were the same as done by Akif Eskalen's team as shown on this link (Elfar et al. 2021, ucanr.edu/sites/eskalenlab/files/355630.pdf) and in Table 10. Eskalen's lab performed 3rd party commercial fungicide trials for the industry.

TABLE 10
Experimental overview - Armstrong site.

| | |
|---|---|
| Experimental design | Randomized complete block design with 4 replicates |
| Experimental unit | 2 adjacent vines = 1 experimental unit |
| Row and tree | 11 ft (row) and 7 ft (vine); Plot unit area 154 ft2 |
| spacing | |
| Area/treatment | 770 ft2 or 0.0176 acre/treatment (5 replicates = |
| | 1 treatment) |
| Volume water/Acre | 100 gallons/acre (April) = 1.76 gal/5 reps |
| | 150 gallons/acre (late May on) = 2.65 gal/5 reps |
| Equipment | Stihl SR 450 blower backpack sprayer |

Modified from Elfar et al. (2021) //ucanr.edu/sites/eskalenlab/files/355630.pdf

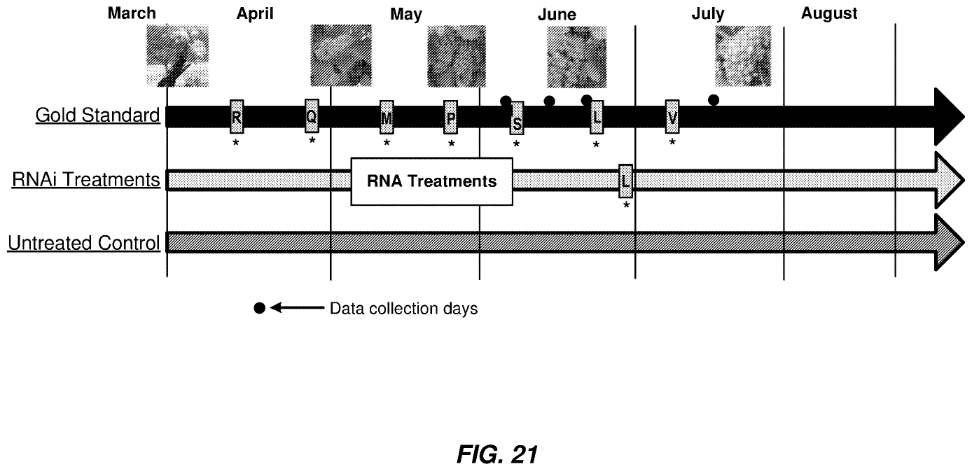

In this field trial, a total of 3 sprays of dsRNA were performed between May and early June (FIG. 21). For the dsRNA treatments, no parallel chemical treatments were performed prior to the dsRNA treatments. For the untreated control, no chemical treatments were employed. Because the dsRNA treatments ended June 2, one treatment of Luna experience, a commonly used chemical fungicide, was sprayed at the end of June for the dsRNA treatment vines. Gold standard treatment was performed as in FIG. 21 according to the powdery mildew index, best practices, and in parallel with Dr. Akif Eskalen (UC Davis) who was performing fungicide trials at an adjacent site. Data was collected at the times marked by gray dots in the FIG. 21, with all data collections following the final dsRNA treatment. The monitoring of powdery mildew index data and manual scouting of disease in the field was routinely done to plan the spray timing (www2.ipm.ucanr.edu/weather/grape-powdery-mildew-risk-assessment-index/?src=redirect2refresh).

### Data Collection and Analysis:

### Powdery Mildew Disease Incidence and Disease Severity:

Total of four Disease Incidence assessments and two Disease Severity assessments were performed (gray circles). No gold standard data was available for direct comparison due to differences in powdery mildew pressure between our vines and Akif Eskalen's vines adjacent to our plot. We also analyzed our treatment data for statistically significant patterns based on position in the field, but field position effects did not impact the findings within our sampled vines. The disease incidence and disease severity data was collected as described above for the Kearney site. Disease incidence was performed for all bunches per replicate unit, ranging from 22 to 86, average 55. For disease severity, 25 bunches were examined.

1. Powdery Mildew Disease Incidence (Percent of Berry Bunches with Powdery Mildew)

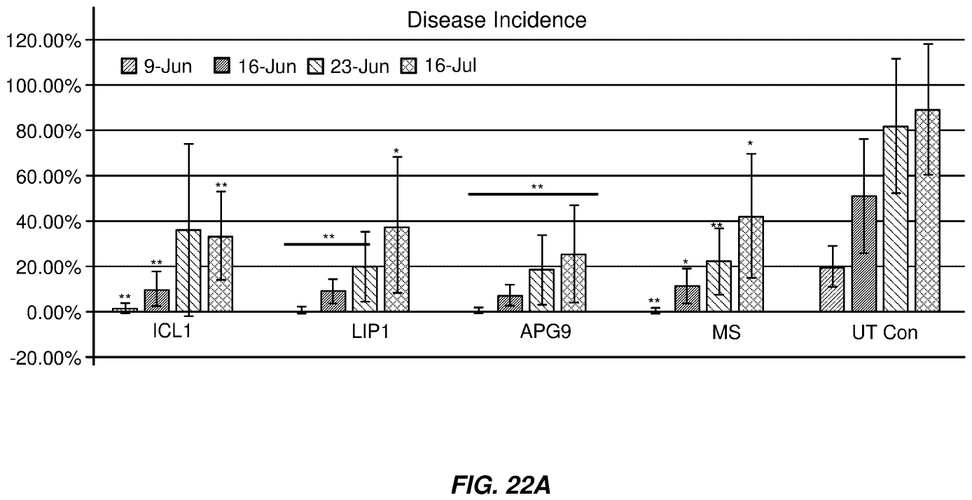

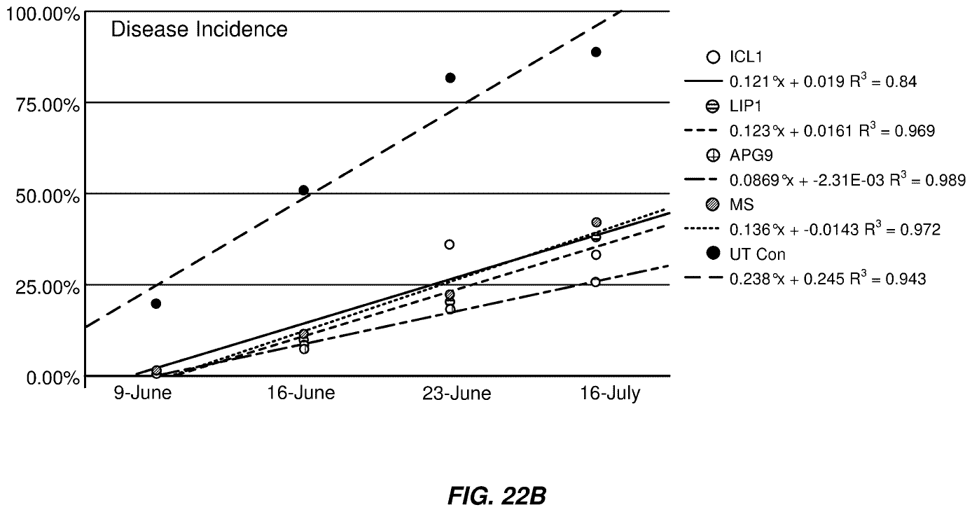

Disease incidence was assessed at 1 week, 2 weeks, 3 weeks, and >6 weeks following the last dsRNA treatment. Powdery mildew disease pressure at the Armstrong site was very high, with untreated samples exhibiting a disease incidence of ˜80% by June 23 (FIG. 22, panel A). The dsRNA treatments reduced powdery mildew disease incidence dramatically compared to the untreated control, with impact extending to 3 weeks after dsRNA treatments. LIP1, APG9, and MS showed the highest efficacy, with powdery mildew disease incidence of ˜20% at 3 weeks after the end of treatments, compared to ˜80% for untreated (June 23 data). Even more than six weeks after the dsRNA treatments, all dsRNA treatments exhibited a dramatic reduction in powdery mildew incidence compared to untreated controls, with APG9 performing best, with a mean disease incidence of 24% on July 16. Examining the slopes of the disease incidence progression for each treatment set (FIG. 22, panel B), shows the untreated control increases in average disease incidence 2.74-fold faster than that of dsRNA treatment for APG9, and ˜1.8-fold faster than the other three dsRNA treatments. Again, this supports continued efficacy of the dsRNA treatments, with APG9 exerting the strongest reduction on powdery mildew proliferation.

2. Powdery Mildew Disease Severity (Percent of Berries with Powdery Mildew Per Bunch)

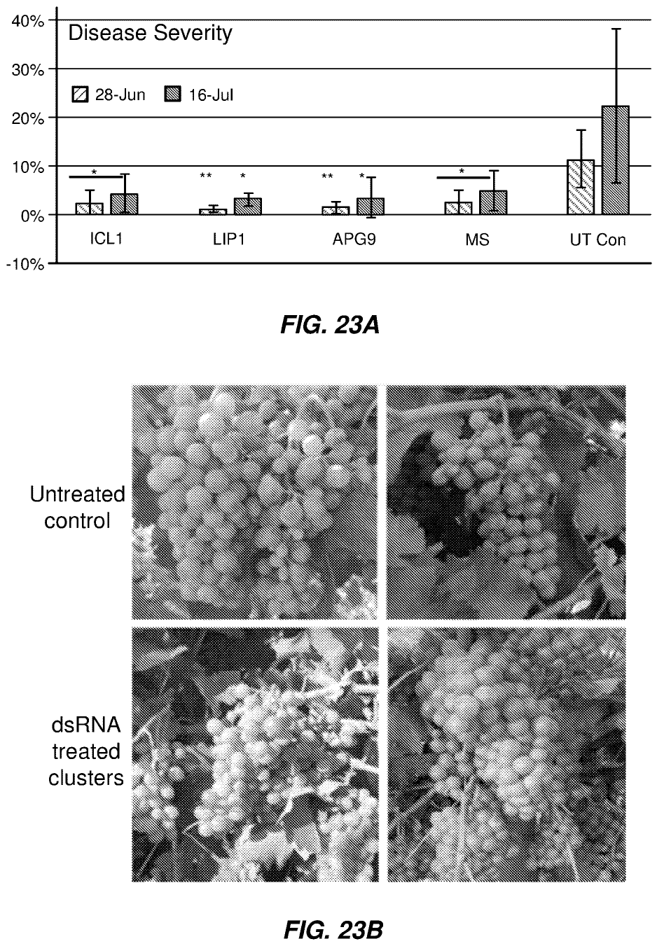

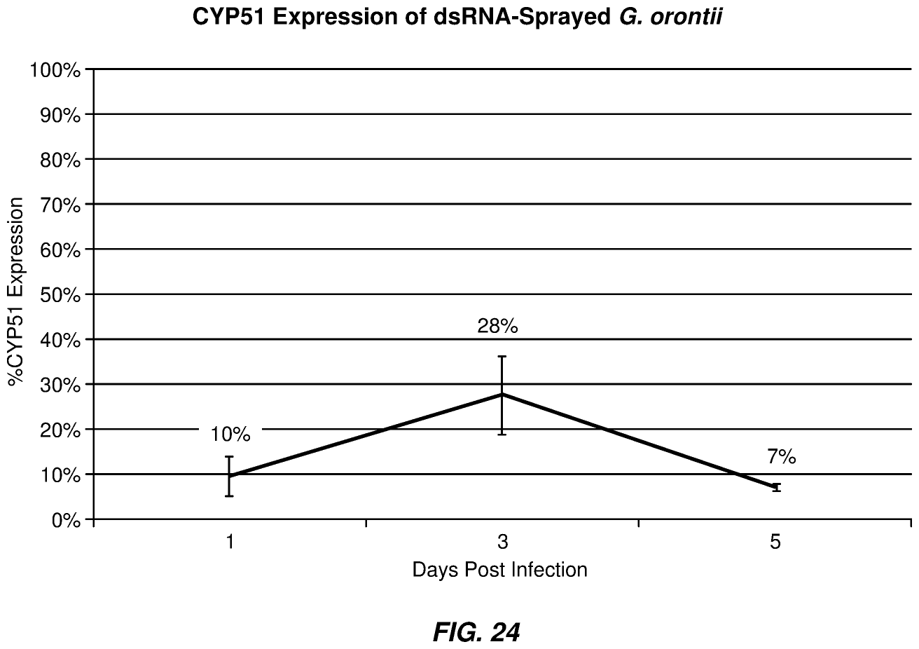

All dsRNA treatments had dramatically reduced powdery mildew disease severity compared to the untreated control (FIG. 23, panel A). At almost 4 weeks post dsRNA treatment (June 28 sampling date), the dsRNA treatments effectively limited powdery mildew, with LIP1 and APG9 mean disease severity of 1% and ICL1 and MS mean disease severity of 2%, compared to 12% for untreated vines. And the dsRNA treatments continued to exert a strong influence on disease severity. Disease severity was kept at or below 5% through the July 16 sampling date (˜6 weeks after dsRNA treatments), compared to untreated control with ˜23% disease severity. Representative berry clusters, shown in FIG. 23B, reflect this difference.

### 3. Plant Characteristics: Canopy Size, Berry Development, Phytotoxicity

Canopy size: The canopy size was visually estimated using the 1-10 scale as above, with a total of 8 readings taken from May 5th to June 22^{nd}. Five replicates per treatment were assessed. There was no statistical difference between the canopy size growth over time in treated vs untreated vines suggesting that dsRNA treatment does not impact the canopy growth.

Phytotoxicity: Visual assessment occurred throughout the growing season and no visible signs of necrosis, aberrant leaf development, blistering, or leaf rollup were observed for any treatment. Note that minimal localized chlorosis was associated with powdery mildew infection sites.

Berry development: Qualitative visual assessment occurred throughout the growing season and no difference associated with a particular dsRNA treatment was noted compared with controls.

### Conclusion

In 2021, we tested the efficacy of selected gene targets to control powdery mildew of susceptible grapevine varietals at two field sites. The topical spraying of dsRNA against all five gene targets (SEQ ID Nos:182, 183, 186, 188, 208) tested in this trial dramatically reduced powdery mildew infection at both trial sites in California. Disease severity was kept at or below 5% and the dsRNA treatments exerted their inhibitory effects beyond 3 weeks. In addition, there was no negative impact of the dsRNA treatments on canopy or berry development, or evidence of phytotoxicity. The field tests were done according to best standard commercial practices in maintaining grapevine with treatments compatible with commercial spraying practices. Our results clearly show that the genes targeted using topical dsRNA to control Arabidopsis and grapevine powdery mildew in lab and greenhouse grape potted plants effectively controlled powdery mildew infection in the field. Three of the gene targets (Lip1 (triacylglycerol lipase), ICL1 (isocitrate lyase) and MS (malate synthase) of the glyoxylate cycle, are expected to function in lipid metabolism/acquisition process category. CYP51 is an essential enzyme involved in sterol biosynthesis, and a known powdery mildew fungicide target; therefore, our successful result with CYP51 confirms that topical dsRNA application can be used an effective alternate approach to inhibit known powdery mildew targets inhibited by chemical fungicides. The effectiveness of topical dsRNA against APG9 which functions in vesicle formation and autophagy highlights the importance of this process to powdery mildew proliferation.

These field tests were conducted with dsRNA dissolved in distilled water using our initially identified dsRNA sequences against a given target. Further optimization and modification of the dsRNA sequence against a given target coupled with optimized formulation and delivery systems would be expected to further increase treatment efficacy and its duration. This could allow for increased intervals between treatments, fewer treatments, and less dsRNA to be required. In addition, dsRNA sequences against multiple targets could be combined in one treatment or combined with other compatible plant growth promoters or pest inhibitors to potentially further increase efficacy.

### Example 5

Disease Reduction Data for Powdery Mildew Gene Targets for which dsRNA Treatment Results in Statistically Reduced Powdery Mildew Proliferation.

Table 11 below shows results for topical dsRNA treatment against powdery mildew in laboratory growth chamber/greenhouse and vineyard field trials in which the mean reduction in powdery mildew disease was statistically significant (p<=0.05) in G. orontii-Arabidopsis assays. Results for subsequent testing with topical dsRNA against these targets in E. necator-Vitis vinifera follows. Finally, field testing of topical dsRNA against E. necator targets at two vineyard sites is presented. Bolded and underlined targets exhibit >=50% reduction in powdery mildew disease assay in at least one assay. Targets labeled with “*” were tested in vineyards.

TABLE 11
Results for topical dsRNA treatment against powdery mildew in laboratory
growth chamber/greenhouse and vineyard field trials in which the
mean reduction in powdery mildew disease was statistically significant
(p <= 0.05) in G. orontii-Arabidopsis assays.

| | | | G. orontii Spore Production on Arabidopsis | | | |
|---|---|---|---|---|---|---|
| Gor | En | | Detached Leaf Assay | | Whole Plant Assay | |
| Seq | Seq | Gene name or PFAM | % | | % | |
| ID | ID | domain | reduction | p-value | reduction | p-value |
| 85 | 182* | Cytochrome P450 | 47% | 3.2E−06 | 46% | 1.7E−05 |
| | | monoxygenase 51 | | | | |
| | | (CYP51) | | | | |
| 1 | 183* | TAG Lipase 1, | 51% | 1.7E−04 | 22% | 2.1E−03 |
| | | carboxyesterase | | | | |
| 2 | 184 | TAG lipase A, | 67% | 6.6E−06 | 41% | 1.4E−02 |
| | | extracellular lipase | | | | |
| 15 | 185 | transaldolase | 38% | 5.6E−03 | | |
| 20 | 186* | isocitrate lyase(ICL) | 64% | 1.8E−03 | 37% | 1.8E−03 |
| | 187 | | | | | |
| 21 | 188* | malate synthase(MS) | 37% | 1.1E−03 | 22% | 1.6E−02 |
| 22 | 189 | Citrate (Si) synthase | 63% | 1.1E−02 | | |
| 32 | 190 | trans-2-enoyl CoA | 50% | 5.6E−05 | | |
| | | reductase | | | | |
| 63 | 191 | glutamate | 60% | 4.4E−03 | | |
| | | dehydrogenase | | | | |
| 64 | 192 | threonine aldolase | 55% | 7.5E−03 | | |
| 70 | 193 | pf02548~Ketopantoate | 46% | 1.8E−05 | | |
| | | hydroxymethyltransferase | | | | |
| 71 | 194 | tetrahydrofolate | 64% | 7.5E−05 | | |
| | | synthase | | | | |
| 79 | 195 | thymidylate synthase | 68% | 6.0E−05 | | |
| 87 | 196 | pf08238~Sel1 repeat | 42% | 9.4E−04 | | |
| 92 | 197 | apoptosis antagonizing | 44% | 6.0E−04 | 21% | 1.5E−01 |
| | | transcription factor | | | | |
| | | (AATF) | | | | |
| 104 | 198 | pf10453~Nuclear fragile | 39% | 1.8E−03 | | |
| | | X mental retardation- | | | | |
| | | interacting protein 1 | | | | |
| | | (NUFIP1) | | | | |
| 122 | 199 | G. orontii effector | | | 33% | 1.6E−04 |
| | | candidate 60/61 | | | | |
| 123 | 200 | G. orontii effector | 38% | 2.5E−03 | 32% | 4.5E−03 |
| | | candidate 70 | | | | |
| 124 | — | G. orontii effector | | | 33% | 1.0E−03 |
| | | candidate 14 | | | | |
| 140 | 201 | effector protein EC2 | 37% | 1.2E−02 | 60% | 1.4E−07 |
| 141 | 202 | Heat shock protein 70 | 76% | 4.5E−06 | | |
| | | family (HSP70) | | | | |
| 145 | 203 | β-carotene 15,15′- | 54% | 6.6E−03 | 50% | 2.7E−03 |
| | | dioxygenase(BCDO) | | | | |
| 146 | 204 | xanthoxin oxidase | 63% | 4.0E−03 | 42% | 1.6E−03 |
| 148 | 205 | ABA G-protein coupled | 54% | 6.2E−06 | 31% | 6.3E−05 |
| | | receptor | | | | |
| 149 | 206 | acyl-coA oxidase (ACX) | 42% | 5.0E−02 | 66% | 5.9E−04 |
| 163 | 207 | anaphase promoting | 48% | 7.6E−03 | | |
| | | complex/20S cyclosome | | | | |
| | | subunit | | | | |
| 164 | 208* | pf04109~Autophagy | 67% | 8.1E−04 | | |
| | | protein Apg9 | | | | |
| 167 | 209 | pf03517, Regulator of | 39% | 6.7E−02 | | |
| | | cell volume after cellular | | | | |
| | | swelling | | | | |
| 169 | 210 | Ctr family Cu2 + | 54% | 7.6E−05 | | |
| | | transporter | | | | |
| 179 | — | Single copy gene, | | | 41% | 1.3E−03 |
| | | unknown function | | | | |
| 180 | 211 | Cyclin-like protein | | | 53% | 1.2E−05 |
| 181 | — | Alanine-glyoxylate | 62% | 6.5E−03 | | |
| | | aminotransferase | | | | |
| | | | E. necator Spore Production on Grapevine | | | |
| Gor | En | | Leaf Disc Assay | | Whole Plant Assay | |
| Seq | Seq | Gene name or PFAM | % | | % | |
| ID | ID | domain | reduction | p-value | reduction | p-value |
| 85 | 182* | Cytochrome P450 | 45% | 3.6E−04 | 62% | 3.8E−03 |
| | | monoxygenase 51 | | | | |
| | | (CYP51) | | | | |
| 1 | 183* | TAG Lipase 1, | | | 64% | 6.4E−04 |
| | | carboxyesterase | | | | |
| 2 | 184 | TAG lipase A, | | | 62% | 5.6E−04 |
| | | extracellular lipase | | | | |
| 15 | 185 | transaldolase | | | | |
| 20 | 186*, | isocitrate lyase(ICL) | 57% | 3.3E−02 | 72% | 7.3E−04 |
| | 187 | | | | | |
| 21 | 188* | malate synthase(MS) | | | 28% | 3.4E−02 |
| 22 | 189 | Citrate (Si) synthase | | | | |
| 32 | 190 | trans-2-enoyl CoA | | | | |
| | | reductase | | | | |
| 63 | 191 | glutamate | | | | |
| | | dehydrogenase | | | | |
| 64 | 192 | threonine aldolase | | | | |
| 70 | 193 | pf02548~Ketopantoate | | | | |
| | | hydroxymethyltransferase | | | | |
| 71 | 194 | tetrahydrofolate | | | | |
| | | synthase | | | | |
| 79 | 195 | thymidylate synthase | | | | |
| 87 | 196 | pf08238~Sel1 repeat | | | | |
| 92 | 197 | apoptosis antagonizing | | | 53% | 8.9E−02 |
| | | transcription factor | | | | |
| | | (AATF) | | | | |
| 104 | 198 | pf10453~Nuclear fragile | | | | |
| | | X mental retardation- | | | | |
| | | interacting protein 1 | | | | |
| | | (NUFIP1) | | | | |
| 122 | 199 | G. orontii effector | 29% | 4.8E−02 | | |
| | | candidate 60/61 | | | | |
| 123 | 200 | G. orontii effector | 40% | 4.8E−02 | | |
| | | candidate 70 | | | | |
| 124 | — | G. orontii effector | | | | |
| | | candidate 14 | | | | |
| 140 | 201 | effector protein EC2 | 47% | 2.3E−05 | | |
| 141 | 202 | Heat shock protein 70 | | | | |
| | | family (HSP70) | | | | |
| 145 | 203 | β-carotene 15,15′- | | | 54% | 4.3E−02 |
| | | dioxygenase(BCDO) | | | | |
| 146 | 204 | xanthoxin oxidase | | | | |
| 148 | 205 | ABA G-protein coupled | | | | |
| | | receptor | | | | |
| 149 | 206 | acyl-coA oxidase (ACX) | | | | |
| 163 | 207 | anaphase promoting | | | | |
| | | complex/20S cyclosome | | | | |
| | | subunit | | | | |
| 164 | 208* | pf04109~Autophagy | | | | |
| | | protein Apg9 | | | | |
| 167 | 209 | pf03517, Regulator of | | | | |
| | | cell volume after cellular | | | | |
| | | swelling | | | | |
| 169 | 210 | Ctr family Cu2 + | | | | |
| | | transporter | | | | |
| 179 | — | Single copy gene, | | | | |
| | | unknown function | | | | |
| 180 | 211 | Cyclin-like protein | | | | |
| 181 | — | Alanine-glyoxylate | | | | |
| | | aminotransferase | | | | |
| | | | E. necator on Grapevine Berries | | | |
| | | | Vineyard Field Trials | | | |
| | | | Vineyard - Armstrong | | | |
| Gor | En | | Disease Severity | | Disease Incidence | |
| Seq | Seq | Gene name or PFAM | % | | % | |
| ID | ID | domain | reduction | p-value | reduction | p-value |
| 85 | 182* | Cytochrome P450 | | | | |
| | | monoxygenase 51 | | | | |
| | | (CYP51) | | | | |
| 1 | 183* | TAG Lipase 1, | 90% | 4.4E−03 | 97% | 1.0E−03 |
| | | carboxyesterase | | | | |
| 2 | 184 | TAG lipase A, | | | | |
| | | extracellular lipase | | | | |
| 15 | 185 | transaldolase | | | | |
| 20 | 186*, | isocitrate lyase(ICL) | 78% | 1.3E−02 | 93% | 2.0E−03 |
| | 187 | | | | | |
| 21 | 188* | malate synthase(MS) | 78% | 1.4E−02 | 98% | 1.0E−03 |
| 22 | 189 | Citrate (Si) synthase | | | | |
| 32 | 190 | trans-2-enoyl CoA | | | | |
| | | reductase | | | | |
| 63 | 191 | glutamate | | | | |
| | | dehydrogenase | | | | |
| 64 | 192 | threonine aldolase | | | | |
| 70 | 193 | pf02548~Ketopantoate | | | | |
| | | hydroxymethyltransferase | | | | |
| 71 | 194 | tetrahydrofolate | | | | |
| | | synthase | | | | |
| 79 | 195 | thymidylate synthase | | | | |
| 87 | 196 | pf08238~Sel1 repeat | | | | |
| 92 | 197 | apoptosis antagonizing | | | | |
| | | transcription factor | | | | |
| | | (AATF) | | | | |
| 104 | 198 | pf10453~Nuclear fragile | | | | |
| | | X mental retardation- | | | | |
| | | interacting protein 1 | | | | |
| | | (NUFIP1) | | | | |
| 122 | 199 | G. orontii effector | | | | |
| | | candidate 60/61 | | | | |
| 123 | 200 | G. orontii effector | | | | |
| | | candidate 70 | | | | |
| 124 | — | G. orontii effector | | | | |
| | | candidate 14 | | | | |
| 140 | 201 | effector protein EC2 | | | | |
| 141 | 202 | Heat shock protein 70 | | | | |
| | | family (HSP70) | | | | |
| 145 | 203 | β-carotene 15,15′- | | | | |
| | | dioxygenase(BCDO) | | | | |
| 146 | 204 | xanthoxin oxidase | | | | |
| 148 | 205 | ABA G-protein coupled | | | | |
| | | receptor | | | | |
| 149 | 206 | acyl-coA oxidase (ACX) | | | | |
| 163 | 207 | anaphase promoting | | | | |
| | | complex/20S cyclosome | | | | |
| | | subunit | | | | |
| 164 | 208* | pf04109~Autophagy | 87% | 5.4E−03 | 91% | 3.0E−03 |
| | | protein Apg9 | | | | |
| 167 | 209 | pf03517, Regulator of | | | | |
| | | cell volume after cellular | | | | |
| | | swelling | | | | |
| 169 | 210 | Ctr family Cu2 + | | | | |
| | | transporter | | | | |
| 179 | — | Single copy gene, | | | | |
| | | unknown function | | | | |
| 180 | 211 | Cyclin-like protein | | | | |
| 181 | — | Alanine-glyoxylate | | | | |
| | | aminotransferase | | | | |
| | | | E. necator on Grapevine Berries | | | |
| | | | Vineyard Field Trials | | | |
| | | | Vineyard - Kearney | | | |
| | | | | | Disease | |
| Gor | En | | Disease Severity | | Incidence | |
| Seq | Seq | Gene name or PFAM | % | | % | |
| ID | ID | domain | reduction | p-value | reduction | p-value |
| 85 | 182* | Cytochrome P450 | 93% | 2.5E−07 | 77% | 9.9E−04 |
| | | monoxygenase 51 | | | | |
| | | (CYP51) | | | | |
| 1 | 183* | TAG Lipase 1, | 71% | 7.1E−03 | 78% | 1.0E−03 |
| | | carboxyesterase | | | | |
| 2 | 184 | TAG lipase A, | | | | |
| | | extracellular lipase | | | | |
| 15 | 185 | transaldolase | | | | |
| 20 | 186*, | isocitrate lyase(ICL) | 94% | 4.9E−07 | 81% | 7.9E−04 |
| | 187 | | | | | |
| 21 | 188* | malate synthase(MS) | 87% | 8.1E−07 | 79% | 5.3E−04 |
| 22 | 189 | Citrate (Si) synthase | | | | |
| 32 | 190 | trans-2-enoyl CoA | | | | |
| | | reductase | | | | |
| 63 | 191 | glutamate | | | | |
| | | dehydrogenase | | | | |
| 64 | 192 | threonine aldolase | | | | |
| 70 | 193 | pf02548~Ketopantoate | | | | |
| | | hydroxymethyltransferase | | | | |
| 71 | 194 | tetrahydrofolate | | | | |
| | | synthase | | | | |
| 79 | 195 | thymidylate synthase | | | | |
| 87 | 196 | pf08238~Sel1 repeat | | | | |
| 92 | 197 | apoptosis antagonizing | | | | |
| | | transcription factor | | | | |
| | | (AATF) | | | | |
| 104 | 198 | pf10453~Nuclear fragile | | | | |
| | | X mental retardation- | | | | |
| | | interacting protein 1 | | | | |
| | | (NUFIP1) | | | | |
| 122 | 199 | G. orontii effector | | | | |
| | | candidate 60/61 | | | | |
| 123 | 200 | G. orontii effector | | | | |
| | | candidate 70 | | | | |
| 124 | — | G. orontii effector | | | | |
| | | candidate 14 | | | | |
| 140 | 201 | effector protein EC2 | | | | |
| 141 | 202 | Heat shock protein 70 | | | | |
| | | family (HSP70) | | | | |
| 145 | 203 | β-carotene 15,15′- | | | | |
| | | dioxygenase(BCDO) | | | | |
| 146 | 204 | xanthoxin oxidase | | | | |
| 148 | 205 | ABA G-protein coupled | | | | |
| | | receptor | | | | |
| 149 | 206 | acyl-coA oxidase (ACX) | | | | |
| 163 | 207 | anaphase promoting | | | | |
| | | complex/20S cyclosome | | | | |
| | | subunit | | | | |
| 164 | 208* | pf04109~Autophagy | 92% | 2.2E−07 | 81% | 7.6E−04 |
| | | protein Apg9 | | | | |
| 167 | 209 | pf03517, Regulator of | | | | |
| | | cell volume after cellular | | | | |
| | | swelling | | | | |
| 169 | 210 | Ctr family Cu2 + | | | | |
| | | transporter | | | | |
| 179 | — | Single copy gene, | | | | |
| | | unknown function | | | | |
| 180 | 211 | Cyclin-like protein | | | | |
| 181 | — | Alanine-glyoxylate | | | | |
| | | aminotransferase | | | | |

Bolded entries in Table 11 have 50% or greater reduction. Entries marked with a were tested in vineyard field trials (See Example 4 for details). n≥3 for the detached leaf/disc and whole plant assays (see Example 1). n=5 for the vineyard treatments (Example 4). The specific dsRNA employed against the targets are indicated in Table 2. These dsRNA sequences are illustrative, but non-limiting. Disease severity is the number of berries with powdery mildew/bunch (Armstrong and Kearney—July 16 sampling date). Disease incidence is the percent of berry bunches with evidence of powdery mildew out of total bunches (Armstrong sampling date 7 days post treatments (June 9); Kearney 7 days post treatments sampling date (July 16)). Example 4, supra, shows complete results from Vineyard field trials.

### Example 6

Illustrative Example of dsRNA Silencing of Gene Expression.

dsRNA targeting CYP51 reduces expression of CYP51 in G. orontii. A. thaliana was infected with G. orontii at day 0, followed by spraying with CYP51-targeting dsRNA. qPCR analysis was performed at 1, 3, and 5 days post infection. β-tubulin was used as a reference gene to normalize CYP51 expression between samples. Average CYP51 expression of dsRNA-sprayed tissue normalized to control CYP51 expression is plotted with n≥2 biological replicates, ±SEM. This illustrative example was performed as part of initial method development associated with Example 1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Lengthy table referenced here
US20260117244A1-20260430-T00001
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260117244A1). An electronic copy of the table will also be available from the USPTO upon request and payment of the fee set forth in 37 CFR 1.19(b)(3).

1. An isolated double stranded RNA (dsRNA) for controlling or preventing an infection of a plant by a phytopathogen, wherein said dsRNA is capable of inhibiting or downregulating (e.g., downregulates) expression of one or more target genes selected from the group consisting of a gene involved in lipid metabolism and/or acquisition, a gene involved in triacylglycerol (TAG)/glycerolipid metabolism, a gene involved in the pentose phosphate pathway (PPP), a gene involved in the tricarboxylic acid (TCA) and/or glyoxylate cycle, a gene involved in fatty acid synthesis and/or transport, a gene involved in lipid catabolism, a gene involved in fatty acid degradation including fatty acid B-oxidation, a gene involved in carbohydrate metabolism, a gene involved in glycogen metabolism, a gene involved in glycolysis, a gene involved in carbohydrate utilization, a gene involved in amino acid and/or nucleotide metabolism and salvage and/or cofactor metabolism, a gene involved in amino acid metabolism and/or salvage, a gene involved in cofactor metabolism, a gene involved in nucleotide metabolism and/or salvage, a gene involved in an essential fungal specific process, a gene involved in sterol biosynthesis, a gene involved in mitochondrial electron transport and/or A TP synthesis, a gene involved in mitochondrial electron transport chain, a gene involved in transcriptional regulation, a gene involved in protein regulation, a gene involved in nucleic acid translation, a gene involved in signaling, a gene involved in producing a secreted protein, a gene associated with the plant TCP interaction network, a gene associated with a plant hormone pathway, a gene involved injasmonic acid (oxylipin) metabolism, acquisition, or signaling, a gene involved in abscisic acid metabolism, acquisition, or signaling, a gene involved in cell cycle and DNA replication, a gene involved in vesicles, autophagy, and phagocytosis, a gene involved in autophagy and phagocytosis, a gene involved in ion/metal transport and osmotic homeostasis, single copy genes in G. orontii MGH1 following recent whole genome duplication including those with unknown function, a gene involved in lipid metabolism, a gene involved in fatty acid (FA) synthesis and/or transport, a gene involved in carbohydrate metabolism, a gene involved in amino acid and/or nucleotide metabolism and salvage and/or cofactor metabolism, a gene involved in nucleotide metabolism/salvage, a gene involved in an essential fungal-specific process, a gene involved in mitochondrial electron transport, a gene involved in transcriptional regulation, a gene involved in nucleic acid translation, a gene involved in signaling, a gene involved in producing a secreted protein, a gene involved in plant hormone metabolism, a gene involved in cell cycle and replication, a gene involved in autophagy and phagocytosis, and a gene involved in cell volume control.

2. (canceled)

3. The isolated dsRNA according to claim 1, wherein said dsRNA inhibits or is capable of downregulating expression of one or more target genes shown in Table 1 or Table 2 (SEQ ID NOS:1-211), and SEQ ID NOs:255-411, and/or orthologues thereof.

4-6. (canceled)

7. The isolated dsRNA of claim 3, wherein said target genes and/or orthologs comprise one or more target genes of SEQ ID Nos: 62-69, 145-148, 164-166, 167-169, and/or orthologs thereof.

8-12. (canceled)

13. The isolated dsRNA according to claim 3, wherein said target genes and/or orthologs comprise one or more G. orontii genes independently selected from the group consisting of Cytochrome P450 monoxygenase (CYP51) (SEQ ID NO:85), TAG lipase 1, caroboxyesterase domain (SEQ ID NO:1), TAG lipase A, extracellular lipase (SEQ ID NO:2), Transaldolase (SEQ ID NO:15), Isocitrate lyase (ICL) (SEQ ID NO:20), Malate synthase (MS) (SEQ ID NO:21), Citrate (Si) synthase (SEQ ID NO:22), Trans-2-enoyl CoA reductase (SEQ ID NO:32), Glutamate dehydrogenase (SEQ ID NO:63), Threonine aldolase (SEQ ID NO:64), Pf02548—Ketopantoate hydroxymethyltransferase (SEQ ID NO:70), Tetrahydrofolate synthase (SEQ ID NO:71), Thymidine synthase (SEQ ID NO:79), Pf8238-Sel1 repeat (SEQ ID NO:87), Apoptosis antagonizing transcription factor (AATF) (SEQ ID NO:92), Pf10453—Nuclear fragile X mental retardation-interacting protein 1 (NUFIP1) (SEQ ID NO:104), G. orontii effector candidate 60/61 (SEQ ID NO:122), G. orontii effector candidate 70 (SEQ ID NO: 123), G. orontii effector candidate 14 (SEQ ID NO: 124), Effector protein EC2 (SEQ ID NO:140), Heat shock protein 70 family (HSP70) (SEQ ID NO:141), J3-carotene 15,15′-dioxygenase (BCDO) (SEQ ID NO:145), Xanthin oxidase (SEQ ID NO:146), ABA G-protein coupled receptor (SEQ ID NO: 148), Acyl-CoA oxidase (ACX_(SEQ ID NO:149), Anaphase promoting complex/20S cyclosome subunit (SEQ ID NO: 163), Pf04109—Autophagy protein Apg9 (SEQ ID NO: 164), Pf03517 Regulator of cell volume after swelling (SEQ ID No. 167), Ctr family Cu2+ transporter (SEQ ID NO: 169), Single copy gene, unknown function (SEQ ID NO: 179), Cyclin-like protein (SEQ ID NO: 180), and Alanine-glyoxylate aminotransferase (SEQ ID NO. 181), and/or orthologs thereof.

14-15. (canceled)

16. The isolated dsRNA according to claim 3, wherein said target genes and/or orthologs comprise one or more target genes selected from the group consisting of CYP51 (Seq ID NO: 85, 182), lipase 1 (Seq ID NO: 1, 183), lipase A (SEQ ID NO: 2, 184), isocitrate lyase (SEQ ID NO. 20, 186-187), malate synthase (SEQ ID: 21, 188), citrate (Si) synthase (SEQ ID NO: 22, 189), trans-2-enoyl-coA reductase (SEQ ID NO: 32, 190), glutamate dehydrogenase (SEQ ID NO: 63, 191), threonine aldolase (SEQ ID NO: 64,192), tetrahydrofolate synthase (SEQ ID NO: 71, 194), thymidylate synthase (SEQ ID NO: 79, 195), apoptosis-antagonizing transcription factor (AATF) (SEQ ID NO: 92, 197), Effector candidate 2 (EC2) (SEQ ID NO: 140,201), f3-carotene 15,15′-dioxygenase (BCDO) (SEQ ID NO: 145, 203), xanthoxin oxidase (SEQ ID NO: 146, 204), ABA G-coupled receptor (SEQ ID NO: 148, 205), acyl-coA oxidase (ACX) (SEQ ID NO: 149, 206), Autophagy protein Apg9 (SEQ ID NO: 164, 208), Ctr family CU2+ transporter (SEQ ID NO: 169,210), cyclinlike protein (SEQ ID NO: 180,211), and alanine-glyoxylate amino transferase (SEQ ID NO: 181), and/or orthologs thereof.

17-51. (canceled)

52. The composition according to claim 3, wherein said ortholog is an ortholog found in a powdery mildew selected from the powdery mildew genus Golovinomyces including G. orontii and G. cichorachearum (of curcubits), Erysiphe including E. necator (or Uncinula necator) (powdery mildew of grapes), Blumeria including B. graminis f. sp. tritici (causes powdery mildew of wheat), f. sp. hordei (powdery mildew of barley), Microsphaera including M. diffusa (powdery mildew of legumes, e.g. soybean), Leveillula including L. taurica (also known as Oidiopsis taurica) (powdery mildew of onion or artichoke), Podosphaera including P. leucotricha (powdery mildew of apples and pears), P. macularis (powdery mildew of hemp and cannabis), P. xanthii (powdery mildew of cucurbits:cucumbers, squashes (including pumpkins), luffas, melons, and watermelons), and P. pannosa (powdery mildew of roses) or wherein said ortholog is an ortholog found in a plant rust selected from the group consisting of Cronartium ribicola (White pine blister rust), Gymnosporangiumjuniperi-virginianae (Cedar-apple rust), Hemileia vastatrix (Coffee rust), Phakopsora meibomiae and P. pachyrhizi (Soybean rust), Puccinia coronata (Crown Rust of Oats and Ryegrass), Puccinia graminis (Stem rust of wheat and Kentucky bluegrass, or black rust of cereals), Puccinia hemerocallidis (Daylily rust), Puccinia triticina (Brown Wheat Rust), Puccinia sorghi (Common Rust of Corn), Puccinia stri˜formis (Yellow Rust) of cereals, Uromyces appendiculatus (Bean Rust), Puccinia melanocephala (Brown Rust of Sugarcane), and Puccinia kuehnii (Orange rust of Sugar cane) or said ortholog is an ortholog found in an ascomycete selected from the group consisting of Botrytis sp. (grey mold), Fusarium spp. (Fusarium wilt disease), Thielaviopsis spp. (canker rot, black root rot, Thielaviopsis root rot), Verticillium spp., Magnaporthe grisea (rice blast), and Sclerotinia sclerotiorum (cottony rot) or said ortholog is an ortholog found in a Basidiomycete selected from the group consisting of Ustilago spp. (smuts) smut of cereals, Tilletia sp. Rhizoctonia spp., and Armillaria spp. (honey fungus species, virulent pathogens of trees).

53-69. (canceled)

70. The isolated dsRNA molecule of claim 3, wherein the dsRNA molecule comprises a single RNA strand comprising an inversely repeated sequence with a spacer in between and wherein the single RNA strand can anneal to itself to form a hairpin loop structure.

71-78. (canceled)

79. The isolated dsRNA molecule of claim 16, wherein said dsRNA comprises a nucleotide sequence selected from the group consisting of SEQ ID NO: 212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, and SEQ ID NO:255.

80. A composition for controlling or preventing an infection of a plant by a phytopathogenic fungus, said composition comprising one or more of said isolated dsRNA molecules according to claim 79.

81-82. (canceled)

83. The composition of claim 80, wherein said composition comprises two or more dsRNA(s) that inhibit or are capable of downregulating expression of the two or more target genes independently selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQID NO:104, SEQ ID NO:l05, SEQ ID NO:l06, SEQ ID NO:107, SEQ ID NO:l08, SEQ ID NO:109, SEQ ID NO:110, SEQ ID NO: 111, SEQ ID NO:112, SEQ ID NO:113, SEQ ID NO:ll4, SEQ ID NO:ll5, SEQ ID NO:116, SEQ ID NO:117, SEQ ID NO:118, SEQ ID NO:119, SEQ ID NO:120, SEQ ID NO:121, SEQ ID NO:122, SEQ ID NO:l23, SEQ ID NO:124, SEQ ID NO:125, SEQ ID NO:126, SEQ ID NO:l27, SEQ ID NO:128, SEQ ID NO:l29, SEQ ID NO:l30, SEQ ID NO:l31, SEQ ID NO:132, SEQ ID NO:l33, SEQ ID NO:134, SEQ ID NO:135, SEQ ID NO:136, SEQ ID NO:137, SEQ ID NO:l38, SEQ ID NO:139, SEQ ID NO:140, SEQ ID NO:141, SEQ ID NO:142, SEQ ID NO:l43, SEQ ID NO:144, SEQ ID NO:145, SEQ ID NO:146, SEQ ID NO:l47, SEQ ID NO:148, SEQ ID NO:l49, SEQ ID NO:l50, SEQ ID NO:l51, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, and SEQ ID NO:255.

84-85. (canceled)

86. The composition according to claim 80, wherein said composition is provided as a spray, drip irrigation, a drench, granules, a seed coating, inoculation in plant growth medium, as a mist or fog, injection in stem or other plant parts or as a plant-incorporated protectant.

87-88. (canceled)

89. The composition according to claim 80, wherein said composition comprises said dsRNA(s) associated with a particle or nanoparticle or said dsRNA(s) incorporated into a lipid or liposome, incorporated into an anucleated cell.

90. The composition of claim 89, wherein said particle or nanoparticle comprise a clay, an inorganic polymer, organic polymer, a chitosan polymer, or a chitosan derived polymer.

91-94. (canceled)

95. The composition according to claim 80, wherein said composition is provided as a component of a microbial cell or microbial fermentation product.

96. The composition according to claim 80, wherein said composition further comprises at least one additive from the group consisting of adjuvants, attractants, growth-regulating substances, insect feed, pheromones, proteins, carbohydrates, polymers, organic compounds, biologics, biostimulants, other biological or synthetic pesticidal agents, and other biological or synthetic growth-regulating agents.

97-117. (canceled)

118. A method of conferring phytopathogenic fungal resistance to a plant or the part thereof, said method comprising:
introducing into the plant or the part thereof:
(a) a DNA encoding double stranded RNA (dsRNA) according to claim 16;
(b) an expression cassette comprising the DNA encoding double stranded RNA (dsRNA) according to claim 16, or
(c) a dsRNA according to claim 16.

119. A method for controlling or preventing an infection of a plant by a phytopathogenic fungus, said method comprising:
(a) contacting said plant and/or said pathogenic fungus with an isolated double-stranded RNA according to claim 16; or
(b) expressing in said plant at least one isolated double-stranded RNA according to claim 16.

120. The method of claim 119, wherein said contacting comprises spraying, dunking, coating, watering, or irrigating said plant.

121-122. (canceled)

123. The method according to claim 120, wherein said contacting comprises applying said dsRNA to a plant selected from the group consisting of grape, wheat, barley, legumes, e.g. soybean, onion, artichoke, apples, peaches, pears, cucurbits, cucumbers, squashes (including pumpkins), luffas, melons, watermelons, hemp, cannabis, roses, strawberry, hops, coffee, oats, ryegrass, a cereal, rice, corn, and sugar cane.

124-147. (canceled)
